(19) United States
(12) Patent Application Publication
(10) Pub. No.: US 2011/0217297 A1
(43) Pub. Date: Sep. 8, 2011

(54) METHODS FOR CLASSIFYING AND TREATING BREAST CANCERS

(75) Inventors: Kuo-Jang Kao, Gainesville, FL (US); Kai-Ming Chang, Taichung (TW); Andrew T. Huang, Durham, NC (US)

(73) Assignee: Koo Foundation Sun Yat-Sen Cancer Center, Taipei (TW)

(21) Appl. No.: 13/040,042

(22) Filed: Mar. 3, 2011

Related U.S. Application Data
(60) Provisional application No. 61/339,425, filed on Mar. 3, 2010.

(51) Int. Cl. A61K 39/395; C40B 30/00; A61K 31/519; A61K 31/704; A61K 31/138; G01N 33/50; A61P 35/00

(52) U.S. Cl. 424/133.1; 506/7; 514/249; 514/34; 514/651; 436/64

(57) ABSTRACT

The present invention relates to methods of treating a breast cancer in a subject, methods of identifying a subject with a breast cancer as a candidate for a therapy having efficacy for treating a breast cancer molecular subtype, and methods of selecting a therapy for a subject with a breast cancer. The methods comprise determining the molecular subtype of the breast cancer in the subject. In some embodiments, the methods further comprise administering to the subject a therapy that is effective for treating the molecular subtype of the breast cancer.

### RELATED APPLICATION(S)

This application claims the benefit of U.S. Provisional Patent Application No. 61/339,425, filed Mar. 3, 2010, which is incorporated by reference in its entirety.

### BACKGROUND OF THE INVENTION

Breast cancer is the most common cancer, and the second leading cause of cancer death, among women in the western world. Traditionally, breast cancer has been regarded as one disease of common etiology with varying features that could affect prognosis and treatment outcomes. In recent years, extensive clinical and biological investigation has led to a gradual recognition of distinctive subtypes of breast cancer. However, clinical trials to date have failed to exploit information about breast cancer subtypes for optimization of treatment. Typically, these trials have classified breast cancer according to a small number (e.g., two or three) of biomarkers. However significant biological heterogeneity among breast cancers renders treatment based on such a small number of biomarkers inadequate and ineffective for many individuals.

Thus, there is a need for the identification of additional molecular subtypes of breast cancer based on a larger number of biomarkers that more accurately reflects the biological heterogeneity of breast cancer. In addition, there is a need to determine therapies that are effective for treating specific breast cancer subtypes.

### SUMMARY OF THE INVENTION

The present invention relates, in one embodiment, to a method of treating a breast cancer in a subject, comprising determining the molecular subtype of the breast cancer in the subject and administering to the subject a therapy that is effective for treating the molecular subtype of the breast cancer. In a particular embodiment, the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer.

In another embodiment, the invention relates to a method of identifying a subject with a breast cancer as a candidate for a therapy having efficacy for treating a breast cancer molecular subtype, comprising determining the molecular subtype of the breast cancer in the subject and identifying the subject as a candidate for a therapy that is effective for treating the molecular subtype. In a particular embodiment, the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer.

In a further embodiment, the invention relates to a method of selecting a therapy for a breast cancer in a subject, comprising determining the molecular subtype of the breast cancer in the subject and selecting a therapy that is effective for treating the molecular subtype. In a particular embodiment, the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer.

In an additional embodiment, the invention relates to a method of classifying a breast cancer, comprising generating a gene expression profile for the breast cancer, comparing the gene expression profile of the breast cancer to one or more reference gene expression profiles for a breast cancer molecular subtype and classifying the breast cancer according to its molecular subtype. In a particular embodiment, the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer.

The present invention provides an alternative method for classifying breast cancers and effective methods for determining individualized and optimized treatments for breast cancer patients based on the molecular subtype of the breast cancer in the patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1a-1c are scatter plots illustrating three examples of how a probe-set was selected from multiple probe-sets to represent each of three pivotal genes. FIG. 1a: For Top2A gene, 201292_at probe-set was selected from three different probe-sets. FIG. 1b: For FOXO1 gene, 202724_s_at was selected. FIG. 1c: For TOX3 gene, 214774_x_at was selected.

FIGS. 2a-2h are scatter plots illustrating examples of probe-sets showing good or poor linear or quadratic correlation with a pivotal gene. FIGS. 2a-2f are examples of probe sets showing good linear (p<1×10⁻¹⁰) or quadratic (p<1×10⁻⁵) correlation. FIGS. 2g and 2h are examples of a probe set showing both poor linear (p=0.07 and 0.08, respectively) and quadratic (p=0.03 and 0.4, respectively) correlation.

FIG. 3 is a dendrogram of hierarchical clustering analysis of 327 breast cancer samples using cluster labels generated by repeating k-mean clustering analyses 2000 times for all samples and the 783 selected probe-sets 2000 times. Six to eight clusters representing molecular subtypes of breast cancer were obtained. Each vertical line at the bottom represents one sample.

FIG. 4a is a density plot for estrogen receptor (ER) using 312 breast cancer samples in cohort 1 to determine the cut-points for positivity and negativity. The cut-point is shown by the intercept (green line). Y-axis represents relative number of samples and X-axis represents expression intensity for ER.

FIG. 4b is a density plot for progesterone receptor (PR) using 312 breast cancer samples in cohort 1 to determine the cut-points for positivity and negativity. The cut-point is shown by the intercept (green line). Y-axis represents relative number of samples and X-axis represents expression intensity for PR.

FIG. 4c is a density plot for HER-2 using 312 breast cancer samples in cohort 1 to determine the cut-points for positivity and negativity. The cut-point is shown by the intercept (green line). Y-axis represents relative number of samples and X-axis represents expression intensity for HER-2.

FIG. 5 are graphs depicting the density distribution of 327 samples according to Jaccard coefficient for six (g=6) and eight (g=8) different molecular subtypes. A Jaccard coefficient of 1 is the most stable. More cases had higher Jaccard coefficient after classification into six different molecular subtypes compared to eight subtypes.

FIGS. 6a and 6b show functional annotation of gene clusters generated by hierarchical clustering analysis using 783 probe sets and 327 samples. Representative genes of interest from each gene cluster are listed.

FIG. 7a depicts a metastasis-free survival curve of six different molecular subtypes of breast cancer (n=327). The numbers in parentheses represent the number of events.

FIG. 7b depicts an overall survival curve of six different molecular subtypes of breast cancer (n=327). The numbers in parentheses represent the number of events.

FIGS. 8a-8c are scatter plots of gene expression intensities according to six molecular subtypes of breast cancer for nine genes known to have different functional and clinical importance in breast cancer. Expression intensities among six different molecular subtypes were compared by ANOVA test. P values of ANOVA test are shown at right upper corner of each scatter plot. Y-axis is logarithm of gene expression intensity to the base 2. X-axis is breast cancer molecular subtypes (n=327) and normal (n=40) breast tissues. FIG. 8a: ESR1 (left); TTK (middle); CAV1 (right). FIG. 8b: GATA3 (left); TYMS (middle); CD10 (right). FIG. 8c: TOP2A (left); DHFR (middle); CDC2 (right).

FIG. 9a depicts a metastasis-free survival curve for molecular subtype IV breast cancer patients treated with CMF or CAF adjuvant chemotherapy regimen. The numbers in parentheses represent number of events. P value was determined by logrank test.

FIG. 9b depicts an overall survival curve for molecular subtype IV breast cancer patients treated with CMF or CAF adjuvant chemotherapy regimen. The numbers in parentheses represent number of events. P value was determined by logrank test.

FIG. 10a are scatter plots depicting estrogen receptor (ESR1) expression intensities (X-axis) vs. epidermal growth factor receptor (ERBB2) (Y-axis) expression intensities for the six different breast cancer subtypes on four independent data sets (KFSYSCC, NKI, TRANSBIG and Uppsala). All subtype V breast cancer samples were positive for ESR1 and negative for ERBB2 and all subtype I samples were negative for both ESR1 and ERBB2. The expression intensities were logarithm of normalized expression intensities to the base 2. Molecular subtypes are depicted in different colors: subtype I—green, II—red, III—brown, IV—orange, V—dark blue and VI—light blue. Vertical and horizontal lines indicate the cut-points for determination of positivity and negativity of ESR1 and ERBB2, respectively.

FIG. 10b are scatter plots depicting estrogen receptor (ESR1) expression intensities (X-axis) vs. progesterone receptor (PGR) expression intensities (Y-axis) for the six different breast cancer subtypes on four independent data sets (KFSYSCC, NKI, TRANSBIG and Uppsala). All subtype V breast cancer samples (dark blue) were positive for ESR1 and PGR. The expression intensities were logarithm of normalized expression intensities to the base 2. Molecular subtypes are depicted in different colors: subtype I—green, II—red, III—brown, IV—orange, V—dark blue and VI—light blue. Vertical and horizontal lines indicate the cut-points for determination of positivity and negativity of ESR1 and PGR, respectively.

FIG. 11 are scatter plots depicting TOP2A expression in six different molecular subtypes of breast cancer. The intensity of TOP2A gene expression shown on Y axis is logarithm of expression intensity to the base 2. X-axis shows six different breast cancer molecular subtypes (I-VI) and normal breast (Normal; n=40) tissues. The filled dots and bars represent means and standard deviations (SD), respectively. P value was determined by ANOVA test for the six different molecular subtypes.

FIG. 12 illustrates possible mechanisms responsible for resistance to methotrexate (MTX), including 1) reduced importation of MTX by solute carrier family 19 member 1 (folate transporter, SLC19A1) and folate receptor1 (FOLR1), 2) reduced polyglutamylation of MTX by folylpolyglutamate synthase (FPGS) and 3) increased dihydrofolate reductase (DHFR) activity. (Adapted from Wood A.J.J. Intrinsic and acquired resistance to methotrexate in acute leukemia. New Eng J Med 335:1041-48, 1996.)

FIG. 13a are scatter plots depicting expression intensities of the DHFR gene for the six different breast cancer molecular subtypes and normal breast tissue samples. High expression of DHFR is related to methotrexate resistance. P values were determined by using ANOVA test.

FIG. 13b are scatter plots depicting the sum of expression intensities of the SLC19A1, FLOR1 and FPGS genes related to methotrexate resistance for the six different breast cancer molecular subtypes and normal breast tissue samples. Reduced expression of SLC19A1, FLOR1 and FPGS is related to methotrexate resistance. P values were determined by using ANOVA test.

FIG. 14a is a metastasis-free survival curve showing no significant differences between patients treated with and without adjuvant chemotherapy for molecular subtype V breast cancer. P value was determined by logrank test.

FIG. 14b is an overall survival curve showing no significant differences between patients treated with and without adjuvant chemotherapy for molecular subtype V breast cancer. P value was determined by logrank test.

FIGS. 15a-15d are metastasis-free survival curves for the six different breast cancer molecular subtypes in the KFSYCC dataset and three other independent datasets (NKI, TRANSBIG and JRH). The results show that molecular subtypes II and IV consistently have high risk for distant metastasis, molecular subtype V consistently has low risk for metastasis, molecular subtype I consistently has intermediate or high risk for distant metastasis depending on receipt of any adjuvant chemotherapy, and molecular subtypes III and VI appear to have intermediate to low risk for metastasis and are more variable. FIG. 15a, KFSYSCC: Koo Foundation SYS Cancer Center (Taiwan); FIG. 15b, NKI: Netherlands Cancer Institute; FIG. 15c, TRANSBIG: TRANSBIG consortium (Jules Bordet Institute, Brussels, Belgium); FIG. 15d, JRH: John Radcliffe Hospital (Oxford, UK).

FIGS. 15e-15h are overall survival curves for the six different breast cancer molecular subtypes in the KFSYSCC dataset and three other independent datasets (NKI, TRANSBIG and Uppsala). The results show that molecular subtypes II and IV consistently have high risk for shorter survival, molecular subtype V consistently has good overall survival, molecular subtype I consistently has poor overall survival depending on receipt of any adjuvant chemotherapy, and molecular subtypes III and VI appear to be more variable. FIG. 15e, KFSYSCC: Koo Foundation SYS Cancer Center (Taiwan); FIG. 15f, NKI: Netherlands Cancer Institute; FIG. 15g, TRANSBIG: TRANSBIG consortium (Jules Bordet Institute, Brussels, Belgium); FIG. 15h, Uppsala: Uppsala-Sweden.

FIGS. 16a-16e are scatter plots depicting gene expression intensities for the six breast cancer molecular subtypes of five genes having known roles in the chemo-sensitivity and biology of breast cancer (CAV1, DHFR, TYMS, VIM and ZEB1), using the KFSYSCC dataset and three other independent datasets (TRANSBIG, JRH and Uppsala). All four datasets shared the same distribution patterns according to the six molecular subtypes, and the expression intensities of the five genes among the six molecular subtypes were significantly different according to ANOVA test. The Y-axis indicates logarithm of gene expression intensity to the base 2. The X-axis indicates breast cancer molecular subtypes determined using the 783 classification probe-sets shown in Table 1.

FIG. 16a. CAV1 gene. P values of ANOVA test for KFSYSCC, TRANSBIG, Oxford (JRH), and Uppsala datasets are 9.3×10⁻³⁵, 2.7×10⁻⁹, 1.1×10⁻⁹ and 2.9×10⁻³⁰, respectively.

FIG. 16b. DHFR Gene. P values of ANOVA test for KFSYSCC, TRANSBIG, Oxford (JRH), and Uppsala datasets are 8.6×10⁻¹⁴, 8.3×10⁻⁶, 4.9×10⁻⁴ and 2.8×10⁻¹¹, respectively.

FIG. 16c. TYMS gene. P values of ANOVA test for KFSYSCC, TRANSBIG, Oxford, and Uppsala datasets are 8.4×10⁻³⁶, 1.5×10⁻²³, 1.3×10⁻¹⁰ and 9.8×10⁻³⁰, respectively.

FIG. 16d. VIM gene. P values of ANOVA test for KFSYSCC, TRANSBIG, Oxford, and Uppsala datasets are 1.8×10⁻¹⁷, 1.3×10⁻⁸, 4.8×10⁻⁶ and 3.1×10⁻¹⁶, respectively.

FIG. 16e. ZEB1 gene. P values of ANOVA test for KFSYSCC, TRANSBIG, Oxford, and Uppsala datasets are 2.1×10⁻¹⁶, 0.05, 6.1×10⁻³ and 6.7×10⁻⁷, respectively.

FIGS. 17a-17h are dendrograms of genes/probe-sets used to characterize six different molecular subtypes of breast cancer for the gene expression signatures of cell cycle/proliferation (17a), stromal response (17b), wound response (17c-17g) and vascular endothelial normalization (17h).

FIGS. 18a and 18b are density plots showing misclassification rates at an r level in the range of 0.1 to 0.9, where r is the fraction of 783 classifier probe-sets randomly selected and used to build a centroid classification model for molecular subtyping. The vertical gray line at 0.13 corresponds to the misclassification rate of the leave-one-out study using all 783 probe-sets.

FIG. 19. Summarizes the analysis of 734 probe-sets for enrichment of genes involved in different canonical pathways using the Ingenuity Pathway Analysis. Orange squares are ratios obtained by dividing the number of our probe-sets that meet the criteria in a given pathway with the total number of genes in the make-up of that pathway.

FIG. 20. Summarizes the results of hierachical clustering analysis when 734 associated probe-sets associated with immune response were used to identify high and low expression subgroups in different molecular subtypes of our 327 breast cancer samples. Each breast cancer molecular subtype (subtype Ito VI) is shown on the top. The black bar represents occurrence of distant metastasis and death in an individual. The red color in heat-map represents high z score above average (increased gene expression), black represents average z score (average gene expression) and green represents z score below average (reduced gene expression).

FIG. 21. Shows Kaplan-Meier plots of metastasis-free survival in different molecular subtypes of our 327 breast cancer patients. Survival difference between the low immune response group (red line) and the high immune response group (black line) was assessed by log-rank test.

FIG. 22: Shows histograms of the Jaccard coefficients given different number of clusters based on 200 paired random sub-sampled hierarchical cluster analyses.

FIG. 23. Shows heatmaps of drawn according to the dendrogram of genes in each signature as shown in FIG. 17 for different cohorts.

FIG. 24 Summarizes correlation studies between immunohistochemistry (IHC) and gene expression results for ER (A), PR(C) and HER2 (B) statuses. The cut-point for determination of positivity and negativity of ER, PR or HER2 was indicated by red dash lines. Numbers of cases above and below the cut-points are shown in each panel. Analyses by Kappa statistics showed significant degree of concordance between Microarray and IHC results.

FIG. 25 (A-E) Shows scatter and box plots of gene expression by different breast cancer molecular subtypes in four independent datasets. The five genes used in this study were chosen for their roles in drug sensitivity and epithelial-mesenchymal transition of breast cancer cells. None of them were part of the genes used for classification of molecular subtypes. As shown in these figures, all four different datasets shared the same differential distribution patterns according to the six molecular subtypes. The expression intensities of these genes among six molecular subtypes were significantly different according to ANOVA except ZEB1 in the EMC dataset. The Y-axis is logarithm of gene expression intensity to base 2. The four datasets are ours (KFSYSCC), TRANSBIG (Desmedt et al., Clin Cancer Res., 13:3207-3214 (2007)), EMC (Chang et al., Proc Natl Acad Sci, USA, 102:3738-3743 (2005)) and Uppsala (Miller et al., Proc Natl Acad Sci, USA, 102:13550-13555 (2005)).

FIG. 25 A. CAV1 gene. P values of ANOVA test for KFSYSCC, TRANSBIG, EMC, and Uppsala datasets are 9.3×10⁻³⁵, 2.7×10⁻⁹, 4.9×10⁻²¹ and 2.9×10⁻³⁰, respectively.

FIG. 25 B. DHFR Gene. P values of ANOVA test for KFSYSCC, TRANSBIG, EMC and Uppsala datasets are 8.6×10⁻¹⁴, 8.3×10⁻⁶, 3.3×10⁻⁴ and 2.8×10⁻¹¹, respectively.

FIG. 25 C. TYMS gene. P values of ANOVA test for KFSYSCC, TRANSBIG, EMC and Uppsala datasets are 8.4×10⁻³⁶, 1.5×10⁻²³, 5.0×10⁻²⁹ and 9.8×10⁻³⁰, respectively.

FIG. 25 D. VIM gene. P values of ANOVA test for KFSYSCC, TRANSBIG, EMC, and Uppsala datasets are 1.8×10⁻¹⁷, 1.3×10⁻⁸, 4.7×10⁻¹⁵ and 3.1×10⁻¹⁶, respectively.

FIG. 25 E. ZEB1 gene. P values of ANOVA test for KFSYSCC, TRANSBIG, EMC and Uppsala datasets are 2.1×10⁻¹⁶, 0.05, 0.07 and 6.7×10⁻⁷, respectively.

FIG. 26 Summarizes differential expression of genes associated with epithelial-mesenchymal transition among breast cancer molecular subtypes of the present study. The solid colored dots and bars represent mean±SD. P values were determined by ANOVA. The expression of each gene is logarithm of expression intensity to base 2.

FIG. 27 Summarizes a comparison of metastasis-free survival between subtypes V and VI breast cancer patients classified as Perou-Sørlie luminal A intrinsic type in patients of the present study.

FIG. 28 Is a heat-map of molecular subtypes of breast cancer described in the present application. The dendrogram of the 783 classification probe-sets is shown on the left and 327 breast cancer samples clustered into six molecular subtypes are shown at the top.

FIG. 29 Shows heap maps that illustrate molecular characteristics of the six different molecular subtypes of breast cancer in our dataset and the other three independent datasets (Wang et al. Lancet, 365:671-679 (2005), Miller et al., Proc Natl Acad Sci, USA, 102:13550-13555 (2005), Desmedt et al., Clin Cancer Res., 13:3207-3214 (2007)). One-way hierarchical clustering analysis was performed on 327 samples in our dataset using genes associated with cell cycle/proliferation, wound-response (Proc Natl Acad Sci, USA 2005, 102:3738-3743), stromal reaction (Nature Med 2008, 14:518-527), and tumor vascular endothelial normalization (Cell 2009, 136:810-812; Cell 2009, 136:839-851) to generate gene clusters and dendrograms. Breast cancer samples were arranged according to their subtype as shown at the top of each panel. Dendrograms of signature genes are shown on the left. The identities of genes in all four dendrograms are listed in FIG. 17. None of the genes used in this study were part of the 783 probe-sets used for molecular subtyping. The heat-maps of our dataset are shown as the top panel for each gene expression signature. The same gene clusters were applied to draw heat-maps on the other three independent datasets. The heat-maps for each signature were generated from top to bottom using datasets of KFSYSCC, EMC, Uppsala, and TRANSBIG. Each molecular subtype shared the same distinctive gene expression pattern among all four datasets. Subtypes I, II and IV had elevated expressions of cell cycle/proliferation genes. Similarly, subtypes I and II breast cancer samples showed a higher expression of the stromal genes known to be associated with poorer survival outcome (Nature Med 2008, 14:518-527). Subtypes III and VI had elevated expression of genes associated with vascular endothelial normalization. The concordance of differential expression of signature genes for the six molecular subtypes between the KFSYSCC dataset and each of the other three independent datasets was analyzed for Pearson correlation coefficient. The p value for each Pearson correlation coefficient was determined by comparing with null distribution based on 10,000 permutations of each public dataset at subtype level. All p values were <0.0001. The Pearson correlation coefficient between KFSYSCC and each dataset of EMC, Uppsala or TRANSBIG was 0.94, 0.92 or 0.87 for cell cycle/proliferation, 0.85, 0.84 or 0.78 for wound response, 0.94, 0.91 or 0.87 for stromal reaction, and 0.86, 0.86 or 0.83 for tumor vascular endothelial normalization.

FIG. 30 Summarizes a comparison of the present molecular subtypes of breast cancer (top) with the Perou-Sørlie intrinsic types (bottom). The top row shows the color-coded molecular subtypes of 327 samples in our dataset, and the lower panel shows how the same cases on top classified into the basal (green), HER2-overexpressing (red), luminal A (blue) and luminal B (brown) intrinsic types using the classification genes of Sørlie, et al. Proc Natl Acad Sci, USA, 98:10869-10874 (2001).

FIG. 31 Summarizes a comparison of survival outcome between molecular subtype V patients who underwent adjuvant chemotherapy and those who did not. Comparisons of survival were conducted for patients in our dataset (upper panels) and the NKI dataset (van de Vijver et al. New Engl J Med, 347:1999-2009 (2002)) (lower panels). The comparison of pertinent clinical parameters showed no differences between the two treatment groups from our KFSYSCC dataset (Table 17). Patients with subtype V breast cancer in the NKI database were identified using the classifier genes established in this study and centroid analysis. All NKI patients with N1 stage disease were selected for comparison. Tumor size distribution and the fraction of patients treated with hormonal therapy were not significantly different between the two treatment groups, with respective p values of 1.0 and 0.32 using Fisher's exact test. The NKI stage N0 patients were not included in this study because an overwhelming number did not receive adjuvant chemotherapy. Their inclusion would have caused an uneven distribution of disease severity. The results show that adjuvant chemotherapy did not provide survival benefit for patients with early stage subtype V breast cancer in either dataset.

FIG. 32 Comparison of overall survival between patients with subtype I breast cancer treated with CAF and CMF adjuvant chemotherapy. Clinical variables including age at diagnosis, TNM stages, positive lymph node number, nuclear grade, hormonal therapy and post-op radiation were compared between these two treatment groups. There were no significant differences (Table 28).

FIG. 33 Summarizes a correlation of molecular subtypes and the risk of distant recurrence predicted by using genes of the Oncotype and MammaPrint predictor. The three different datasets used in this study included ours (KFSYSCC), the EMC (Lancet 2005, 365:671-679) and the NKI (New Engl J Med 2002, 347:1999-2009). The number of cases in each subtype for the KFSYSCC, EMC, and NKI datasets were 37, 49, and 10 for subtype I; 34, 24, and 18 for subtype II; 41, 24, and 4 for subtype III; 81, 80, and 52 for subtype IV; 41, 39 and 172 for subtype V; and 93, 70 and 9 for subtype VI, respectively. For prediction of recurrence risk by genes of the Oncotype predictor, a higher score means a higher risk of recurrence. The negative correlation scores predicted by the MammaPrint predictor shown on the y axis represent a higher risk of distant recurrence. A score of <0 can be defined as high risk for recurrence and a score of=or >0 as low risk.

FIG. 34 Average expression intensity of TOP2A and FLOR1 genes in six different molecular subtypes of breast cancer. All patients (n=327) in our dataset were included in the study. The average expression of each gene is shown as mean±SEM. Student t test was conducted between subtype IV and other subtypes following logarithmic transformation of expression intensities to base of 2. TOP2A expression of subtype IV was significantly higher than subtype II, III, V and VI with p values of <0.0001 (*). There was no significant difference between subtype IV and I. For expression of FLOR1, subtype IV was significantly lower than subtypes I with p <0.0001(*). The number of samples in each subtype is available in Table 11.

### DETAILED DESCRIPTION OF THE INVENTION

The present invention is based, in part, on the identification of six molecular subtypes of breast cancer and optimized therapies that are effective for treating each of these subtypes. As described herein, a gene expression profiling study was conducted using samples from 327 breast cancer patients and the genes best suited for classification of breast cancer into different molecular subtypes (Table 1). The different molecular subtypes of breast cancer classified according to this approach were shown to have distinct clinical characteristics and biology and were determined to respond to treatment very differently. These features were used to determine an optimized therapy for each breast cancer subtype that can be employed effectively to treat breast cancer patients from different geographical areas and ethnic groups.

### DEFINITIONS

As used herein, “molecular subtype” and “breast cancer molecular subtype” are used interchangeably and refer to a breast cancer subtype (e.g., a subset of breast cancers) that is characterized by differential expression of a set (e.g., plurality) of genes, each of which displays either an elevated (e.g., increased) or reduced (e.g., decreased) level of expression in a breast cancer sample relative to a suitable control (e.g., a non-cancerous tissue or cell sample, a reference standard). Genes that are differentially expressed in a breast cancer can be, for example, genes that are known, or have been previously determined, to be differentially expressed in a breast cancer. The terms “molecular subtype” and “breast cancer molecular subtype” include the six breast cancer molecular subtypes described herein (subtypes, I, II, III, IV, V and VI as defined herein).

As used herein, “gene expression” refers to the translation of information encoded in a gene into a gene product (e.g., RNA, protein). Expressed genes include genes that are transcribed into RNA (e.g., mRNA) that is subsequently translated into protein, as well as genes that are transcribed into non-coding RNA molecules that are not translated into protein (e.g., transfer RNA (tRNA), ribosomal RNA (rRNA), microRNA, ribozymes).

“Level of expression,” “expression level” or “expression intensity” refers to the level (e.g., amount) of one or more gene products (e.g., mRNA, protein) encoded by a given gene in a sample or reference standard.

As used herein, “differentially expressed” or “differential expression” refers to any reproducible and detectable difference in the level of expression of a gene between two samples (e.g., two biological samples), or between a sample and a reference standard. Preferably, the difference in the level of gene expression is statistically-significant (p<0.05). Whether a difference in expression between two samples is statistically significant can be determined using an appropriate t-test (e.g., one-sample t-test, two-sample t-test, Welch's t-test) or other statistical test known to those of skill in the art.

A “gene expression profile” or “expression profile” refers to a set of genes which have expression levels that are associated with a particular biological activity (e.g., cell proliferation, cell cycle regulation, metastasis), cell type, disease state (e.g., breast cancer), state of cell differentiation or condition (e.g., a breast cancer subtype).

A “reference gene expression profile,” as used herein, refers to a representative (e.g., typical) gene expression profile for a given breast cancer molecular subtype or normal sample.

As used herein, “substantially similar” when used in reference to a gene expression profile refers two or more gene expression profiles (e.g., a gene expression profile of a breast cancer test sample and a reference gene expression profile for a particular breast cancer molecular subtype) that are either identical or at least 90% similar in terms of the identity of the genes in each profile that are differentially expressed at a statistically significant level relative to normal samples.

The term “probe set” refers to probes on an array (e.g., a microarray) that are complementary to the same target gene or gene product. A probe set can consist of one or more probes.

As used herein, “probe oligonucleotide” or “probe oligodeoxynucleotide” refers to an oligonucleotide on an array (e.g., a microarray) that is capable of hybridizing to a target oligonucleotide.

The term “oligonucleotide” as used herein refers to a nucleic acid molecule (e.g., RNA, DNA) that is about 5 to about 150 nucleotides in length. The oligonucleotide can be a naturally occurring oligonucleotide or a synthetic oligonucleotide. Oligonucleotides can be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art.

“Target oligonucleotide” or “target oligodeoxynucleotide” refers to a molecule to be detected (e.g., via hybridization).

“Detectable label” as used herein refers to a moiety that is capable of being specifically detected, either directly or indirectly, and therefore, can be used to distinguish a molecule that comprises the detectable label from a molecule that does not comprise the detectable label.

The phrase “specifically hybridizes” refers to the specific association of two complementary nucleotide sequences (e.g., DNA, RNA or a combination thereof) in a duplex under stringent conditions. The association of two nucleic acid molecules in a duplex occurs as a result of hydrogen bonding between complementary base pairs.

“Stringent conditions” or “stringency conditions” refer to a set of conditions under which two complementary nucleic acid molecules having at least 70% complementarity can hybridize. However, stringent conditions do not permit hybridization of two nucleic acid molecules that are not complementary (two nucleic acid molecules that have less than 70% sequence complementarity).

As used herein, “low stringency conditions” include, for example, hybridization in 6× sodium chloride/sodium citrate (SSC) at about 45° C., followed by two washes in 0.2×SSC, 0.1% SDS at least at 50° C. (the temperature of the washes can be increased to 55.0 for low stringency conditions).

“Medium stringency conditions” include, for example, hybridization in 6×SSC at about 45° C., followed by one or more washes in 0.2×SSC, 0.1% SDS at 60° C.

As used herein, “high stringency conditions” include, for example, hybridization in 6×SSC at about 45° C., followed by one or more washes in 0.2×SSC, 0.1% SDS at 65° C.;

“Very high stringency conditions” include, but are not limited to, hybridization in 0.5M sodium phosphate, 7% SDS at 65° C., followed by one or more washes at 0.2×SSC, 1% SDS at 65° C.

As used herein, the term “polypeptide” refers to a polymer of amino acids of any length and encompasses proteins, peptides, and oligopeptides.

As used herein, the term “sample” refers to a biological sample (e.g., a tissue sample, a cell sample, a fluid sample) that expresses genes that display differential levels of expression when cancer cells (e.g., breast cancer cells) of a particular molecular subtype are present in the sample versus when cancer cells of that subtype are absent from the sample.

“Distant metastasis” refers to cancer cells that have spread from the original (i.e., primary) tumor to distant organs or distant lymph nodes.

As used herein, a “subject” refers to a human. Examples of suitable subjects include, but are not limited to, both female and male human patients that have, or are at risk for developing, a breast cancer.

The terms “prevent,” “preventing,” or “prevention,” as used herein, mean reducing the probability/likelihood or risk of breast cancer tumor formation or progression in a subject, delaying the onset of a condition related to breast cancer in the subject, lessening the severity of one or more symptoms of a breast cancer-related condition in the subject, or any combination thereof. In general, the subject of a preventative regimen most likely will be categorized as being “at-risk”, e.g., the risk for the subject developing breast cancer is higher than the risk for an individual represented by the relevant baseline population.

As used herein, the terms “treat,” “treating,” or “treatment,” mean to counteract a medical condition (e.g., a condition related to breast cancer) to the extent that the medical condition is improved according to a clinically-acceptable standard (e.g., reduced number and/or size of breast cancer tumors in a subject).

As defined herein a “treatment regimen” is a regimen in which one or more therapeutic and/or prophylactic agents are administered to a subject at a particular dose (e.g., level, amount, quantity) and on a particular schedule and/or at particular intervals (e.g., minutes, days, weeks, months).

As defined herein, “therapy” is the administration of a particular therapeutic or prophylactic agent to a subject (e.g., a non-human mammal, a human), which results in a desired therapeutic or prophylactic benefit to the subject.

As defined herein, a “therapeutically effective amount” is an amount sufficient to achieve the desired therapeutic or prophylactic effect under the conditions of administration, such as an amount sufficient to inhibit (i.e., reduce, prevent) tumor formation, tumor growth (proliferation, size), tumor vascularization and/or tumor progression (invasion, metastasis) in a patient with a breast cancer. The effectiveness of a therapy (e.g., the reduction/elimination of a tumor and/or prevention of tumor growth) can be determined by any suitable method (e.g., in situ immunohistochemistry, imaging (ultrasound, CT scan, MRI, NMR), ³H-thymidine incorporation).

As used herein, “adjuvant therapy” refers to additional treatment (e.g., chemotherapy, radiotherapy), usually given after a primary treatment such as surgery (e.g., surgery for breast cancer), where all detectable disease has been removed, but where there remains a statistical risk of relapse due to occult disease. Typically, statistical evidence is used to assess the risk of disease relapse before deciding on a specific adjuvant therapy. The aim of adjuvant treatment is to improve disease-specific and overall survival. Because the treatment is essentially for a risk, rather than for provable disease, it is accepted that a proportion of patients who receive adjuvant therapy will already have been cured by their primary surgery. The primary goal of adjuvant chemotherapy is to control systemic relapse of a disease to improve long-term survival. Adjuvant radiotherapy is given to control local and/or regional recurrence.

As used herein, “adjuvant chemotherapy” refers to chemotherapy that is provided in addition to (e.g., subsequent to) a primary cancer treatment, such as surgery or radiation therapy.

As used herein, “high intensity chemotherapy” refers to a chemotherapy comprising administration of a high dose of a chemotherapeutic agent(s) and/or administration of a more potent chemotherapeutic agent(s). “High intensity chemotherapy” can also mean a more dose-intense chemotherapy.

As used herein, “dose-dense chemotherapy” refers to a chemotherapy regimen in which a chemotherapeutic agent(s) is given successively with short time intervals between successive treatments relative to a standard chemotherapy treatment regimen.

As used herein, “dose-intense chemotherapy” is a dose-dense chemotherapy regimen that includes administration of high doses of a chemotherapeutic agent(s).

As used herein, “anti-estrogen therapy” refers to a hormone therapy involving administration of one or more anti-estrogen therapeutic agents (e.g., aromatase inhibitors, Selective Estrogen Receptor Modulators (SERMs), Estrogen Receptor Downregulators (ERDs)). An “anti-estrogen therapy” typically works by lowering the amount of the hormone estrogen in the body or by blocking the action of estrogen on breast cancer cells.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

### Methods for Determining a Breast Cancer Molecular Subtype; Methods of Classifying a Breast Cancer According to a Molecular Subtype; Methods of Determining Immune Response Score

The methods described herein can be used to determine the molecular subtype of a breast cancer in a subject and to classify a breast cancer according to one of six different molecular subtypes identified herein. These molecular subtypes are referred to as a molecular subtype I breast cancer, a molecular subtype II breast cancer, a molecular subtype III breast cancer, a molecular subtype IV breast cancer, a molecular subtype V breast cancer and a molecular subtype VI breast cancer.

As described herein, it has been discovered that subsets of genes and gene products represented by the probe sets listed in Table 1 are differentially expressed in each of six newly identified breast cancer molecular subtypes. Thus, for a given breast cancer sample, a breast cancer molecular subtype can be determined, for example, by analyzing the expression in the breast cancer sample of all, or a characteristic subset, of genes and/or probe sets listed in Table 1, relative to a suitable control. Preferably, the expression levels of all genes/probe sets listed in Table 1 are analyzed to determine the particular molecular subtype to which a breast cancer belongs. This approach is particularly useful if the cancer has an unknown molecular subtype and/or is not suspected of belonging to a particular molecular subtype, or if multiple breast cancer samples are being tested. However, it is not always necessary to analyze all of the genes/probe sets listed in Table 1 to determine whether a breast cancer is a molecular subtype I, II, III, IV, V or VI breast cancer. For example, in some cases, the breast cancer molecular subtype (i.e., a molecular subtype I, II, III, IV, V or VI) can be determined by analyzing the expression of at least about 30% of the genes/probe sets in Table 1. For example, in some cases, the breast cancer molecular subtype can be determined by analyzing the expression of at least about 40%, at least about 50%, at least about 60%, at least about 70%, at least about 80%, at least about 90%, at least about 95% or 100% of the genes in Table 1. Preferably the expression of at least about 70%, more preferably at least about 80%, even more preferably at least about 90% of the genes in Table 1 are analyzed to determine the breast cancer molecular subtype.

TABLE 1
Genes/Probe Sets that are Differentially-expressed in One or More Breast
Cancer Molecular Subtypes (Molecular Subtypes I-VI)
(*indicates no Gene Symbol has been assigned)

| Affymetrix | | Representative Public ID* or | Gene |
|---|---|---|---|
| Probe Set ID | Gene Symbol* | RefSeq Transcript ID/Accession Number | Cluster # |
| 1554007_at | — | BC036488 | Group 9 |
| 1555893_at | — | AI918054 | Group 9 |
| 1556221_a_at | — | BM992214 | Group 7 |
| 1557810_at | — | BM352108 | Group 5 |
| 1557843_at | — | BC036114 | Group 9 |
| 1558686_at | — | BM983749 | Group 7 |
| 1559949_at | — | T56980 | Group 8 |
| 1560049_at | — | AI125337 | Group 13 |
| 1560550_at | — | BC037972 | Group 7 |
| 1560850_at | — | BC016831 | Group 7 |
| 1561938_at | — | AL832704 | Group 9 |
| 1562821_a_at | — | AF401033 | Group 9 |
| 1565595_at | — | AU144979 | Group 2 |
| 1567101_at | — | AF147347 | Group 7 |
| 1567997_x_at | — | D17262 | Group 9 |
| 217191_x_at | — | AF042163 | Group 9 |
| 220898_at | — | NM_024972 | Group 8 |
| 222326_at | — | AW973834 | Group 4 |
| 224989_at | — | AI824013 | Group 7 |
| 225123_at | — | BE883841 | Group 13 |
| 226034_at | — | BE222344 | Group 7 |
| 227762_at | — | AW244016 | Group 13 |
| 227929_at | — | AU151342 | Group 7 |
| 227952_at | — | AI580142 | Group 12 |
| 228175_at | — | AL137310 | Group 7 |
| 228273_at | — | BG165011 | Group 3 |
| 228390_at | — | AA489100 | Group 7 |
| 228528_at | — | AI927692 | Group 9 |
| 228750_at | — | AI693516 | Group 13 |
| 229072_at | — | BF968097 | Group 7 |
| 229659_s_at | — | BE501712 | Group 13 |
| 230130_at | — | AI692523 | Group 13 |
| 230491_at | — | BF111884 | Group 9 |
| 230570_at | — | AI702465 | Group 9 |
| 230791_at | — | AU146924 | Group 1 |
| 231034_s_at | — | AI871589 | Group 1 |
| 231098_at | — | BF939996 | Group 10 |
| 231291_at | — | AI694139 | Group 9 |
| 232105_at | — | AU148391 | Group 1 |
| 232210_at | — | AU146384 | Group 9 |
| 232290_at | — | BE815259 | Group 7 |
| 232614_at | — | AU146963 | Group 9 |
| 232850_at | — | AU147577 | Group 9 |
| 232935_at | — | AA569225 | Group 13 |
| 233059_at | — | AK026384 | Group 9 |
| 233273_at | — | AU146834 | Group 9 |
| 233388_at | — | AK022350 | Group 9 |
| 233413_at | — | AU156421 | Group 9 |
| 233691_at | — | AK025359 | Group 4 |
| 234785_at | — | AK025047 | Group 11 |
| 235501_at | — | AW961576 | Group 7 |
| 235609_at | — | BF056791 | Group 3 |
| 235771_at | — | BF594722 | Group 9 |
| 235786_at | — | AI806781 | Group 9 |
| 235856_at | — | AI660245 | Group 7 |
| 236114_at | — | AI798118 | Group 9 |
| 236256_at | — | AW993690 | Group 11 |
| 236307_at | — | AA085906 | Group 13 |
| 236445_at | — | AI820661 | Group 9 |
| 237112_at | — | R59908 | Group 9 |
| 238827_at | — | BE843544 | Group 13 |
| 239066_at | — | AW364675 | Group 7 |
| 239638_at | — | AI608696 | Group 7 |
| 239723_at | — | AA588092 | Group 7 |
| 239907_at | — | BF508839 | Group 7 |
| 240247_at | — | AI653240 | Group 3 |
| 240724_at | — | AI668629 | Group 13 |
| 240733_at | — | W92005 | Group 7 |
| 240788_at | — | AI076834 | Group 3 |
| 241310_at | — | AI685841 | Group 7 |
| 241466_at | — | AI275776 | Group 9 |
| 241577_at | — | AI732794 | Group 9 |
| 241929_at | — | AV760302 | Group 13 |
| 242022_at | — | BF883581 | Group 9 |
| 242657_at | — | AI078033 | Group 9 |
| 242671_at | — | BF055144 | Group 1 |
| 242836_at | — | AI800470 | Group 12 |
| 242868_at | — | T70087 | Group 13 |
| 243168_at | — | AI916532 | Group 9 |
| 243241_at | — | AW341473 | Group 9 |
| 243806_at | — | AW015140 | Group 7 |
| 243907_at | — | AW117383 | Group 9 |
| 243929_at | — | H15261 | Group 7 |
| 244375_at | — | AW873606 | Group 9 |
| 244579_at | — | AI086336 | Group 8 |
| 244696_at | — | AI033582 | Group 9 |
| 244697_at | — | AI833064 | Group 13 |
| 209459_s_at | ABAT | NM_000663 /// NM_001127448 /// | Group 9 |
| | | NM_020686 | |
| 209460_at | ABAT | NM_000663 /// NM_001127448 /// | Group 9 |
| | | NM_020686 | |
| 224146_s_at | ABCC11 | NM_032583 /// NM_033151 /// | Group 10 |
| | | NM_145186 | |
| 1553410_a_at | ABCC12 | NM_033226 | Group 10 |
| 215559_at | ABCC6 | NM_001079528 /// NM_001171 | Group 11 |
| 205355_at | ACADSB | NM_001609 | Group 9 |
| 226030_at | ACADSB | NM_001609 | Group 9 |
| 201963_at | ACSL1 | NM_001995 | Group 10 |
| 232570_s_at | ADAM33 | NM_025220 /// NM_153202 | Group 13 |
| 237411_at | ADAMTS6 | NM_197941 | Group 12 |
| 235049_at | ADCY1 | NM_021116 | Group 9 |
| 207175_at | ADIPOQ | NM_004797 | Group 13 |
| 243967_at | AFF3 | NM_001025108 /// NM_002285 | Group 9 |
| 228241_at | AGR3 | NM_176813 | Group 9 |
| 223075_s_at | AIF1L | NM_031426 | Group 1 |
| 222862_s_at | AK5 | NM_012093 /// NM_174858 | Group 13 |
| 216381_x_at | AKR7A3 | NM_012067 | Group 9 |
| 204942_s_at | ALDH3B2 | NM_000695 /// NM_001031615 | Group 10 |
| 202920_at | ANK2 | NM_001127493 /// NM_001148 /// | Group 13 |
| | | NM_020977 | |
| 223864_at | ANKRD30A | NM_052997 | Group 7 |
| 230238_at | ANKRD43 | NM_175873 | Group 7 |
| 1552619_a_at | ANLN | NM_018685 | Group 3 |
| 222608_s_at | ANLN | NM_018685 | Group 3 |
| 210085_s_at | ANXA9 | NM_003568 | Group 9 |
| 211712_s_at | ANXA9 | NM_003568 | Group 9 |
| 201525_at | APOD | NM_001647 | Group 13 |
| 207542_s_at | AQP1 | NM_198098 | Group 13 |
| 209047_at | AQP1 | NM_198098 | Group 13 |
| 205568_at | AQP9 | NM_020980 | Group 3 |
| 205239_at | AREG | NM_001657 | Group 9 |
| 219918_s_at | ASPM | NM_018136 | Group 3 |
| 219087_at | ASPN | NM_017680 | Group 12 |
| 224396_s_at | ASPN | NM_017680 | Group 12 |
| 207076_s_at | ASS1 | NM_000050 /// NM_054012 | Group 2 |
| 218782_s_at | ATAD2 | NM_014109 | Group 3 |
| 222740_at | ATAD2 | NM_014109 | Group 3 |
| 228401_at | ATAD2 | NM_014109 | Group 3 |
| 219359_at | ATHL1 | NM_025092 | Group 9 |
| 243585_at | ATP13A5 | NM_198505 | Group 2 |
| 1558612_a_at | ATP1A4 | NM_001001734 /// NM_144699 | Group 7 |
| 1552532_a_at | ATP6V1C2 | NM_001039362 /// NM_144583 | Group 1 |
| 1553989_a_at | ATP6V1C2 | NM_001039362 /// NM_144583 | Group 1 |
| 213745_at | ATRNL1 | NM_207303 | Group 7 |
| 204092_s_at | AURKA | NM_003600 /// NM_198433 /// | Group 3 |
| | | NM_198434 /// NM_198435 /// | |
| | | NM_198436 /// NM_198437 | |
| 208079_s_at | AURKA | NM_003600 /// NM_198433 /// | Group 3 |
| | | NM_198434 /// NM_198435 /// | |
| | | NM_198436 /// NM_198437 | |
| 217013_at | AZGP1P1 | XR_017216 /// XR_037935 /// | Group 7 |
| | | XR_039311 /// XR_039317 | |
| 218899_s_at | BAALC | NM_001024372 /// NM_024812 | Group 13 |
| 204966_at | BAI2 | NM_001703 | Group 9 |
| 216356_x_at | BAIAP3 | NM_003933 | Group 9 |
| 203304_at | BAMBI | NM_012342 | Group 4 |
| 204378_at | BCAS1 | NM_003657 | Group 7 |
| 203685_at | BCL2 | NM_000633 /// NM_000657 | Group 9 |
| 215440_s_at | BEX4 | NM_001080425 /// NM_001127688 | Group 12 |
| 202094_at | BIRC5 | NM_001012270 /// NM_001012271 /// | Group 3 |
| | | NM_001168 | |
| 202095_s_at | BIRC5 | NM_001012270 /// NM_001012271 /// | Group 3 |
| | | NM_001168 | |
| 210523_at | BMPR1B | NM_001203 | Group 9 |
| 229975_at | BMPR1B | NM_001203 | Group 9 |
| 238478_at | BNC2 | NM_017637 | Group 12 |
| 1553072_at | BNIPL | NM_001159642 /// NM_138278 | Group 7 |
| 204531_s_at | BRCA1 | NM_007294 /// NM_007295 /// | Group 8 |
| | | NM_007296 /// NM_007297 /// | |
| | | NM_007298 /// NM_007299 /// | |
| | | NM_007300 /// NM_007302 /// | |
| | | NM_007303 /// NM_007304 /// | |
| | | NM_007305 /// NR_027676 | |
| 203755_at | BUB1B | NM_001211 | Group 3 |
| 231084_at | C10orf79 | NM_025145 | Group 7 |
| 231859_at | C14orf132 | NR_023938 /// XM_001724179 /// | Group 9 |
| | | XM_001724602 /// XM_001726369 /// | |
| | | XR_040536 /// XR_040537 /// | |
| | | XR_040538 | |
| 220173_at | C14orf45 | NM_025057 | Group 7 |
| 224447_s_at | C17orf37 | NM_032339 | Group 2 |
| 228066_at | C17orf96 | NM_001130677 | Group 2 |
| 223631_s_at | C19orf33 | NM_033520 | Group 9 |
| 219010_at | C1orf106 | NM_001142569 /// NM_018265 | Group 2 |
| 223125_s_at | C1orf21 | NM_030806 | Group 7 |
| 229381_at | C1orf64 | NM_178840 | Group 9 |
| 224443_at | C1orf97 | NR_026761 /// XR_040057 /// | Group 9 |
| | | XR_040058 /// XR_040059 | |
| 202357_s_at | C2 /// CFB | NM_000063 /// NM_001145903 /// | Group 7 |
| | | NM_001710 | |
| 226067_at | C20orf114 | NM_033197 | Group 7 |
| 236222_at | C3orf15 | NM_033364 | Group 9 |
| 208451_s_at | C4A /// C4B | NM_000592 /// NM_001002029 /// | Group 7 |
| | | NM_007293 /// XM_001722806 | |
| 214428_x_at | C4A /// C4B | NM_000592 /// NM_001002029 /// | Group 7 |
| | | NM_007293 /// XM_001722806 | |
| 218195_at | C6orf211 | NM_024573 | Group 9 |
| 218541_s_at | C8orf4 | NM_020130 | Group 9 |
| 230661_at | C8orf84 | NM_153225 | Group 13 |
| 1557867_s_at | C9orf117 | NM_001012502 | Group 7 |
| 225777_at | C9orf140 | NM_178448 | Group 3 |
| 213900_at | C9orf61 | NM_001127608 /// NM_004816 | Group 13 |
| 210735_s_at | CA12 | NM_001218 /// NM_206925 | Group 9 |
| 215867_x_at | CA12 | NM_001218 /// NM_206925 | Group 9 |
| 225915_at | CAB39L | NM_001079670 /// NM_030925 | Group 7 |
| 221585_at | CACNG4 | NM_014405 | Group 9 |
| 220414_at | CALML5 | NM_017422 | Group 2 |
| 200935_at | CALR | NM_004343 | Group 3 |
| 211483_x_at | CAMK2B | NM_001220 /// NM_172078 /// | Group 9 |
| | | NM_172079 /// NM_172080 /// | |
| | | NM_172081 /// NM_172082 /// | |
| | | NM_172083 /// NM_172084 | |
| 212551_at | CAP2 | NM_006366 | Group 9 |
| 202965_s_at | CAPN6 | NM_014289 | Group 1 |
| 236085_at | CAPSL | NM_001042625 /// NM_144647 | Group 7 |
| 228323_at | CASC5 | NM_144508 /// NM_170589 | Group 3 |
| 207317_s_at | CASQ2 | NM_001232 | Group 13 |
| 203324_s_at | CAV2 | NM_001233 /// NM_198212 | Group 13 |
| 227966_s_at | CCDC74A /// | NM_138770 /// NM_207310 | Group 9 |
| | CCDC74B | | |
| 238759_at | CCDC88A | NM_001135597 /// NM_018084 | Group 1 |
| 239233_at | CCDC88A | NM_001135597 /// NM_018084 | Group 1 |
| 213226_at | CCNA2 | NM_001237 | Group 3 |
| 214710_s_at | CCNB1 | NM_031966 | Group 3 |
| 228729_at | CCNB1 | NM_031966 | Group 3 |
| 202705_at | CCNB2 | NM_004701 | Group 3 |
| 205034_at | CCNE2 | NM_057749 | Group 3 |
| 202769_at | CCNG2 | NM_004354 | Group 7 |
| 202770_s_at | CCNG2 | NM_004354 | Group 7 |
| 211559_s_at | CCNG2 | NM_004354 | Group 7 |
| 208650_s_at | CD24 | NM_013230 /// XM_001725629 | Group 4 |
| 228766_at | CD36 | NM_000072 /// NM_001001547 /// | Group 13 |
| | | NM_001001548 /// NM_001127443 /// | |
| | | NM_001127444 | |
| 1565868_at | CD44 | NM_000610 /// NM_001001389 /// | Group 5 |
| | | NM_001001390 /// NM_001001391 /// | |
| | | NM_001001392 | |
| 203214_x_at | CDC2 | NM_001130829 /// NM_001786 /// | Group 3 |
| | | NM_033379 | |
| 210559_s_at | CDC2 | NM_001130829 /// NM_001786 /// | Group 3 |
| | | NM_033379 | |
| 202870_s_at | CDC20 | NM_001255 | Group 3 |
| 204695_at | CDC25A | NM_001789 /// NM_201567 | Group 4 |
| 223307_at | CDCA3 | NM_031299 | Group 3 |
| 1555758_a_at | CDKN3 | NM_001130851 /// NM_005192 | Group 3 |
| 209714_s_at | CDKN3 | NM_001130851 /// NM_005192 | Group 3 |
| 211883_x_at | CEACAM1 | NM_001024912 /// NM_001712 | Group 5 |
| 201884_at | CEACAM5 | NM_004363 | Group 11 |
| 203757_s_at | CEACAM6 | NM_002483 | Group 11 |
| 211657_at | CEACAM6 | NM_002483 | Group 11 |
| 213006_at | CEBPD | NM_005195 | Group 13 |
| 207828_s_at | CENPF | NM_016343 | Group 3 |
| 209172_s_at | CENPF | NM_016343 | Group 3 |
| 214804_at | CENPI | NM_006733 | Group 3 |
| 222848_at | CENPK | NM_022145 | Group 3 |
| 232065_x_at | CENPL | NM_001127181 /// NM_033319 | Group 3 |
| 228559_at | CENPN | NM_001100624 /// NM_001100625 /// | Group 3 |
| | | NM_018455 | |
| 226611_s_at | CENPV | NM_181716 | Group 1 |
| 218542_at | CEP55 | NM_001127182 /// NM_018131 | Group 3 |
| 1555564_a_at | CFI | NM_000204 | Group 13 |
| 206869_at | CHAD | NM_001267 | Group 7 |
| 1559739_at | CHPT1 | NM_020244 | Group 9 |
| 221675_s_at | CHPT1 | NM_020244 | Group 9 |
| 230364_at | CHPT1 | NM_020244 | Group 9 |
| 209763_at | CHRDL1 | NM_001143981 /// NM_001143982 /// | Group 13 |
| | | NM_001143983 /// NM_145234 | |
| 224400_s_at | CHST9 | NM_031422 | Group 1 |
| 226736_at | CHURC1 | NM_145165 | Group 9 |
| 223961_s_at | CISH | NM_013324 /// NM_145071 | Group 9 |
| 207144_s_at | CITED1 | NM_001144885 /// NM_001144886 /// | Group 9 |
| | | NM_001144887 /// NM_004143 | |
| 201897_s_at | CKS1B | NM_001826 /// NR_024163 | Group 3 |
| 204170_s_at | CKS2 | NM_001827 | Group 3 |
| 206164_at | CLCA2 | NM_006536 | Group 13 |
| 206165_s_at | CLCA2 | NM_006536 | Group 13 |
| 217528_at | CLCA2 | NM_006536 | Group 13 |
| 218182_s_at | CLDN1 | NM_021101 | Group 5 |
| 227742_at | CLIC6 | NM_053277 | Group 9 |
| 242913_at | CLIC6 | NM_053277 | Group 9 |
| 212358_at | CLIP3 | NM_015526 | Group 13 |
| 226425_at | CLIP4 | NM_024692 | Group 1 |
| 213839_at | CLMN | NM_024734 | Group 7 |
| 222043_at | CLU | NM_001831 /// NM_203339 | Group 13 |
| 229084_at | CNTN4 | NM_175607 /// NM_175612 /// | Group 12 |
| | | NM_175613 | |
| 219300_s_at | CNTNAP2 | NM_014141 | Group 11 |
| 219301_s_at | CNTNAP2 | NM_014141 | Group 11 |
| 204345_at | COL16A1 | NM_001856 | Group 12 |
| 204636_at | COL17A1 | NM_000494 | Group 13 |
| 212489_at | COL5A1 | NM_000093 | Group 12 |
| 213290_at | COL6A2 | NM_001849 /// NM_058174 /// | Group 12 |
| | | NM_058175 | |
| 204724_s_at | COL9A3 | NM_001853 | Group 1 |
| 214336_s_at | COPA | NM_001098398 /// NM_004371 | Group 5 |
| 227177_at | CORO2A | NM_003389 /// NM_052820 | Group 7 |
| 1558034_s_at | CP | NM_000096 | Group 4 |
| 204846_at | CP | NM_000096 | Group 4 |
| 228143_at | CP | NM_000096 | Group 4 |
| 205509_at | CPB1 | NM_001871 | Group 9 |
| 205350_at | CRABP1 | NM_004378 | Group 1 |
| 209522_s_at | CRAT | NM_000755 /// NM_004003 | Group 7 |
| 226455_at | CREB3L4 | NM_130898 | Group 11 |
| 204573_at | CROT | NM_001143935 /// NM_021151 /// | Group 7 |
| | | NR_026585 | |
| 206994_at | CST4 | NM_001899 | Group 12 |
| 226960_at | CXCL17 | NM_198477 | Group 11 |
| 207843_x_at | CYB5A | NM_001914 /// NM_148923 | Group 7 |
| 209366_x_at | CYB5A | NM_001914 /// NM_148923 | Group 7 |
| 215726_s_at | CYB5A | NM_001914 /// NM_148923 | Group 7 |
| 214622_at | CYP21A2 | NM_000500 /// NM_001128590 | Group 7 |
| 217133_x_at | CYP2B6 | NM_000767 | Group 9 |
| 206754_s_at | CYP2B6 /// | NM_000767 /// NR_001278 | Group 9 |
| | CYP2B7P1 | | |
| 210272_at | CYP2B7P1 | NR_001278 | Group 9 |
| 1553977_a_at | CYP39A1 | NM_016593 | Group 1 |
| 227702_at | CYP4X1 | NM_178033 | Group 7 |
| 237395_at | CYP4Z1 | NM_178134 | Group 10 |
| 1553434_at | CYP4Z2P | NR_002788 /// XR_042146 | Group 10 |
| 205471_s_at | DACH1 | NM_004392 /// NM_080759 /// | Group 7 |
| | | NM_080760 | |
| 228915_at | DACH1 | NM_004392 /// NM_080759 /// | Group 7 |
| | | NM_080760 | |
| 218094_s_at | DBNDD2 /// | NM_001048221 /// NM_001048222 /// | Group 9 |
| | SYS1- | NM_001048223 /// NM_001048224 /// | |
| | DBNDD2 | NM_001048225 /// NM_001048226 /// | |
| | | NR_003189 | |
| 232603_at | DCDC5 | NM_198462 | Group 9 |
| 222958_s_at | DEPDC1 | NM_001114120 /// NM_017779 | Group 3 |
| 235545_at | DEPDC1 | NM_001114120 /// NM_017779 | Group 3 |
| 206463_s_at | DHRS2 | NM_005794 /// NM_182908 | Group 7 |
| 214079_at | DHRS2 | NM_005794 /// NM_182908 | Group 7 |
| 206457_s_at | DIO1 | NM_000792 /// NM_001039715 /// | Group 7 |
| | | NM_001039716 /// NM_213593 | |
| 203764_at | DLGAP5 | NM_001146015 /// NM_014750 | Group 3 |
| 207147_at | DLX2 | NM_004405 | Group 9 |
| 232381_s_at | DNAH5 | NM_001369 | Group 7 |
| 1558080_s_at | DNAJC3 | NM_006260 | Group 5 |
| 240633_at | DOK7 | NM_173660 | Group 9 |
| 216918_s_at | DST | NM_001144769 /// NM_001144770 /// | Group 13 |
| | | NM_001144771 /// NM_001723 /// | |
| | | NM_015548 /// NM_020388 /// | |
| | | NM_183380 | |
| 218585_s_at | DTL | NM_016448 | Group 3 |
| 222680_s_at | DTL | NM_016448 | Group 3 |
| 201041_s_at | DUSP1 | NM_004417 | Group 13 |
| 204014_at | DUSP4 | NM_001394 /// NM_057158 | Group 7 |
| 204015_s_at | DUSP4 | NM_001394 /// NM_057158 | Group 7 |
| 208891_at | DUSP6 | NM_001946 /// NM_022652 | Group 13 |
| 208892_s_at | DUSP6 | NM_001946 /// NM_022652 | Group 13 |
| 228033_at | E2F7 | NM_203394 | Group 3 |
| 206101_at | ECM2 | NM_001393 | Group 12 |
| 219787_s_at | ECT2 | NM_018098 | Group 3 |
| 208399_s_at | EDN3 | NM_000114 /// NM_207032 /// | Group 1 |
| | | NM_207033 /// NM_207034 | |
| 204540_at | EEF1A2 | NM_001958 | Group 9 |
| 223608_at | EFCAB2 | NM_001143943 /// NM_032328 /// | Group 9 |
| | | NR_026586 /// NR_026587 /// | |
| | | NR_026588 | |
| 201984_s_at | EGFR | NM_005228 /// NM_201282 /// | Group 1 |
| | | NM_201283 /// NM_201284 | |
| 227404_s_at | EGR1 | NM_001964 | Group 13 |
| 206115_at | EGR3 | NM_004430 | Group 9 |
| 225827_at | EIF2C2 | NM_012154 | Group 5 |
| 220624_s_at | ELF5 | NM_001422 /// NM_198381 | Group 1 |
| 208788_at | ELOVL5 | NM_021814 | Group 7 |
| 231713_s_at | ELP2 | NM_018255 | Group 9 |
| 227874_at | EMCN | NM_001159694 /// NM_016242 | Group 13 |
| 228256_s_at | EPB41L4A | NM_022140 | Group 7 |
| 216836_s_at | ERBB2 | NM_001005862 /// NM_004448 | Group 2 |
| 224576_at | ERGIC1 | NM_001031711 /// NM_020462 | Group 11 |
| 231944_at | ERO1LB | NM_019891 | Group 9 |
| 38158_at | ESPL1 | NM_012291 | Group 3 |
| 205225_at | ESR1 | NM_000125 /// NM_001122740 /// | Group 9 |
| | | NM_001122741 /// NM_001122742 | |
| 211235_s_at | ESR1 | NM_000125 /// NM_001122740 /// | Group 9 |
| | | NM_001122741 /// NM_001122742 | |
| 215551_at | ESR1 | NM_000125 /// NM_001122740 /// | Group 9 |
| | | NM_001122741 /// NM_001122742 | |
| 217838_s_at | EVL | NM_016337 | Group 9 |
| 227232_at | EVL | NM_016337 | Group 9 |
| 203305_at | F13A1 | NM_000129 | Group 13 |
| 207300_s_at | F7 | NM_000131 /// NM_019616 | Group 7 |
| 202862_at | FAH | NM_000137 | Group 7 |
| 241031_at | FAM148A | NM_207322 | Group 11 |
| 238018_at | FAM150B | NM_001002919 | Group 13 |
| 227194_at | FAM3B | NM_058186 /// NM_206964 | Group 12 |
| 228069_at | FAM54A | NM_001099286 /// NM_138419 | Group 3 |
| 225834_at | FAM72A /// | NM_001100910 /// NM_001123168 /// | Group 3 |
| | FAM72B /// | NM_207418 /// XM_001128582 /// | |
| | FAM72D | XM_001133363 /// XM_001133364 /// | |
| | | XM_001133365 | |
| 225687_at | FAM83D | NM_030919 | Group 3 |
| 212218_s_at | FASN | NM_004104 | Group 7 |
| 203088_at | FBLN5 | NM_006329 | Group 13 |
| 227641_at | FBXL16 | NM_153350 | Group 9 |
| 218796_at | FERMT1 | NM_017671 | Group 1 |
| 203638_s_at | FGFR2 | NM_000141 /// NM_001144913 /// | Group 9 |
| | | NM_001144914 /// NM_001144915 /// | |
| | | NM_001144916 /// NM_001144917 /// | |
| | | NM_001144918 /// NM_001144919 /// | |
| | | NM_022970 | |
| 203639_s_at | FGFR2 | NM_000141 /// NM_001144913 /// | Group 9 |
| | | NM_001144914 /// NM_001144915 /// | |
| | | NM_001144916 /// NM_001144917 /// | |
| | | NM_001144918 /// NM_001144919 /// | |
| | | NM_022970 | |
| 208228_s_at | FGFR2 | NM_000141 /// NM_001144913 /// | Group 9 |
| | | NM_001144914 /// NM_001144915 /// | |
| | | NM_001144916 /// NM_001144917 /// | |
| | | NM_001144918 /// NM_001144919 /// | |
| | | NM_022970 | |
| 211237_s_at | FGFR4 | NM_002011 /// NM_022963 /// | Group 10 |
| | | NM_213647 | |
| 1552388_at | FLJ30901 | — | Group 9 |
| 226184_at | FMNL2 | NM_052905 | Group 5 |
| 205776_at | FMO5 | NM_001144829 /// NM_001144830 /// | Group 7 |
| | | NM_001461 | |
| 215300_s_at | FMO5 | NM_001144829 /// NM_001144830 /// | Group 7 |
| | | NM_001461 | |
| 204667_at | FOXA1 | NM_004496 | Group 9 |
| 1553613_s_at | FOXC1 | NM_001453 | Group 1 |
| 202723_s_at | FOXO1 | NM_002015 | Group 13 |
| 1553622_a_at | FSIP1 | NM_152597 | Group 9 |
| 203988_s_at | FUT8 | NM_004480 /// NM_178154 /// | Group 7 |
| | | NM_178155 /// NM_178156 /// | |
| | | NM_178157 | |
| 230906_at | GALNT10 | NM_017540 /// NM_198321 | Group 11 |
| 222773_s_at | GALNT12 | NM_024642 | Group 13 |
| 219271_at | GALNT14 | NM_024572 | Group 2 |
| 205696_s_at | GFRA1 | NM_001145453 /// NM_005264 /// | Group 9 |
| | | NM_145793 | |
| 227550_at | GFRA1 | NM_001145453 /// NM_005264 /// | Group 9 |
| | | NM_145793 | |
| 230163_at | GFRA1 | NM_001145453 /// NM_005264 /// | Group 9 |
| | | NM_145793 | |
| 203560_at | GGH | NM_003878 | Group 4 |
| 205582_s_at | GGT5 | NM_001099781 /// NM_001099782 /// | Group 13 |
| | | NM_004121 | |
| 206102_at | GINS1 | NM_021067 | Group 3 |
| 201667_at | GJA1 | NM_000165 | Group 9 |
| 200648_s_at | GLUL | NM_001033044 /// NM_001033056 /// | Group 9 |
| | | NM_002065 | |
| 1554712_a_at | GLYATL2 | NM_145016 | Group 2 |
| 209576_at | GNAI1 | NM_002069 | Group 13 |
| 208798_x_at | GOLGA8A | NM_181077 /// NR_027409 /// | Group 13 |
| | | XM_001714558 | |
| 218692_at | GOLSYN | NM_001099743 /// NM_001099744 /// | Group 7 |
| | | NM_001099745 /// NM_001099746 /// | |
| | | NM_001099747 /// NM_001099748 /// | |
| | | NM_001099749 /// NM_001099750 /// | |
| | | NM_001099751 /// NM_001099752 /// | |
| | | NM_001099753 /// NM_001099754 /// | |
| | | NM_001099755 /// NM_001099756 /// | |
| | | NM_017786 | |
| 208473_s_at | GP2 | NM_001007240 /// NM_001007241 /// | Group 7 |
| | | NM_001007242 /// NM_001502 | |
| 214324_at | GP2 | NM_001007240 /// NM_001007241 /// | Group 7 |
| | | NM_001007242 /// NM_001502 | |
| 213094_at | GPR126 | NM_001032394 /// NM_001032395 /// | Group 2 |
| | | NM_020455 /// NM_198569 | |
| 219936_s_at | GPR87 | NM_023915 | Group 1 |
| 210761_s_at | GRB7 | NM_001030002 /// NM_005310 | Group 2 |
| 202554_s_at | GSTM3 | NM_000849 /// NR_024537 | Group 9 |
| 200824_at | GSTP1 | NM_000852 | Group 1 |
| 204318_s_at | GTSE1 | NM_016426 | Group 3 |
| 237339_at | hCG_25653 | XM_001724231 /// XM_933553 /// | Group 7 |
| | | XM_944750 | |
| 226446_at | HES6 | NM_001142853 /// NM_018645 | Group 8 |
| 205221_at | HGD | NM_000187 /// XM_001713606 | Group 11 |
| 214307_at | HGD | NM_000187 /// XM_001713606 | Group 11 |
| 214308_s_at | HGD | NM_000187 /// XM_001713606 | Group 11 |
| 215933_s_at | HHEX | NM_002729 | Group 13 |
| 209911_x_at | HIST1H2BD | NM_021063 /// NM_138720 | Group 9 |
| 205967_at | HIST1H4C | NM_003542 | Group 5 |
| 206074_s_at | HMGA1 | NM_002131 /// NM_145899 /// | Group 4 |
| | | NM_145901 /// NM_145902 /// | |
| | | NM_145903 /// NM_145904 /// | |
| | | NM_145905 | |
| 203744_at | HMGB3 | NM_005342 | Group 3 |
| 204607_at | HMGCS2 | NM_005518 | Group 7 |
| 207165_at | HMMR | NM_001142556 /// NM_001142557 /// | Group 3 |
| | | NM_012484 /// NM_012485 | |
| 209709_s_at | HMMR | NM_001142556 /// NM_001142557 /// | Group 3 |
| | | NM_012484 /// NM_012485 | |
| 217755_at | HN1 | NM_001002032 /// NM_001002033 /// | Group 4 |
| | | NM_016185 | |
| 222222_s_at | HOMER3 | NM_001145721 /// NM_001145722 /// | Group 3 |
| | | NM_001145724 /// NM_004838 /// | |
| | | NR_027297 | |
| 205453_at | HOXB2 | NM_002145 | Group 7 |
| 204818_at | HSD17B2 | NM_002153 | Group 2 |
| 211538_s_at | HSPA2 | NM_021979 | Group 7 |
| 213931_at | ID2 /// ID2B | NM_002166 /// NR_026582 | Group 12 |
| 202411_at | IFI27 | NM_001130080 /// NM_005532 | Group 3 |
| 242903_at | IFNGR1 | NM_000416 | Group 5 |
| 209540_at | IGF1 | NM_000618 /// NM_001111283 /// | Group 13 |
| | | NM_001111284 /// NM_001111285 | |
| 209541_at | IGF1 | NM_000618 /// NM_001111283 /// | Group 13 |
| | | NM_001111284 /// NM_001111285 | |
| 202410_x_at | IGF2 /// INS- | NM_000612 /// NM_001007139 /// | Group 12 |
| | IGF2 | NM_001042376 /// NM_001127598 /// | |
| | | NR_003512 | |
| 221926_s_at | IL17RC | NM_032732 /// NM_153460 /// | Group 5 |
| | | NM_153461 | |
| 202948_at | IL1R1 | NM_000877 | Group 13 |
| 212195_at | IL6ST | NM_002184 /// NM_175767 | Group 7 |
| 212196_at | IL6ST | NM_002184 /// NM_175767 | Group 7 |
| 213446_s_at | IQGAP1 | NM_003870 | Group 5 |
| 229538_s_at | IQGAP3 | NM_178229 | Group 3 |
| 227314_at | ITGA2 | NM_002203 | Group 6 |
| 208084_at | ITGB6 | NM_000888 | Group 6 |
| 213832_at | KCND3 | NM_004980 /// NM_172198 | Group 7 |
| 222379_at | KCNE4 | NM_080671 | Group 9 |
| 214595_at | KCNG1 | NM_002237 /// NM_172318 | Group 4 |
| 207142_at | KCNJ3 | NM_002239 | Group 9 |
| 220540 at | KCNK15 | NM_022358 | Group 9 |
| 223658 at | KCNK6 | NM_004823 | Group 9 |
| 219545_at | KCTD14 | NM_023930 | Group 1 |
| 238077_at | KCTD6 | NM_001128214 /// NM_153331 | Group 9 |
| 212492_s_at | KDM4B | NM_015015 | Group 9 |
| 212495_at | KDM4B | NM_015015 | Group 9 |
| 212496_s_at | KDM4B | NM_015015 | Group 9 |
| 211713_x_at | KIAA0101 | NM_001029989 /// NM_014736 | Group 3 |
| 225327_at | KIAA1370 | NM_019600 | Group 7 |
| 223600_s_at | KIAA1683 | NM_001145304 /// NM_001145305 /// | Group 9 |
| | | NM_025249 | |
| 204444_at | KIF11 | NM_004523 | Group 3 |
| 202962_at | KIF13B | NM_015254 | Group 7 |
| 206364_at | KIF14 | NM_014875 | Group 3 |
| 219306_at | KIF15 | NM_020242 | Group 3 |
| 232083_at | KIF16B | NM_024704 | Group 9 |
| 218755_at | KIF20A | NM_005733 | Group 3 |
| 204709_s_at | KIF23 | NM_004856 /// NM_138555 | Group 3 |
| 244427_at | KIF23 | NM_004856 /// NM_138555 | Group 3 |
| 209408_at | KIF2C | NM_006845 | Group 3 |
| 218355_at | KIF4A | NM_012310 | Group 3 |
| 209680_s_at | KIFC1 | NM_002263 | Group 3 |
| 221841_s_at | KLF4 | NM_004235 | Group 13 |
| 231195_at | KLRG2 | NM_198508 | Group 4 |
| 205306_x_at | KMO | NM_003679 | Group 4 |
| 211138_s_at | KMO | NM_003679 | Group 4 |
| 212236_x_at | KRT17 | NM_000422 | Group 1 |
| 213680_at | KRT6B | NM_005555 | Group 1 |
| 213711_at | KRT81 | NM_002281 | Group 1 |
| 217388_s_at | KYNU | NM_001032998 /// NM_003937 | Group 4 |
| 216641_s_at | LAD1 | NM_005558 | Group 2 |
| 209270_at | LAMB3 | NM_000228 /// NM_001017402 /// | Group 1 |
| | | NM_001127641 | |
| 208029_s_at | LAPTM4B | NM_018407 | Group 4 |
| 208767_s_at | LAPTM4B | NM_018407 | Group 4 |
| 214039_s_at | LAPTM4B | NM_018407 | Group 4 |
| 201030_x_at | LDHB | NM_002300 | Group 1 |
| 213564_x_at | LDHB | NM_002300 | Group 1 |
| 203276_at | LMNB1 | NM_005573 | Group 3 |
| 242350_s_at | LOC100128098 | XM_001721625 /// XM_001722654 /// | Group 2 |
| | | XM_001725654 | |
| 243837_x_at | LOC100128500 | XM_001719603 /// XM_001720777 /// | Group 9 |
| | | XM_001720893 | |
| 1563367_at | LOC100128977 | NR_024559 /// XM_001715841 /// | Group 9 |
| | | XM_001717446 /// XM_001719146 | |
| 236656_s_at | LOC100130506 | XM_001720083 /// XM_001724500 | Group 13 |
| 244655_at | LOC100132798 | XM_001721122 /// XM_001722414 /// | Group 13 |
| | | XM_001722478 | |
| 235167_at | LOC100190986 | NR_024456 | Group 5 |
| 226809_at | LOC100216479 | — | Group 9 |
| 240838_s_at | LOC145837 | NR_026979 /// XR_040650 /// | Group 7 |
| | | XR_040651 /// XR_040652 | |
| 232034_at | LOC203274 | — | Group 9 |
| 231518_at | LOC283867 | NM_001101346 | Group 9 |
| 1560260_at | LOC285593 | NR_027108 /// NR_027109 | Group 9 |
| 1564786_at | LOC338667 | XM_001715277 /// XM_001726523 /// | Group 7 |
| | | XM_294675 | |
| 239337_at | LOC400768 | XM_378883 | Group 9 |
| 202779_s_at | LOC731049 /// | NM_014501 /// XM_001724228 | Group 3 |
| | UBE2S | | |
| 234016_at | LOC90499 | XR_042126 /// XR_042127 | Group 7 |
| 206953_s_at | LPHN2 | NM_012302 | Group 13 |
| 214109_at | LRBA | NM_006726 | Group 9 |
| 211596_s_at | LRIG1 | NM_015541 | Group 7 |
| 205710_at | LRP2 | NM_004525 | Group 9 |
| 230863_at | LRP2 | NM_004525 | Group 9 |
| 205282_at | LRP8 | NM_001018054 /// NM_004631 /// | Group 4 |
| | | NM_017522 /// NM_033300 | |
| 205381_at | LRRC17 | NM_001031692 /// NM_005824 | Group 12 |
| 220622_at | LRRC31 | NM_024727 | Group 11 |
| 222068_s_at | LRRC50 | NM_178452 | Group 7 |
| 241368_at | LSDP5 | NM_001013706 | Group 9 |
| 202728_s_at | LTBP1 | NM_000627 /// NM_206943 | Group 4 |
| 227764_at | LYPD6 | NM_194317 | Group 7 |
| 203362_s_at | MAD2L1 | NM_002358 | Group 3 |
| 212741_at | MAOA | NM_000240 | Group 9 |
| 225927_at | MAP3K1 | NM_005921 | Group 7 |
| 228262_at | MAP7D2 | NM_152780 | Group 3 |
| 203928_x_at | MAPT | NM_001123066 /// NM_001123067 /// | Group 9 |
| | | NM_005910 /// NM_016834 /// | |
| | | NM_016835 /// NM_016841 | |
| 203929_s_at | MAPT | NM_001123066 /// NM_001123067 /// | Group 9 |
| | | NM_005910 /// NM_016834 /// | |
| | | NM_016835 /// NM_016841 | |
| 206401_s_at | MAPT | NM_001123066 /// NM_001123067 /// | Group 9 |
| | | NM_005910 /// NM_016834 /// | |
| | | NM_016835 /// NM_016841 | |
| 225379_at | MAPT | NM_001123066 /// NM_001123067 /// | Group 9 |
| | | NM_005910 /// NM_016834 /// | |
| | | NM_016835 /// NM_016841 | |
| 206091_at | MATN3 | NM_002381 | Group 9 |
| 227832_at | MBD6 | NM_052897 | Group 7 |
| 227379_at | MBOAT1 | NM_001080480 | Group 9 |
| 223570_at | MCM10 | NM_018518 /// NM_182751 | Group 3 |
| 202107_s_at | MCM2 | NM_004526 | Group 3 |
| 212142_at | MCM4 | NM_005914 /// NM_182746 | Group 4 |
| 222037_at | MCM4 | NM_005914 /// NM_182746 | Group 4 |
| 205375_at | MDFI | NM_005586 | Group 1 |
| 204058_at | ME1 | NM_002395 | Group 3 |
| 204059_s_at | ME1 | NM_002395 | Group 3 |
| 204663_at | ME3 | NM_001014811 /// NM_006680 | Group 9 |
| 204825_at | MELK | NM_014791 | Group 3 |
| 203510_at | MET | NM_000245 /// NM_001127500 | Group 1 |
| 219051_x_at | METRN | NM_024042 | Group 9 |
| 232269_x_at | METRN | NM_024042 | Group 9 |
| 207761_s_at | METTL7A | NM_014033 | Group 13 |
| 226346_at | MEX3A | NM_001093725 | Group 4 |
| 227512_at | MEX3A | NM_001093725 | Group 4 |
| 225316_at | MFSD2 | NM_001136493 /// NM_032793 | Group 2 |
| 211026_s_at | MGLL | NM_001003794 /// NM_007283 | Group 13 |
| 203637_s_at | MID1 | NM_000381 /// NM_001098624 /// | Group 1 |
| | | NM_033290 | |
| 212022_s_at | MKI67 | NM_001145966 /// NM_002417 | Group 3 |
| 218883_s_at | MLF1IP | NM_024629 | Group 3 |
| 229305_at | MLF1IP | NM_024629 | Group 3 |
| 203435_s_at | MME | NM_000902 /// NM_007287 /// | Group 13 |
| | | NM_007288 /// NM_007289 | |
| 204475_at | MMP1 | NM_001145938 /// NM_002421 | Group 3 |
| 214614_at | MNX1 | NM_005515 | Group 2 |
| 218398_at | MRPS30 | NM_016640 | Group 9 |
| 243579_at | MSI2 | NM_138962 /// NM_170721 | Group 7 |
| 210319_x_at | MSX2 | NM_002449 | Group 7 |
| 212859_x_at | MT1E | NM_175617 | Group 1 |
| 216336_x_at | MT1E /// | NM_005951 /// NM_175617 /// | Group 1 |
| | MT1H /// | NM_176870 | |
| | MT1M /// | | |
| | MT1P2 | | |
| 204745_x_at | MT1G | NM_005950 | Group 1 |
| 206461_x_at | MT1H | NM_005951 | Group 1 |
| 211456_x_at | MT1P2 | — | Group 1 |
| 233436_at | MTBP | NM_022045 | Group 3 |
| 211695_x_at | MUC1 | NM_001018016 /// NM_001018017 /// | Group 7 |
| | | NM_001044390 /// NM_001044391 /// | |
| | | NM_001044392 /// NM_001044393 /// | |
| | | NM_002456 | |
| 227238_at | MUC15 | NM_001135091 /// NM_001135092 /// | Group 1 |
| | | NM_145650 | |
| 220196_at | MUC16 | NM_024690 | Group 1 |
| 1553436_at | MUC19 | XM_001126166 /// XM_001714368 /// | Group 11 |
| | | XM_001715215 /// XM_001724478 /// | |
| | | XM_497341 /// XM_936590 | |
| 213432_at | MUC5B | NM_002458 /// XM_001719349 | Group 1 |
| 1553602_at | MUCL1 | NM_058173 | Group 13 |
| 204798_at | MYB | NM_001130172 /// NM_001130173 /// | Group 9 |
| | | NM_005375 | |
| 201710_at | MYBL2 | NM_002466 | Group 3 |
| 231947_at | MYCT1 | NM_025107 | Group 13 |
| 210341_at | MYT1 | NM_004535 | Group 9 |
| 243296_at | NAMPT | NM_005746 | Group 12 |
| 228523_at | NANOS1 | NM_199461 | Group 2 |
| 214440_at | NAT1 | NM_000662 /// NM_001160170 /// | Group 9 |
| | | NM_001160171 /// NM_001160172 /// | |
| | | NM_001160173 /// NM_001160174 /// | |
| | | NM_001160175 /// NM_001160176 /// | |
| | | NM_001160179 | |
| 1553910_at | NBPF4 | NM_001143989 /// XR_040171 | Group 9 |
| 218662_s_at | NCAPG | NM_022346 | Group 3 |
| 1563369_at | NCRNA00173 | NM_207436 /// NR_027345 /// | Group 9 |
| | | NR_027346 | |
| 204162_at | NDC80 | NM_006101 | Group 3 |
| 209550_at | NDN | NM_002487 | Group 12 |
| 204412_s_at | NEFH | NM_021076 | Group 12 |
| 230291_s_at | NFIB | NM_005596 | Group 1 |
| 228278_at | NFIX | NM_002501 | Group 1 |
| 242352_at | NIPBL | NM_015384 /// NM_133433 | Group 5 |
| 219438_at | NKAIN1 | NM_024522 | Group 9 |
| 206023_at | NMU | NM_006681 | Group 4 |
| 1563512_at | NOS1AP | NM_001126060 /// NM_014697 | Group 9 |
| 215153_at | NOS1AP | NM_001126060 /// NM_014697 | Group 9 |
| 225911_at | NPNT | NM_001033047 | Group 7 |
| 205440_s_at | NPY1R | NM_000909 | Group 9 |
| 209959_at | NR4A3 | NM_006981 /// NM_173198 /// | Group 12 |
| | | NM_173199 /// NM_173200 | |
| 227971_at | NRK | NM_198465 | Group 10 |
| 218051_s_at | NT5DC2 | NM_001134231 /// NM_022908 | Group 4 |
| 203675_at | NUCB2 | NM_005013 | Group 7 |
| 229838_at | NUCB2 | NM_005013 | Group 7 |
| 223381_at | NUF2 | NM_031423 /// NM_145697 | Group 3 |
| 218039_at | NUSAP1 | NM_001129897 /// NM_016359 /// | Group 3 |
| | | NM_018454 | |
| 213125_at | OLFML2B | NM_015441 | Group 12 |
| 233446_at | ONECUT2 | NM_004852 | Group 2 |
| 239911_at | ONECUT2 | NM_004852 | Group 2 |
| 219032_x_at | OPN3 | NM_014322 | Group 4 |
| 219105_x_at | ORC6L | NM_014321 | Group 3 |
| 242912_at | P704P | NM_001145442 /// XR_040579 /// | Group 9 |
| | | XR_040580 | |
| 231018_at | PALM3 | NM_001145028 /// XM_001726585 /// | Group 9 |
| | | XM_292820 /// XM_937298 | |
| 203059_s_at | PAPSS2 | NM_001015880 /// NM_004670 | Group 4 |
| 219148_at | PBK | NM_018492 | Group 3 |
| 228905_at | PCM1 | NM_006197 | Group 9 |
| 242662_at | PCSK6 | NM_002570 /// NM_138319 /// | Group 9 |
| | | NM_138320 /// NM_138321 /// | |
| | | NM_138322 /// NM_138323 /// | |
| | | NM_138324 /// NM_138325 | |
| 202731_at | PDCD4 | NM_014456 /// NM_145341 | Group 7 |
| 212593_s_at | PDCD4 | NM_014456 /// NM_145341 | Group 7 |
| 212594_at | PDCD4 | NM_014456 /// NM_145341 | Group 7 |
| 203708_at | PDE4B | NM_001037339 /// NM_001037340 /// | Group 4 |
| | | NM_001037341 /// NM_002600 | |
| 211302_s_at | PDE4B | NM_001037339 /// NM_001037340 /// | Group 4 |
| | | NM_001037341 /// NM_002600 | |
| 205380_at | PDZK1 | NM_002614 | Group 9 |
| 208305_at | PGR | NM_000926 | Group 9 |
| 228554_at | PGR | NM_000926 | Group 9 |
| 209803_s_at | PHLDA2 | NM_003311 | Group 2 |
| 226846_at | PHYBD1 | NM_001100876 /// NM_001100877 /// | Group 7 |
| | | NM_174933 | |
| 226147_s_at | PIGR | NM_002644 | Group 13 |
| 206509_at | PIP | NM_002652 | Group 7 |
| 207469_s_at | PIR | NM_001018109 /// NM_003662 | Group 3 |
| 208502_s_at | PITX1 | NM_002653 | Group 3 |
| 209587_at | PITX1 | NM_002653 | Group 3 |
| 223551_at | PKIB | NM_032471 /// NM_181794 /// | Group 9 |
| | | NM_181795 | |
| 219702_at | PLAC1 | NM_021796 | Group 8 |
| 201860_s_at | PLAT | NM_000930 /// NM_033011 | Group 9 |
| 218640_s_at | PLEKHF2 | NM_024613 | Group 7 |
| 222699_s_at | PLEKHF2 | NM_024613 | Group 7 |
| 205913_at | PLIN | NM_001145311 /// NM_002666 | Group 13 |
| 202240_at | PLK1 | NM_005030 | Group 3 |
| 201939_at | PLK2 | NM_006622 | Group 7 |
| 204886_at | PLK4 | NM_014264 | Group 3 |
| 204887_s_at | PLK4 | NM_014264 | Group 3 |
| 204519_s_at | PLLP | NM_015993 | Group 13 |
| 225421_at | PM20D2 | NM_001010853 | Group 1 |
| 225431_x_at | PM20D2 | NM_001010853 | Group 1 |
| 239392_s_at | POGK | NM_017542 | Group 5 |
| 207746_at | POLQ | NM_199420 | Group 3 |
| 214858_at | PP14571 | NR_024014 /// XM_001719668 /// | Group 7 |
| | | XM_001722120 /// XM_001724543 | |
| 212686_at | PPM1H | NM_020700 | Group 9 |
| 226907_at | PPP1R14C | NM_030949 | Group 1 |
| 225165_at | PPP1R1B | NM_032192 /// NM_181505 | Group 2 |
| 204284_at | PPP1R3C | NM_005398 | Group 7 |
| 221088_s_at | PPP1R9A | NM_017650 | Group 8 |
| 233002_at | PPP4R4 | NM_020958 /// NM_058237 | Group 9 |
| 222158_s_at | PPPDE1 | NM_016076 | Group 5 |
| 218009_s_at | PRC1 | NM_003981 /// NM_199413 /// | Group 3 |
| | | NM_199414 | |
| 224909_s_at | PREX1 | NM_020820 | Group 9 |
| 224925_at | PREX1 | NM_020820 | Group 9 |
| 225984_at | PRKAA1 | NM_006251 /// NM_206907 | Group 10 |
| 206346_at | PRLR | NM_000949 | Group 7 |
| 204304_s_at | PROM1 | NM_001145847 /// NM_001145848 /// | Group 1 |
| | | NM_001145849 /// NM_001145850 /// | |
| | | NM_001145851 /// NM_001145852 /// | |
| | | NM_006017 | |
| 202458_at | PRSS23 | NM_007173 | Group 9 |
| 223062_s_at | PSAT1 | NM_021154 /// NM_058179 | Group 1 |
| 203355_s_at | PSD3 | NM_015310 /// NM_206909 | Group 7 |
| 209815_at | PTCH1 | NM_000264 /// NM_001083602 /// | Group 1 |
| | | NM_001083603 /// NM_001083604 /// | |
| | | NM_001083605 /// NM_001083606 /// | |
| | | NM_001083607 | |
| 225363_at | PTEN | NM_000314 | Group 9 |
| 210374_x_at | PTGER3 | NM_000957 /// NM_001126044 /// | Group 9 |
| | | NM_198712 /// NM_198713 /// | |
| | | NM_198714 /// NM_198715 /// | |
| | | NM_198716 /// NM_198717 /// | |
| | | NM_198718 /// NM_198719 | |
| 213933_at | PTGER3 | NM_000957 /// NM_001126044 /// | Group 9 |
| | | NM_198712 /// NM_198713 /// | |
| | | NM_198714 /// NM_198715 /// | |
| | | NM_198716 /// NM_198717 /// | |
| | | NM_198718 /// NM_198719 | |
| 217777_s_at | PTPLAD1 | NM_016395 | Group 6 |
| 205948_at | PTPRT | NM_007050 /// NM_133170 | Group 9 |
| 203554_x_at | PTTG1 | NM_004219 | Group 3 |
| 225418_at | PVRL2 | NM_001042724 /// NM_002856 | Group 9 |
| 242414_at | QPRT | NM_014298 | Group 2 |
| 50965_at | RAB26 | NM_014353 | Group 7 |
| 217764_s_at | RAB31 | NM_006868 | Group 9 |
| 225064_at | RABEP1 | NM_001083585 /// NM_004703 | Group 9 |
| 225092_at | RABEP1 | NM_001083585 /// NM_004703 | Group 9 |
| 222077_s_at | RACGAP1 | NM_001126103 /// NM_001126104 /// | Group 3 |
| | | NM_013277 | |
| 204146_at | RAD51AP1 | NM_001130862 /// NM_006479 | Group 3 |
| 204558_at | RAD54L | NM_001142548 /// NM_003579 | Group 3 |
| 210051_at | RAPGEF3 | NM_001098531 /// NM_001098532 /// | Group 13 |
| | | NM_006105 | |
| 218657_at | RAPGEFL1 | NM_016339 | Group 9 |
| 204070_at | RARRES3 | NM_004585 | Group 7 |
| 235004_at | RBM24 | NM_001143941 /// NM_001143942 /// | Group 9 |
| | | NM_153020 | |
| 208370_s_at | RCAN1 | NM_004414 /// NM_203417 /// | Group 13 |
| | | NM_203418 | |
| 226021_at | RDH10 | NM_172037 | Group 4 |
| 204364_s_at | REEP1 | NM_022912 | Group 7 |
| 204365_s_at | REEP1 | NM_022912 | Group 7 |
| 205645_at | REPS2 | NM_001080975 /// NM_004726 | Group 9 |
| 227425_at | REPS2 | NM_001080975 /// NM_004726 | Group 9 |
| 244745_at | RERG | NM_032918 | Group 9 |
| 215771_x_at | RET | NM_020630 /// NM_020975 | Group 9 |
| 243481_at | RHOJ | NM_020663 | Group 13 |
| 223168_at | RHOU | NM_021205 | Group 13 |
| 201785_at | RNASE1 | NM_002933 /// NM_198232 /// | Group 13 |
| | | NM_198234 /// NM_198235 | |
| 212724_at | RND3 | NM_005168 | Group 13 |
| 227722_at | RPS23 | NM_001025 | Group 9 |
| 204803_s_at | RRAD | NM_001128850 /// NM_004165 | Group 13 |
| 217728_at | S100A6 | NM_014624 | Group 1 |
| 205916_at | S100A7 | NM_002963 | Group 2 |
| 202917_s_at | S100A8 | NM_002964 | Group 2 |
| 203535_at | S100A9 | NM_002965 | Group 2 |
| 209686_at | S100B | NM_006272 | Group 13 |
| 204351_at | S100P | NM_005980 | Group 11 |
| 228653_at | SAMD5 | NM_001030060 | Group 13 |
| 229839_at | SCARA5 | NM_173833 | Group 13 |
| 235849_at | SCARA5 | NM_173833 | Group 13 |
| 201825_s_at | SCCPDH | NM_016002 | Group 9 |
| 201826_s_at | SCCPDH | NM_016002 | Group 9 |
| 206799_at | SCGB1D2 | NM_006551 | Group 11 |
| 206378_at | SCGB2A2 | NM_002411 | Group 11 |
| 219197_s_at | SCUBE2 | NM_020974 | Group 9 |
| 230290_at | SCUBE3 | NM_152753 | Group 8 |
| 240024_at | SEC14L2 | NM_012429 /// NM_033382 | Group 7 |
| 217276_x_at | SERHL2 | NM_014509 | Group 10 |
| 217284_x_at | SERHL2 | NM_014509 | Group 10 |
| 209443_at | SERPINA5 | NM_000624 | Group 9 |
| 206325_at | SERPINA6 | NM_001756 | Group 9 |
| 205933_at | SETBP1 | NM_001130110 /// NM_015559 | Group 7 |
| 202036_s_at | SFRP1 | NM_003012 | Group 1 |
| 202037_s_at | SFRP1 | NM_003012 | Group 1 |
| 235425_at | SGOL2 | NM_001160033 /// NM_001160046 /// | Group 5 |
| | | NM_152524 | |
| 221268_s_at | SGPP1 | NM_030791 | Group 13 |
| 201311_s_at | SH3BGRL | NM_003022 | Group 7 |
| 201312_s_at | SH3BGRL | NM_003022 | Group 7 |
| 219493_at | SHCBP1 | NM_024745 | Group 3 |
| 239435_x_at | SHROOM1 | NM_133456 | Group 7 |
| 209339_at | SIAH2 | NM_005067 | Group 9 |
| 206558_at | SIM2 | NM_005069 /// NM_009586 | Group 4 |
| 222939_s_at | SLC16A10 | NM_018593 | Group 4 |
| 209681_at | SLC19A2 | NM_006996 | Group 9 |
| 206396_at | SLC1A1 | NM_004170 | Group 7 |
| 213664_at | SLC1A1 | NM_004170 | Group 7 |
| 205896_at | SLC22A4 | NM_003059 | Group 7 |
| 225305_at | SLC25A29 | NM_001039355 | Group 7 |
| 232280_at | SLC25A29 | NM_001039355 | Group 7 |
| 206143_at | SLC26A3 | NM_000111 | Group 9 |
| 205769_at | SLC27A2 | NM_001159629 /// NM_003645 | Group 9 |
| 219932_at | SLC27A6 | NM_001017372 /// NM_014031 | Group 1 |
| 219215_s_at | SLC39A4 | NM_017767 /// NM_130849 | Group 3 |
| 1556551_s_at | SLC39A6 | NM_001099406 /// NM_012319 | Group 9 |
| 223044_at | SLC40A1 | NM_014585 | Group 7 |
| 233123_at | SLC40A1 | NM_014585 | Group 7 |
| 209884_s_at | SLC4A7 | NM_003615 | Group 9 |
| 207056_s_at | SLC4A8 | NM_001039960 /// NM_004858 | Group 7 |
| 1569940_at | SLC6A16 | NM_014037 | Group 2 |
| 201195_s_at | SLC7A5 | NM_003486 | Group 4 |
| 202752_x_at | SLC7A8 | NM_012244 /// NM_182728 | Group 7 |
| 216092_s_at | SLC7A8 | NM_012244 /// NM_182728 | Group 7 |
| 216603_at | SLC7A8 | NM_012244 /// NM_182728 | Group 7 |
| 201349_at | SLC9A3R1 | NM_004252 | Group 7 |
| 203021_at | SLPI | NM_003064 | Group 1 |
| 215623_x_at | SMC4 | NM_001002800 /// NM_005496 | Group 3 |
| 210057_at | SMG1 | NM_015092 | Group 5 |
| 222784_at | SMOC1 | NM_001034852 /// NM_022137 | Group 1 |
| 223235_s_at | SMOC2 | NM_022138 | Group 9 |
| 213139_at | SNAI2 | NM_003068 | Group 13 |
| 225728_at | SORBS2 | NM_001145670 /// NM_001145671 /// | Group 13 |
| | | NM_001145672 /// NM_001145673 /// | |
| | | NM_001145674 /// NM_001145675 /// | |
| | | NM_003603 /// NM_021069 | |
| 213456_at | SOSTDC1 | NM_015464 | Group 1 |
| 209842_at | SOX10 | NM_006941 | Group 1 |
| 228214_at | SOX6 | NM_001145811 /// NM_001145819 /// | Group 1 |
| | | NM_017508 /// NM_033326 | |
| 203145_at | SPAG5 | NM_006461 | Group 3 |
| 200795_at | SPARCL1 | NM_001128310 /// NM_004684 | Group 13 |
| 212558_at | SPRY1 | NM_005841 /// NM_199327 | Group 13 |
| 227725_at | ST6GALNAC1 | NM_018414 | Group 13 |
| 223103_at | STARD10 | NM_006645 | Group 9 |
| 232322_x_at | STARD10 | NM_006645 | Group 9 |
| 205542_at | STEAP1 | NM_012449 | Group 13 |
| 225987_at | STEAP4 | NM_024636 | Group 13 |
| 205339_at | STIL | NM_001048166 /// NM_003035 | Group 3 |
| 219686_at | STK32B | NM_018401 | Group 7 |
| 234310_s_at | SUSD2 | NM_019601 | Group 2 |
| 227182_at | SUSD3 | NM_145006 | Group 9 |
| 206546_at | SYCP2 | NM_014258 | Group 8 |
| 212730_at | SYNM | NM_015286 /// NM_145728 | Group 1 |
| 203998_s_at | SYT1 | NM_001135805 /// NM_001135806 /// | Group 7 |
| | | NM_005639 | |
| 1563658_a_at | SYT9 | NM_175733 | Group 7 |
| 225496_s_at | SYTL2 | NM_032379 /// NM_032943 /// | Group 7 |
| | | NM_206927 /// NM_206928 /// | |
| | | NM_206929 /// NM_206930 | |
| 232914_s_at | SYTL2 | NM_032379 /// NM_032943 /// | Group 7 |
| | | NM_206927 /// NM_206928 /// | |
| | | NM_206929 /// NM_206930 | |
| 212956_at | TBC1D9 | NM_015130 | Group 9 |
| 212960_at | TBC1D9 | NM_015130 | Group 9 |
| 219682_s_at | TBX3 | NM_005996 /// NM_016569 | Group 7 |
| 229576_s_at | TBX3 | NM_005996 /// NM_016569 | Group 7 |
| 233320_at | TCAM1 | NR_002947 | Group 1 |
| 205766_at | TCAP | NM_003673 | Group 2 |
| 204045_at | TCEAL1 | NM_001006639 /// NM_001006640 /// | Group 9 |
| | | NM_004780 | |
| 221016_s_at | TCF7L1 | NM_031283 | Group 1 |
| 223530_at | TDRKH | NM_001083963 /// NM_001083964 /// | Group 3 |
| | | NM_001083965 /// NM_006862 | |
| 1553394_a_at | TFAP2B | NM_003221 | Group 10 |
| 214451_at | TFAP2B | NM_003221 | Group 10 |
| 229341_at | TFCP2L1 | NM_014553 | Group 1 |
| 205009_at | TFF1 | NM_003225 | Group 9 |
| 204623_at | TFF3 | NM_003226 | Group 9 |
| 207332_s_at | TFRC | NM_001128148 /// NM_003234 | Group 4 |
| 204731_at | TGFBR3 | NM_003243 | Group 13 |
| 226625_at | TGFBR3 | NM_003243 | Group 13 |
| 214920_at | THSD7A | NM_015204 | Group 13 |
| 210130_s_at | TM7SF2 | NM_003273 | Group 11 |
| 219580_s_at | TMC5 | NM_001105248 /// NM_001105249 /// | Group 10 |
| | | NM_024780 | |
| 222904_s_at | TMC5 | NM_001105248 /// NM_001105249 /// | Group 10 |
| | | NM_024780 | |
| 220240_s_at | TMCO3 | NM_017905 | Group 6 |
| 226931_at | TMTC1 | NM_175861 | Group 13 |
| 214581_x_at | TNFRSF21 | NM_014452 | Group 1 |
| 215271_at | TNN | NM_022093 | Group 13 |
| 213201_s_at | TNNT1 | NM_001126132 /// NM_001126133 /// | Group 9 |
| | | NM_003283 | |
| 201292_at | TOP2A | NM_001067 | Group 3 |
| 214774_x_at | TOX3 | NM_001080430 /// NM_001146188 | Group 11 |
| 229764_at | TPRG1 | NM_198485 | Group 9 |
| 210052_s_at | TPX2 | NM_012112 | Group 3 |
| 211002_s_at | TRIM29 | NM_012101 | Group 1 |
| 204033_at | TRIP13 | NM_004237 | Group 3 |
| 224218_s_at | TRPS1 | NM_014112 | Group 8 |
| 234351_x_at | TRPS1 | NM_014112 | Group 8 |
| 206827_s_at | TRPV6 | NM_018646 | Group 2 |
| 202242_at | TSPAN7 | NM_004615 | Group 13 |
| 213122_at | TSPYL5 | NM_033512 | Group 1 |
| 237350_at | TTC36 | NM_001080441 | Group 9 |
| 204822_at | TTK | NM_003318 | Group 3 |
| 202954_at | UBE2C | NM_007019 /// NM_181799 /// | Group 3 |
| | | NM_181800 /// NM_181801 /// | |
| | | NM_181802 /// NM_181803 | |
| 223229_at | UBE2T | NM_014176 | Group 3 |
| 238657_at | UBXN10 | NM_152376 | Group 7 |
| 203343_at | UGDH | NM_003359 | Group 7 |
| 235003_at | UHMK1 | NM_175866 | Group 5 |
| 225655_at | UHRF1 | NM_001048201 /// NM_013282 | Group 3 |
| 241755_at | UQCRC2 | NM_003366 | Group 5 |
| 219211_at | USP18 | NM_017414 | Group 3 |
| 226029_at | VANGL2 | NM_020335 | Group 1 |
| 224221_s_at | VAV3 | NM_001079874 /// NM_006113 | Group 6 |
| 215729_s_at | VGLL1 | NM_016267 | Group 1 |
| 219001_s_at | WDR32 | NM_024345 | Group 7 |
| 222804_x_at | WDR32 | NM_024345 | Group 7 |
| 226511_at | WDR32 | NM_024345 | Group 7 |
| 230679_at | WDR32 | NM_024345 | Group 7 |
| 229158_at | WNK4 | NM_032387 | Group 9 |
| 208606_s_at | WNT4 | NM_030761 | Group 9 |
| 221029_s_at | WNT5B | NM_030775 /// NM_032642 | Group 1 |
| 221609_s_at | WNT6 | NM_006522 | Group 1 |
| 212637_s_at | WWP1 | NM_007013 | Group 9 |
| 206373_at | ZIC1 | NM_003412 | Group 1 |
| 229551_x_at | ZNF367 | NM_153695 | Group 3 |
| 1555800_at | ZNF385B | NM_001113397 /// NM_001113398 /// | Group 7 |
| | | NM_152520 | |
| 214761_at | ZNF423 | NM_015069 | Group 12 |
| 219741_x_at | ZNF552 | NM_024762 | Group 9 |
| 231820_x_at | ZNF587 | NM_032828 | Group 9 |
| 207494_s_at | ZNF76 | NM_003427 | Group 9 |
| 204026_s_at | ZWINT | NM_001005413 /// NM_007057 /// | Group 3 |
| | | NM_032997 | |

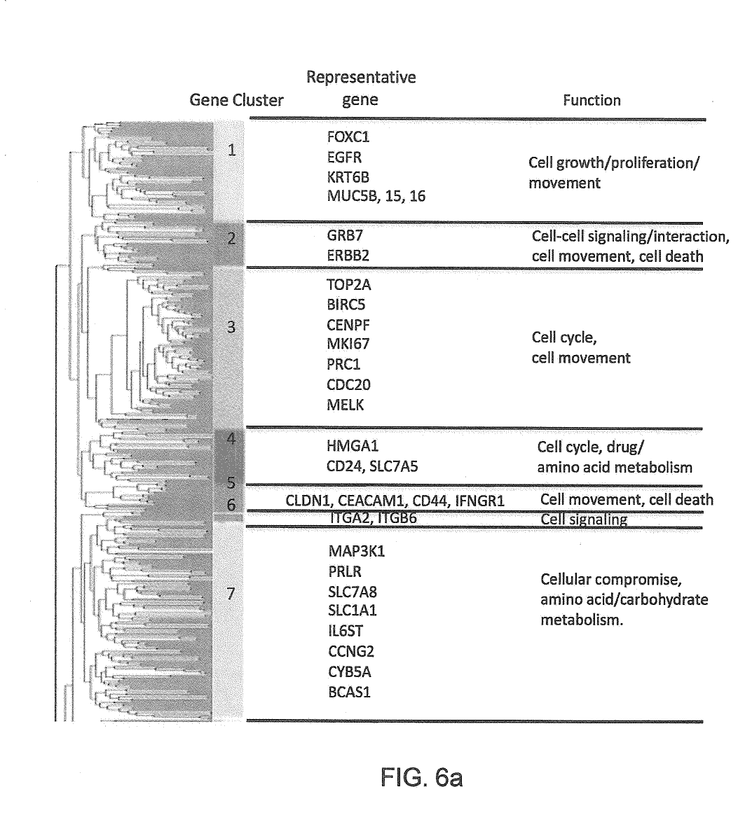

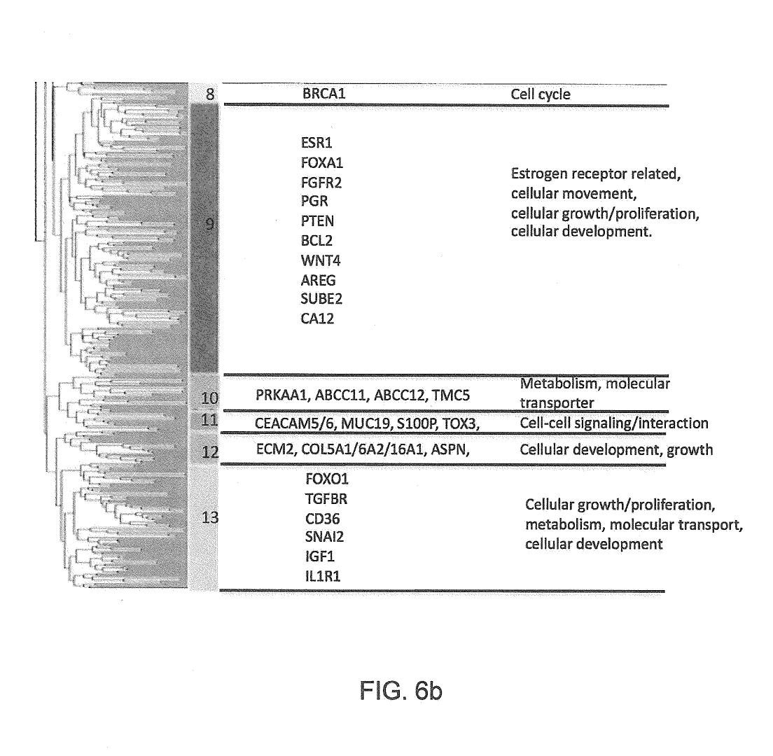

*Representative Public IDs are indicated in bold text.
# Gene clusters according to functional annotation shown in FIGS. 6a and 6b.

Alternatively, the expression levels of genes that are uniquely associated with (e.g., are differentially expressed in) one of the six molecular subtypes described herein, also referred to as a “characteristic subset” or a “molecular subtype signature,” can be analyzed to determine whether the breast cancer belongs to a particular molecular subtype. For example, to determine whether a breast cancer is a molecular subtype I breast cancer, the expression levels of genes belonging to a molecular subtype I characteristic subset (i.e., a molecular subtype I signature) (see Table 2) can be analyzed to determine whether the breast cancer is a molecular subtype I breast cancer.

As used herein, a “molecular subtype I breast cancer” refers to a breast cancer that is characterized by differential expression of the genes listed in Table 2 in a breast cancer sample relative to a normal sample (e.g., a non-cancerous control sample). Molecular subtype I breast cancers are typically chemosensitive and can be treated with adjuvant chemotherapy with or without methotrexate and/or anthracyclines according to clinical risk.

TABLE 2
Differentially-expressed Genes/Probe Sets Unique to Molecular Subtype I
Breast cancer molecular subtype I signature genes/characteristic subset

| | | Expression Compared to |
|---|---|---|
| | | Normal Breast |
| | | Tissue (“Up” indicates |
| | | up-regulation, or |
| | | increased expression; |
| | | “Down” indicates |
| Affymetrix | | down-regulation, or |
| Probeset ID | Gene Symbol | decreased expression) |
| 1438_at | EPHB3 | Up |
| 1552283_s_at | ZDHHC11 | Down |
| 1552473_at | GAMT | Down |
| 1553430_a_at | EDARADD | Down |
| 1553997_a_at | ASPHD1 | Up |
| 1554242_a_at | COCH | Up |
| 1554576_a_at | ETV4 | Up |
| 1555310_a_at | PAK6 | Up |
| 1555497_a_at | CYP4B1 | Down |
| 1555997_s_at | IGFBP5 | Down |
| 1556012_at | KLHDC7A | Down |
| 1557263_s_at | LOC100131731 | Down |
| 1558686_at | — | Down |
| 1559028_at | C21orf15 | Down |
| 1559280_a_at | — | Down |
| 200831_s_at | SCD | Down |
| 201468_s_at | NQO1 | Down |
| 201939_at | PLK2 | Down |
| 202017_at | EPHX1 | Down |
| 202219_at | SLC6A8 | Up |
| 202687_s_at | TNFSF10 | Down |
| 202862_at | FAH | Down |
| 202935_s_at | SOX9 | Up |
| 203032_s_at | FH | Up |
| 203426_s_at | IGFBP5 | Down |
| 203722_at | ALDH4A1 | Down |
| 203917_at | CXADR | Up |
| 204124_at | SLC34A2 | Up |
| 204268_at | S100A2 | Up |
| 204365_s_at | REEP1 | Down |
| 204720_s_at | DNAJC6 | Up |
| 204836_at | GLDC | Up |
| 204885_s_at | MSLN | Up |
| 204941_s_at | ALDH3B2 | Down |
| 204942_s_at | ALDH3B2 | Down |
| 204989_s_at | ITGB4 | Up |
| 205104_at | SNPH | Down |
| 205184_at | GNG4 | Up |
| 205364_at | ACOX2 | Down |
| 205375_at | MDFI | Up |
| 205402_x_at | PRSS2 | Up |
| 205697_at | SCGN | Down |
| 206204_at | GRB14 | Up |
| 206307_s_at | FOXD1 | Up |
| 206339_at | CARTPT | Down |
| 206378_at | SCGB2A2 | Down |
| 206463_s_at | DHRS2 | Down |
| 206582_s_at | GPR56 | Up |
| 207103_at | KCND2 | Down |
| 208962_s_at | FADS1 | Up |
| 209267_s_at | SLC39A8 | Up |
| 209437_s_at | SPON1 | Down |
| 209631_s_at | GPR37 | Up |
| 209909_s_at | TGFB2 | Up |
| 209975_at | CYP2E1 | Down |
| 210130_s_at | TM7SF2 | Down |
| 210297_s_at | MSMB | Down |
| 210328_at | GNMT | Down |
| 210576_at | CYP4F8 | Down |
| 212935_at | MCF2L | Down |
| 212938_at | COL6A1 | Up |
| 213107_at | TNIK | Down |
| 213385_at | CHN2 | Down |
| 213742_at | SFRS11 | Up |
| 214079_at | DHRS2 | Down |
| 214097_at | RPS21 | Up |
| 214597_at | SSTR2 | Down |
| 214798_at | ATP2C2 | Down |
| 215033_at | TM4SF1 | Up |
| 215856_at | SIGLEC15 | Down |
| 216604_s_at | SLC7A8 | Down |
| 216850_at | SNRPN | Down |
| 218309_at | CAMK2N1 | Down |
| 218704_at | RNF43 | Down |
| 218745_x_at | TMEM161A | Up |
| 218975_at | COL5A3 | Down |
| 219225_at | PGBD5 | Up |
| 219250_s_at | FLRT3 | Down |
| 219736_at | TRIM36 | Down |
| 220277_at | CXXC4 | Down |
| 220407_s_at | TGFB2 | Up |
| 220467_at | — | Down |
| 220559_at | EN1 | Up |
| 220979_s_at | ST6GALNAC5 | Up |
| 221646_s_at | ZDHHC11 | Down |
| 223218_s_at | NFKBIZ | Down |
| 223582_at | GPR98 | Down |
| 223948_s_at | TMPRSS3 | Up |
| 225667_s_at | FAM84A | Up |
| 226125_at | — | Down |
| 226649_at | PANK1 | Up |
| 226706_at | FLJ23867 /// QSOX1 | Up |
| 227259_at | CD47 | Up |
| 227285_at | C1orf51 | Up |
| 227384_s_at | LOC727820 | Down |
| 227475_at | FOXQ1 | Up |
| 228619_x_at | TIPRL | Up |
| 228708_at | RAB27B | Down |
| 228731_at | — | Down |
| 228790_at | FAM110B | Down |
| 228834_at | TOB1 | Down |
| 228977_at | LOC729680 | Up |
| 229352_at | SPESP1 | Down |
| 229927_at | LEMD1 | Up |
| 230214_at | MRVI1 | Down |
| 230337_at | SOS1 | Up |
| 230493_at | SHISA2 | Down |
| 231173_at | PYROXD1 | Up |
| 231841_s_at | KIAA1462 | Down |
| 232067_at | C6orf168 | Up |
| 232346_at | LOC388692 | Down |
| 232370_at | LOC254057 | Down |
| 232417_x_at | ZDHHC11 | Down |
| 232478_at | — | Up |
| 232573_at | — | Up |
| 233907_s_at | SERTAD4 | Up |
| 235059_at | RAB12 | Up |
| 235153_at | RNF183 | Down |
| 235318_at | FBN1 | Down |
| 235763_at | SLC44A5 | Down |
| 236417_at | — | Up |
| 236892_s_at | — | Down |
| 236947_at | — | Down |
| 237395_at | CYP4Z1 | Down |
| 237452_at | — | Up |
| 239653_at | — | Up |
| 239847_at | — | Down |
| 240052_at | ITPR1 | Down |
| 242338_at | TMEM64 | Up |
| 242874_at | — | Down |
| 244022_at | — | Up |
| 244536_at | — | Up |
| 33322_i_at | SFN | Up |

A “molecular subtype II breast cancer” refers to a breast cancer that is characterized by differential expression of the genes listed in Table 3 in a breast cancer sample relative to a normal sample (e.g., a non-cancerous control sample). Molecular subtype II breast cancers typically over-express ERBB2 and many cancers of this subtype can be treated with a therapeutic monoclonal antibody to HER2, inhibitors of the HER2/EGFR pathway, and/or high intensity chemotherapy. Molecular subtype II breast cancers typically have a high risk of developing distant metastasis and a poor survival prognosis.

TABLE 3
Differentially-expressed Genes/Probe Sets Unique to Molecular Subtype II
Breast cancer molecular subtype II signature genes/characteristic subset

| | | Expression Compared to |
|---|---|---|
| | | Normal Breast Tissue |
| | | (“Up” indicates up- |
| | | regulation, or increased |
| | | expression; “Down” |
| Affymetrix | | indicates down-regulation, |
| Probeset ID | Gene Symbol | or decreased expression) |
| 1553946_at | DCD | Up |
| 1556190_s_at | PRNP | Up |
| 1556527_a_at | — | Up |
| 201367_s_at | ZFP36L2 | Up |
| 204348_s_at | AK3L1 | Up |
| 205197_s_at | ATP7A | Up |
| 205872_x_at | PDE4DIP | Down |
| 205957_at | PLXNB3 | Up |
| 206022_at | NDP | Down |
| 207126_x_at | UGT1A1 /// UGT1A10 | Up |
| | /// UGT1A4 /// UGT1A6 | |
| | /// UGT1A8 /// UGT1A9 | |
| 208083_s_at | ITGB6 | Up |
| 208084_at | ITGB6 | Up |
| 208596_s_at | UGT1A1 /// UGT1A10 | Up |
| | /// UGT1A3 /// UGT1A4 | |
| | /// UGT1A5 /// UGT1A6 | |
| | /// UGT1A7 /// UGT1A8 | |
| | /// UGT1A9 | |
| 210262_at | CRISP2 | Up |
| 210399_x_at | FUT6 | Up |
| 211708_s_at | SCD | Up |
| 214612_x_at | MAGEA6 | Up |
| 214624_at | UPK1A | Up |
| 215125_s_at | UGT1A1 /// UGT1A10 | Up |
| | /// UGT1A3 /// UGT1A4 | |
| | /// UGT1A5 /// UGT1A6 | |
| | /// UGT1A7 /// UGT1A8 | |
| | /// UGT1A9 | |
| 217404_s_at | COL2A1 | Down |
| 219288_at | C3orf14 | Up |
| 224189_x_at | EHF | Up |
| 226271_at | GDAP1 | Down |
| 227174_at | WDR72 | Down |
| 227253_at | CP | Up |
| 230381_at | C1orf186 | Down |
| 231951_at | GNAO1 | Down |
| 234269_at | — | Up |
| 235136_at | ORMDL3 | Up |
| 239010_at | FLJ39632 | Down |
| 239605_x_at | — | Up |
| 239994_at | — | Down |
| 242343_x_at | — | Up |
| 243824_at | — | Down |
| 244508_at | 7-Sep | Up |

A “molecular subtype III breast cancer” refers to a breast cancer that is characterized by differential expression of the genes listed in Table 4 in a breast cancer sample relative to a normal sample (e.g., a non-cancerous control sample). Molecular subtype III breast cancers are typically ER-positive and, therefore, can be treated using current therapies that are effective for ER-positive breast cancers. Molecular subtype III breast cancers have an intermediate risk for distant metastasis and an intermediate survival prognosis.

TABLE 4
Differentially-expressed Genes/Probe Sets Unique to
Molecular Subtype III
Breast cancer molecular subtype III signature genes/characteristic subset

| | | Expression Compared to |
|---|---|---|
| | | Normal Breast Tissue |
| | | (“Up” indicates up-regulation, or |
| | | increased expression; “Down” |
| Affymetrix | | indicates down-regulation, |
| Probeset ID | Gene Symbol | or decreased expression) |
| 1557803_at | — | Down |
| 1567628_at | CD74 | Up |
| 1569522_at | LOC100132767 | Up |
| 201654_s_at | HSPG2 | Up |
| 202498_s_at | SLC2A3 | Up |
| 204174_at | ALOX5AP | Up |
| 204596_s_at | STC1 | Down |
| 204879_at | PDPN | Up |
| 204959_at | MNDA | Up |
| 205287_s_at | TFAP2C | Down |
| 205481_at | ADORA1 | Down |
| 205825_at | PCSK1 | Up |
| 205844_at | VNN1 | Up |
| 205987_at | CD1C | Up |
| 205997_at | ADAM28 | Up |
| 206785_s_at | KLRC1 /// KLRC2 | Up |
| 206983_at | CCR6 | Up |
| 209901_x_at | AIF1 | Up |
| 209906_at | C3AR1 | Up |
| 211990_at | HLA-DPA1 | Up |
| 212091_s_at | COL6A1 | Up |
| 212999_x_at | HLA-DQB1 | Up |
| 213095_x_at | AIF1 | Up |
| 213537_at | HLA-DPA1 | Up |
| 213830_at | TRD@ | Up |
| 213831_at | HLA-DQA1 | Up |
| 216005_at | TNC | Up |
| 217080_s_at | HOMER2 | Down |
| 217362_x_at | HLA-DRB6 | Up |
| 218345_at | TMEM176A | Up |
| 219666_at | MS4A6A | Up |
| 219759_at | ERAP2 | Up |
| 219804_at | SYNPO2L | Down |
| 220532_s_at | TMEM176B | Up |
| 221268_s_at | SGPP1 | Up |
| 221690_s_at | NLRP2 | Up |
| 222013_x_at | FAM86A | Down |
| 223280_x_at | MS4A6A | Up |
| 223820_at | RBP5 | Up |
| 223922_x_at | MS4A6A | Up |
| 223952_x_at | DHRS9 | Up |
| 224009_x_at | DHRS9 | Up |
| 224356_x_at | MS4A6A | Up |
| 226811_at | FAM46C | Up |
| 227462_at | ERAP2 | Up |
| 227860_at | CPXM1 | Up |
| 228367_at | ALPK2 | Up |
| 229674_at | SERTAD4 | Down |
| 230064_at | — | Down |
| 230312_at | — | Down |
| 231928_at | HES2 | Up |
| 232024_at | GIMAP2 | Up |
| 232170_at | S100A7A | Up |
| 235102_x_at | — | Up |
| 235104_at | ERAP2 | Up |
| 235337_at | — | Down |
| 235780_at | PRKACB | Up |
| 241272_at | — | Up |
| 243313_at | SYNPO2L | Down |
| 243366_s_at | — | Up |

A “molecular subtype IV breast cancer” refers to a breast cancer that is characterized by differential expression of the genes listed in Table 5 in a breast cancer sample relative to a normal sample (e.g., a non-cancerous control sample). Molecular subtype IV breast cancers are typically ER-positive and should be treated with an anti-estrogen therapy. Molecular subtype IV breast cancers do not respond well to methotrexate-containing chemotherapy regimen (e.g., CMF) and, therefore, should be treated with anthracycline-containing regimens (e.g., CAF) to gain better systemic control for prevention of distant metastasis and better survival. The use of Herceptin® as frontline treatment in subtype IV breast cancer with over-expression of ERBB2 is not necessary.

TABLE 5
Differentially-expressed Genes/Probe Sets Unique to
Molecular Subtype IV
Breast cancer molecular subtype IV signature genes/characteristic subset

| | | Expression Compared to Normal Breast |
|---|---|---|
| | | Tissue (“Up” indicates up-regulation, or |
| Affymetrix | Gene | increased expression; “Down” indicates |
| Probeset ID | Symbol | down-regulation, or decreased expression) |
| 1554544_a_at | MBP | Down |
| 1554819_a_at | ITGA11 | Up |
| 1556682_s_at | — | Down |
| 1564050_at | LOC642808 | Up |
| 1564233_at | FLJ33534 | Up |
| 202203_s_at | AMFR | Up |
| 202286_s_at | TACSTD2 | Down |
| 203424_s at | IGFBP5 | Up |
| 203913_s_at | HPGD | Down |
| 204933_s_at | TNFRSF11B | Down |
| 205833_s_at | PART1 | Down |
| 206697_s_at | HP | Down |
| 207929_at | GRPR | Up |
| 209030_s_at | CADM1 | Down |
| 210136_at | MBP | Down |
| 213280_at | GARNL4 | Down |
| 213462_at | NPAS2 | Down |
| 217715_x_at | — | Down |
| 218445_at | H2AFY2 | Down |
| 219823_at | LIN28 | Up |
| 219973_at | ARSJ | Down |
| 219995_s_at | ZNF750 | Down |
| 223642_at | ZIC2 | Up |
| 224840_at | FKBP5 | Down |
| 226707_at | NAPRT1 | Up |
| 226884_at | LRRN1 | Down |
| 228072_at | SYT12 | Up |
| 228676_at | ORAOV1 | Up |
| 229546_at | LOC653602 | Down |
| 230030_at | HS6ST2 | Down |
| 230563_at | RASGEF1A | Down |
| 231849_at | KRT80 | Up |
| 232360_at | EHF | Down |
| 232361_s_at | EHF | Down |
| 232567_at | ARHGAP8 | Up |
| 234331_s_at | FAM84A | Down |
| 235205_at | LOC346887 | Down |
| 235419_at | — | Down |
| 236215_at | — | Up |
| 236617_at | — | Up |
| 236926_at | TBX1 | Up |
| 243200_at | — | Down |
| 243454_at | — | Down |
| 243546_at | — | Down |
| 244216_at | — | Down |
| 39249_at | AQP3 | Down |
| 39549_at | NPAS2 | Down |

A “molecular subtype V breast cancer” refers to a breast cancer that is characterized by differential expression of the genes listed in Table 6 in a breast cancer sample relative to a normal sample (e.g., a non-cancerous control sample). Molecular subtype V breast cancers typically express high levels of estrogen receptor (ESR1) and many breast cancers of this subtype can be managed effectively with anti-estrogen hormonal therapy, without adjuvant chemotherapy, if the disease is at early stage (T<or =2; and positive node number<or =3). Molecular subtype V breast cancers typically have low risk of distant metastasis and a good survival prognosis.

TABLE 6
Differentially-expressed Genes/Probe Sets Unique to Molecular Subtype V
Breast cancer molecular subtype V signature genes/characteristic subset

| | | Expression Compared to |
|---|---|---|
| | | Normal Breast Tissue |
| | | (“Up” indicates up-regulation, |
| | | or increased expression; |
| Affymetrix | | “Down” indicates down-regulation, |
| Probeset ID | Gene Symbol | or decreased expression) |
| 1553982_a_at | RAB7B | Down |
| 1554726_at | ZNF655 | Up |
| 1560014_s_at | PDXDC1 | Up |
| 1564573_at | LOC402778 | Up |
| 1566764_at | MACC1 | Up |
| 1566869_at | — | Up |
| 1569112_at | SLC44A5 | Up |
| 201141_at | GPNMB | Down |
| 201235_s_at | BTG2 | Up |
| 201242_s_at | ATP1B1 | Up |
| 202800_at | SLC1A3 | Down |
| 202833_s_at | SERPINA1 | Up |
| 203223_at | RABEP1 | Up |
| 203423_at | RBP1 | Down |
| 203747_at | AQP3 | Up |
| 203889_at | SCG5 | Down |
| 204007_at | FCGR3B | Down |
| 204013_s_at | LCMT2 | Up |
| 204298_s_at | LOX | Down |
| 206359_at | SOCS3 | Down |
| 207718_x_at | CYP2A7 | Up |
| 210032_s_at | SPAG6 | Up |
| 210321_at | GZMH | Down |
| 211429_s_at | SERPINA1 | Up |
| 211470_s_at | SULT1C2 | Down |
| 211655_at | IGL@ | Down |
| 212094_at | PEG10 | Down |
| 213793_s_at | HOMER1 | Down |
| 214251_s_at | NUMA1 | Up |
| 214358_at | ACACA | Up |
| 215175_at | PCNX | Down |
| 215199_at | CALD1 | Down |
| 215356_at | TDRD12 | Down |
| 215777_at | IGLV4-60 | Down |
| 216430_x_at | IGL@ /// IGLV1- | Down |
| | 44 /// | |
| | LOC100290557 | |
| 216573_at | IGL@ /// IGLV1- | Down |
| | 44 /// | |
| | LOC100290557 | |
| 217320_at | LOC100293211 /// | Down |
| | LOC646057 | |
| 218792_s_at | BSPRY | Up |
| 220197_at | ATP6V0A4 | Down |
| 221261_x_at | MAGED4 /// | Down |
| | MAGED4B | |
| 221551_x_at | ST6GALNAC4 | Up |
| 221560_at | MARK4 | Up |
| 221618_s_at | TAF9B | Up |
| 221926_s_at | IL17RC | Up |
| 223217_s_at | NFKBIZ | Up |
| 223313_s_at | MAGED4 /// | Down |
| | MAGED4B | |
| 224357_s_at | MS4A4A | Down |
| 225974_at | TMEM64 | Down |
| 226622_at | MUC20 | Up |
| 227059_at | GPC6 | Down |
| 227697_at | SOCS3 | Down |
| 228705_at | CAPN12 | Down |
| 229026_at | — | Down |
| 229638_at | IRX3 | Up |
| 230051_at | C10orf47 | Up |
| 230318_at | SERPINA1 | Up |
| 230626_at | TSPAN12 | Down |
| 230664_at | H2BFM /// | Down |
| | H2BFXP | |
| 231104_at | TDRD5 | Up |
| 232280_at | SLC25A29 | Up |
| 233127_at | — | Down |
| 235501_at | — | Up |
| 235564_at | ZNF117 | Up |
| 236439_at | — | Up |
| 236517_at | MEGF10 | Up |
| 237054_at | ENPP5 | Up |
| 238717_at | — | Down |
| 238878_at | ARX | Down |
| 238884_at | — | Up |
| 240690_at | — | Up |
| 240991_at | — | Down |
| 242009_at | SLC6A4 | Up |
| 242546_at | FLJ39632 | Down |
| 243713_at | — | Up |
| 244050_at | PTPLAD2 | Up |

A “molecular subtype VI breast cancer” refers to a breast cancer that is characterized by differential expression of the genes listed in Table 7 in a breast cancer sample relative to a normal sample (e.g., a non-cancerous control sample). Molecular subtype VI breast cancers are typically ER-positive and, therefore, can be treated using current therapies that are effective for ER-positive breast cancers. Molecular subtype VI breast cancers have an intermediate risk for distant metastasis and an intermediate survival prognosis.

TABLE 7
Differentially-expressed Genes/Probe Sets
Unique to Molecular Subtype VI
Breast cancer molecular subtype VI signature genes/characteristic subset

| | | Expression Compared to Normal Breast |
|---|---|---|
| | | Tissue (“Up” indicates up-regulation, or |
| Affymetrix | Gene | increased expression; “Down” indicates |
| Probeset ID | Symbol | down-regulation, or decreased expression) |
| 1553655_at | CDC20B | Up |
| 1569399_at | — | Up |
| 200884_at | CKB | Down |
| 203946_s_at | ARG2 | Down |
| 204412_s_at | NEFH | Up |
| 204854_at | GPR162 /// | Up |
| | LEPREL2 | |
| 205990_s_at | WNT5A | Up |
| 206326_at | GRP | Up |
| 213425_at | WNT5A | Up |
| 219659_at | ATP8A2 | Up |
| 220356_at | CORIN | Up |
| 220591_s_at | EFHC2 | Up |
| 222288_at | — | Up |
| 224694_at | ANTXR1 | Up |
| 225275_at | EDIL3 | Up |
| 226085_at | CBX5 | Down |
| 229669_at | LOC440416 | Up |
| 232034_at | LOC203274 | Up |
| 235371_at | GLT8D4 | Up |
| 241864_x_at | — | Up |
| 33767_at | NEFH | Up |

Although preferable, it is not always necessary to determine the expression levels of all of the genes in a molecular subtype signature (e.g., a molecular subtype characteristic subset) to determine whether a breast cancer should be classified according to a particular molecular subtype. For example, in some cases, a breast cancer molecular subtype (e.g., a molecular subtype I) can be determined by analyzing the expression of at least about 30% of the genes in a particular molecular subtype signature. For example, in some cases, the breast cancer molecular subtype can be determined by analyzing the expression of at least about 40%, at least about 50%, at least about 60%, at least about 70%, at least about 80%, at least about 90%, at least about 95% or 100% of the genes in a molecular subtype signature described herein. Preferably the expression of at least about 70%, more preferably at least about 80%, even more preferably at least about 90% of the genes in a particular molecular subtype signature are analyzed to determine whether the breast cancer belongs to the particular breast cancer molecular subtype for which the sample is being tested.

An “immune response score” can be determined using the same basic methodology described above for molecular subtypes of a breast cancer, using the expression level of the 734 “immune response related genes” in Table 22, as well as subsets thereof, e.g., at least about 5, 10, 25, 50, 100, 200, 400, or 600 genes, or about 1, 5, 10, 20, 30, 40, 50, 60, 70, 80, 90, 95, or 99% of the 734 genes in Table 22. For example, in particular embodiments, the methods provided by the invention include the step of determining an immune response score by analyzing the expression of at least about 30% of the immune response related genes in Table 22. An immune response score of a subject can be determined from the expression levels of immune response related genes by averaging Z scores (i.e., mean, standard deviation normalized) intensities of all immune response related genes in Table 22, or a subset thereof, as described above. Cutoff values for classifying a subject as low or high immune response curve can be determined using methods known in the art, such as ROC analysis. Cutoff values can be adjusted to achieve the desired specificity (e.g., at least about 40, 50, 60, 70, 80, 82, 84, 86, 88, 90, 92, 94, 96, 98, 99%) and sensitivity (e.g., at least about 40, 50, 60, 70, 80, 82, 84, 86, 88, 90, 92, 94, 96, 98, 99%). In some embodiments, an immune response score of a subject is determined concurrently with the molecular subtype of the breast cancer, e.g., on a single microarray with a single tissue source, such as a biopsy of a breast cancer. In other embodiments, the expression levels of immune response related genes are determined from a second tissue sample from a subject—that is, other than the breast cancer biopsy. As illustrated in the examples, Applicants have demonstrated that immune response scores can be classified as high and low, respectively, where high immune response scores are predictive of improved clinical indications, such as metastasis-free survival. In particular embodiments, an immune response score is predictive (positively correlated) with the metastasis-free survival of type I and type II molecular subtypes.

Additional classification of a sample, e.g., a breast cancer, can be made either before, concurrently, or after determining the molecular subtype and/or immune response score. In some embodiments, the ERBB2 (HER2 or ERB) status (i.e., phenotype) of a sample is determined. In certain embodiments, the ER (estrogen receptor, ESR1), PR (progesterone receptor, PGR), and ERB status of a sample is determined. In particular embodiments, the ER, PR, and ERB status is determined and/or is known before determining a molecular phenotype and/or immune response score of a sample. In other embodiments, the ER, PR, and ERB status is determined concurrently with the molecular phenotype and/or immune response score of a sample. In some embodiments, ER, PR, and ERB status are determined at the nucleic acid level (e.g., by microarray). In other embodiments, they are determined at the protein level (e.g., by immunochemistry, as described in, for example, the exemplification).

A difference (e.g., an increase, a decrease) in gene expression can be determined by comparison of the level of expression of one or more genes in a sample from a subject to that of a suitable control or reference standard. Suitable controls include, for instance, a non-neoplastic tissue sample (e.g., a non-neoplastic tissue sample from the same subject from which the cancer sample has been obtained), a sample of non-cancerous cells, non-metastatic cancer cells, non-malignant (benign) cells or the like, or a suitable known or determined reference standard. The reference standard can be a typical, normal or normalized range of levels, or a particular level, of expression of a protein or RNA (e.g., an expression standard). The standards can comprise, for example, a zero gene expression level, the gene expression level in a standard cell line, or the average level of gene expression previously obtained for a population of normal human controls. Thus, the method does not require that expression of the gene/gene product be assessed in, or compared to, a control sample.

A statistically significant difference (e.g., an increase, a decrease) in the level of expression of a gene between two samples, or between a sample and a reference standard, can be determined using an appropriate statistical test(s), several of which are known to those of skill in the art. In a particular embodiment, a t-test (e.g., a one-sample t-test, a two-sample t-test) is employed to determine whether a difference in gene expression is statistically significant. For example, a statistically significant difference in the level of expression of a gene between two samples can be determined using a two-sample t-test (e.g., a two-sample Welch's t-test). A statistically significant difference in the level of expression of a gene between a sample and a reference standard can be determined using a one-sample t-test. Other useful statistical analyses for assessing differences in gene expression include a Chi-square test, Fisher's exact test, and log-rank and Wilcoxon tests.

The skilled artisan will appreciate that any of the genes disclosed herein, such as in Tables 1-7 and Table 22 include both gene names and/or reference accession numbers, such as GeneIDs, mRNA sequence accession numbers, protein sequence accession numbers, and Affymetrix ID. These identifiers may be used to retrieve, inter alia publicly-available annotated mRNA or protein sequences from sources such as the NCBI website, which may be found at the following uniform resource locator (URL): http://www.ncbi.nlm.nih.gov. The information associated with these identifiers, including reference sequences and their associated annotations, are all incorporated by reference. Useful tools for converting and/or identifying annotation IDs or obtaining additional information on a gene are known in the art and include, for example, DAVID, Clone/GeneID converter and SNAD. See Huang et al., Nature Protoc. 4(1):44-57 (2009), Huang et al., Nucleic Acids Res. 37(1)1-13 (2009), Alibes et al., BMC Bioinformatics 8:9 (2007), Sidorov et al., BMC Bioinformatics 10:251 (2009). These corresponding identifiers and reference sequences, including their annotations, are incorporated by reference.

Suitable samples for use in the methods of the invention include a tissue sample, a biological fluid sample, a cell (e.g., a tumor cell) sample, and the like. Various means of sampling from a subject, for example, by tissue biopsy, blood draw, spinal tap, tissue smear or scrape can be used to obtain a sample. Thus, the sample can be a biopsy specimen (e.g., tumor, polyp, mass (solid, cell)), aspirate, smear or blood sample.

In a preferred embodiment, the sample is a tissue sample (e.g., a biopsy of a breast tissue). The tissue sample can include all or part of a tumor (e.g., cancerous growth) and/or tumor cells. For example, a tumor biopsy can be obtained in an open biopsy in which an entire (excisional biopsy) or partial (incisional biopsy) mass is removed from a target area. Alternatively, a tumor sample can be obtained through a percutaneous biopsy, a procedure performed with a needle-like instrument through a small incision or puncture (with or without the aid of an imaging device) to obtain individual cells or clusters of cells (e.g., a fine needle aspiration (FNA)) or a core or fragment of tissues (core biopsy). The biopsy samples can be examined cytologically (e.g., smear), histologically (e.g., frozen or paraffin section) or using any other suitable method (e.g., molecular diagnostic methods). A tumor sample can also be obtained by in vitro harvest of cultured human cells derived from an individual's tissue. Tumor samples can, if desired, be stored before analysis by suitable storage means that preserve a sample's protein and/or nucleic acid in an analyzable condition, such as quick freezing, or a controlled freezing regime. If desired, freezing can be performed in the presence of a cryoprotectant, for example, dimethyl sulfoxide (DMSO), glycerol, or propanediol-sucrose. Tumor samples can be pooled, as appropriate, before or after storage for purposes of analysis.

Many suitable techniques for measuring gene expression in a sample are known to those of ordinary skill in the art and include, for example, gene expression profiling techniques, Northern blot analysis, RT-PCR, and in situ hybridization, among others. In a particular embodiment, the methods of the invention comprise generating a gene expression profile for a breast cancer and comparing the gene expression profile of the breast cancer to one or more reference gene expression profiles (e.g., a gene expression profile for a normal, non-cancerous sample; a standard or typical gene expression profile for a breast cancer molecular subtype) to determine the molecular subtype of the breast cancer.

Various well known methods for obtaining a gene expression profile can be employed. For example, a library of oligonucleotides in microchip format (e.g., a gene chip, a microarray) can be constructed to contain a set of probe oligodeoxynucleotides that are specific for a set of genes (e.g., genes from one or more of the molecular subtype signatures described herein). For example, probe oligonucleotides of an appropriate length can be 5′-amine modified at position C6 and printed using commercially available microarray systems, e.g., the GeneMachine OmniGrid™ 100 Microarrayer and Amersham CodeLink™ activated slides. Labeled cDNA oligomers corresponding to the target RNAs are prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g. 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding gene products, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Images intensities of each spot on the array are proportional to the abundance of the corresponding gene product in the patient sample.

In particular embodiments, gene expression levels are determined using an AFFYMETRIX™ microarray, such as an Exon 1.0 ST, Gene 1.0 ST, U 95, U133, U133A 2.0, or U133 Plus 2.0 microarray. In more particular embodiments, the microarray is an AFFYMETRIX™ U133A 2.0 or U133 Plus 2.0 array.

Using a gene chip or microarray, the expression level of multiple RNA transcripts in a sample from a subject can be determined by extracting RNA (e.g., total RNA) from a sample from the subject, reverse transcribing the RNAs from the sample to generate a set of target oligodeoxynucleotides and hybridizing target oligodeoxynucleotides to probe oligodeoxynucleotides on the gene chip or microarray to generate a gene expression profile (also referred to as a hybridization profile). The gene expression profile comprises the signal from the binding of the target oligodeoxynucleotides from the sample to the gene-specific probe oligonucleotides on the microarray. The profile can be recorded as the presence or absence of binding (signal vs. zero signal). More preferably, the profile recorded includes the intensity of the signal from each hybridization. Gene expression on an array or gene chip can be assessed using an appropriate algorithm (e.g., statistical algorithm). Suitable software applications for assessing gene expression levels using a microarray or gene chip are known in the art. In a particular embodiment, gene expression on a microarray is assessed using Affymetrix Microarray Analysis Suite (MAS) 5.0 software and/or DNA Chip Analyzer (dChip) software.

The resulting gene expression profile, or hybridization profile, serves as a fingerprint that is unique to the state of the sample. That is, breast cancer tissue can be distinguished from normal tissue, and within breast cancer tissue, different molecular subtypes (e.g., molecular subtypes I-VI) can be distinguished. The identification of genes that are differentially expressed in breast cancer tissue versus normal tissue, as well as differentially expressed in the six molecular subtypes of breast cancer identified herein, can be used to select an effective and/or optimal treatment regimen for the subject. For example, a particular treatment regime can be evaluated (e.g., to determine whether a chemotherapeutic drug acts to improve the long-term prognosis in a particular patient). Similarly, diagnosis can be done or confirmed by comparing patient samples with the known expression profiles. Furthermore, these gene expression profiles (or individual genes) allow screening of drug candidates that suppress the breast cancer expression profile or convert a poor prognosis profile to a better prognosis profile.

The gene expression profile of the breast cancer sample can be compared to a control or reference profile to determine the molecular subtype of the breast cancer in the test sample. In one embodiment, the control or reference profile is a gene expression profile obtained from one or more normal (e.g., non-cancerous, non-malignant) samples, such as a normal breast tissue sample. By comparing the gene expression profile of the breast cancer sample to the gene expression profile of a normal control sample, one of ordinary skill in the art can readily identify which genes are differentially expressed (e.g., upregulated, downregulated) in the breast cancer sample relative to the normal sample(s). Once the genes that are differentially expressed in the breast cancer sample relative to the normal sample are identified, the molecular subtype of the breast cancer can be determined by comparing the differentially expressed genes in the breast cancer sample to one or more of the molecular subtype signatures described herein (Tables 2-7). The molecular subtype signature that most closely matches the differentially expressed genes in the breast cancer sample corresponds to the molecular subtype of the breast cancer sample.

In another embodiment, the control or reference profile is a gene expression profile obtained from one or more samples belonging to one of the six breast cancer molecular subtypes described herein. Preferably, the control or reference profile is a typical or average gene expression profile for one of the six breast cancer molecular subtypes described herein (e.g., a gene expression profile obtained from several representative samples of a particular breast cancer molecular subtype). A gene expression profile for a breast cancer sample that is substantially similar to a control or reference gene expression profile for a particular molecular subtype indicates that the breast cancer in the sample has the same molecular subtype as the control or reference profile. Thus, by comparing the gene expression profile of the breast cancer sample to a control or reference gene expression profile for a particular molecular subtype, one of ordinary skill in the art can readily determine whether the breast cancer in the sample belongs to the molecular subtype of the control or reference profile.

Other well known techniques for measuring gene expression in a sample include, for example, Northern blot analysis, RT-PCR, in situ hybridization. Such techniques can also be employed in the methods of the invention to determine the molecular subtype of a breast cancer. For example, the level of at least one gene product can be detected using Northern blot analysis. For Northern blot analysis, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes for Northern blot hybridization include nucleic acid probes that are complementary to the nucleotide sequences of the RNA (e.g., mRNA) and/or cDNA sequences of the genes of the CNS. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference. For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like. Probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are herein incorporated by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of gene transcript levels. Using another approach, gene transcript levels can be quantified by computerized imaging systems, such the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

The levels of RNA transcripts can also be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given gene product can be produced, for example, from the nucleic acid sequences of the RNA products of the CNS genes described herein.

Levels of a nucleic acid (e.g., mRNA transcript) in a sample from a subject can also be assessed using any standard nucleic acid amplification technique, such as, for example, polymerase chain reaction (PCR) (e.g., direct PCR, quantitative real time PCR (qRT-PCR), reverse transcriptase PCR (RT-PCR)), ligase chain reaction, self sustained sequence replication, transcriptional amplification system, Q-Beta Replicase, or the like, and visualized, for example, by labeling of the nucleic acid during amplification, exposure to intercalating compounds/dyes, probes, etc. In a particular embodiment, the relative number of gene transcripts in a sample is determined by reverse transcription of gene transcripts (e.g., mRNA), followed by amplification of the reverse-transcribed products by polymerase chain reaction (e.g., RT-PCR). The levels of gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art.

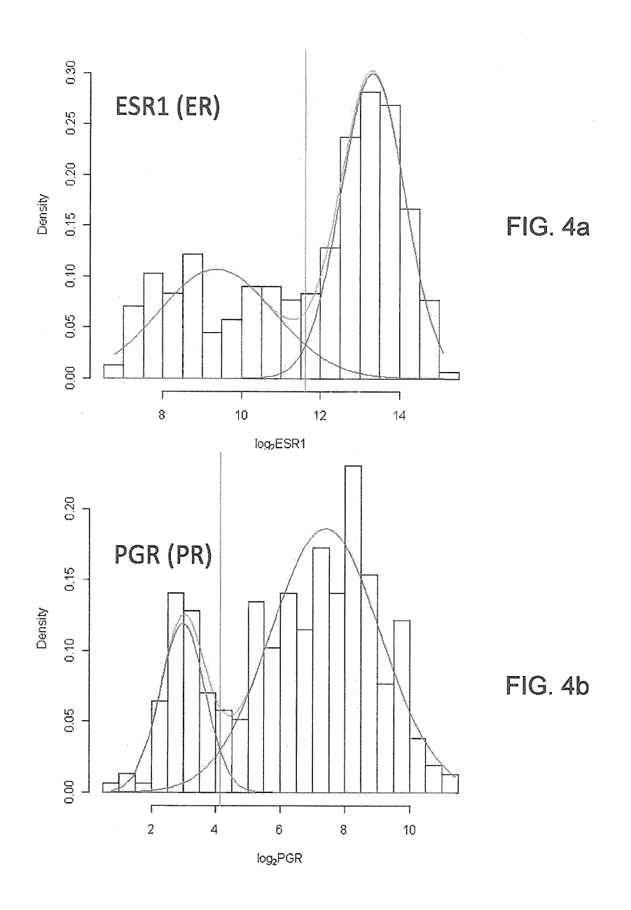

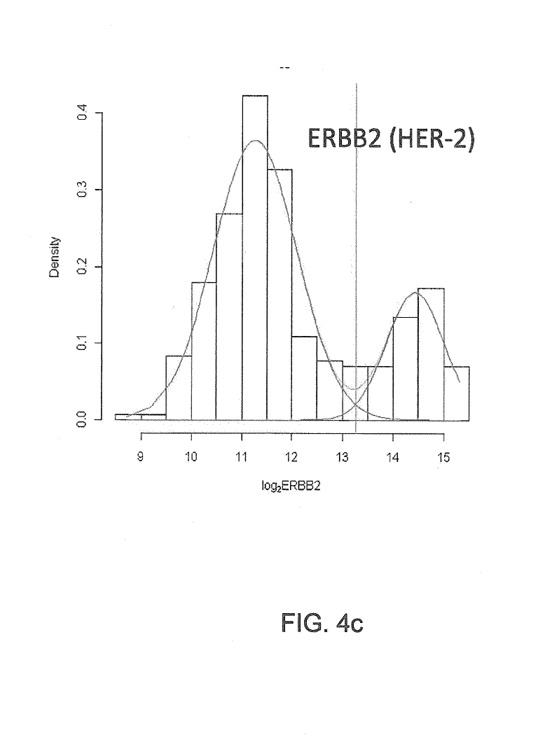

In a particular embodiment, fragments of RNA transcripts for any of the 55 tumor-specific genes described herein (see FIG. 4) can be identified in the blood (e.g., blood plasma) or other bodily fluids (e.g., blood or other body fluids that contain cancer cells) of a subject and quantified, e.g., by performing reverse transcription, PCR and parallel sequencing as described by Palacios G, et al., New Eng. J. Med. 358: 991-998 (2008). The identity of any RNA fragment can be determined by matching its sequence to one of the cDNA sequences of the 55 tumor specific genes. RNA fragments of the 55 tumor-specific genes can also be quantified according to the frequency with which a fragment having a particular DNA sequence from among the 55 tumor-specific genes is detected among all the sequenced PCR fragments from the sample. This approach can be used to screen and identify subjects that are positive for cancer cells. Alternatively, the identities of fragments of RNA transcripts for any of the 55 tumor-specific genes in a blood or biological fluid sample from a subject can be determined and quantified, for example, by performing reverse transcription of the RNA fragment(s), followed by PCR amplification and hybridization of the PCR product(s) to an array (e.g., a microarray, a gene chip).

Other techniques for measuring gene expression in a sample are also known to those of skill in the art, and include various techniques for measuring rates of RNA transcription and degradation.

Alternatively, the level of expression of a gene in a sample can be determined by assessing the level of a protein(s) encoded by the gene. Methods for detecting a protein product of a gene include, for example, immunological and immunochemical methods, such as flow cytometry (e.g., FACS analysis), enzyme-linked immunosorbent assays (ELISA), chemiluminescence assays, radioimmunoassay, immunoblot (e.g., Western blot), immunohistochemistry (IHC), and mass spectrometry. For instance, antibodies to a protein product of a gene can be used to determine the presence and/or expression level of the protein in a sample either directly or indirectly e.g., using immunohistochemistry (IHC). For example, paraffin sections can be taken from a biopsy, fixed to a slide and combined with one or more antibodies by suitable methods.

Methods for Determining a Prognosis for a Patient with a Breast Cancer

As described herein, it has also been found that an association exists between certain breast cancer molecular subtypes and a patient prognosis (e.g., survival, risk of metastases/distant metastases (see, e.g., Example 2). Specifically, molecular subtype II breast cancer is associated with the highest risk of distant metastasis and poor survival prospects, followed by molecular subtype IV breast cancer. Molecular subtypes III and VI breast cancers are associated with an intermediate risk for distant metastasis and intermediate survival prospects. In contrast, molecular subtype V breast cancer is associated with a low risk for distant metastasis and more favorable survival prospects. Accordingly, a prognosis for a subject with a breast cancer can be determined by classifying the breast cancer according to one of the molecular subtypes described herein. In particular embodiments, the breast cancer in the subject is classified by any of the methods provided by the invention and the prognosis is based on the classification of the breast cancer, wherein the prognosis is for one or more clinical indicators selected from metastasis risk, T stage, TNM stage, metastasis-free survival, and overall survival.

### Methods of Treatment

In one embodiment, the present invention relates to a method of treating a breast cancer in a subject, comprising determining the molecular subtype of the breast cancer in the subject and administering to the subject a therapy that is effective for treating the molecular subtype of the breast cancer. Methods described herein for determining the molecular subtype of a breast cancer in a subject can be employed in the treatment methods described herein.

In a particular embodiment, the molecular subtype of the breast cancer in the subject is a molecular subtype I breast cancer and a therapy that is effective for treating a molecular subtype I breast cancer is administered to the subject. Therapies that are effective for treating a molecular subtype I breast cancer include, for example, a therapy that includes at least one adjuvant therapy. Exemplary adjuvant therapies include adjuvant chemotherapy (e.g., tamoxifen, cisplatin, mitomycin, 5-fluorouracil, doxorubicin, sorafenib, octreotide, dacarbazine (DTIC), Cis-platinum, cimetidine, cyclophophamide), adjuvant radiation therapy (e.g., proton beam therapy), adjuvant hormone therapy (e.g., anti-estrogen therapy, androgen deprivation therapy (ADT), luteinizing hormone-releasing hormone (LH-RH) agonists, aromatase inhibitors (AIs, such as anastrozole, exemestane, letrozole), estrogen receptor modulators (e.g., tamoxifen, raloxifene, toremifene)), and adjuvant biological therapy, among others. In a particular embodiment, the adjuvant therapy is an adjuvant chemotherapy. In clinically low risk patients (i.e., those having a tumor with a size less than or equal to T2 and a positive node number less than or equal to 3), the adjuvant chemotherapy for a molecular subtype I breast cancer is preferably equivalent in intensity to a standard methotrexate chemotherapy (CMF). In clinically high risk patients, defined as having a tumor with a grade higher than T2 and a positive node number higher than N2, the adjuvant chemotherapy for a molecular subtype I breast cancer is preferably higher in intensity than a standard methotrexate chemotherapy.

In another embodiment, the molecular subtype of the breast cancer in the subject is a molecular subtype II breast cancer and a therapy that is effective for treating a molecular subtype II breast cancer is administered to the subject. Therapies that are effective for treating a molecular subtype II breast cancer include, for example, administration of one or more HER2/EGFR signaling pathway antagonists, a high intensity chemotherapy and a dose-dense chemotherapy. Suitable HER2/EGFR signaling pathway antagonists for a molecular subtype II breast cancer therapy include lapatinib (Tykerb®) and trastuzumab (Herceptin®). In particular embodiments, a HER2/EGFR signaling pathway antagonist is administered to the subject. In still more particular embodiments, the breast cancer overexpresses HER2.

In some embodiments, an adjuvant chemotherapy is administered to a subject. In more particular embodiments, the adjuvant chemotherapy comprises methotrexate. In still more particular embodiments, before determining the molecular subtype of the breast cancer, the subject is a candidate for receiving adjuvant chemotherapy comprising one or more anthracyclines (e.g., such a candidate as determined using previously standard criteria for recommending adjuvant therapy) and after determining the molecular subtype an anthracycline is not administered. In yet more particular embodiments, the breast cancer is determined to be a molecular subtype I, II, III, V, or VI and in still more particular embodiments, the breast cancer is a molecular subtype I.

In an additional embodiment, the molecular subtype of the breast cancer in the subject is a molecular subtype IV breast cancer and a therapy that is effective for treating a molecular subtype IV breast cancer is administered to the subject. Therapies that are effective for treating a molecular subtype IV breast cancer include, for example, anti-estrogen therapies, such as an adjuvant chemotherapy that comprises administration of at least one anthracycline compound. Suitable anthracycline compounds for use in a molecular subtype IV breast cancer therapy include doxorubicin (Adriamycin®), epirubicin (Ellence®), daunomycin and idarubicin. In a particular embodiment, a molecular subtype IV breast cancer therapy includes an adjuvant chemotherapy that comprises administration of doxorubicin (Adriamycin®). Molecular subtype IV breast cancers do not respond well to methotrexate-containing chemotherapy, which should not be used to treat molecular subtype IV breast cancers. Accordingly, in some embodiments, before determining the molecular subtype of the breast cancer the subject is a candidate for therapy comprising administering methotrexate and not an anthracycline, but after determining the molecular subtype, the subject is a candidate for receiving an anthracycline. In other embodiments, before determining the molecular subtype, the subject is a candidate for receiving a HER2/EGFR signaling pathway antagonist, but after determining the molecular subtype, the subject is not candidate for a HER2/EGFR signaling pathway antagonist. In more particular embodiments, the breast cancer overexpresses HER2 and in still more particular embodiments, the HER2 phenotype of the breast cancer is known before determining its molecular subtype.

In a further embodiment, the molecular subtype of the breast cancer in the subject is a molecular subtype V breast cancer and a therapy that is effective for treating a molecular subtype V breast cancer is administered to the subject. Therapies that are effective for treating a molecular subtype V breast cancer include, for example, anti-estrogen therapies. Preferably, the therapy does not include an adjuvant chemotherapy when the breast cancer is at an early stage (i.e., a tumor with size less than or equal to T2 and a positive node number less than or equal to 3). Anti-estrogen therapies that are useful for treating a molecular subtype V breast cancer include therapies that lower the amount of the hormone estrogen in the body (e.g., administration of aromatase inhibitors) or therapies that block the action of estrogen on breast cancer cells (e.g., administration of tamoxifen). Typically, anti-estrogen therapies for a molecular subtype V breast cancer therapy include administration of one or more antiestrogen agents. Exemplary antiestrogen agents for the methods of the invention include, but are not limited to, antiestrogen compounds (e.g., indole derivatives, such as indolo carbazole (ICZ)), aromatase inhibitors (e.g., Arimidex® (chemical name: anastrozole), Aromasin® (chemical name: exemestane), Femara® (chemical name: letrozole)); Selective Estrogen Receptor Modulators (SERMs) (e.g., Nolvadex® (chemical name: tamoxifen), Evista® (chemical name: raloxifene), Fareston® (chemical name: toremifene)); and Estrogen Receptor Downregulators (ERDs) (e.g., Faslodex® (chemical name: fulvestrant)).

In yet another embodiment, the molecular subtype of the breast cancer in the subject is a molecular subtype III or a molecular subtype VI breast cancer and a therapy that is effective for treating a molecular subtype III or VI breast cancer is administered to the subject. Therapies that are effective for treating a molecular subtype III or VI breast cancer include, for example, therapies that include anti-estrogen therapies, such as the anti-estrogen therapies described herein.

In certain embodiments, the methods of treatment provided by the invention include the step of determining an immune response score of the subject. In more particular embodiments, the breast cancer in the subject is molecular subtype I or molecular subtype II. In still more particular embodiments, the breast cancer in the subject is molecular subtype I or molecular subtype II and the subject has a low immune response score. In still more particular embodiments, the breast cancer in the subject is molecular subtype I or molecular subtype II, the subject has a low immune response score and an adjuvant therapy, such as a chemotherapy, such as one or more anthracyclines, is administered and/or prescribed. In other embodiments, the invention provides methods where a subject is determined to have a high immune response score and a less aggressive course of treatment is administered,

An effective therapy for a given breast cancer molecular subtype typically includes a primary therapy (e.g., as the principal therapeutic agent in a therapy or treatment regimen, such as surgery or radiotherapy); and, optionally, an adjunct therapy (e.g., as a therapeutic agent used together with another therapeutic agent in a therapy or treatment regime, wherein the combination of therapeutic agents provides the desired treatment; “adjunct therapy” is also referred to as “adjunctive therapy”). In some embodiments, an effective therapy for a given breast cancer molecular subtype can include an adjuvant therapy (e.g., a therapeutic agent that is given to the subject in need thereof after the principal therapeutic agent in a therapy or treatment regimen has been given). Suitable adjuvant therapies include, but are not limited to, chemotherapy (e.g., tamoxifen, cisplatin, mitomycin, 5-fluorouracil, doxorubicin, sorafenib, octreotide, dacarbazine (DTIC), Cis-platinum, cimetidine, cyclophophamide), radiation therapy (e.g., proton beam therapy), hormone therapy (e.g., anti-estrogen therapy, androgen deprivation therapy (ADT), luteinizing hormone-releasing hormone (LH-RH) agonists, aromatase inhibitors (AIs, such as anastrozole, exemestane, letrozole), estrogen receptor modulators (e.g., tamoxifen, raloxifene, toremifene)), and biological therapy. Numerous other therapies can also be administered during a cancer treatment regime to mitigate the effects of the disease and/or side effects of the cancer treatment including therapies to manage pain (narcotics, acupuncture), gastric discomfort (antacids), dizziness (anti-vertigo medications), nausea (anti-nausea medications), infection (e.g., medications to increase red/white blood cell counts) and the like, all of which are readily appreciated by the person skilled in the art.

In the methods of the invention, an adjuvant therapy can be administered before, after or concurrently with a primary therapy like radiation therapy and/or the surgical removal of a tumor(s). If more than one adjuvant therapy is employed (e.g., a chemotherapeutic agent and a targeted therapeutic agent) the adjuvant therapies can be co-administered simultaneously (e.g., concurrently) as either separate formulations or as a joint formulation. Alternatively, the adjuvant therapies can be administered sequentially, as separate compositions, within an appropriate time frame (e.g., a cancer treatment session/interval such as 1.5 to 5 hours) as determined by the skilled clinician (e.g., a time sufficient to allow an overlap of the pharmaceutical effects of the therapies). The adjuvant therapies and/or the primary therapy can be administered in a single dose or multiple doses in an order and on a schedule suitable to achieve a desired therapeutic effect (e.g., inhibition of tumor growth, inhibition of angiogenesis, and/or inhibition of cancer metastasis).

Thus, one or more therapeutic agents can be administered in single or multiple doses. Suitable dosing and regimens of administration can be determined by a skilled clinician and are dependent on the agent(s) chosen, the pharmaceutical formulation and the route of administration, as well as various patient factors and other considerations. The amount of a therapeutic agent to be administered (e.g., a therapeutically effective amount) can be determined by a clinician using the guidance provided herein and other methods known in the art and is dependent on several factors including, for example, the particular agent chosen, the subject's age, sensitivity, tolerance to drugs and overall well-being. For example, suitable dosages for a small molecule can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Suitable dosages for an antibody can be from about 0.01 mg/kg to about 300 mg/kg body weight per treatment and preferably from about 0.01 mg/kg to about 100 mg/kg, from about 0.01 mg/kg to about 10 mg/kg, from about 1 mg/kg to about 10 mg/kg body weight per treatment. When the agent is a polypeptide (linear, cyclic, mimetic), the preferred dosage will result in a plasma concentration of the peptide from about 0.1 μg/mL to about 200 μg/mL. Determining the dosage for a particular agent, patient and breast cancer is well within the abilities of one of skill in the art. Preferably, the dosage does not cause or produces minimal adverse side effects (e.g., immunogenic response, nausea, dizziness, gastric upset, hyperviscosity syndromes, congestive heart failure, stroke, pulmonary edema

In one aspect, an effective therapy for a breast cancer molecular subtype is administered to a subject in need thereof to inhibit breast cancer tumor growth or kill breast cancer tumor cells. For example, agents which directly inhibit tumor growth (e.g., chemotherapeutic agents) are conventionally administered at a particular dosing schedule and level to achieve the most effective therapy (e.g., to best kill tumor cells). Generally, about the maximum tolerated dose is administered during a relatively short treatment period (e.g., one to several days), which is followed by an off-therapy period. In a particular example, the chemotherapeutic cyclophosphamide is administered at a maximum tolerated dose of 150 mg/kg every other day for three doses, with a second cycle given 21 days after the first cycle. (Browder et al. Can Res 60:1878-1886, 2000).

An effective therapy for a given breast cancer molecular subtype can be administered, for example, in a first cycle in which about the maximum tolerated dose of a therapeutic agent is administered in one interval/dose, or in several closely spaced intervals (minutes, hours, days) with another/second cycle administered after a suitable off-therapy period (e.g., one or more weeks). Suitable dosing schedules and amounts for a therapeutic agent can be readily determined by a clinician of ordinary skill. Decreased toxicity of a particular targeted therapeutic agent as compared to chemotherapeutic agents can allow for the time between administration cycles to be shorter. When used as an adjuvant therapy (to, e.g., surgery, radiation therapy, other primary therapies), a therapeutically-effective amount of a therapeutic agent is preferably administered on a dosing schedule determined by the skilled clinician to be more/most effective at inhibiting (reducing, preventing) breast cancer tumor growth.

In another aspect, an effective therapy for a given breast cancer molecular subtype can be administered in a metronomic dosing regime, whereby a lower dose is administered more frequently relative to maximum tolerated dosing. A number of preclinical studies have demonstrated superior anti-tumor efficacy, potent antiangiogenic effects, and reduced toxicity and side effects (e.g., myelosuppression) of metronomic regimes compared to maximum tolerated dose (MTD) counterparts (Bocci, et al., Cancer Res, 62:6938-6943, (2002); Bocci, et al., Proc. Natl. Acad. Sci., 100(22):12917-12922, (2003); and Bertolini, et al., Cancer Res, 63(15):4342-4346, (2003)). Metronomic chemotherapy appears to be effective in overcoming some of the shortcomings associated with chemotherapy.

An effective therapy for a given breast cancer molecular subtype can be administered in a metronomic dosing regime to inhibit (reduce, prevent) angiogenesis in a patient in need thereof as part of an anti-angiogenic therapy. Such anti-angiogenic therapy can indirectly affect (inhibit, reduce) tumor growth by blocking the formation of new blood vessels that supply tumors with nutrients needed to sustain tumor growth and enable tumors to metastasize. Starving the tumor of nutrients and blood supply in this manner can eventually cause the cells of the tumor to die by necrosis and/or apoptosis. Previous work has indicated that the clinical outcomes (inhibition of endothelial cell-mediated tumor angiogenesis and tumor growth) of cancer therapies that involve the blocking of angiogenic factors (e.g., VEGF, bFGF, TGF-α, IL-8, PDGF) or their signaling have been more efficacious when lower dosage levels are administered more frequently, providing a continuous blood level of the antiangiogenic agent. (See Browder et al. Can. Res. 60:1878-1886, 2000; Folkman J., Sem. Can. Biol. 13:159-167, 2003). An anti-angiogenic treatment regimen has been used with a targeted inhibitor of angiogenesis (thrombospondin 1 and platelet growth factor-4 (TNP-470)) and the chemotherapeutic agent cyclophosphamide. Every 6 days, TNP-470 was administered at a dose lower than the maximum tolerated dose and cyclophosphamide was administered at a dose of 170 mg/kg. Id. This treatment regimen resulted in complete regression of the tumors. Id. In fact, anti-angiogenic treatments are most effective when administered in concert with other anti-cancer therapeutic agents, for example, those agents that directly inhibit tumor growth (e.g., chemotherapeutic agents). Id.

A variety of routes of administration can be used for therapeutic agents employed in the methods of the invention including, for example, oral, topical, transdermal, rectal, parenteral (e.g., intraaterial, intravenous, intramuscular, subcutaneous injection, intradermal injection), intravenous infusion and inhalation (e.g., intrabronchial, intranasal or oral inhalation, intranasal drops) routes of administration, depending on the agent and the particular breast cancer molecular subtype to be treated. Administration can be local or systemic as indicated. The preferred mode of administration can vary depending on the particular agent chosen.

In many cases it will be preferable to administer a large loading dose of a therapeutic agent followed by periodic (e.g., weekly) maintenance doses over the treatment period. Therapeutic agents can also be delivered by slow-release delivery systems, pumps, and other known delivery systems for continuous infusion. Dosing regimens can be varied to provide the desired circulating levels of a particular therapeutic agent based on its pharmacokinetics. Thus, doses will be calculated so that the desired therapeutic level is maintained.

The actual dose and treatment regimen can be determined by a skilled physician, taking into account the nature of the cancer (primary or metastatic), the number and size of tumors, other therapies being employed, and patient characteristics. In view of the life-threatening nature of certain breast cancer molecular subtypes, large doses with significant side effects can be employed.

### Kits of the Invention

The present invention also encompasses kits for classifying a breast cancer according to one of the six molecular subtypes described herein. Kits of the invention include a collection (e.g., a plurality) of probes capable of detecting the expression level of multiple genes in a molecular subtype signature described herein (i.e., a molecular subtype I signature, a molecular subtype II signature, a molecular subtype III signature, a molecular subtype IV signature, a molecular subtype V signature, a molecular subtype VI signature, as well as the immune response score). For example, the kits can include a collection of probes capable of detecting the level of expression of the majority of genes in a molecular subtype signature described herein, for example about 55, 60, 65, 70, 75, 80, 85, 90, 95, 99 or 100% of the genes in a molecular subtype signature described herein. In one embodiment, the kit encompasses a collection of probes capable of detecting the level of expression of each gene in a molecular subtype signature described herein. In particular embodiments, the kits provided by the invention comprise a collection of probes capable of detecting the level of expression of about 30% of the genes in Table 1. In more particular embodiments, the kits may further comprise a collection of probes capable of detecting the level of expression of about 30% of the genes in Table 22.

The probes employed in the kits of the invention include, but are not limited to, nucleic acid probes and antibodies. Accordingly, in one embodiment, the kit comprises nucleic acid probes (e.g., oligonucleotide probes, polynucleotide probes) that specifically hybridize to an RNA transcript (e.g., mRNA, hnRNA) of a gene in a molecular subtype signature described herein. Such probes are capable of binding (i.e., hybridizing) to a target nucleic acid of complementary sequence through one or more types of chemical bonds, usually through complementary base pairing via hydrogen bond formation. As used herein, a nucleic acid probe can include natural (i.e., A, G, U, C or T) or modified bases (7-deazaguanosine, inosine, etc.). In addition, the bases in the nucleic acid probes can be joined by a linkage other than a phosphodiester bond, so long as the linkage does not interfere with hybridization. Thus, probes can be peptide nucleic acids in which the constituent bases are joined by peptide bonds rather than phosphodiester linkages.

Guidance for performing hybridization reactions can be found in Current Protocols in Molecular Biology, John Wiley & Sons, N.Y. (1989), 6.3.1-6.3.6, the relevant teachings of which are incorporated herein by reference in their entirety. Suitable hybridization conditions resulting in specific hybridization vary depending on the length of the region of homology, the GC content of the region, and the melting temperature (“Tm”) of the hybrid. Thus, hybridization conditions can vary in salt content, acidity, and temperature of the hybridization solution and the washes. Complementary hybridization between a probe nucleic acid and a target nucleic acid involving minor mismatches can be accommodated by reducing the stringency of the hybridization media to achieve the desired detection of the target nucleic acid. In a particular embodiment, the nucleic acid probes in the kits of the invention are capable of hybridizing to RNA (e.g., mRNA) transcripts under conditions of high stringency.

In another embodiment, the kits include pairs of oligonucleotide primers that are capable of specifically hybridizing to an RNA transcript of a gene in a molecular subtype signature described herein, or a corresponding cDNA. Such primers can be used in any standard nucleic acid amplification procedure (e.g., polymerase chain reaction (PCR), for example, RT-PCR, quantitative real time PCR) to determine the level of the RNA transcript in the sample. As used herein, the term “primer” refers to an oligonucleotide, which is complementary to the template polynucleotide sequence and is capable of acting as a point for the initiation of synthesis of a primer extension product. In one embodiment, the primer is complementary to the sense strand of a polynucleotide sequence and acts as a point of initiation for synthesis of a forward extension product. In another embodiment, the primer is complementary to the antisense strand of a polynucleotide sequence and acts as a point of initiation for synthesis of a reverse extension product. The primer can occur naturally, as in a purified restriction digest, or be produced synthetically. The appropriate length of a primer depends on the intended use of the primer, but typically ranges from about 5 to about 200; from about 5 to about 100; from about 5 to about 75; from about 5 to about 50; from about 10 to about 35; from about 18 to about 22 nucleotides. A primer need not reflect the exact sequence of the template but must be sufficiently complementary to hybridize with a template for primer elongation to occur, i.e., the primer is sufficiently complementary to the template polynucleotide sequence such that the primer will anneal to the template under conditions that permit primer extension.

In another embodiment, the kits of the invention include antibodies that specifically bind a protein encoded by a gene in a molecular subtype signature described herein. Such antibody probes can be polyclonal, monoclonal, human, chimeric, humanized, primatized, veneered, or single chain antibodies, as well as fragments of antibodies (e.g., Fv, Fc, Fd, Fab, Fab′, F(ab′), scFv, scFab, dAb), among others. (See e.g., Harlow et al., Antibodies A Laboratory Manual, Cold Spring Harbor Laboratory, 1988). Antibodies that specifically bind to protein encoded by a gene in a molecular subtype signature described herein can be produced, constructed, engineered and/or isolated by conventional methods or other suitable techniques (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991); Chuntharapai et al., J. Immunol., 152:1783-1789 (1994); Chuntharapai et al. U.S. Pat. No. 5,440,021)). Other suitable methods of producing or isolating antibodies of the requisite specificity can be used, including, for example, methods which select a recombinant antibody or antibody-binding fragment (e.g., dAbs) from a library (e.g., a phage display library), or which rely upon immunization of transgenic animals (e.g., mice). Transgenic animals capable of producing a repertoire of human antibodies are well-known in the art (e.g., Xenomouse® (Abgenix, Fremont, Calif.)) and can be produced using suitable methods (see e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807; Lonberg et al., WO 97/13852).

Once produced, an antibody specific for a protein encoded by a gene in a molecular subtype signature described herein can be readily identified using methods for screening and isolating specific antibodies that are well known in the art. See, for example, Paul (ed.), Fundamental Immunology, Raven Press, 1993; Getzoff et al., Adv. in Immunol. 43:1-98, 1988; Goding (ed.), Monoclonal Antibodies: Principles and Practice, Academic Press Ltd., 1996; Benjamin et al., Ann. Rev. Immunol. 2:67-101, 1984. A variety of assays can be utilized to detect antibodies that specifically bind to proteins encoded by the CNS genes described herein. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assays, inhibition or competition assays, and sandwich assays.

The probes in the kits of the invention can be conjugated to one or more labels (e.g., detectable labels). Numerous suitable detectable labels for probes are known in the art and include any of the labels described herein. Suitable detectable labels for use in the methods of the present invention include, but are not limited to, chromophores, fluorophores, haptens, radionuclides (e.g., ³H, ¹²⁵I, ¹³¹I, ³²P, ³³P, ³⁵S, ¹⁴C, ⁵¹Cr, ³⁶Cl, ⁵⁷Co, ⁵⁸Co, ⁵⁹Fe and ⁷⁵Se), fluorescence quenchers, enzymes, enzyme substrates, affinity tags (e.g., biotin, avidin, streptavidin, etc.), mass tags, electrophoretic tags and epitope tags that are recognized by an antibody (e.g., digoxigenin (DIG), hemagglutinin (HA), myc, FLAG). In certain embodiments, the label is present on the 5 carbon position of a pyrimidine base or on the 3 carbon deaza position of a purine base of a nucleic acid probe.

In a particular embodiment, the label that is conjugated to the probes is a fluorophore. Suitable fluorophores can be provided as fluorescent dyes, including, but not limited to Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), CAL dyes, Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, Carboxy-fluorescein (FAM), Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Oyster dyes, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

Probes can also be labeled using fluorescence emitting metals such as ¹⁵²Eu, or others of the lanthanide series. These metals can be attached to the antibody molecule using such metal chelating groups as diethylenetriaminepentaacetic acid (DTPA), tetraaza-cyclododecane-tetraacetic acid (DOTA) or ethylenediaminetetraacetic acid (EDTA).

In addition to the various detectable moieties mentioned above, the probes in the kits of the invention can also be conjugated to other types of labels, such as spectrally resolvable quantum dots, metal nanoparticles or nanoclusters, etc., which can be directly attached to a nucleic acid probe. As mentioned above, detectable moieties need not themselves be directly detectable. For example, they can act on a substrate which is detected, or they can require modification to become detectable.

For in vivo detection, probes can be conjugated to radionuclides either directly or by using an intermediary functional group. An intermediary group which is often used to bind radioisotopes, which exist as metallic cations, to antibodies is diethylenetriaminepentaacetic acid (DTPA) or tetraaza-cyclododecane-tetraacetic acid (DOTA). Typical examples of metallic cations which are bound in this manner are ⁹⁹Tc ¹²³I, ¹¹¹In, ¹³¹I, ⁹⁷Ru, ⁶⁷Cu, ⁶⁷Ga, and ⁶⁸Ga.

Moreover, probes can be tagged with an NMR imaging agent which include paramagnetic atoms. The use of an NMR imaging agent allows the in vivo diagnosis of the presence of and the extent of the cancer in a patient using NMR techniques. Elements which are particularly useful in this manner are ¹⁵⁷Gd, ⁵⁵Mn, ¹⁶²Dy, ⁵²Cr, and ⁵⁶Fe.

Detection of the labeled probes can be accomplished by a scintillation counter, for example, if the detectable label is a radioactive gamma emitter, or by a fluorometer, for example, if the label is a fluorescent material. In the case of an enzyme label, the detection can be accomplished by colorimetric methods which employ a substrate for the enzyme. Detection can also be accomplished by visual comparison of the extent of the enzymatic reaction of a substrate to similarly prepared standards.

### EXEMPLIFICATION

### Materials and Methods

The following materials and methods were employed in Examples 1-8 provided herein.

### Patients and Samples:

Patients who had been diagnosed, treated and followed for breast cancer progression between 1991 and 2003 at the Koo Foundation Sun Yat-Sen Cancer Center (KFSYSCC), and had their fresh breast cancer tissue frozen in liquid nitrogen at the institutional tumor bank were identified. Patients who did not have follow-up for more than three years at KFSYSCC were excluded, with the exception of those who died within three years after receipt of initial treatment. The study was approved by the institutional review board. Samples deposited in the tumor bank were randomly selected. A total of 447 cases were available. Samples of insufficient RNA (n=1), poor RNA quality (n=116) or unacceptable microarray quality (n=18) were excluded from the study, leaving 312 random samples available (Cohort-1). Gene expression profiles of 15 additional lobular carcinomas of breast collected between 1999 and 2004 were also included in the study (Cohort 2). Thus, the total number of samples was 327.

The clinical characteristics of the 327 patients in Cohorts 1 (n=312) and 2 (n=15) are summarized in Table 8. All 312 samples in cohort 1 were randomly selected and represented a general breast cancer population. The fifteen samples of Cohort 2 were patients with histological diagnosis of lobular carcinoma. Consequently, most patients were positive for estrogen receptor (ER) and progesterone receptor (PR) (Table 8). Because ER+breast cancer tends to be better differentiated, there were less high nuclear grade patients and less HER2 positive in the fifteen patients of cohort 2 (Table 8).

TABLE 8
Clinical characteristics of patients included in the study.

| | |
|---|---|
| Cohort 1 | Cohort 2 |
| (n = 312) | (n = 15) |

| | No. | | No. | |
|---|---|---|---|---|
| Age at diagnosis | | | | |
| <50 yr | 197 | 63% | 6 | 40% |
| >=50 yr | 115 | 37% | 9 | 60% |
| Before 1997 | 125 | 40% | 0 | 0% |
| After 1997 | 187 | 60% | 15 | 100% |
| TNM Stage | | | | |
| I + II | 220 | 71% | 11 | 73% |
| III + IV | 89 | 29% | 4 | 27% |
| Positive Lymph Node No. | | | | |
| 0 | 131 | 42% | 5 | 33% |
| 1-3 | 83 | 27% | 5 | 33% |
| 4-9 | 58 | 19% | 3 | 20% |
| >=10 | 35 | 11% | 2 | 13% |
| Nuclear Grade | | | | |
| I | 23 | 7% | 8 | 53% |
| II | 68 | 22% | 7 | 47% |
| III | 196 | 63% | 0 | 0% |
| ER status* | | | | |
| ER+ | 190 | 61% | 14 | 93% |
| ER− | 122 | 39% | 1 | 7% |
| HER2 status* | | | | |
| HER2+ | 74 | 24% | 1 | 7% |
| HER2− | 238 | 76% | 14 | 93% |
| PR status* | | | | |
| PR+ | 244 | 78% | 14 | 93% |
| PR− | 68 | 22% | 1 | 7% |
| Treatment | | | | |
| Neoadjuvant Chemotherapy | 31 | 10% | 0 | 0% |
| Adjuvant Chemotherapy | 220 | 71% | 12 | 80% |
| Radiation Therapy | 133 | 11% | 8 | 53% |
| Hormonal Rx | 210 | 67% | 14 | 93% |
| No chemotherapy | 50 | 16% | 3 | 20% |

*ER, HER2 and PR status were determined according to microarray data.

mRNA Transcript Profiling Study:

Total RNA from frozen fresh tumor tissues was isolated using Trizol® reagents (Invitrogen, Carlsbad, Calif.) according to the instruction of the manufacturer. The isolated RNA was further purified using RNeasy® Mini Kit (Qiagen, Valencia, Calif.), and the quality was assessed by using RNA 6000 Nano kit and Agilent 2100 Bioanalyzer (Agilent Technologies, Waldbronn, Germany). All RNA samples used for gene expression profiling had an RNA Integrity Number (RIN) of 7.850.99 (mean±SD). Hybridization targets were prepared from total RNA according to the array manufacturer's protocol (Affymetrix) and hybridized to an Affymetrix human genome U133 plus 2.0 array. The U133 Plus 2.0 array contains 54,675 probe-sets for more than 39,000 human genes. Affymetrix One-Cycle Target Labeling Kit was used to prepare biotin-labeled cRNA fragments (hybridization targets). Briefly, double stranded cDNA was synthesized from 5 μg of total RNA per sample. Biotin-labeled complementary RNA (cRNA) was generated by in vitro transcription from cDNA templates. The cRNA was purified and chemically fragmented before hybridization. A cocktail was prepared by combining the specific amounts of fragmented cRNA, probe array controls, bovine serum albumin, and herring sperm DNA according to the protocol of the manufacturer. The cRNA cocktail was hybridized to oligonucleotide probes on the U133 Plus 2.0 array for 16 hours at 45° C. Immediately following hybridization, the hybridized probe array underwent an automated washing and staining in an Affymetrix GeneChip Fluidics Station 450 using the protocol EukGE-WS2v5. Thereafter, U133 Plus 2.0 arrays were scanned using an Affymetrix GeneChip Scanner 3000.

### Scaling and Normalization of Microarray Data:

The expression intensity of each gene was determined by scaling to a trimmed-mean of 500 using the Affymetrix Microarray Analysis Suite (MAS) 5.0 software. The scaled expression intensities of all human genes on a U133 P2.0 array were logarithmically transformed to the base 2, and normalized using quantile normalization (40). The reference standard for quantile normalization was established with microarray data from 327 breast cancer samples.

### Selection of Probe-Sets for Classification of Breast Cancer Molecular Subtypes:

To define breast cancer molecular subtype according to gene expression profiling, the following five steps were performed to select appropriate probe-sets for classification.

Step 1. Genes that have been reported to play important roles in human breast cancer in the literature were identified as pivotal genes (n=23) (Table 9) (41-99).

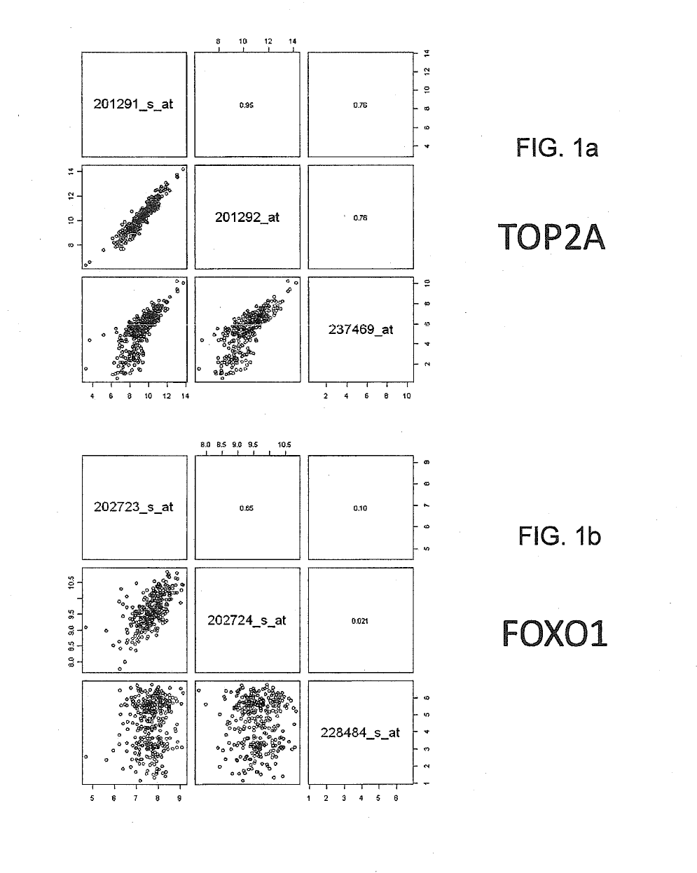

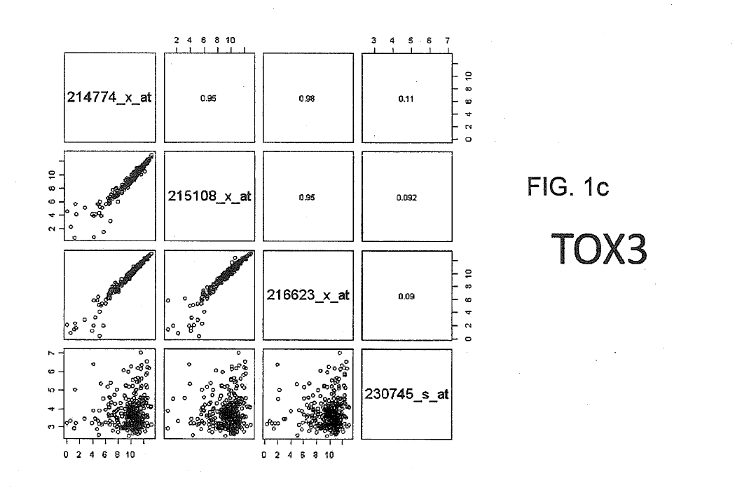

Step 2. An Affymetrix probe-set was chosen to represent each pivotal gene (Table 9). If there were more than one probe-set for a pivotal gene, a representing probe-set was chosen according to the following two criteria: i) a probe-set should express higher intensity and a wider range among 312 samples (Cohort 1); and ii) the same probe-set should show good linear correlation with most of the other probe-sets representing the same gene (FIGS. 1a-1c).

TABLE 9
Pivotal genes used to identify linearly or quadratically correlated genes.

| Gene | | |
|---|---|---|
| Symbol | Probe-set | References |
| BIRC5 | 202094_at | 41-43 |
| BRCA1 | 204531_s_at | 44-46 |
| CD24 | 208650_s_at | 47-50 |
| CEACAM6 | 203757_s_at | 51, 52 |
| CENPF | 207828_s_at | 53 |
| CLDN1 | 218182_s_at | 54, 55 |
| EGFR | 201984_s_at | 56-58 |
| ERBB2 | 216836_s_at | 18, 20, 59-63 |
| ESR1 | 205225_at | 15, 17, 64 |
| FGFR2 | 203638_s_at | 65, 66 |
| FOXA1 | 204667_at | 67-70 |
| FOXC1 | 1553613_s_at | 71, 72 |
| FOXO1 | 202723_s_at | 73, 74 |
| GRB7 | 210761_s_at | 75 |
| HMGA1 | 206074_s_at | 76-78 |
| MAP3K1 | 225927_at | 79, 80 |
| MKI67 | 212022_s_at | 81-85 |
| PGR | 208305_at | 86, 87 |
| PRC1 | 218009_s_at | 88, 89 |
| PRKAA1 | 225984_at | 90 |
| PTEN | 225363_at | 91-94 |
| TOP2A | 201292_at | 95-97 |
| TOX3 | 214774_x_at | 98, 99 |

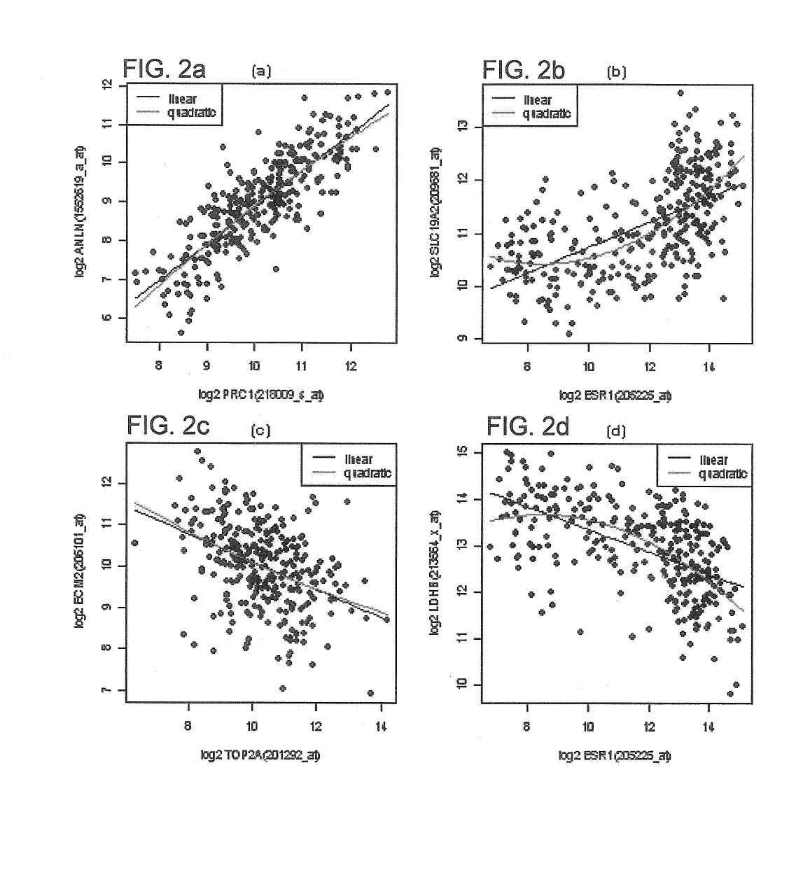

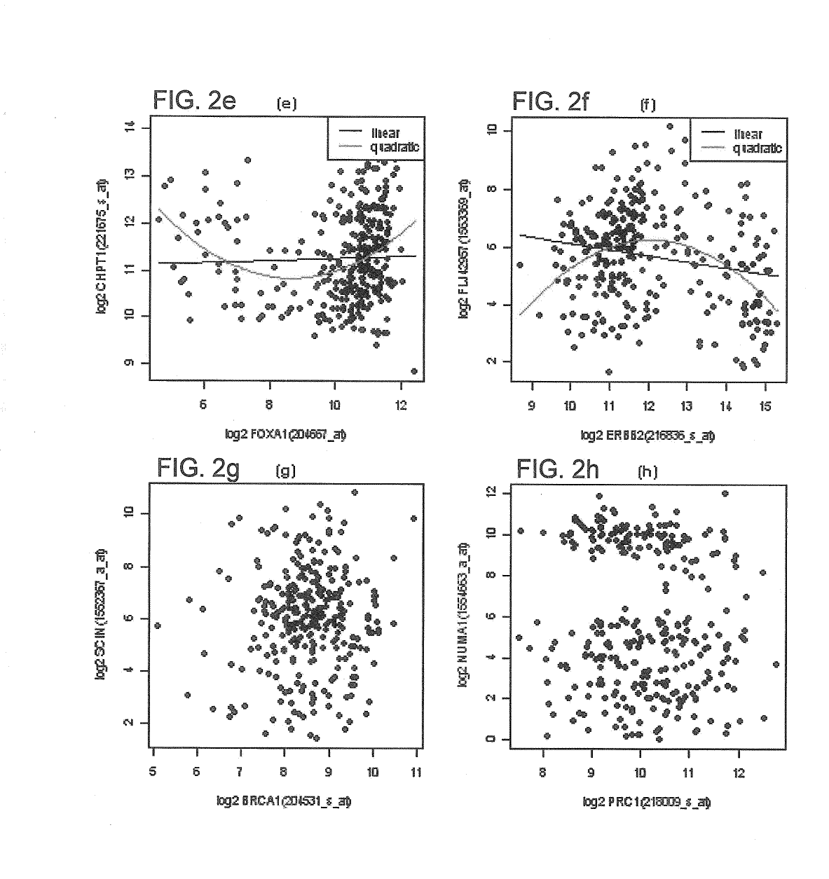

Step 3. A linear and a quadratic correlation were conducted between the representative probe-set of each pivotal gene and all other probe-sets on the U133 Plus 2.0 array in all 312 samples of Cohort 1. Probe-sets showing good proportional or reverse linear (p<10⁻¹⁰) or nonlinear quadratic correlation (p<10⁻⁵) with the probe set of each pivotal gene were identified and selected (FIGS. 2a-2h).

Step 4. The identified probe-sets were further selected according to the following four criteria: i) normalized expression intensities of a selected probe-set must be >512 in at least 5 out of a total of 312 arrays; ii) fold change of normalized expression intensities between the samples at 10% quantile and 90% quantile must be >4; iii) kurtosis of distribution of normalized expression intensities for a probe set in all 312 samples has to be smaller than zero (determination of kurtosis is detailed herein below); iv) the number of peaks on the first derivative of the density function of 312 samples should be greater than 1 (determination of peak is detailed herein below). These four criteria were used to identify highly robust probes-sets with potential to differentiate different subtypes of breast cancer. 1,144 probe-sets that met these criteria were identified.

Step 5. Immune response likely varies between different individuals within the same molecular subtype. Inclusion of immune response genes for subtyping could further split a major molecular subtype and complicate classification. For this reason, immune response genes were identified as those probe-sets with their expression linearly or quadratically correlated with the expression intensities of CD19 (a major marker for B lymphocytes) (Affymetrix probe set ID 206398_s_at) and CD3D (a major marker for T lymphocytes) (Affymetrix probe set ID 213539_at). These genes are likely associated with B-cell or T-cell immune responses, and were excluded from the 1,144 selected probe-sets.

After exclusion of the immune response genes, a total of 768 probe-sets were obtained. The 768 probe-sets included 8 probe-sets from the 23 pivotal genes that passed the intensity filters (Step 4). The remaining 15 pivotal genes that didn't meet the intensity filter of Step 4 were added back to the 768 genes. The final number of total probe-sets available for classification of breast cancer was 783 (Table 1).

### Kurtosis and Peak:

Kurtosis measures how peaked or flat data are relative to a normal distribution. Small kurtosis indicates heavily tailed data having a flatter distribution, while large kurtosis indicates lightly tailed data having a sharper peak (100). The kurtosis of a normal distribution under this definition is 0. Therefore, genes with kurtosis <0 were selected because they have broader distribution.

The density curve of gene expression among samples was approximated using the density function (default setting) in R statistical package from Bioconductor. The curve was smoothed by a Gaussian kernel.

Peaks were defined as the local maxima if a data curve (xi, yi), i=1, . . . , p. First, a window width 2k+1, where 1≦(2k+1)≦p; (xⱼ, yⱼ) is a peak if yⱼ is the maximum amongst yⱼ₋ₖ, yⱼ₋ₖ₊₁, . . . , yⱼ₊₋₁, yⱼ₊₆ for all k≦i≦(p−k), and xⱼ is the location of the peak. In practice, if there are several maxima within a window, the maximum at left was considered the local maximum. The local maximum of within a window is a peak only when it locates at the middle of the window. In this case, k=25. These criteria were used to pick genes with distributions that have more than one peak.

### Clustering Analysis for Identification of Breast Cancer Molecular Subtypes:

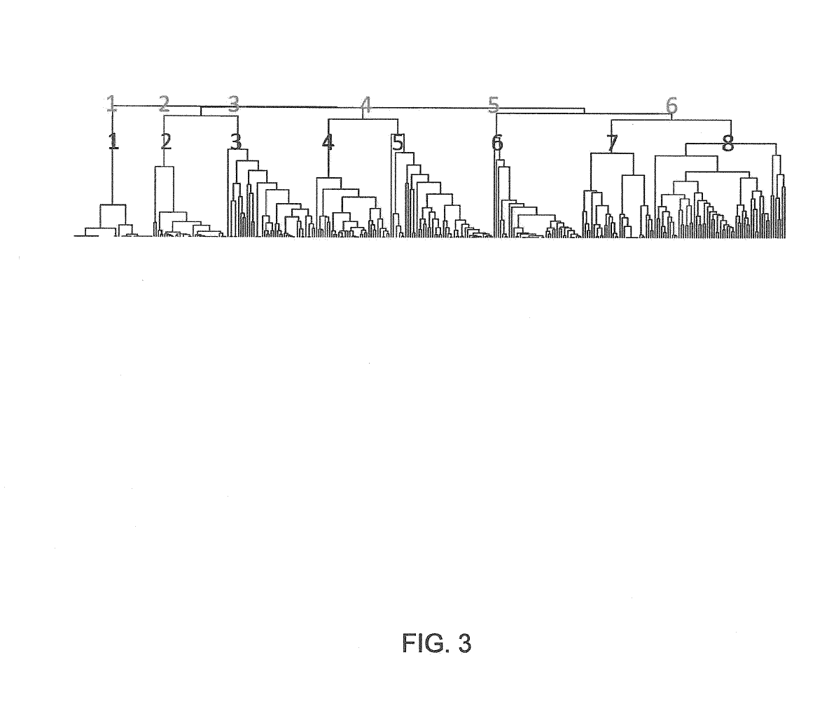

For the study, a hierarchical cluster analysis was run using the 783 described probe-sets on all 327 samples in the Cohorts 1 and 2, resulting in 6 or 8 potential different major subtypes of breast cancer (FIG. 3). k means clustering analyses was then conducted using a 2-step method. The 2-step method was implemented using built-in default “kmeans” and “hclust” function in the R software package (v2.6) from Bioconductor. Average linkage and (1-Pearson correlation coefficient) as distance matrix were set for k means clustering analysis. The 2-step method was conducted as following:

Step 1—k means clustering was run in R software for a given k of 8. After a k means clustering analysis, an integer cluster label from 1 to 8 could be assigned to each breast cancer sample. The cluster analysis was repeated 2000 times using random initial group center assigned by R package. Consequently, each sample had a secondary set of data consisting of 2000 k-means cluster labels as integer numbers from 1 to 8 for each sample.

Step 2. Three hundred and twenty seven breast cancer samples were hierarchical clustered based on 2,000 cluster labels of each sample. The purpose of this step was to obtain a stable breast cancer sample clusters based on 2000 k-means clustering results. The dendrogram generated for 327 breast cancer samples is shown in FIG. 3. The dendrogram indicates that there are 6 or 8 different molecular subtypes of breast cancer depending on the node level chosen for classification. Next, a one-way hierachical clustering analysis was conducted using the selected 783 probe-sets and 327 samples. The arrangement of samples was kept the same as the dendrogram shown in FIG. 3.

The method proposed by Smolkin and Ghosh (101) was then applied to assess the stability of 6 and 8 breast cancer sample clusters derived from the dendrogram shown in FIG. 3. The assessment was done by conducting 200 hierarchical cluster analyses using random sampling of 80% of 327 samples and cluster labels generated from two thousands k-mean analyses. The consistency for cases remain in the same group was calculated as average percentage. The average consistencies for 6 and 8 subtype clusters were 93% and 91%, respectively. Jaccard coefficient for consistency and stability was calculated for each sample.

### Determination of Cut-Point Values for Positivity of Estrogen Receptor (ER), Progesterone Receptor (PR) and HER2:

For determination of gene expression cut-point values that can be used to decide whether a breast cancer sample is positive or negative for ER, PR or HER2, a density plot of all 312 samples from cohort 1 was generated (FIGS. 4a-4c). The results showed bimodal distributions (negative vs. positive). The following statistical method was then applied to determine the cut-point values (C):

Suppose x is the observed expression of a marker for a sample. The posterior probabilities of the case being from the negative population and the positive populations are denoted as P(−|x) and P(+|x), respectively. Let D(x)=P(+|x)/P(−|x), the decision function is:

$$δ ( x ) = \{ \begin{matrix}positive status & if \frac{P ( + \vert x )}{P ( - \vert x )} > d or D ( x ) > d \\ negative status & Otherwise ,\end{matrix}$$

where d is a constant. In this case, d was set to be 1. That is, if the probability of the case being in the positive population is greater than the probability of the case of being in the negative population, than the case is said to be of positive status; otherwise, the case is said to be of negative status.

According to the Bayes rule,

P(k|x)=πₖP(x|k)/p(x)

where k is either + or −, and P(x|k) is the probability of x being observed (if the case is truly from population k), πₖ is the prior probability of the case being from population k (πₖ₊+πₖ₋=1), and p(x) is the marginal probability of observing x.

As a result,

$$D ( x ) = \frac{{π}_{+} P ( x \vert + )}{{π}_{-} P ( x \vert - )} .$$

it is assumed x follows a normal distribution with mean μₖ and variance σₖ², where k is either + or −. A cut-point C can be derived so that the decision function is equivalent to:

$$δ ( x ) = \{ \begin{matrix}positive status & if x > C \\ negative status & Otherwise\end{matrix}$$

That is, if x is smaller than the cut-point, the case is then decided to be from the negative population; otherwise, the case is from the positive population. The prior probability π₋ is reparameterized as 1/[1+exp(−t)] for computational purpose.

Thus,

$$C = \frac{- b - \sqrt{{b}^{2} - 4 a c}}{2 a} if a > 0 and$$

$$C = \frac{- b + \sqrt{{b}^{2} - 4 a c}}{2 a} if a < 0$$

$$where$$

$$a = {σ}_{-}^{2} - {σ}_{+}^{2} , \\ b = 2 \times ( {μ}_{-} {σ}_{+}^{2} - {μ}_{+} {σ}_{-}^{2} ) , \\ c = {σ}_{-}^{2} {μ}_{+}^{2} - {σ}_{+}^{2} {μ}_{-}^{2} - 2 {σ}_{-}^{2} {σ}_{+}^{2} [ - t + ln ( \frac{{σ}_{-}}{{σ}_{+}} ) ] .$$

In this case, μ₋, μ₊, σ₋², σₖ₊², and t are unknown and are estimated by their maximum likelihood estimators (MLEs). The MLEs of μ⁻, μ₊, σ₋², σₖ₊², and t were derived using the default non-linear minimization (nlm) function (Newton-type method) in R package software (v2.6.0) based on 312 cases in the cohort 1. Initial point for the nlm function was subjectively selected to ensure a reasonable solution.

In addition, ER, PR and HER2 (a type 2 epidermal growth factor receptor) status of the breast cancer samples was determined. ER, PR and HER2 were represented by the probe-sets 205225_at, 208305_at and 216836_s_at, respectively.

The cut-point and the estimation for the parameters were:

| | cut-point | μ− | σ− | μ+ | σ+ | τ |
|---|---|---|---|---|---|---|
| ER | 11.61956 | 9.3574 | 1.4737 | 13.3138 | 0.8059 | −0.4281 |
| Her2 | 13.26387 | 11.2639 | 0.8321 | 14.432 | 0.569 | 1.1612 |
| PR | 4.141207 | 2.9724 | 0.6992 | 7.3942 | 1.6947 | −1.3304 |

Initial points for fitting the MLEs for the parameters

| | μ− | σ− | μ+ | σ+ | τ |
|---|---|---|---|---|---|
| ER | 8 | 1 | 14 | 1 | −1 |
| Her2 | 8 | 1 | 14 | 1 | 1 |
| PR | 2 | 1 | 10 | 1 | 1 |

The cut-point values to determine statuses of ER, PR and HER2 as listed above are 11.62, 4.14 and 13.26, respectively. The values are logarithm of normalized expression intensity to a base of 2.

### Molecular Subtyping of Breast Cancer Samples in Other Independent Datasets:

The classification genes identified herein were used to subtype breast cancer in other independent datasets. Genes corresponding to these classification genes we first identified in other independent datasets according to gene symbol, Unigene ID and/or Affymetrix probe-set ID. Then, centroid analysis (102) was applied to subtype breast cancer samples in the independent breast cancer microarray datasets. This was achieved by calculating the Pearson correlation between each sample and each centroid profile of the six breast cancer molecular subtypes described herein. Samples were then assigned to the subtype of the centroid with the largest correlation coefficient.

For instance, 473 out of 783 probe-sets were identified that could be mapped to the dataset from the Netherlands Cancer Institute (NM) based on Unigene ID. If one probe-set in the classification signature is mapped to multiple Unigene IDs on the NKI microarray dataset, the average intensity of multiple Unigene IDs was calculated and used as the corresponding measurement for that probe-set in the classification signature. Each of the NKI samples was then assigned to one of the six molecular subtypes according to the centroid analysis (102).

### Statistical Methods:

All statistical analyses were conducted using SAS/STAT software (ver. 9.1.3) (SAS Institute, Inc.) and R software package (v2.6) from Bioconductor. Fisher's exact test was conducted to determine statistical correlation between molecular subtypes and various clinical phenotypes. The exact p values were estimated by Monte Carlo simulation. Log-rank test was used to analyze survival differences between different molecular subtypes or treatment groups.

### Example 1

### Classification of Breast Cancer into Six Different Molecular Subtypes

In order to have a reliable method to classify breast cancer into different subtypes, 23 genes known to play different important roles in the development and the biology of breast cancer were selected from the literature (Table 9). These 23 genes were called “pivotal genes.” Next, a statistical linear and quadratic correlation study was conducted to select probe-sets that were positively and negatively correlated with each of the 23 pivotal genes as described herein above. Examples of good or poor linear and quadratic correlation are shown in FIGS. 2a-2h. The selected probe-sets were further analyzed for kurtosis and peaks of their density distribution. This approach was based on the assumption that genes showing good correlation with pivotal genes were likely associated with the pivotal genes, and genes that had <0 kurtosis and more than one peak in density distribution could better discriminate different subtypes of breast cancer. 783 probe-sets (Table 1) were identified and used to classify breast cancer samples.

For classification of breast cancer, hierarchical clustering analysis was first conducted using the selected 783 probe-sets on 327 samples of Cohorts 1 and 2. The results suggested that there might be 6 or 8 different subtypes of breast cancer (FIG. 3). k-means clustering analysis was then conducted using k=8. The analysis was repeated 2000 times to generate k-mean label profiles. Thus, each sample had 2000 k-mean labels from 1 to 8. Next, the k-mean label dataset was analyzed with hierachical cluster to generate a dendrogram of 327 breast cancer samples (FIG. 3). The expression intensities of the 783 probe-sets of all 327 samples were then analyzed by one-way hierachical clustering analysis in which the relationship of breast cancer samples clusters was kept the same as shown in FIG. 3.

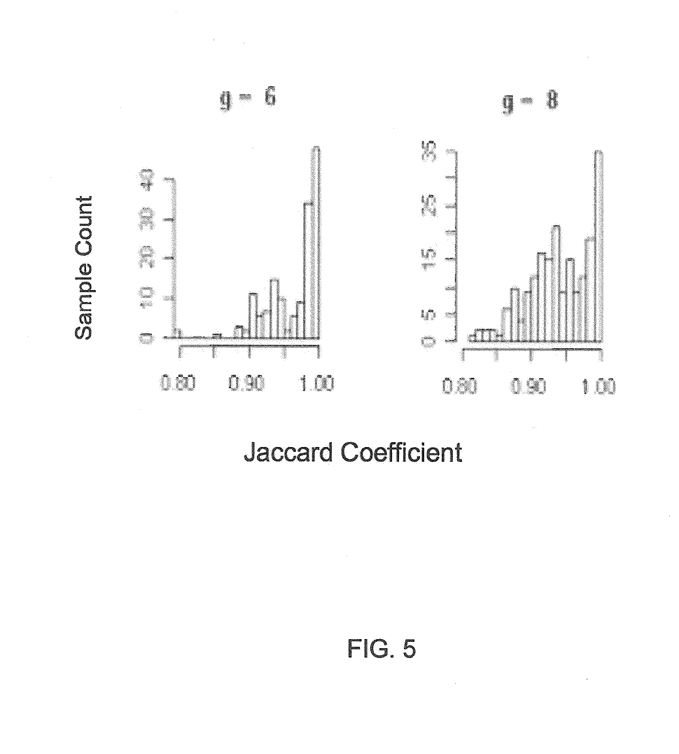

As shown in FIG. 3, there were 6 or 8 major subtypes of breast cancer based on clusters in the dendrogram. Under classification of 8 different subtypes, subtypes 4 and 5, and subtypes 7 and 8 were noted to be under the same node (FIG. 3). The differences of gene expression between subtypes 4 and 5, and between subtypes 7 and 8 were small. Furthermore, comparison of clinical characteristics (e.g., metastasis free survival, overall survival, TNM stage) between these subtypes did not reveal any significant differences (Table 10). Therefore subtypes 4 and 5 were combined into one group, and subtypes 7 and 8 were combined into another. In addition, the method of Smolkin and Ghosh (101) was applied to determine whether the six or eight group classification was more stable. The results showed that the classification into six molecular subtypes is slightly more stable than the classification of eight subtypes (FIG. 5). For these reasons, the six different molecular subtypes were chosen for breast cancer classification.

TABLE 10
Comparison between cluster 4 and 5, and between cluster 7 and 8 for
metastasis-free survival, overall survival and tumor TNM stage.

p value

| Clinical Phenotype | Cluster 4 vs. 5 | Cluster 7 vs. 8 |
|---|---|---|
| Metastasis-free survival* | 0.39 | 0.69 |
| Overall survival* | 0.46 | 0.60 |
| Overall TNM stage** | 0.66 | 0.77 |

*Log-rank test;
**Fisher exact test.

As shown in FIGS. 6a and 6b, 783 probe-sets were clustered into 13 different groups according to the dendrogram of hierachical clustering analysis. We analyzed these 13 groups of probe-sets for enrichment of certain biological functions using Ingenuity Pathway Analysis. The results of Ingenuity Pathway Analyses revealed that the probe-sets used for classification are involved in cell cycle, cellular development/growth/proliferation, cell-to-cell signaling, molecular transport and metabolism (FIGS. 6a,b).

### Example 2

### Breast Cancer Molecular Subtypes Correlate with Clinical Features

To determine whether the six molecular subtypes of breast cancer identified in Example 1 have any distinct clinical features, a series of correlation studies between breast cancer molecular subtypes and different clinical parameters was conducted. The clinical parameters included in our study were age at diagnosis, pathological TNM stage (T: tumor size; N: positive lymph nodes for metastatic tumor; M: presence of distant metastasis), number of lymph nodes positive for metastatic breast cancer, nuclear grade (103), ER status, PR status, HER2 status, loco-regional recurrence during follow-up, development of distant metastasis during follow-up, and survival status.

The results summarized in Table 11 indicate that the six molecular subtypes have significant differences in T-stage, overall TNM stage, nuclear grade, ER positivity, HER-2 positivity, PR positivity, and occurrence of distant metastasis. The results show that subtype V and VI patients had more breast cancers that were small in size (e.g., T1 stage <or =2 cm), while subtype II, III and IV patients had more breast cancers that were large in size (e.g., T2 stage or higher). The majority of patients in subtypes IV, V and VI were positive for estrogen receptor (ER) and progesterone receptor (PR). Notably, subtype V breast cancer patients were 100% positive for ER and PR and 100% negative for HER2. In contrast, all subtype I breast cancer patients were negative for ER. Most subtype II breast cancer patients were negative for ER (97%) and positive for HER2 (76.5%). Subtype III breast cancers were either positive or negative for ER, PR and HER2. Subtype IV breast cancer also had a significant number of HER2 positive cases (27%). Moreover, subtype II had greater propensity to develop distant metastasis (47%), followed by subtype IV (36%) and VI (24%). Subtype V was least likely to develop distant metastasis (5%).

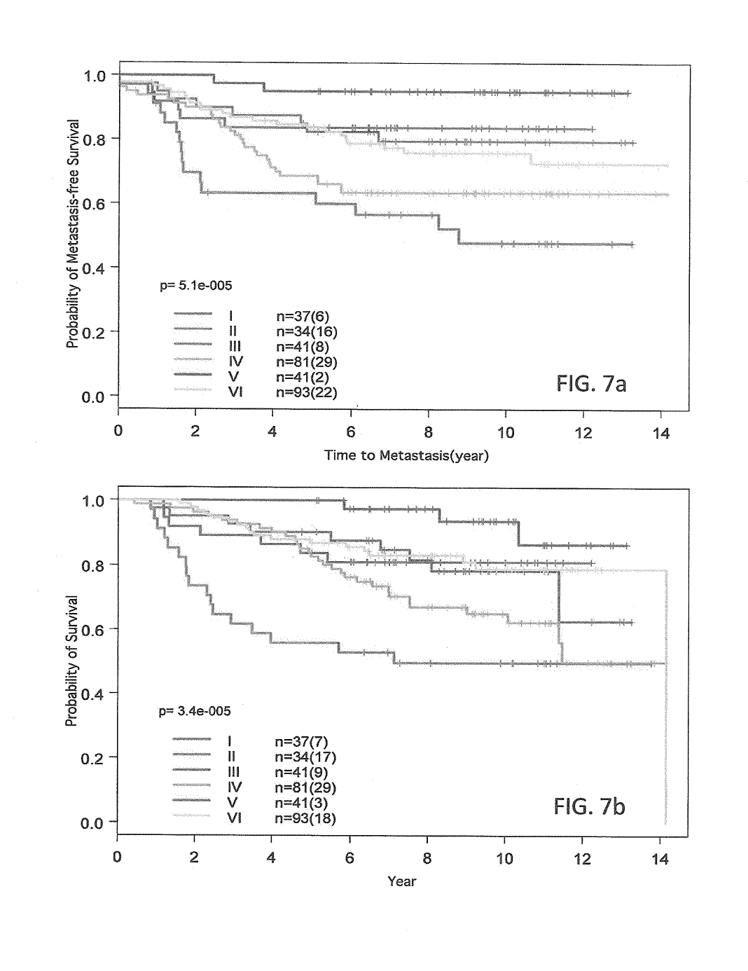

Further comparison of metastasis-free and overall survival among six subtypes was performed by Kaplan-Myer plot and log-rank test. The results depicted in FIGS. 7a and 7b reveal that subtype II had the worst metastasis-free and overall survival followed by subtype IV. Subtype V had the best survival among all six subtypes. Subtypes I, III and VI had intermediate risk. The results of statistical comparison for metastasis-free and overall survival between any two of the six subtypes are summarized in Tables 12a and 12b and show that molecular subtype II has the worst survival outcomes followed by molecular subtype IV. Subtypes I, III and VI have similar intermediate survival outcomes. Subtype V has the best survival outcomes (FIGS. 7a,b).

TABLE 11
Correlation of breast cancer molecular subtypes with clinical phenotypes.

| | Subtype I | | Subtype II | | Subtype III | | Subtype IV | | Subtype V | | Subtype VI | | Fisher exact |
|---|---|---|---|---|---|---|---|---|---|---|---|---|---|
| | N = 37 | | N = 34 | | N = 41 | | N = 81 | | N = 41 | | N = 93 | | test p value |
| Age at diagnosis | | | | | | | | | | | | | |
| <50 yr | 27 | 73.0% | 16 | 47.1% | 30 | 73.2% | 54 | 66.7% | 22 | 53.7% | 54 | 58.1% | |
| >=50 yr | 10 | 27.0% | 18 | 52.9% | 11 | 26.8% | 27 | 33.3% | 19 | 46.3% | 39 | 41.9% | 0.08 |
| T stage | | | | | | | | | | | | | |
| 1 | 8 | 21.6% | 4 | 11.8% | 10 | 24.4% | 16 | 19.8% | 22 | 53.7% | 41 | 44.1% | |
| 2 | 28 | 75.7% | 23 | 67.6% | 20 | 48.8% | 56 | 69.1% | 17 | 41.5% | 44 | 47.3% | |
| 3 | 1 | 2.7% | 5 | 14.7% | 7 | 17.1% | 5 | 6.2% | 1 | 2.4% | 7 | 7.5% | |
| 4 | 0 | 0.0% | 2 | 5.9% | 4 | 9.8% | 4 | 4.9% | 1 | 2.4% | 1 | 1.1% | 2.00E−05 |
| N stage | | | | | | | | | | | | | |
| 0 | 20 | 54.1% | 7 | 20.6% | 16 | 39.0% | 31 | 38.3% | 20 | 48.8% | 43 | 46.2% | |
| 1 | 10 | 27.0% | 10 | 29.4% | 8 | 19.5% | 25 | 30.9% | 12 | 29.3% | 22 | 23.7% | |
| 2 | 4 | 10.8% | 11 | 32.4% | 11 | 26.8% | 14 | 17.3% | 7 | 17.1% | 16 | 17.2% | |
| 3 | 3 | 8.1% | 6 | 17.6% | 6 | 14.6% | 11 | 13.6% | 2 | 4.9% | 12 | 12.9% | 0.26 |
| Pos. Lym. Nodes | | | | | | | | | | | | | |
| 0 | 20 | 54.1% | 6 | 17.6% | 16 | 39.0% | 31 | 38.3% | 20 | 48.8% | 43 | 46.2% | |
| 1-3 | 10 | 27.0% | 10 | 29.4% | 8 | 19.5% | 26 | 32.1% | 12 | 29.3% | 22 | 23.7% | |
| 4-9 | 4 | 10.8% | 11 | 32.4% | 10 | 24.4% | 13 | 16.0% | 7 | 17.1% | 16 | 17.2% | |
| >=10 | 3 | 8.1% | 5 | 14.7% | 6 | 14.6% | 9 | 11.1% | 2 | 4.9% | 12 | 12.9% | 0.30 |
| M stage | | | | | | | | | | | | | |
| 0 | 36 | 97.3% | 33 | 97.1% | 40 | 97.6% | 78 | 96.3% | 41 | 100.0% | 91 | 97.8% | |
| 1 | 1 | 2.7% | 1 | 2.9% | 1 | 2.4% | 3 | 3.7% | 0 | 0.0% | 2 | 2.2% | 0.94 |
| TNM Stage | | | | | | | | | | | | | |
| I | 6 | 16.2% | 2 | 5.9% | 10 | 24.4% | 9 | 11.1% | 12 | 29.3% | 28 | 30.1% | |
| II | 23 | 62.2% | 13 | 38.2% | 11 | 26.8% | 46 | 56.8% | 18 | 43.9% | 36 | 38.7% | |
| II | 6 | 16.2% | 18 | 52.9% | 19 | 46.3% | 23 | 28.4% | 10 | 24.4% | 27 | 29.0% | |
| IV | 1 | 2.7% | 1 | 2.9% | 1 | 2.4% | 3 | 3.7% | 0 | 0.0% | 2 | 2.2% | 7.60E−04 |
| Nuclear Grade | | | | | | | | | | | | | |
| 1 | 1 | 2.7% | 0 | 0.0% | 2 | 4.9% | 2 | 2.5% | 9 | 22.0% | 17 | 18.3% | |
| 2 | 3 | 8.1% | 1 | 2.9% | 4 | 9.8% | 11 | 13.6% | 18 | 43.9% | 38 | 40.9% | |
| 3 | 30 | 81.1% | 28 | 82.4% | 33 | 80.5% | 62 | 76.5% | 10 | 24.4% | 33 | 35.5% | 0 |
| ER | | | | | | | | | | | | | |
| positive | 0 | 0.0% | 1 | 2.9% | 10 | 24.4% | 70 | 86.4% | 41 | 100.0% | 82 | 88.2% | |
| negative | 37 | 100.0% | 33 | 97.1% | 31 | 75.6% | 11 | 13.6% | 0 | 0.0% | 11 | 11.8% | 6.31E−51 |
| HER2 | | | | | | | | | | | | | |
| positive | 4 | 10.8% | 26 | 76.5% | 18 | 43.9% | 22 | 27.2% | 0 | 0.0% | 5 | 5.4% | |
| negative | 33 | 89.2% | 8 | 23.5% | 23 | 56.1% | 59 | 72.8% | 41 | 100.0% | 88 | 94.6% | 9.09E−20 |
| PR | | | | | | | | | | | | | |
| positive | 19 | 51.4% | 14 | 41.2% | 23 | 56.1% | 73 | 90.1% | 41 | 100.0% | 88 | 94.6% | |
| negative | 18 | 48.6% | 20 | 58.8% | 18 | 43.9% | 8 | 9.9% | 0 | 0.0% | 5 | 5.4% | 2.26E−18 |
| Local Relapse | | | | | | | | | | | | | |
| No | 31 | 83.8% | 27 | 79.4% | 39 | 95.1% | 68 | 84.0% | 34 | 82.9% | 86 | 92.5% | |
| Yes | 6 | 16.2% | 4 | 11.8% | 1 | 2.4% | 8 | 9.9% | 3 | 7.3% | 6 | 6.5% | 0.29 |
| Regional Relapse | | | | | | | | | | | | | |
| No | 32 | 86.5% | 26 | 76.5% | 37 | 90.2% | 67 | 82.7% | 36 | 87.8% | 84 | 90.3% | |
| Yes | 2 | 5.4% | 5 | 14.7% | 3 | 7.3% | 6 | 7.4% | 1 | 2.4% | 8 | 8.6% | 0.54 |
| Distant metastasis | | | | | | | | | | | | | |
| No | 31 | 83.8% | 15* | 44.1% | 33 | 80.5% | 50* | 61.7% | 39 | 95.1% | 70* | 75.3% | |
| Yes | 6 | 16.2% | 16 | 47.1% | 8 | 19.5% | 29 | 35.8% | 2 | 4.9% | 22 | 23.7% | 2.51E−05 |

Fisher exact test was used to determine differences among molecular subtypes for each clinical feature.

Tables 12a and 12b. P values of log-rank test for metastasis-free (12a) and overall (12b) survival between any two molecular subtypes. The results show that molecular subtype II has the worst survival followed by subtype IV (FIGS. 7a,b). Subtypes I, III and VI have intermediate survival out come (FIGS. 7a,b). Subtype V has the best survival outcomes (FIGS. 7a,b). P values <0.05 are shown in bold. P values ≧0.05 and <0.10 are shown in italics. P values ≧0.10 are shown in regular font.

TABLE 12a
Metastasis-free survival comparison

| | p values of log rank test between molecular | | | | |
|---|---|---|---|---|---|
| | subtypes | | | | |
| | II | III | IV | V | VI |
| I | 0.0072 | 0.7554 | 0.0467 | 0.0910 | 0.4455 |
| II | | 0.0081 | 0.1431 | 6.434E−06 | 0.0039 |
| III | | | 0.0727 | 0.0400 | 0.6582 |
| IV | | | | 0.0003 | 0.0704 |
| V | | | | | 0.0094 |

TABLE 12b
Overall survival comparison

| | p values of log rank test between molecular | | | | |
|---|---|---|---|---|---|
| | subtypes | | | | |
| | II | III | IV | V | VI |
| I | 0.0062 | 0.9855 | 0.1702 | 0.0947 | 0.8725 |
| II | | 0.0066 | 0.0521 | 1.607E−05 | 0.0001 |
| III | | | 0.1534 | 0.0484 | 0.6917 |
| IV | | | | 0.0009 | 0.0335 |
| V | | | | | 0.0778 |

### Example 3

### Breast Cancer Molecular Subtypes have Distinctive Molecular Features

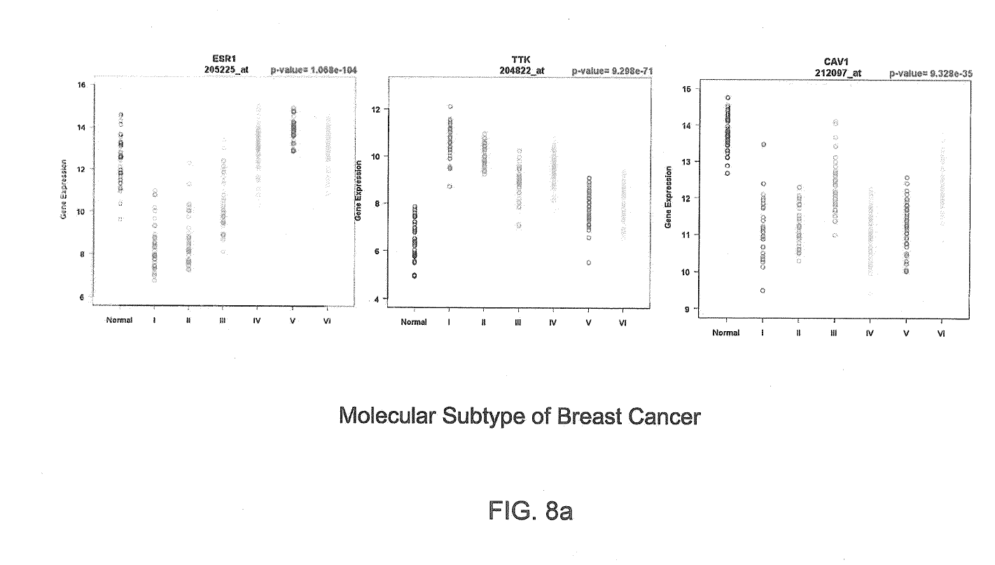

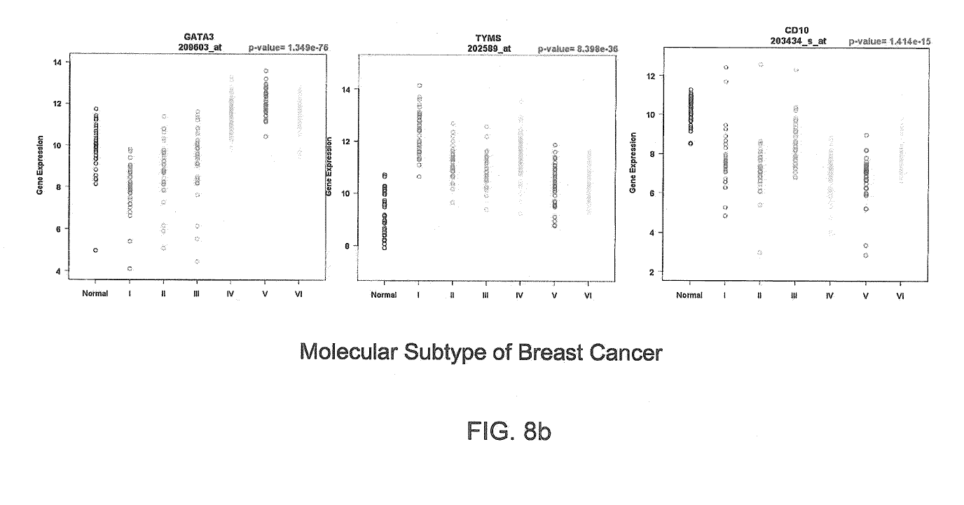

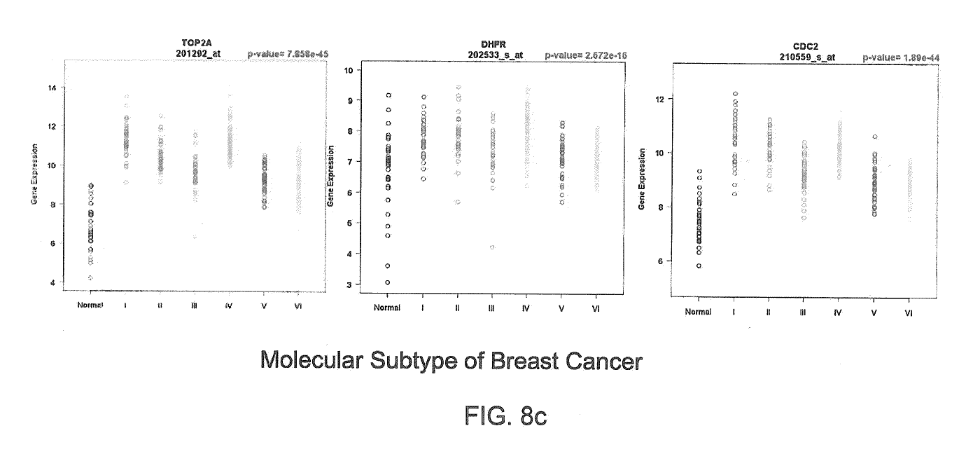

To demonstrate further the distinctiveness of the six different molecular subtypes of breast cancer, 9 genes known to play important roles in tumorigenesis and biology of breast cancer were selected: ESR1 (15, 17, 64), GATA3 (104), TTK (105), TYMS (106, 107), TOP2A (95-97), DHFR (108), CDC2 (109), CAV1 (110) and MME (CD10) (111). Scatter plots of gene expression intensities on 327 breast cancer samples according to their molecular subtypes were prepared (FIGS. 8a-8c). Forty normal breast samples were also included for comparison. The results demonstrated the distinctive distribution of expression of these nine genes among six subtypes of breast cancer.

To further highlight the distinction, one-way hierarchical clustering analysis was conducted using the expression intensities of these nine genes on 327 samples according to the six molecular subtypes. In addition, gene expression data for 40 normal breast tissues were included. The results revealed that the six molecular subtypes of breast cancer have different cell cycle/proliferation activities. Subtypes I, II and IV had high activities of cell cycle/proliferation signature genes. Subtype III had intermediate degree of activity and subtypes V and VI had low expression of the cell cycle/proliferation signature genes.

These results illustrate that all six different subtypes of breast cancer have distinctive molecular characteristics. The distinctive clinical and molecular features are summarized in Table 13.

TABLE 13
Summary of distinct phenotypes of six different molecular subtypes of breast cancer.

| Phenotypical | Breast Cancer Molecular Subtype | | | | | |
|---|---|---|---|---|---|---|
| Characteristics | I | II | III | IV | V | VI |
| ER status | Low | Low | Intermediate | Intermediate | High | Intermediate |
| | | | low | | | |
| PR status | Intermediate | Intermediate | Intermediate | Intermediate | High | Intermediate |
| | low | low | low | | | |
| HER2 status | Intermediate | High | Intermediate | Intermediate | Low | Low |
| | | | high | | | |
| Nuclear Grade | High | High | High | High | Low | Low |
| Metastasis Risk | Intermediate | High | Intermediate | High | Low | Intermediate |
| T stage | High | High | Intermediate | High | Low | Low |
| TNM stage | Intermediate | High | High | Intermediate | Low | Low |
| Metastasis-free | Intermediate | Worst | Intermediate | Poor | Best | Intermediate |
| survival | | | | | | |
| Overall Survival | Intermediate | Worst | Intermediate | Poor | Best | Intermediate |
| Proliferation | High | High | Intermediate | High | Reduced | Reduced |
| signature | | | | | | |

### Example 4

### Breast Cancer Molecular Subtypes Respond Differently to Treatment

The breast cancer samples used in this study were collected over a period of more than 10 years. The period covered a major shift of chemotherapy regimen from CMF (cyclophosphamide-methotrexate-fluorouracil) therapy to CAF (cyclophosphamide-adriamycin-fluorouracil) therapy around 1997 and 1998. The cohorts in this study offered a precious opportunity to investigate how different molecular subtypes of breast cancer responded differently to this change of adjuvant chemotherapy regimen.

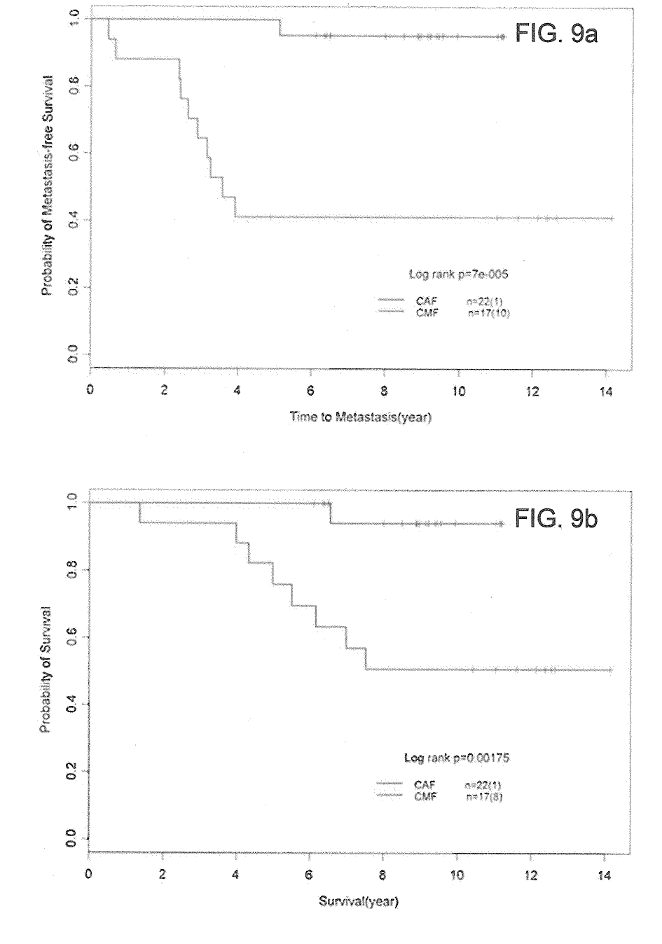

Metastasis-free and overall survival were compared for patients treated with CMF and CAF for adjuvant therapy in each molecular subtype. The results revealed that treatment outcomes between CMF and CAF are very different for subtype IV breast cancer patients (Table 14). The survival curves between the two treatment groups for subtype IV breast cancer indicate that the switch of methotrexate to adriamycin had a dramatic impact on metastasis-free and the overall survival for subtype IV breast cancer patients (FIGS. 9a and 9b). When severity of disease (e.g., TNM stage, numbers of lymph nodes positive for metastatic tumor and nuclear grade) was compared between patients of these two treatment groups for each subtype, no significant differences were noted, except for N stage in the molecular subtype IV breast cancer (p=0.047) (Table 15a). Nevertheless, the CAF group had more N stage=1 patients and the CMF group had more N stage=0 patients (Table 15b). Despite of the fact that N stage favored the CMF group (more N stage=0 patients), the treatment results were far superior for the CAF group that consisted of more patients with N stage=1 (FIGS. 9a,b).

TABLE 14
Survival differences between patients treated with CMF and CAF
adjuvant chemotherapy for each molecular subtype of breast cancer.

| | |
|---|---|
| | p value of Log-rank test |
| Breast | (CAF vs. CMF) |

| | | | |
|---|---|---|---|
| cancer | Patient No. | Metastasis.- | Overall |

| subtype | CAF | CMF | free survival | survival |
|---|---|---|---|---|
| I | 10 | 13 | 0.823 | 0.823 |
| II | 5 | 6 | 0.620 | 0.757 |
| III | 16 | 4 | 0.576 | 0.511 |
| IV | 22 | 17 | 7.00E−05 | 0.002 |
| V | 12 | 8 | 0.414 | 0.963 |
| VI | 22 | 11 | 0.226 | 0.062 |

TABLE 15a
Comparison of the clinical parameters selected for disease severity
between patients treated with CMF and CAF adjuvant chemotherapy
in each molecular subtype (Table 14).

| | P values of Fisher exact test | | | | |
|---|---|---|---|---|---|
| | | | | Positive | |
| Molecular | T | N | Overall | Lymph | Nuclear |
| subtype | stage | stage | TNM stage | Nodes | Grade |
| I | 0.379 | 0.169 | 0.162 | 0.169 | 0.479 |
| II | 0.455 | 0.546 | 0.303 | 0.546 | 1.000 |
| III | 0.610 | 0.625 | 1.000 | 0.625 | 0.718 |
| IV | 0.612 | 0.047 | 0.109 | 0.067 | 0.703 |
| V | 1.000 | 0.418 | 0.666 | 0.418 | 0.666 |
| VI | 1.000 | 0.326 | 0.594 | 0.546 | 0.172 |

The two treatment groups in each molecular subtype was compared by Fisher exact test for each clinical parameter and p values are summarized in the table. TNM stages were determined according to 2002 AJCC Cancer Staging Manual. No patients had distant metastasis at the time of diagnosis. The results indicate that the disease severity was quite similar between the two treatment groups (CMF vs. CAF) except for N stage in molecular subtype IV breast cancer (p=0.047).

TABLE 15b
Comparison of N stage distribution between patients treated with
CMF and CAF in the molecular subtype IV breast cancer patients.

Molecular
subtype IV

| N Stage | CAF | CMF | Total |
|---|---|---|---|
| 0 | 9 | 11 | 20 |
| 1 | 12 | 3 | 15 |
| 2 | 1 | 2 | 3 |
| 3 | 0 | 1 | 1 |
| Total | 22 | 17 | 39 |

As shown in Table 15b, the CAF group had more N stage=1 patients and the CMF group had more N stage=0 patients. P value by Fisher exact test was 0.047. Despite of that N stage favored the CMF group, the treatment results was far more superior for the CAF group (FIGS. 9a,b).

The results of this study (FIGS. 9a,b, Tables 14, 15a and 15b) indicate that molecular subtype IV breast cancer was relatively insensitive to methotrexate and very sensitive to adriamycin. Replacement of adriamycin with methotrexate significantly improved both metastasis-free survival and overall survival. Thus, it is critical to identify molecular subtype IV breast cancer patients and select adriamycin containing adjuvant chemotherapy regimen for their treatment. The clinical importance of this finding is further underscored by recent comments from various medical experts regarding the use of anthracyclines (e.g., adriamycin) for treatment of breast cancer. Experts have been baffled by not having a reliable method to identify a subset of patients that are responsive to adjuvant treatment containing anthracyclines (113). As demonstrated by the results of this study, the subset of patients responsive to anthracycline is molecular subtype IV breast cancer and can be readily identified by the molecular subtyping method described herein.

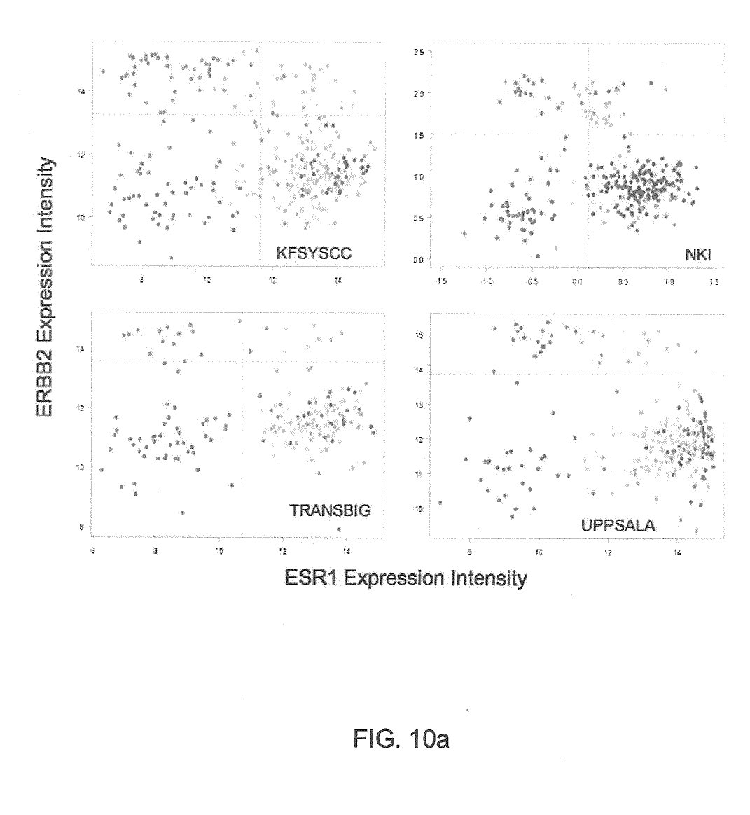

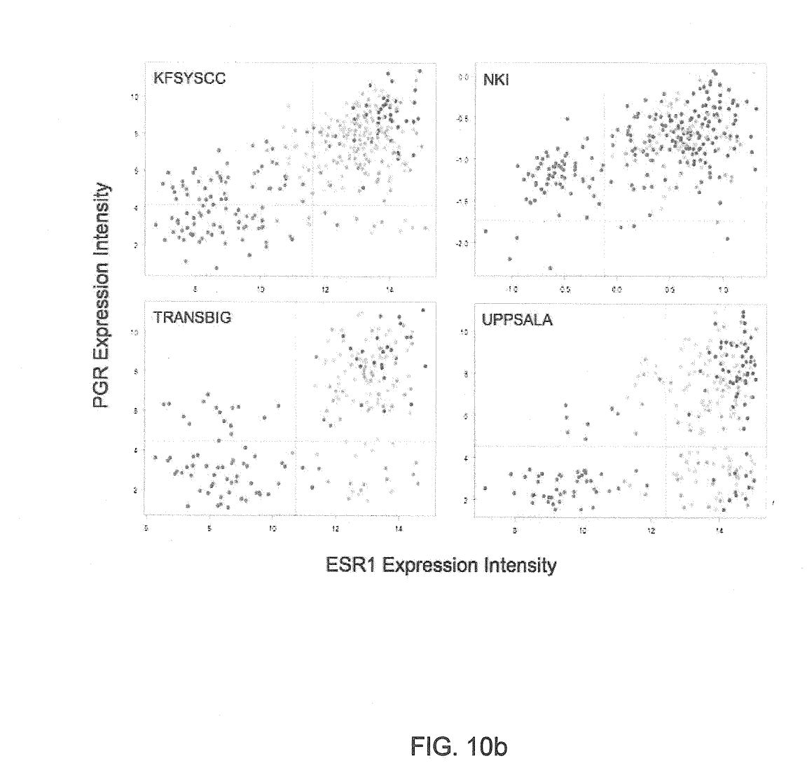

The results of this study also demonstrated that there were no significant differences in metastasis-free and overall survival for molecular subtype I breast cancers treated with CAF or CMF adjuvant chemotherapy after surgery (Table 14). All molecular subtype I patients had excellent long-term survival. There was no difference in disease severity between the two treatment groups (Tables 15a,b and 16). As shown in FIG. 10a, subtype I breast cancer was mostly negative for ER and HER2. This phenotype is consistent with basal-like breast cancer which is known to have aggressive clinical course (121) and to be sensitive to chemotherapy (122, 123). Thus, subtype I breast cancer must be treated with adjuvant chemotherapy and is responds equally well to CAF and CMF adjuvant chemotherapy.

TABLE 16
Comparison of disease severity between patients treated with
and without adjuvant chemotherapy in each molecular subtype.

| | Patient No. | | P values of Fisher exact test | | | | |
|---|---|---|---|---|---|---|---|
| Breast cancer | No adjuvant | Adjuvant | T | N | Overall | Positive | Nuclear |
| subtype | chemo-Rx | chemo-Rx | stage | stage | TNM stage | lymph nodes | grade |
| I | 0 | 0 | * | * | * | * | * |
| II | 4 | 23 | * | * | * | * | * |
| III | 3 | 30 | * | * | * | * | * |
| IV | 9 | 63 | 0.256 | 0.874 | 0.016 | 0.837 | 0.122 |
| V | 12 | 28 | 0.144 | 0.857 | 0.267 | 0.857 | 0.171 |
| VI | 25 | 56 | 0.018 | 0.095 | 0.034 | 0.095 | 0.857 |

* Insufficient number of patients for statistical analyses.

The comparison between two treatment groups was conducted by Fisher exact test and p-values are summarized in the table. TNM stages were determined according to 2002 AJCC Cancer Staging Manual. No patients had distant metastasis at the time of diagnosis. Disease severity was quite similar between two groups (no adjuvant chemotherapy vs. adjuvant chemotherapy) for the subtype V patients. More detailed comparison for the subtype V patients is summarized in Table 17.

### Example 5

### Molecular Basis for Insensitivity to Methotrexate and Sensitivity to Anthracycline in Subtype IV Breast Cancer

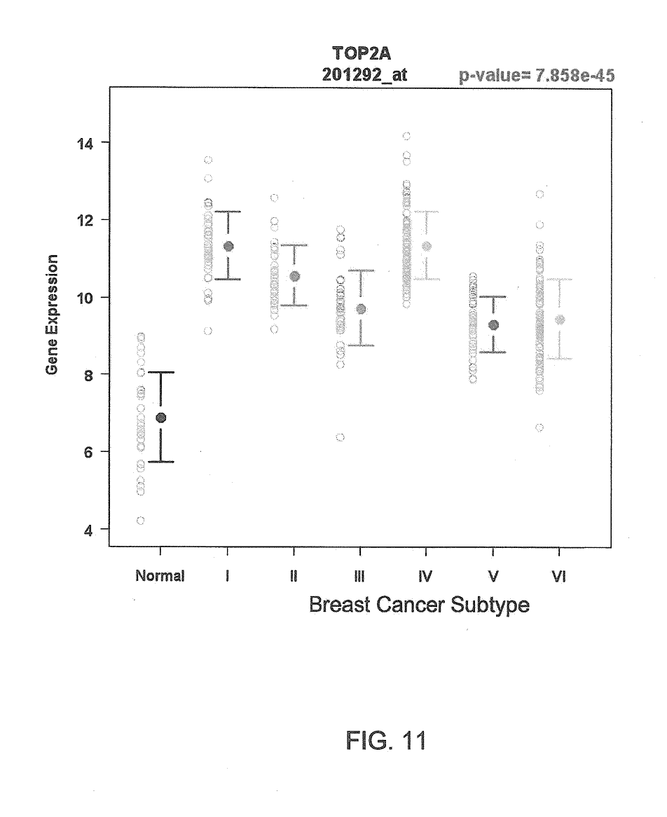

As discussed in Example 4, molecular subtype IV breast cancer is relatively insensitive to methotrexate and sensitive to anthracycline (e.g., adriamycin). Topoisomerase 2A (TOP2A) is a known drug target for anthracyclines (96, 114). It has been widely reported in the literature that increased expression of TOP2A makes breast cancer more sensitive to anthracycline (96, 115). As shown in FIG. 11, subtypes I and IV breast cancers have the highest levels of TOP2A among the six molecular subtypes and both subtypes should respond well to anthracyclines (e.g., adriamycin).

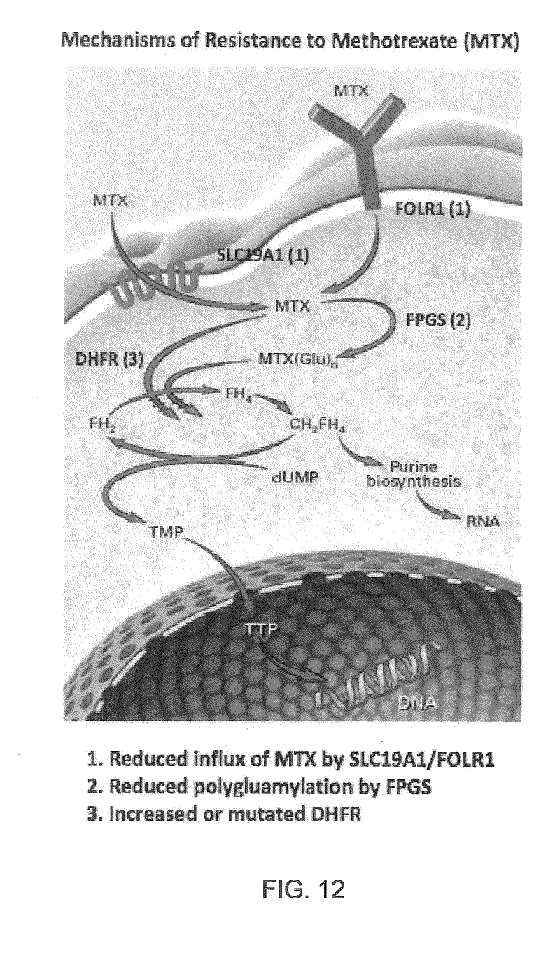

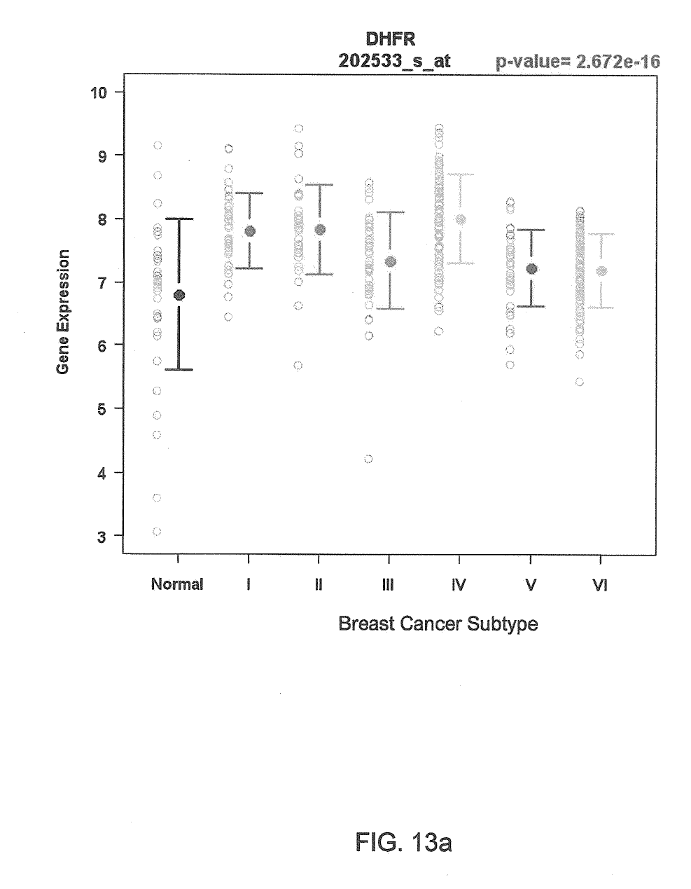

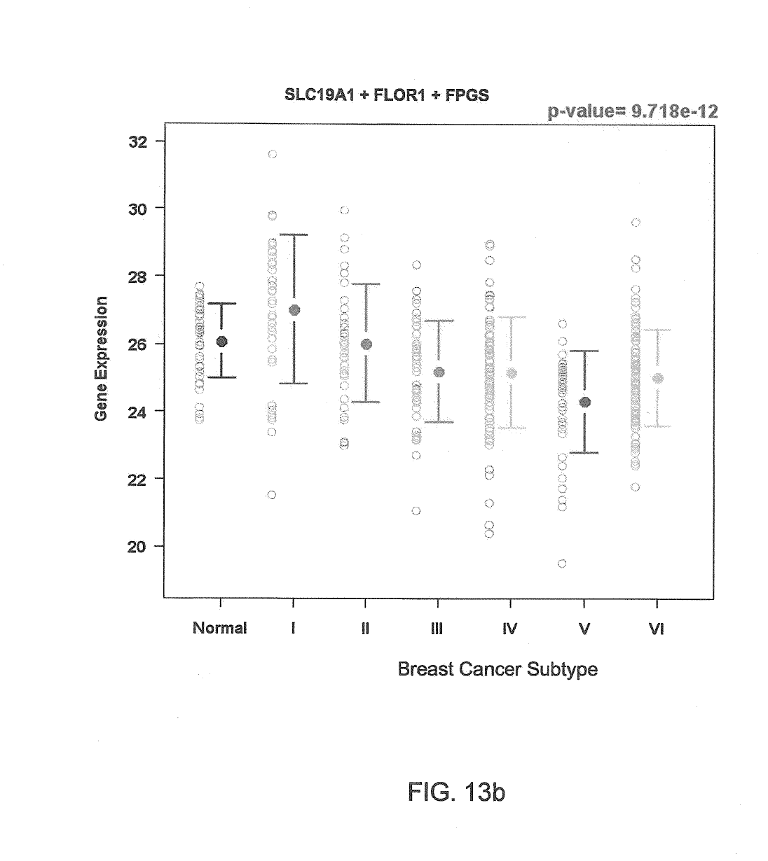

Regarding insensitivity to methotrexate, it has been well documented that multiple mechanisms are responsible for methotrexate-resistance. These mechanisms include: 1) reduced level of transporters (SLC19A1 and FOLR1) to move methotrexate into cells; 2) reduced activity of folylpolyglutamate synthase (FPGS) for retention of methotrexate in cells, and 3) increased dihydrofolate reductase (DHFR) activity for methotrexate to inhibit (FIG. 12) (ref. 116). As shown in FIGS. 13a and 13b, the expression of DHFR is high (FIG. 13a) and the combined expression of SLC19A1, FLOR1 and FPGS was low (FIG. 13b) in subtype IV breast cancer. These results help explain why subtype IV breast cancer does not respond well to methotrexate-containing CMF regimen and why the substitution of adriamycin for methotrexate in CAF regimen drastically changes the treatment outcome.

### Example 6

### Molecular Subtyping Identifies Breast Cancers that do not Require Adjuvant Chemotherapy

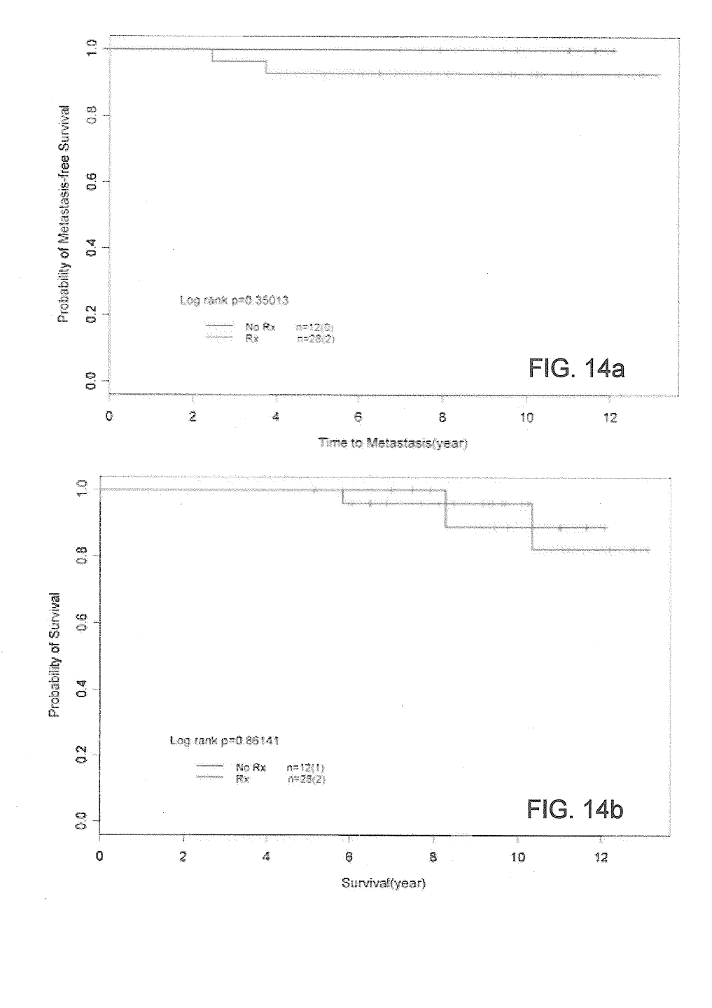

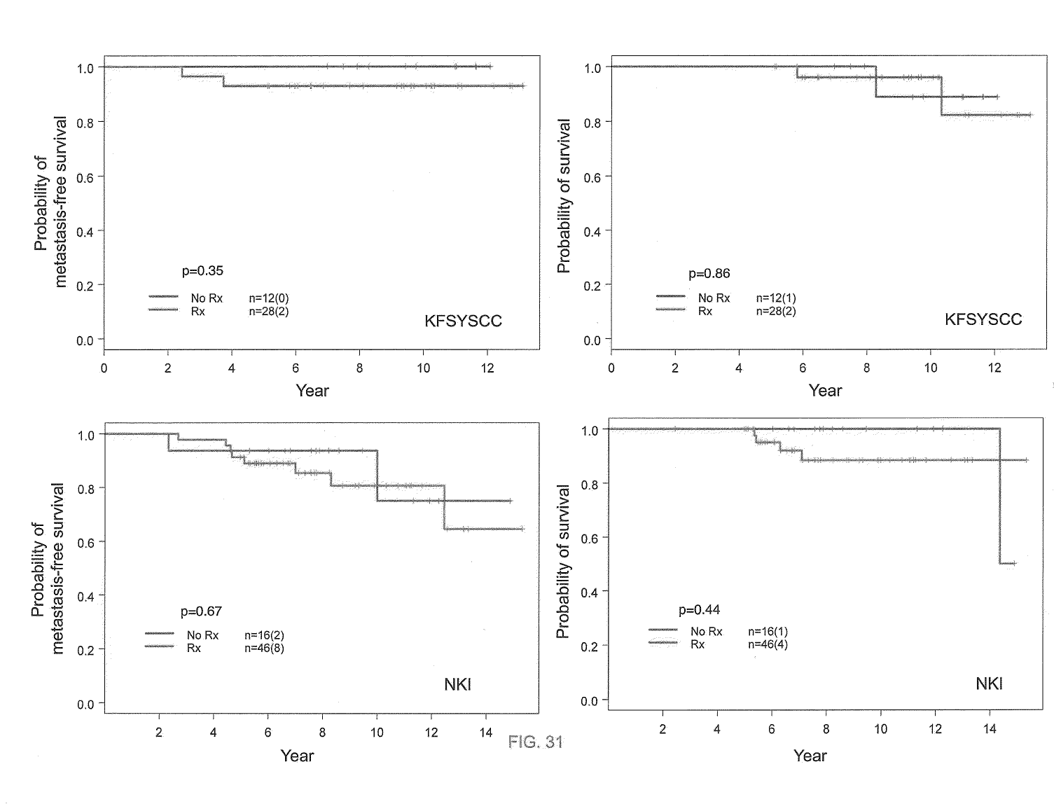

In the cohorts in this study, a significant number of patients chose not to receive adjuvant chemotherapy. These patients provided an opportunity to determine how omission of adjuvant chemotherapy would have impacted their long-term survival according to molecular subtypes of breast cancer. Among the 327 patients in the study, only subtypes IV, V, and VI had a sufficient number of patients treated with (n=63, 28 and 56, respectively) and without (n=9, 12 and 25, respectively) adjuvant chemotherapy for a comparison study (Table 16). However, only molecular subtype V patients did not have significant differences in disease severity between patients with and without adjuvant chemotherapy (Table 16). We then compared metastasis-free and overall survival between patients with and without adjuvant chemotherapy for molecular subtype V breast cancers. The results showed no difference between these two groups of patients for both metastasis-free and overall survival (FIGS. 14a,b; see also FIG. 31, which includes data for the independent NKI dataset).

A more detailed comparison of clinical characteristics between these two groups of subtype V patients is shown in Table 17. There were no significant differences between these two groups of patients for all relevant clinical parameters tested. It is noteworthy that most of these patients had an early stage of the disease (T≦2 and positive node no. ≦3). As pointed out above, molecular subtype V is a highly selective subtype of breast cancer. All subtype V patients were positive for ER and PR, and negative for ERBB2 (Table 11). Unfortunately, one can not rely on these three markers to identify subtype V patients, because patients of other molecular subtypes (i.e., subtypes IV and VI) also could share the same ER, PR and HER2 status (FIGS. 10a,b). Thus, a molecular subtyping by gene expression profiling, such as the approach described herein, is necessary to identify this unique subtype of breast cancer patients who require only hormonal therapy without adjuvant chemotherapy for long-term survival if the disease is at early stage (T≦2 and positive node no. ≦3) (FIGS. 14a,b and Table 17).

TABLE 17
Comparison of clinical characteristics for molecular subtype V breast
cancer patients treated with and without adjuvant chemotherapy.

| | Molecular subtype V breast cancer | | | | |
|---|---|---|---|---|---|
| | Rx | | No-Rx | | |
| | (n = 28) | | (n = 12) | | |
| | (patient | | (patient | | p values of Fisher |
| | no.) | | no.) | | exact test |
| T stage | | | | | 0.144 |
| 1 | 14 | 50% | 8 | 67% | |
| 2 | 14 | 50% | 3 | 25% | |
| 3 | 0 | 0% | 0 | 0% | |
| 4 | 0 | 0% | 1 | 8% | |
| N stage | | | | | 0.857 |
| 0 | 13 | 46% | 7 | 58% | |
| 1 | 8 | 29% | 4 | 33% | |
| 2 | 5 | 17% | 1 | 8% | |
| 3 | 2 | 8% | 0 | 0% | |
| M stage | | | | | |
| 0 | 28 | 100% | 12 | 100% | |
| Positive Lymph | | | | | 0.857 |
| Nodes | | | | | |
| 0 | 13 | 46% | 7 | 58% | |
| 1-3 | 8 | 29% | 4 | 33% | |
| 4-9 | 5 | 18% | 1 | 8% | |
| >=10 | 2 | 7% | 0 | 0% | |
| TNM Stage | | | | | 0.274 |
| I | 6 | 25% | 6 | 50% | |
| II | 14 | 57% | 4 | 33% | |
| III | 7 | 18% | 2 | 17% | |
| Nuclear Grade | | | | | 0.1706 |
| 1 | 4 | 14% | 5 | 42% | |
| 2 | 13 | 46% | 4 | 33% | |
| 3 | 8 | 29% | 2 | 17% | |
| Hormonal Therapy | | | | | 0.627 |
| No | 3 | 11% | 2 | 17% | |
| Yes | 25 | 89% | 10 | 83% | |
| Post-op Radiation | | | | | 0.9999 |
| Therapy | | | | | |
| No | 20 | 71% | 9 | 75% | |
| Yes | 8 | 29% | 3 | 25% | |

### Example 7

### Validation of Molecular Subtyping Using Independent Breast Cancer Datasets

To validate the method of molecular subtyping described herein, the classification genes were applied to four independent breast cancer datasets. All four datasets are available publicly (117-120). These datasets included metastasis-free and/or overall survival data, and more than 100 samples in each dataset. The characteristics of these four datasets are summarized in Table 18. All patients were from different European countries. The classification genes identified herein and centroid analysis were used to classify breast cancer samples of each dataset into the same six molecular subtypes.

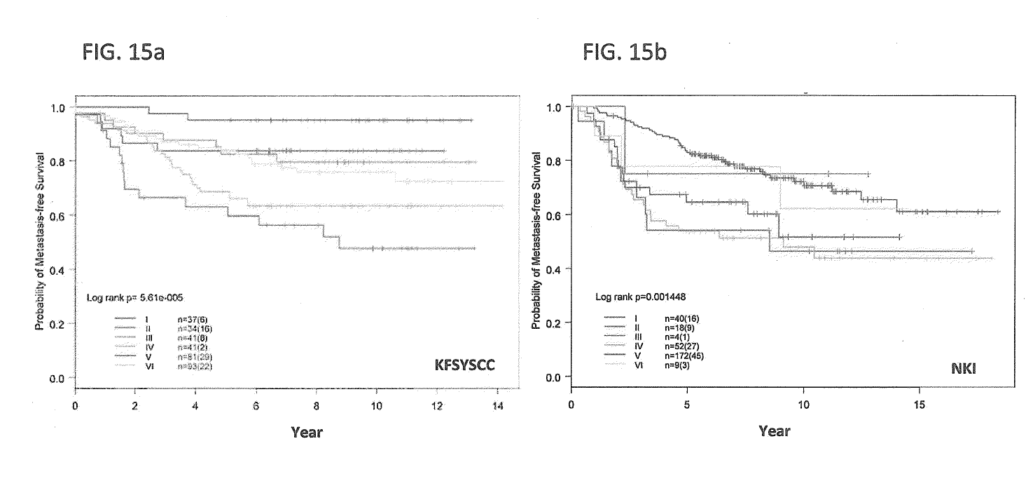

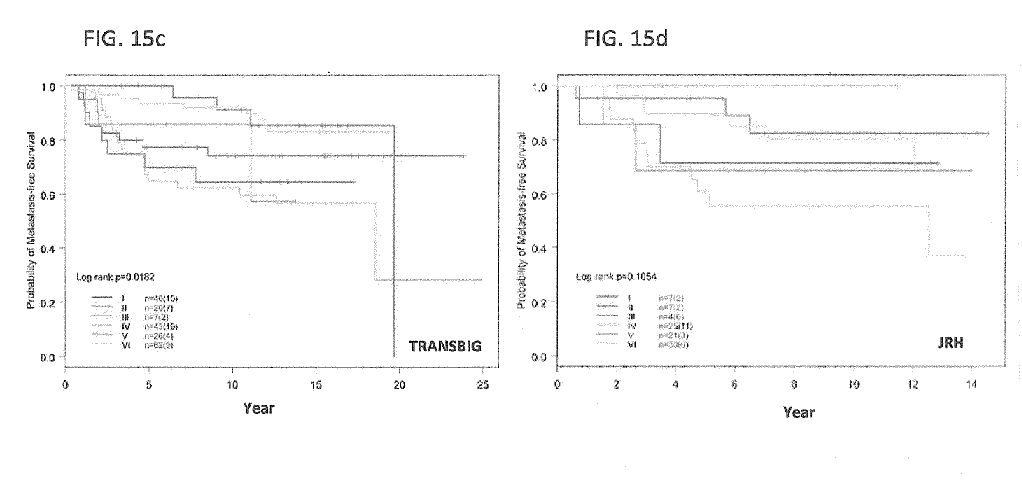

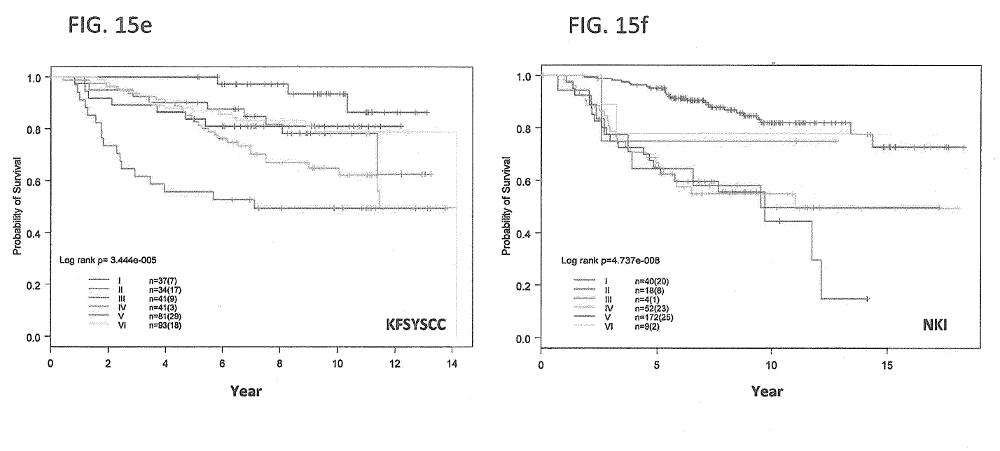

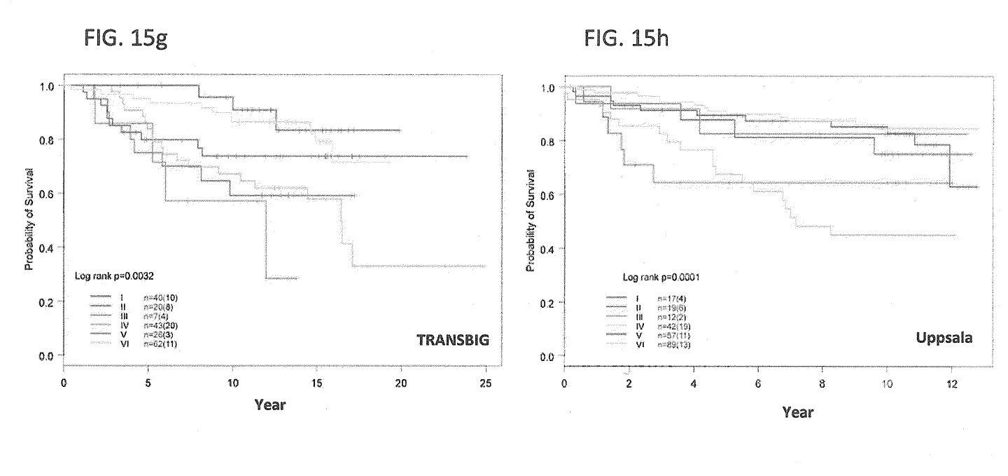

First, the metastasis-free and the overall survival of all patients from the four independent datasets were classified according to their breast cancer molecular subtypes. The survival curves from all four datasets, including KFSYSCC, are depicted in FIGS. 15a-15h. The results support that the six molecular subtypes of breast cancer from patients of different geographic regions and ethnic backgrounds share the same survival characteristics. Like the KFSYSCC breast cancer patients, molecular subtypes II and IV consistently had a higher risk for distant metastasis (FIGS. 15a-15d) and shorter overall survival (FIGS. 15e-15h) in the independent datasets. Molecular subtype V consistently had a low risk for metastasis and good overall survival. In addition, almost all subtype V breast cancer patients in the independent data sets were positive for ER and PR, and negative for HER2 (FIGS. 10a and 10b), just as for the KFSYSCC breast cancer patients. Therefore, molecular subtype V patients who are highly positive for ER should be responsive to anti-estrogen hormonal therapy. Molecular subtype I patients consistently had intermediate risk for metastasis and intermediate overall survival, except for patients from the Netherlands Cancer Institute (NKI). Molecular subtypes III and VI appeared to have intermediate to low risk for metastasis and intermediate survival. However, the data appear to be more variable due to the smaller number of patients.

As discussed above, the molecular subtype I patients from NKI, unlike those from the other datasets, had a higher risk for metastasis and poorer survival. A possible reason for this discrepancy is that molecular subtype I breast cancer is similar to the so-called basal-like breast cancer that is known to have aggressive course and negative for ER and HER2 (FIG. 10a) (ref. 121). Molecular subtype I breast cancer is also highly sensitive to chemotherapy (122, 123). Most of the subtype I breast cancer patients (95%) at KFSYSCC received chemotherapy. In contrast, only 35% of subtype I patients in the NKI dataset received chemotherapy. Therefore, it is expected that the survival of subtype I patients in the NKI dataset would not have been as high. The results underscore the importance of identifying molecular subtype I breast cancer patients and the need to administer adjuvant chemotherapy to these patients in order to obtain a better survival outcome.

TABLE 18
Characteristics of breast cancer gene expression datasets used for independent validation.

| | | | Availability of | | | | |
|---|---|---|---|---|---|---|---|
| | | | Survival Data | | | | |
| | Sample | Microarray | Overall | Metastasis- | | Year of | |
| Dataset | Size | platform | Survival | free | Clinical data | diagnosis | Ref. |
| JRH | 101 | Affymetrix | No | Yes | Age; adjuvant chemotherapy | Not | 119 |
| | | U133A | | | (n = 40); TNM; N0(n = 61); no patient | available | |
| | | | | | selection | | |
| TRANSBIG | 198 | Affymetrix | Yes | Yes | Age: <61 yo; TNM: ≦T2 (<5 cm) and | 1980-1998 | 120 |
| | | U133A | | | N = 0; no RX information | | |
| Uppsala | 251 | Affymetrix | Yes | No | No patient selection; no TNM and | 1987-1989 | 118 |
| | | U133A + B | | | RX information | | |
| NKI | 295 | Two color | Yes | Yes | Age: <52 yo; TNM: ≦T2 (<5 cm) and | 1984-1995 | 117 |
| | | oligo. array | | | N = 0 (n = 151); surgery ± radiation | | |
| | | | | | (n = 144); chemotherapy (n = 20), | | |
| | | | | | hormonal Rx (n = 20), both (n = 20) | | |

There were no overall survival data for the data set from JRH (Oxford, UK). There were no metastasis-free survival data for the dataset from Uppsala, Sweden.

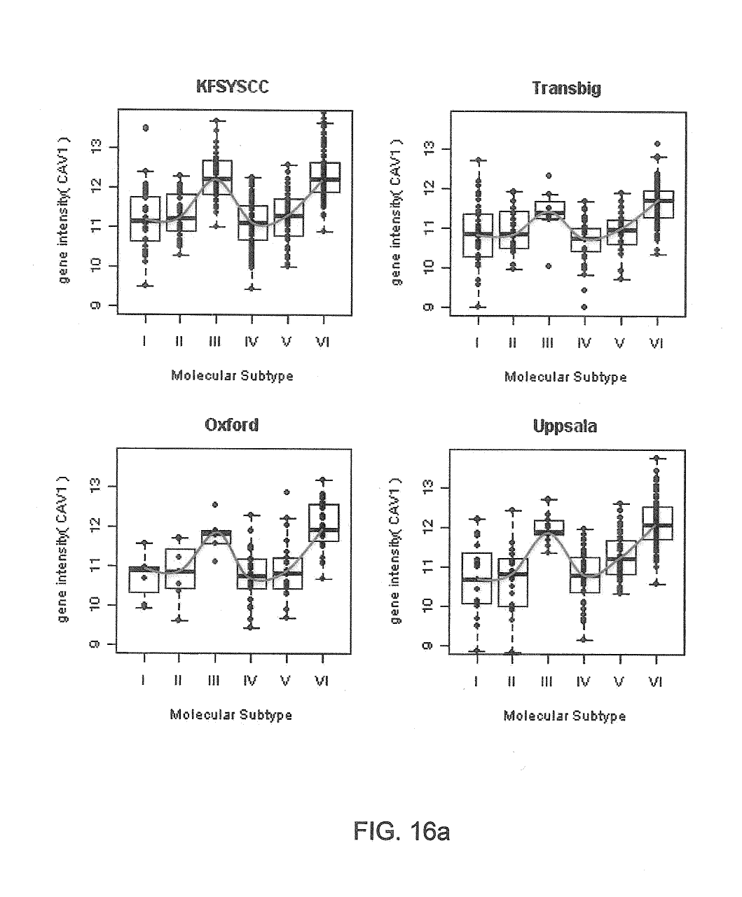

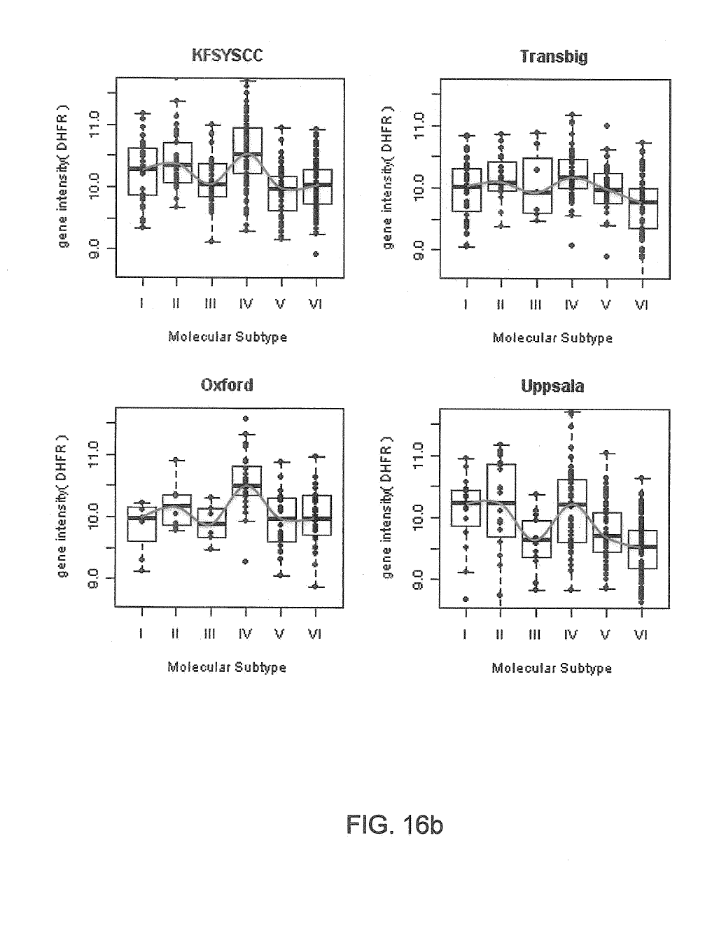

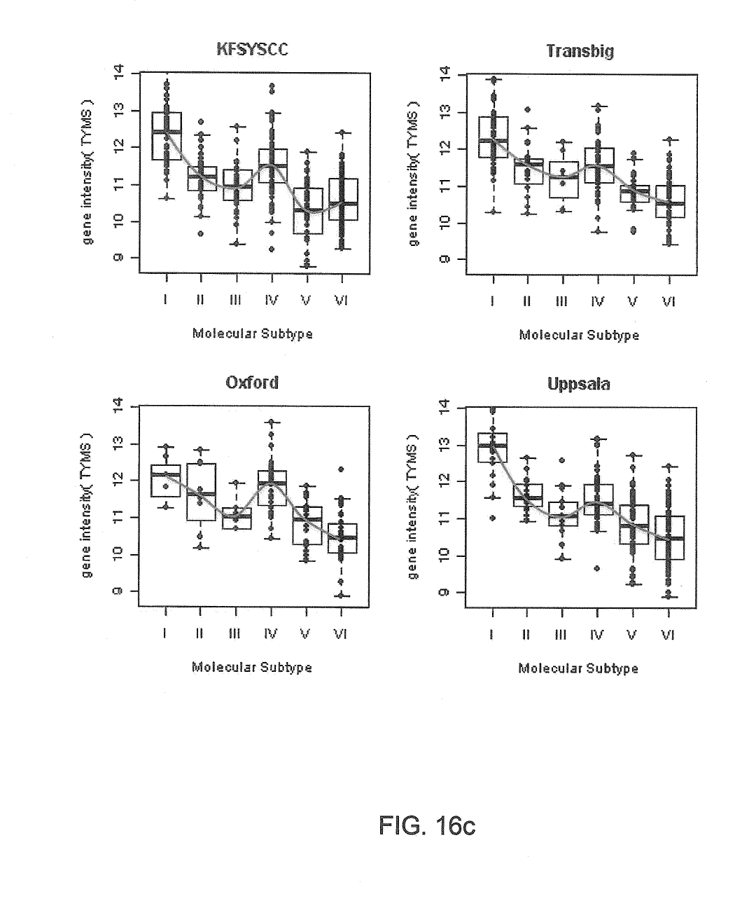

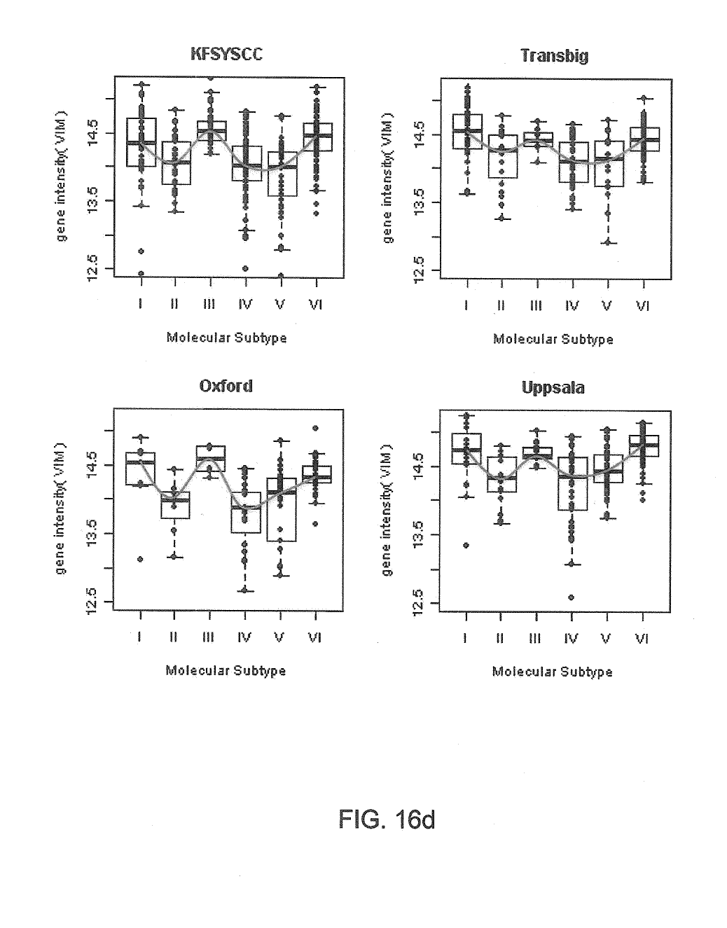

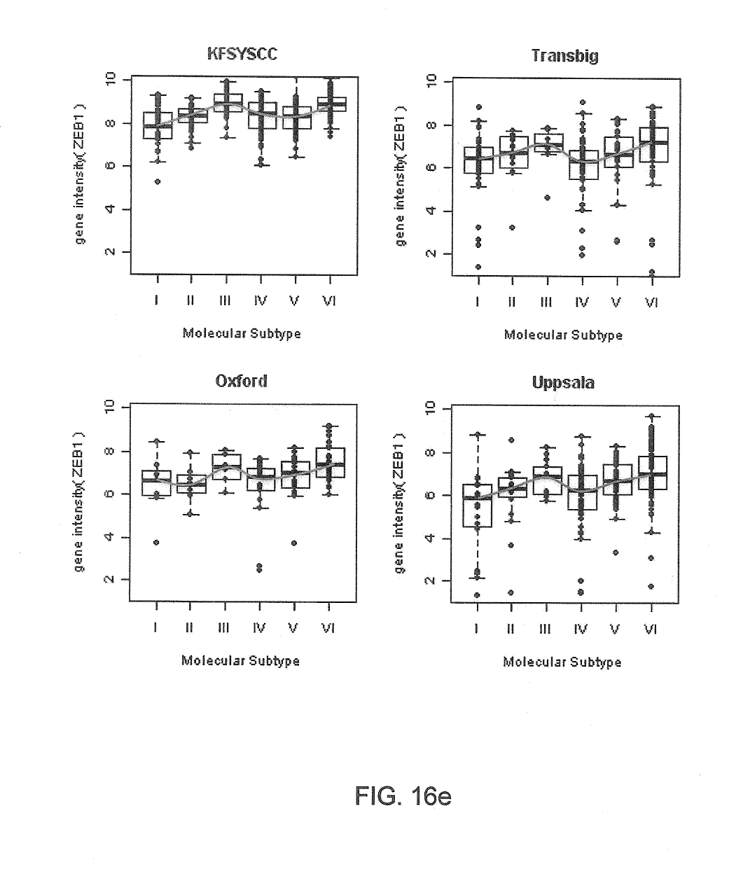

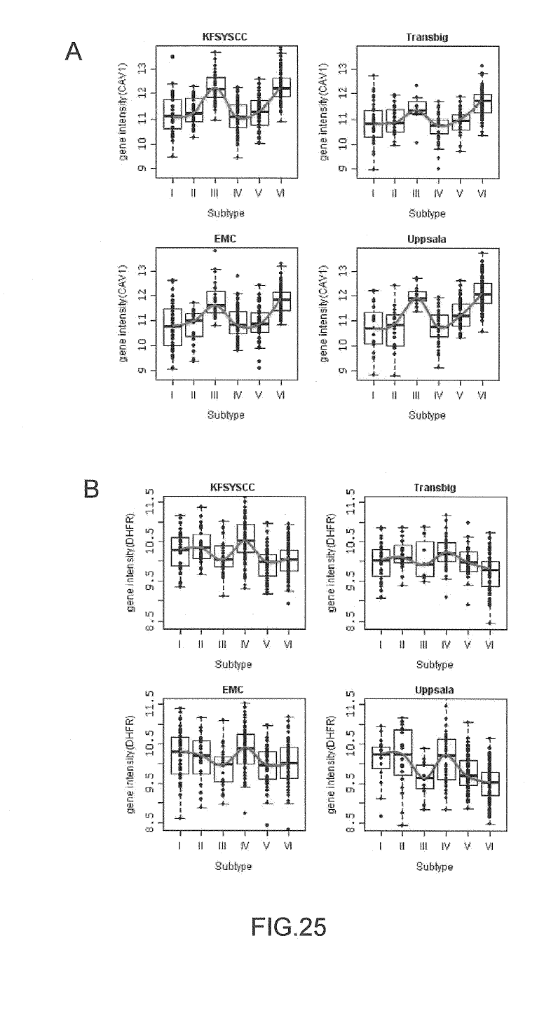

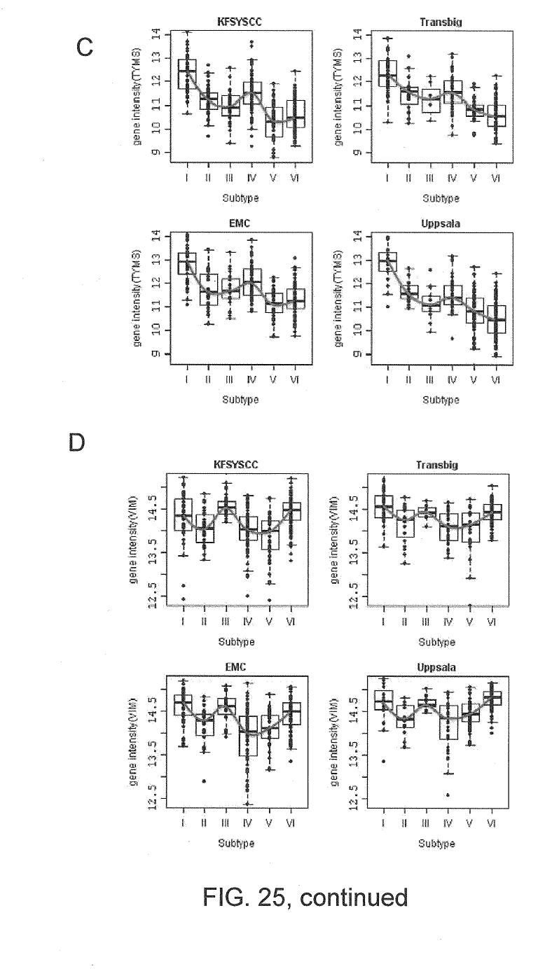

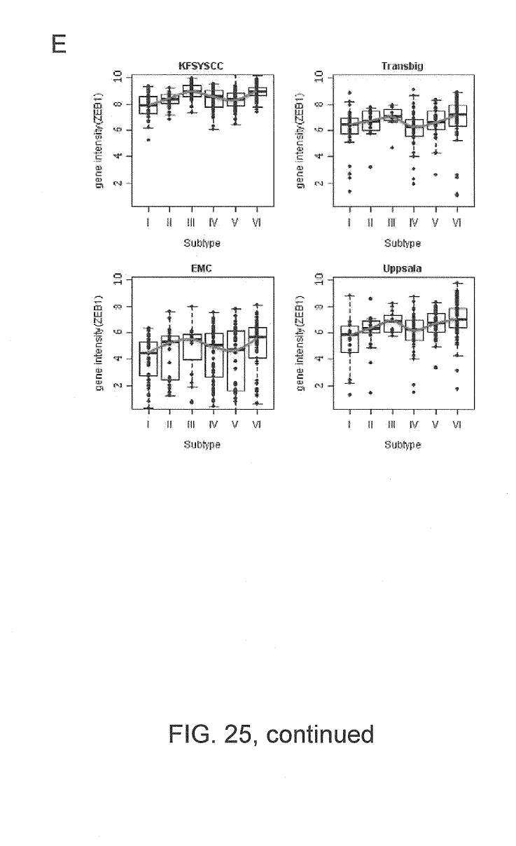

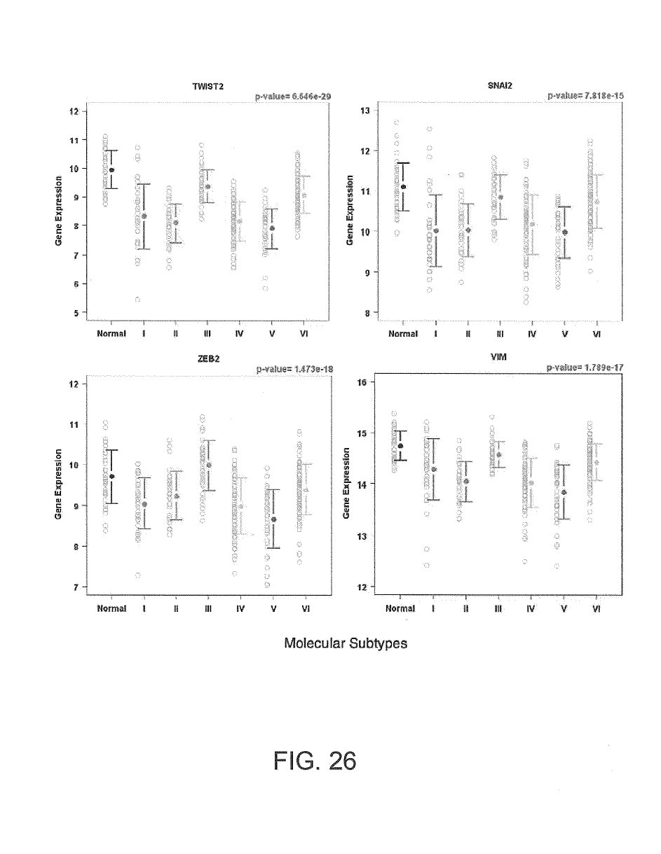

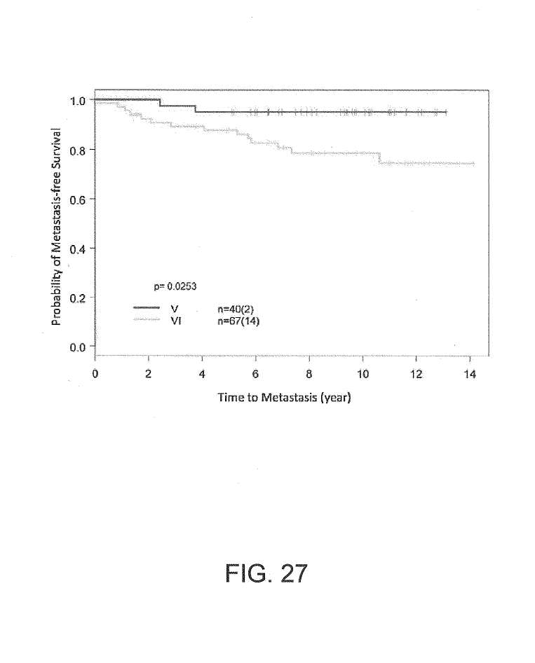

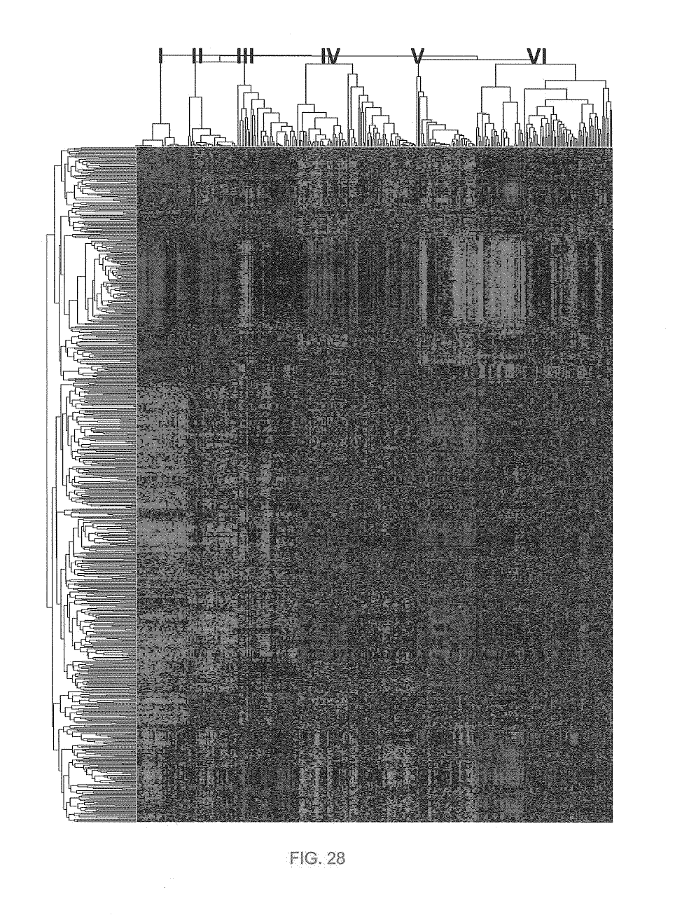

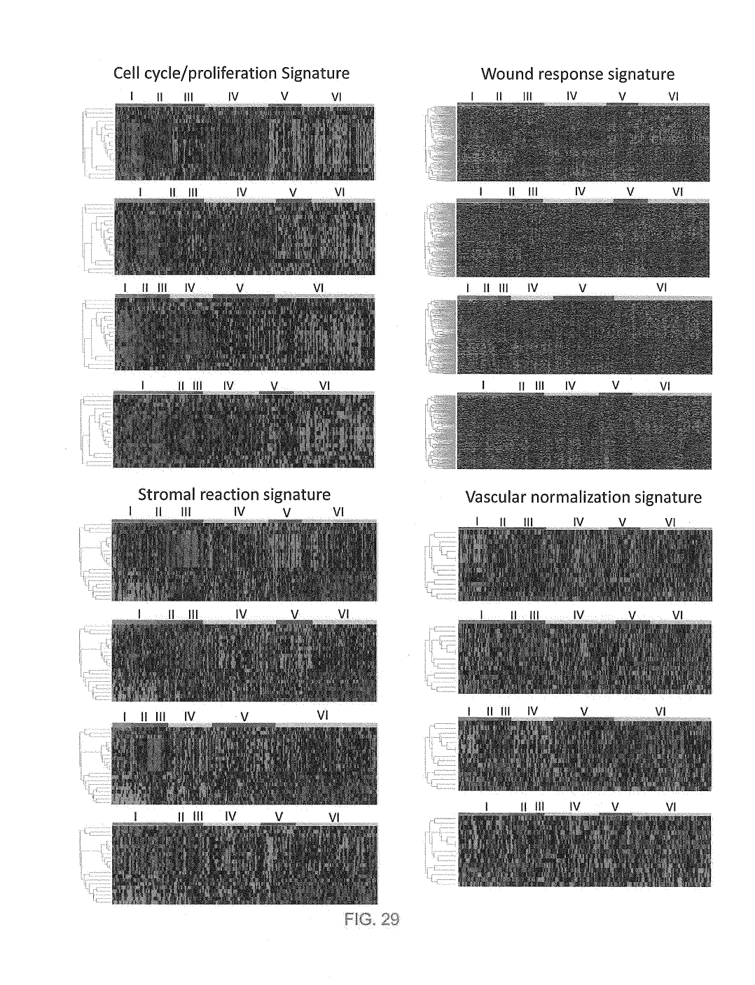

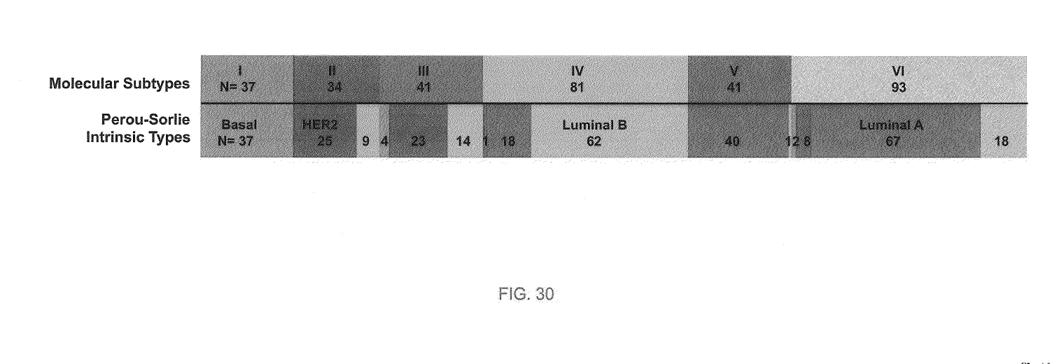

To demonstrate further that corresponding subtypes of breast cancer from different independent datasets share the same molecular characteristics, five genes (CAV1, DHFR, TYMS, VIM, ZEB1) were selected for their known roles in determining chemo-sensitivities and biology of breast cancer (106-108, 110, 124, 125). None of these genes are part of the classification signature described herein. When the expression intensity of these genes were plotted according to the predicted molecular subtypes, it was found that their distribution patterns were highly similar to the genes of the classification signature (FIGS. 16a-16e; see also FIGS. 25A-E, which includes the EMC dataset). These results indicate that breast cancers from different geographic regions share the same molecular characteristics and can be classified according to the six different molecular subtypes described herein. These results also indicate that the classification genes identified herein can be applied to gene expression data collected across different platform technologies (e.g. Affymetrix U133 GeneChips vs. two color microarray of NKI). In addition, thymidylate synthase (TYMS) is known to be the target of fluorouracil. Higher expression of the TYMS gene is associated with higher sensitivity to fluorouracil included in CMF or CAF adjuvant chemotherapy regimens (126, 127). The finding of the highest level of TYMS expression in subtype I breast cancer (FIG. 16c) supports that subtype I breast cancer has high sensitivity to adjuvant chemotherapy, as discussed above, and the emphasizes the critical importance of administering adjuvant chemotherapy to these patients.

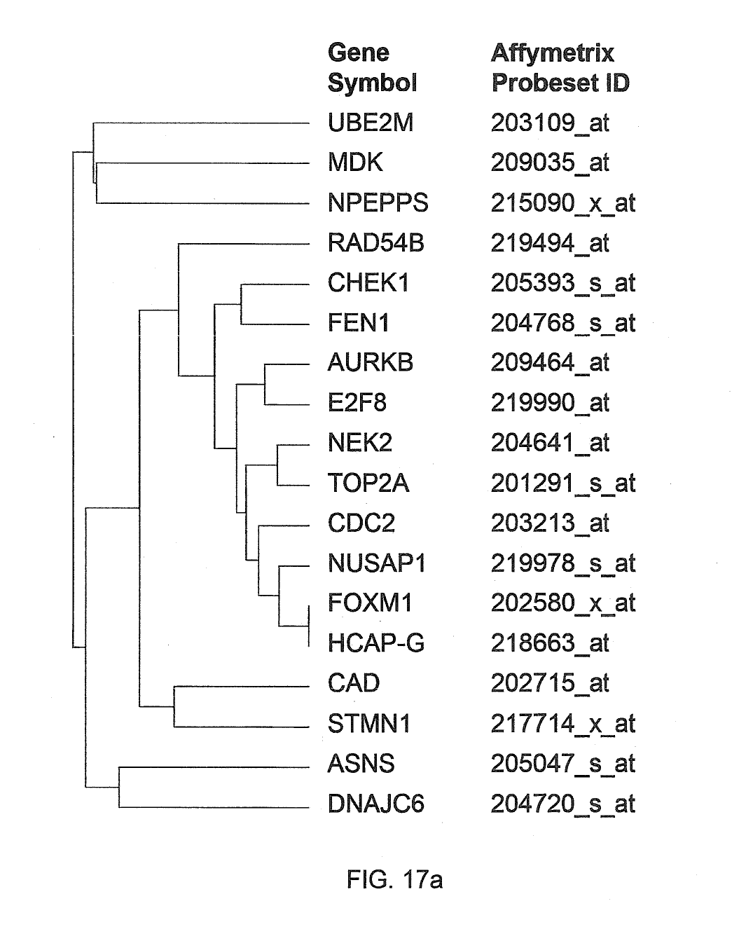

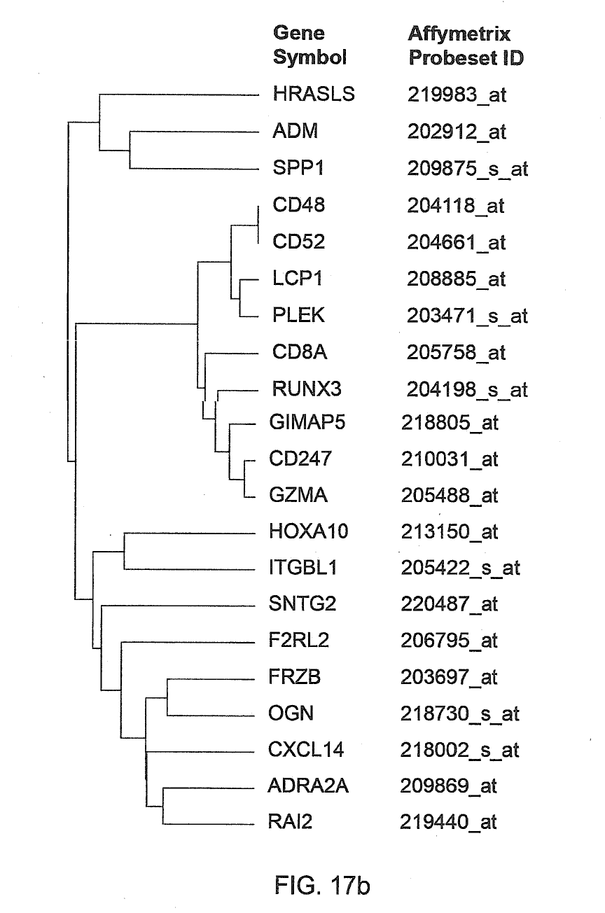

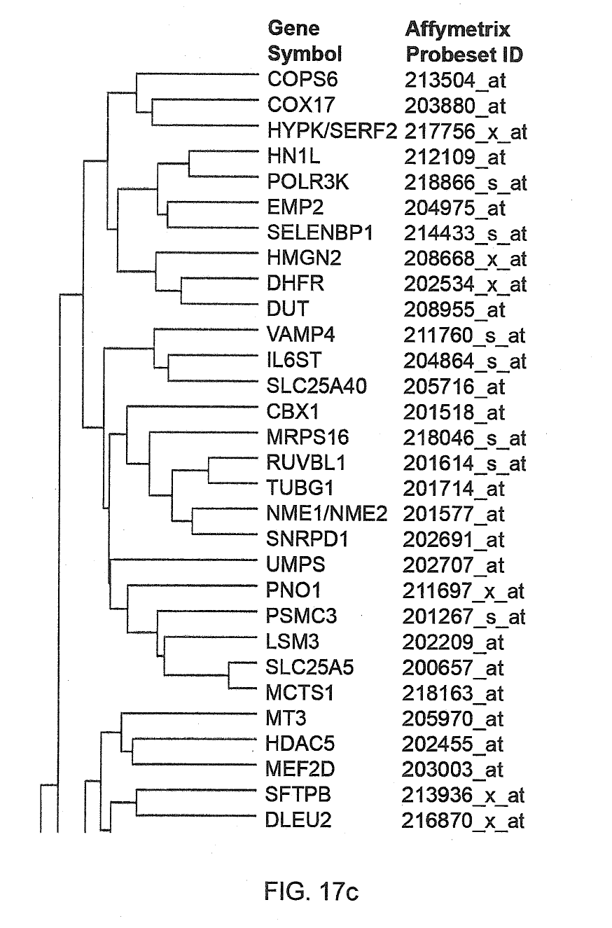

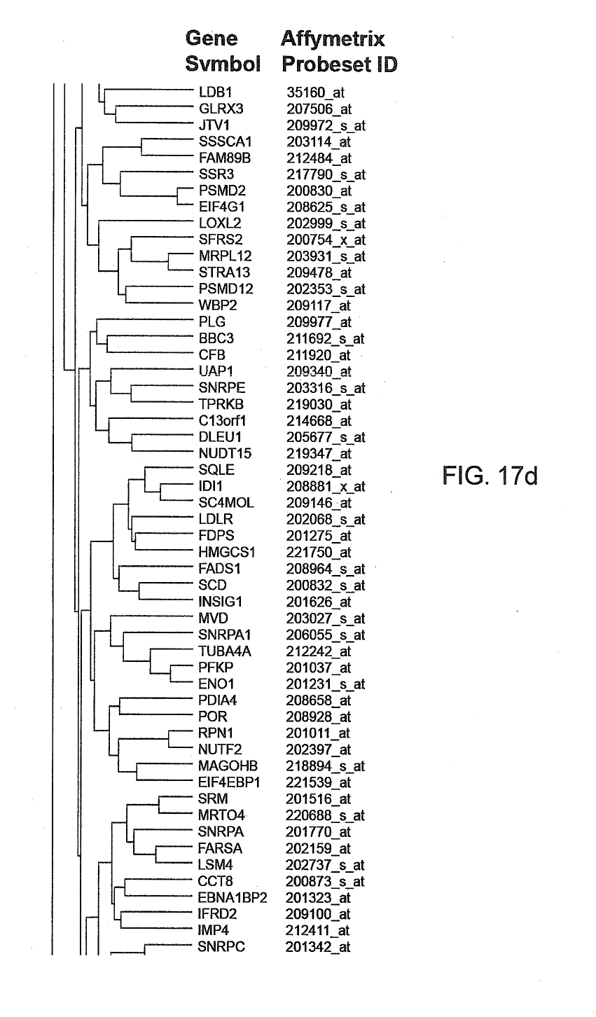

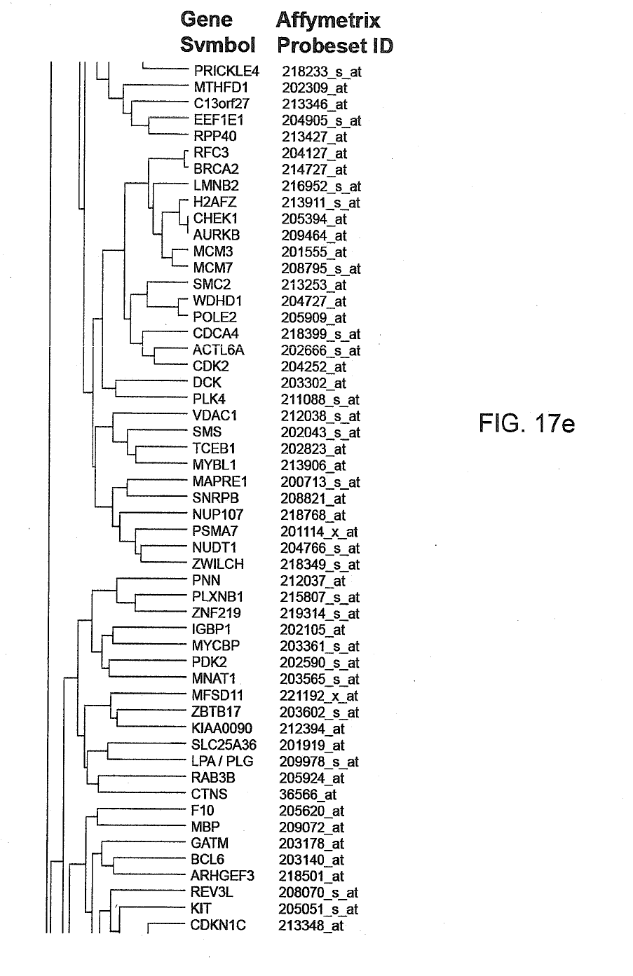

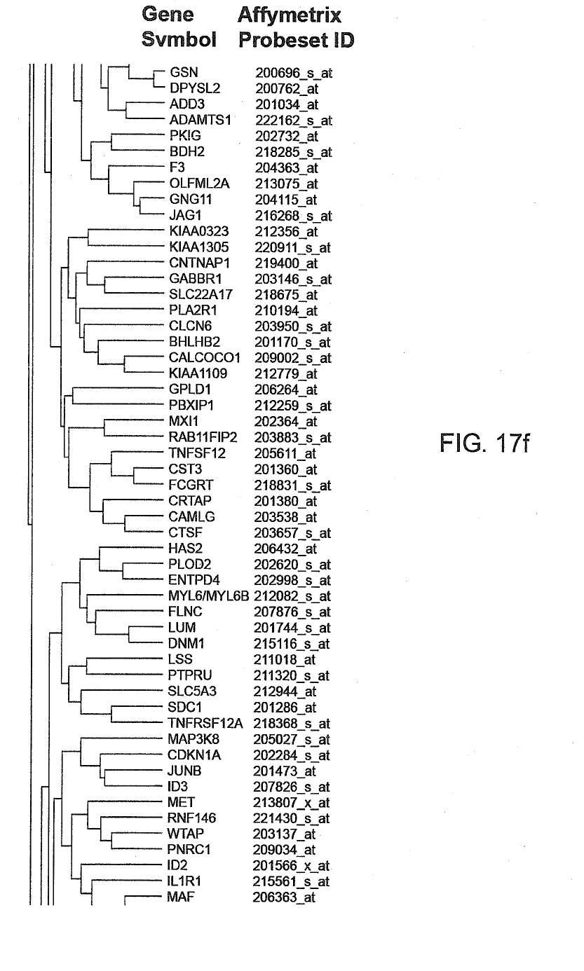

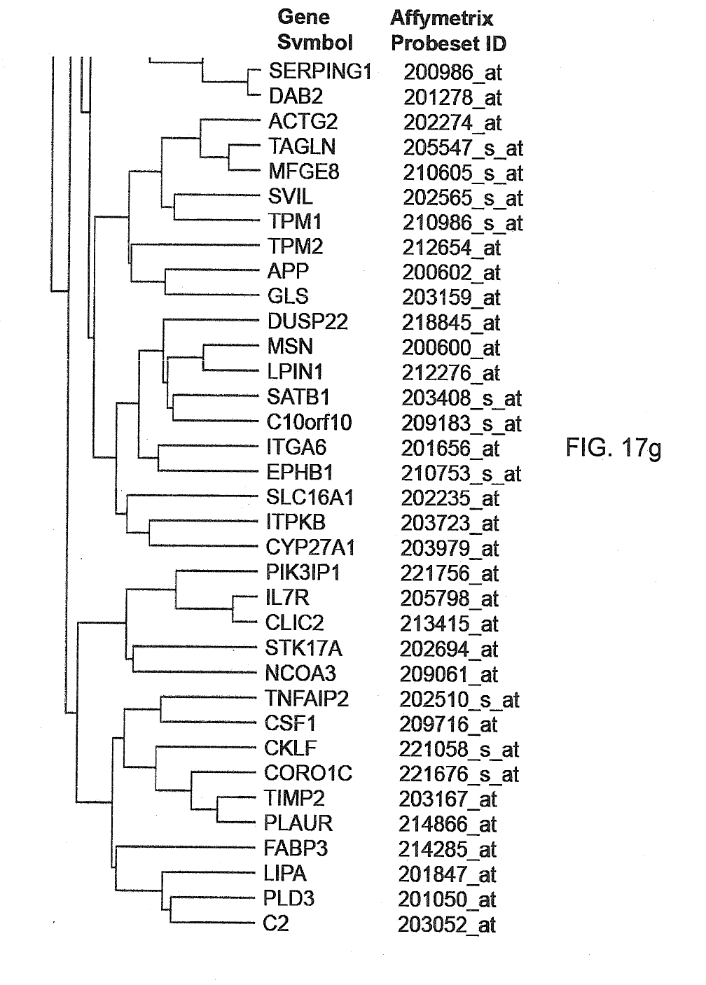

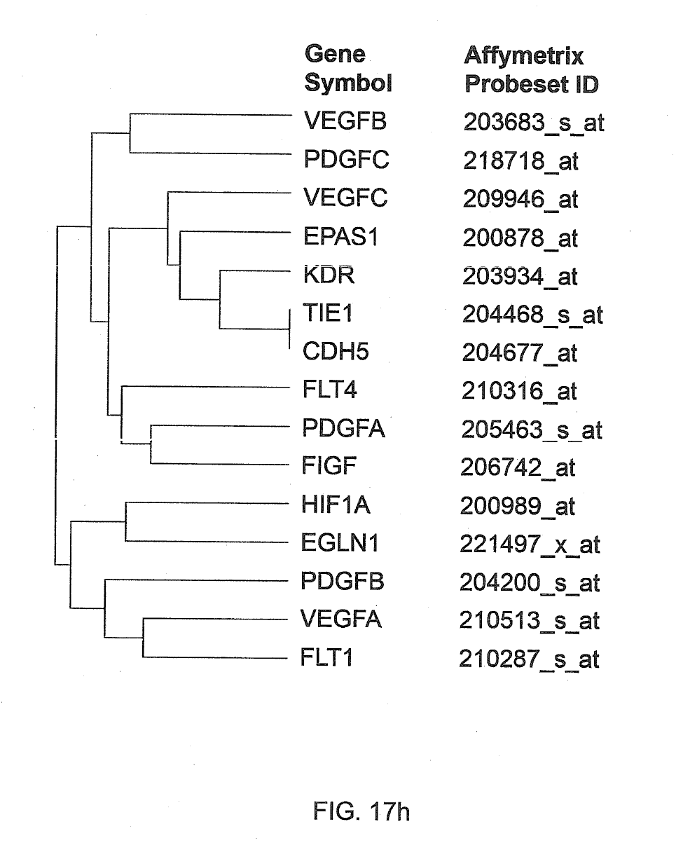

Another approach was also taken to validate the breast cancer molecular subtyping approach described herein. The subtyping genes were applied to determine breast cancer subtypes in three different independent datasets (34, 118 and 120) using centroid analysis. Whether the same molecular subtypes of breast cancer in the independent datasets shared the same gene expression characteristics for gene-expression signatures of wound-response (33), tumor stromal response (128), vascular endothelial normalization (129, 130) and cell cycle/proliferation was determined by hierarchical analyses to generate heat maps. None of the genes were used for molecular subtyping. All six molecular subtypes in the different breast cancer datasets shared the same distinct differential gene expression patterns according to the assigned molecular subtypes as demonstrated by heat maps. Thus, the classification genes can successfully distinguish the six different molecular subtypes of breast cancer in patients of different datasets. The same breast cancer molecular subtypes from different datasets shared the same molecular characteristics. The genes used to characterize cell cycle/proliferation, wound response, tumor stromal response, and vascular normal endothelial normalization are listed in FIGS. 17a-h.

### Example 8

### Identification of Differentially Expressed Genes Between Breast Cancer and Normal Breast Tissue for Each of Breast Cancer Molecular Subtypes I-VI

Microarray data of 367 breast samples including 327 breast cancer and 40 normal breast tissues were used for the study. Informative probe-sets were selected using the following two criteria: (a) Probe-sets with expression intensity greater than 9 (logarithm of normalized expression intensity with base 2) in at least 10 out of 367 samples; and (b) Probe-sets with fold-changes greater than 2 between the 90% quantile and the 10% quantile. All the selected probe-sets met both criteria. There were 5817 probe-sets that met both criteria.

Next, a two-sample t test between the breast cancer samples of each subtype and the normal breast samples was conducted to select probe-sets showing significant differences. Due to the large number of comparisons, a Benjamini & Hochberg method was used to adjust p-values for multiple comparisons. The purpose was to reduce false discovery rate (FDR). FDR was set at a level of <or =0.01 to identify probe-sets significantly different between each breast cancer subtype and normal breast tissues.

Differentially expressed genes were obtained for each of six breast cancer subtypes. The number of differentially expressed genes for each subtype is summarized in Table 19. However, many differentially expressed genes are shared between different subtypes of breast cancer. After eliminating probe-sets shared between different breast cancer molecular subtypes, probe-sets that are truly differentially expressed and unique to each molecular subtype of breast cancer were identified. The numbers of probe-sets unique to each molecular subtype are summarized in Table 20. The names of these genes and the probe-set IDs are listed in Tables 2-7 herein.

TABLE 19
Numbers of differentially expressed probe-sets between
each breast cancer subtype and normal breast tissue.

| | Breast Cancer Molecular Subtypes | | | | | |
|---|---|---|---|---|---|---|
| | I | II | III | IV | V | VI |
| Number of Differentially | 4110 | 4174 | 3990 | 4439 | 4057 | 3992 |
| Expressed Probe-sets | | | | | | |

TABLE 20
Numbers of differentially and uniquely expressed probe-sets
between each breast cancer subtype and normal breast tissue.

| | Breast Cancer Molecular Subtypes | | | | | |
|---|---|---|---|---|---|---|
| | I | II | III | IV | V | VI |
| Number of Differentially | 133 | 35 | 60 | 47 | 75 | 21 |
| Expressed Probe-sets | | | | | | |
| Unique to Each Subtype | | | | | | |

### Example 9

### Determination of the Minimum Number of Probe-Sets Needed to Yield Reliable Breast Cancer Molecular Subtype Classification Results

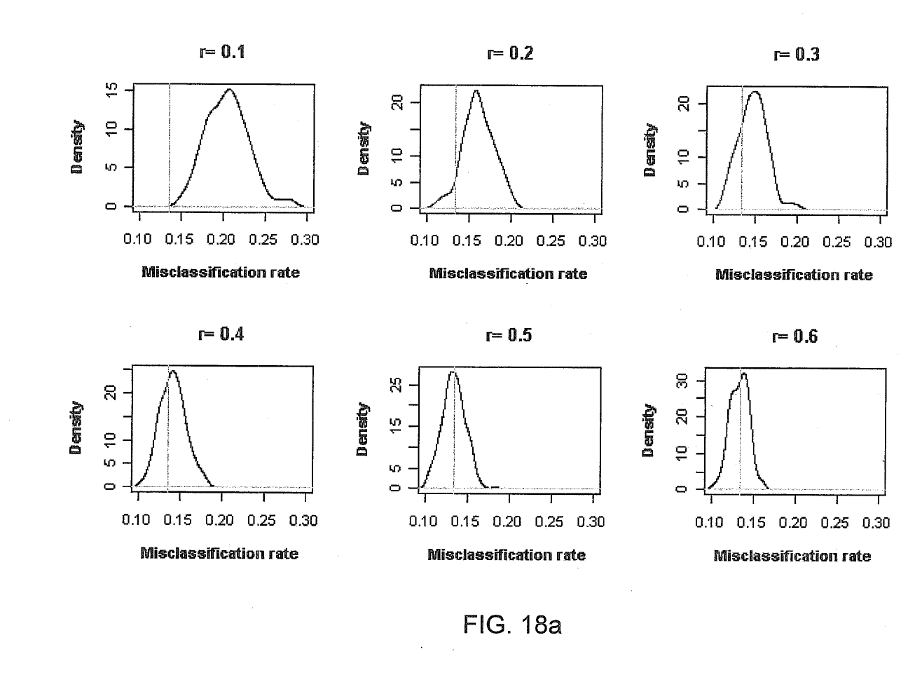

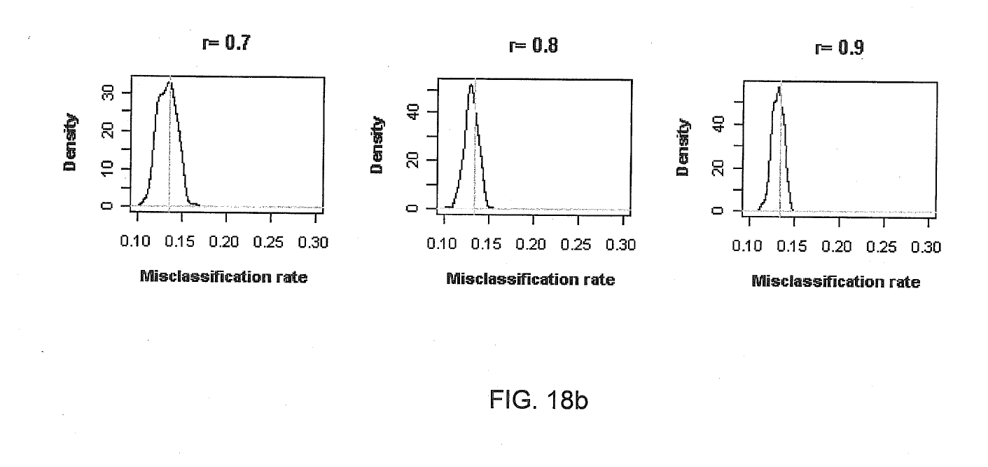

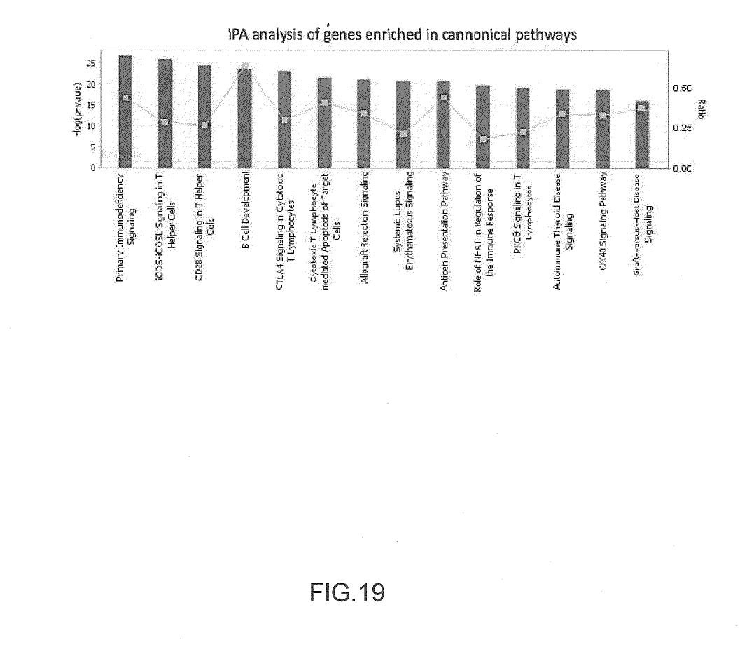

In this study, different numbers of randomly selected probe-sets from the 783 classification probe-sets described in Table 1 were evaluated to determine the number of probe-sets needed to reliably classify molecular subtypes of breast cancer samples. A centroid classification model, leave-one-out approach and different numbers of randomly selected probe-sets were used to classify each of the 327 breast cancer samples according to molecular subtype and to determine misclassification rates. The centroid model was employed because it is less restrictive and easy to apply. The following steps were performed in this study:
1. Different fractions (“r”) of the 783 classification probe-sets shown in Table 1 were randomly selected for the study. Thus, r=the number of randomly selected probe-sets divided by 783 (the total number of classification probe-sets). For this study, r was chosen to equal 0.1, 0.2, 0.3, 0.4, 0.5, 0.6, 0.7, 0.8 or 0.9.
2. A leave-one-out cross-validation was performed using a centroid model and the randomly selected probe-sets to subtype each of the 327 breast cancer samples for each r and determine the misclassification rate for each r.
3. Steps 1 and 2 were repeated 200 times, and 200 misclassification rates were obtained for each r.
4. Density plots of 200 misclassification rates for each r were generated (see FIG. 18).

All 783 classification probe-sets in Table 1 were initially used to conduct a leave-one-out study on each of the 327 samples. Using all 783 probe-sets yielded 44 misclassified samples, or a misclassification rate of 0.13 (13%).

To compare the misclassification rate of the centroid model at each r relative to the misclassification rate when all 783 probe-sets are used, an empirical 90% confidence interval (CI) of the misclassification rate was determined for each r. If the misclassification rate of the model using all 783 probe-sets (0.13) was smaller than or equal to the misclassification rate at the 5% quantile (lower bond of the 90% CI) for a specific r, the model was deemed worse than the model of using all 783 probe-sets. The results of the study are summarized in Table 21.

TABLE 21
Misclassification rates at the 5% and 95% quantiles using different numbers
of randomly selected probe-sets ranging from r = 0.1 to r = 0.9.

| | | Misclassification rate | | | | | | | | |
|---|---|---|---|---|---|---|---|---|---|---|
| | quantile | r = 0.1 | r = 0.2 | r = 0.3 | r = 0.4 | r = 0.5 | r = 0.6 | r = 0.7 | r = 0.8 | r = 0.9 |
| 90% | 5% | 0.17 | 0.13 | 0.12 | 0.12 | 0.11 | 0.12 | 0.12 | 0.12 | 0.12 |
| CI | 95% | 0.25 | 0.19 | 0.17 | 0.17 | 0.16 | 0.15 | 0.15 | 0.14 | 0.14 |

“r” is the fraction of the 783 classification probe-sets randomly selected for building a
“CI” is confidence interval.

The results show that the misclassification rate is not significantly worse when r is greater than or equal to 0.3. Moreover, 95% of all 200 classifications at each specific r yielded a misclassification rate that was no greater than 0.17. Therefore, 30% of the 783 probe-sets were sufficient to reliably classify the molecular subtype of a breast cancer.

### Example 10

### Immune Response Score is Predictive of Overall Survival

During our study of using Affymetrix Human GeneChips to classify breast cancer into different molecular subtypes, we observed immune response related genes were differentially expressed in the same molecular subtypes. This finding prompted us to investigate how different degrees of expressions of immune response genes may affect the survival outcome in different molecular subtypes of breast cancer.

10.1: Methods

Clinical and microarray data: The gene expression profiles and the clinical data from the same 327 patients used to discover different molecular subtypes of breast cancer were studied. To confirm our findings, we also included gene expression profiles of additional 180 breast cancer samples that we assayed recently.

Selection of immune response genes: For selection of immune response related genes, we first selected the probe-sets of CD3 (a specific cell surface marker for T lymphocytes) (Affymetrix probe-set ID: 213539_at) and CD19 (a specific cell surface marker for B lymphocytes) (Affymetrix probe-set ID: 206398_s_at) to represent key genes for humoral and cellular-mediated immune responses, respectively. The expression intensities of each probe-set in each of the 327 breast cancer samples was correlated with the intensities of the CD3 and CD19 probe-sets of the same breast cancer sample, separately. Pearson correlation was used to identify probe-sets correlated with the CD3 or the CD19 probe-sets. Only those probe-sets showing a Pearson correlation of 0.6 and above were selected.

The selected probe-sets were further filtered by choosing those probe-sets that had met the following two criteria. First, the selected probe-set should have gene expression intensity greater than 512 at least in 10 breast cancer samples. Second, the selected probe-set should show 2-fold change between 10th (top) and low 90th (bottom) percentiles in 327 samples.

Hierarchical clustering analysis: For hierachical clustering analysis, the average-linkage function and the complete linkage function were used on the breast cancer samples and the probe-sets, respectively.

Immune response score: The intensities of a probe-set across all samples in our dataset were calculated for their z scores. Z score is defined as [(expression intensity) minus (mean of a probe-set)] divided by (standard deviation). The immune score of a sample is the average of z-scored intensities of all immune response probe-sets of this breast cancer sample.

Molecular subtyping of the independent datasets: The molecular subtype of each breast cancer sample in an independent dataset was determined by using genes corresponding to our classification probe-sets and Centroid analysis (see Calza et al., “Intrinsic molecular signature of breast cancer in a population-based cohort of 412 patients” Breast Cancer Res, 8:R34 (2006)). The centroid model was created using our 327 breast cancer samples. If one probe-set was mapped to multiple genes in the independent datasets, the average intensity was calculated and applied.

Validation: For validation of our findings, we applied our immune response signature genes to breast cancer cases of the following five published independent datasets including TRANSBIG (GSE7390), MSKCC (GSE2603), Oxford (GSE2990), EMC (GSE2034), and Mainz (GSE11121). These datasets were available on GEO database and they were chosen because the same microarray platform (Affymetrix GeneChip) was used for gene expression profiling. The immune response score was determined for each case as described.

Statistical methods: All statistical analyses including hierarchical clustering, generation of heat maps, survival analysis by log-rank test, and other statistical testing were performed using R 2.11.0 software (http://www.r-project.org/).

10.2: Results

Immune response related probe-sets. Using the approach as described above, we identified 734 probe-sets related to immune response. All 734 probe-sets were analyzed by Ingenuity Pathway Analysis software from Ingenuity Systems (Redwood City, Calif.) to confirm that genes of these probe-sets are involved in immune responses. As shown in FIG. 18, the selected probe-sets are indeed enriched for various immunological functions with high degrees of statistical significance. The 734 probe-sets selected to assess immune response are summarized in Table 22.

TABLE 22

| Probe Set ID | Gene Symbol |
|---|---|
| 1405_i_at | CCL5 |
| 1552316_a_at | GIMAP1 |
| 1552318_at | GIMAP1 |
| 1552497_a_at | SLAMF6 |
| 1552584_at | IL12RB1 |
| 1552701_a_at | CARD16 |
| 1552703_s_at | CARD16 /// |
| | CASP1 |
| 1553102_a_at | CCDC69 |
| 1553681_a_at | PRF1 |
| 1553856_s_at | P2RY10 |
| 1553906_s_at | FGD2 |
| 1554208_at | MEI1 |
| 1554240_a_at | ITGAL |
| 1555349_a_at | ITGB2 |
| 1555355_a_at | ETS1 |
| 1555526_a_at | SEPT6 |
| 1555613_a_at | ZAP70 |
| 1555638_a_at | SAMSN1 |
| 1555691_a_at | KLRK1 |
| 1555759_a_at | CCL5 |
| 1555779_a_at | CD79A |
| 1555852_at | — |
| 1556657_at | — |
| 1556658_a_at | — |
| 1557116_at | APOL6 |
| 1557632_at | — |
| 1557718_at | PPP2R5C |
| 1558111_at | MBNL1 |
| 1558662_s_at | BANK1 |
| 1558972_s_at | THEMIS |
| 1559101_at | FYN |
| 1559263_s_at | PPIL4 /// |
| | ZC3H12D |
| 1559425_at | — |
| 1559584_a_at | C16orf54 |
| 1560332_at | — |
| 1560396_at | KLHL6 |
| 1560706_at | — |
| 1562194_at | — |
| 1563357_at | — |
| 1563473_at | — |
| 1563674_at | FCRL2 |
| 1564077_at | — |
| 1564139_at | LOC144571 |
| 1565705_x_at | — |
| 1565752_at | FGD2 |
| 1565754_x_at | FGD2 |
| 1568943_at | INPP5D |
| 1569040_s_at | FLJ40330 |
| 1569225_a_at | SCML4 |
| 200628_s_at | WARS |
| 200629_at | WARS |
| 200887_s_at | STAT1 |
| 200904_at | HLA-E |
| 200905_x_at | HLA-E |
| 201137_s_at | HLA-DPB1 |
| 201153_s_at | MBNL1 |
| 201487_at | CTSC |
| 201720_s_at | LAPTM5 |
| 201721_s_at | LAPTM5 |
| 201858_s_at | SRGN |
| 201859_at | SRGN |
| 202156_s_at | CELF2 |
| 202157_s_at | CELF2 |
| 202269_x_at | GBP1 |
| 202270_at | GBP1 |
| 202307_s_at | TAP1 |
| 202524_s_at | SPOCK2 |
| 202531_at | IRF1 |
| 202625_at | LYN |
| 202626_s_at | LYN |
| 202643_s_at | TNFAIP3 |
| 202644_s_at | TNFAIP3 |
| 202659_at | PSMB10 |
| 202663_at | WIPF1 |
| 202664_at | WIPF1 |
| 202665_s_at | WIPF1 |
| 202693_s_at | STK17A |
| 202748_at | GBP2 |
| 202803_s_at | ITGB2 |
| 202901_x_at | CTSS |
| 202902_s_at | CTSS |
| 202910_s_at | CD97 |
| 202957_at | HCLS1 |
| 203047_at | STK10 |
| 203110_at | PTK2B |
| 203185_at | RASSF2 |
| 203332_s_at | INPP5D |
| 203385_at | DGKA |
| 203402_at | KCNAB2 |
| 203416_at | CD53 |
| 203470_s_at | PLEK |
| 203471_s_at | PLEK |
| 203508_at | TNFRSF1B |
| 203523_at | LSP1 |
| 203528_at | SEMA4D |
| 203547_at | CD4 |
| 203741_s_at | ADCY7 |
| 203760_s_at | SLA |
| 203761_at | SLA |
| 203828_s_at | IL32 |
| 203845_at | KAT2B |
| 203868_s_at | VCAM1 |
| 203879_at | PIK3CD |
| 203915_at | CXCL9 |
| 203922_s_at | CYBB |
| 203923_s_at | CYBB |
| 203932_at | HLA-DMB |
| 204057_at | IRF8 |
| 204116_at | IL2RG |
| 204118_at | CD48 |
| 204153_s_at | MFNG |
| 204192_at | CD37 |
| 204197_s_at | RUNX3 |
| 204198_s_at | RUNX3 |
| 204205_at | APOBEC3G |
| 204220_at | GMFG |
| 204236_at | FLI1 |
| 204265_s_at | GPSM3 |
| 204269_at | PIM2 |
| 204279_at | PSMB9 |
| 204502_at | SAMHD1 |
| 204513_s_at | ELMO1 |
| 204529_s_at | TOX |
| 204533_at | CXCL10 |
| 204562_at | IRF4 |
| 204563_at | SELL |
| 204588_s_at | SLC7A7 |
| 204613_at | PLCG2 |
| 204639_at | ADA |
| 204655_at | CCL5 |
| 204661_at | CD52 |
| 204670_x_at | HLA-DRB1 /// |
| | HLA-DRB4 |
| 204674_at | LRMP |
| 204683_at | ICAM2 |
| 204774_at | EVI2A |
| 204789_at | FMNL1 |
| 204806_x_at | HLA-F |
| 204820_s_at | BTN3A2 /// |
| | BTN3A3 |
| 204821_at | BTN3A3 |
| 204834_at | FGL2 |
| 204852_s_at | PTPN7 |
| 204882_at | ARHGAP25 |
| 204890_s_at | LCK |
| 204891_s_at | LCK |
| 204897_at | PTGER4 |
| 204912_at | IL10RA |
| 204923_at | SASH3 |
| 204949_at | ICAM3 |
| 204959_at | MNDA |
| 204960_at | PTPRCAP |
| 204961_s_at | NCF1 /// |
| | NCF1B /// |
| | NCF1C |
| 204982_at | GIT2 |
| 205039_s_at | IKZF1 |
| 205049_s_at | CD79A |
| 205101_at | CIITA |
| 205147_x_at | NCF4 |
| 205153_s_at | CD40 |
| 205159_at | CSF2RB |
| 205213_at | ACAP1 |
| 205214_at | STK17B |
| 205255_x_at | TCF7 |
| 205267_at | POU2AF1 |
| 205269_at | LCP2 |
| 205270_s_at | LCP2 |
| 205285_s_at | FYB |
| 205291_at | IL2RB |
| 205297_s_at | CD79B |
| 205298_s_at | BTN2A2 |
| 205404_at | HSD11B1 |
| 205419_at | GPR183 |
| 205456_at | CD3E |
| 205484_at | SIT1 |
| 205488_at | GZMA |
| 205495_s_at | GNLY |
| 205504_at | BTK |
| 205544_s_at | CR2 |
| 205569_at | LAMP3 |
| 205639_at | AOAH |
| 205671_s_at | HLA-DOB |
| 205681_at | BCL2A1 |
| 205685_at | CD86 |
| 205686_s_at | CD86 |
| 205692_s_at | CD38 |
| 205758_at | CD8A |
| 205798_at | IL7R |
| 205801_s_at | RASGRP3 |
| 205804_s_at | TRAF3IP3 |
| 205821_at | KLRK1 |
| 205831_at | CD2 |
| 205861_at | SPIB |
| 205885_s_at | ITGA4 |
| 205890_s_at | GABBR1 /// |
| | UBD |
| 205988_at | CD84 |
| 205992_s_at | IL15 |
| 206011_at | CASP1 |
| 206060_s_at | PTPN22 |
| 206118_at | STAT4 |
| 206134_at | ADAMDEC1 |
| 206150_at | CD27 |
| 206206_at | CD180 |
| 206219_s_at | VAV1 |
| 206296_x_at | MAP4K1 |
| 206332_s_at | IFI16 |
| 206337_at | CCR7 |
| 206366_x_at | XCL1 |
| 206398_s_at | CD19 |
| 206478_at | KIAA0125 |
| 206486_at | LAG3 |
| 206513_at | AIM2 |
| 206584_at | LY96 |
| 206637_at | P2RY14 |
| 206641_at | TNFRSF17 |
| 206666_at | GZMK |
| 206682_at | CLEC10A |
| 206687_s_at | PTPN6 |
| 206707_x_at | FAM65B |
| 206715_at | TFEC |
| 206785_s_at | KLRC1 /// |
| | KLRC2 |
| 206914_at | CRTAM |
| 206974_at | CXCR6 |
| 206978_at | CCR2 |
| 206991_s_at | CCR5 |
| 207238_s_at | PTPRC |
| 207339_s_at | LTB |
| 207375_s_at | IL15RA |
| 207419_s_at | RAC2 |
| 207485_x_at | BTN3A1 |
| 207536_s_at | TNFRSF9 |
| 207551_s_at | MSL3 |
| 207571_x_at | C1orf38 |
| 207651_at | GPR171 |
| 207677_s_at | NCF4 |
| 207697_x_at | LILRB2 |
| 207734_at | LAX1 |
| 207777_s_at | SP140 |
| 207957_s_at | PRKCB |
| 208018_s_at | HCK |
| 208146_s_at | CPVL |
| 208206_s_at | RASGRP2 |
| 208268_at | ADAM28 |
| 208296_x_at | TNFAIP8 |
| 208306_x_at | HLA-DRB1 |
| 208442_s_at | ATM |
| 208450_at | LGALS2 |
| 208729_x_at | HLA-B |
| 208885_at | LCP1 |
| 208894_at | HLA-DRA |
| 208965_s_at | IFI16 |
| 208966_x_at | IFI16 |
| 209083_at | CORO1A |
| 209138_x_at | IGL@ |
| 209201_x_at | CXCR4 |
| 209310_s_at | CASP4 |
| 209312_x_at | HLA-DRB1 /// |
| | HLA-DRB4 /// |
| | HLA-DRB5 |
| 209374_s_at | IGHM |
| 209584_x_at | APOBEC3C |
| 209606_at | CYTIP |
| 209619_at | CD74 |
| 209670_at | TRAC |
| 209671_x_at | TRA@/// |
| | TRAC |
| 209685_s_at | PRKCB |
| 209723_at | SERPINB9 |
| 209732_at | CLEC2B |
| 209734_at | NCKAP1L |
| 209770_at | BTN3A1 |
| 209795_at | CD69 |
| 209813_x_at | TARP |
| 209827_s_at | IL16 |
| 209829_at | FAM65B |
| 209846_s_at | BTN3A2 |
| 209879_at | SELPLG |
| 209939_x_at | CFLAR |
| 209969_s_at | STAT1 |
| 209970_x_at | CASP1 |
| 209995_s_at | TCL1A |
| 210029_at | IDO1 |
| 210031_at | CD247 |
| 210038_at | PRKCQ |
| 210072_at | CCL19 |
| 210105_s_at | FYN |
| 210113_s_at | NLRP1 |
| 210116_at | SH2D1A |
| 210140_at | CST7 |
| 210146_x_at | LILRB2 |
| 210163_at | CXCL11 |
| 210164_at | GZMB |
| 210260_s_at | TNFAIP8 |
| 210279_at | GPR18 |
| 210288_at | KLRG1 |
| 210321_at | GZMH |
| 210356_x_at | MS4A1 |
| 210439_at | ICOS |
| 210448_s_at | P2RX5 |
| 210514_x_at | HLA-G |
| 210538_s_at | BIRC3 |
| 210555_s_at | NFATC3 |
| 210563_x_at | CFLAR |
| 210644_s_at | LAIR1 |
| 210681_s_at | USP15 |
| 210754_s_at | LYN |
| 210785_s_at | C1orf38 |
| 210786_s_at | FLI1 |
| 210858_x_at | ATM |
| 210895_s_at | CD86 |
| 210915_x_at | TRBC1 |
| 210972_x_at | TRA@/// |
| | TRAC /// |
| | TRAJ17 /// |
| | TRAV20 |
| 210982_s_at | HLA-DRA |
| 211005_at | LAT /// SPNS1 |
| 211122_s_at | CXCL11 |
| 211144_x_at | TARP /// |
| | TRGC2 |
| 211339_s_at | ITK |
| 211366_x_at | CASP1 |
| 211367_s_at | CASP1 |
| 211368_s_at | CASP1 |
| 211430_s_at | IGH@/// |
| | IGHG1 /// |
| | IGHG2 /// |
| | IGHM /// |
| | IGHV4-31 /// |
| | LOC100290146 |
| | /// |
| | LOC100294459 |
| 211582_x_at | LST1 |
| 211633_x_at | — |
| 211634_x_at | IGHM /// |
| | LOC100133862 |
| 211635_x_at | IGH@/// |
| | IGHA1 /// |
| | IGHA2 /// IGHD |
| | /// IGHG1 /// |
| | IGHG3 /// |
| | IGHG4 /// |
| | IGHM /// |
| | IGHV4-31 /// |
| | LOC100133862 |
| | /// |
| | LOC100290146 |
| | /// |
| | LOC100290528 |
| 211637_x_at | IGH@/// |
| | IGHA1 /// |
| | IGHA2 /// IGHD |
| | /// IGHG1 /// |
| | IGHG3 /// |
| | IGHG4 /// |
| | IGHM /// |
| | IGHV3-23 /// |
| | LOC100126583 |
| | /// |
| | LOC100290146 |
| | /// LOC652128 |
| 211639_x_at | IGH@/// |
| | IGHA1 /// |
| | IGHA2 /// IGHD |
| | /// IGHG1 /// |
| | IGHG3 /// |
| | IGHG4 /// |
| | IGHM /// |
| | IGHV4-31 /// |
| | LOC100126583 |
| | /// LOC652128 |
| 211640_x_at | IGHG1 /// |
| | IGHM /// |
| | LOC100133862 |
| 211641_x_at | IGH@/// |
| | IGHA1 /// |
| | IGHA2 /// IGHD |
| | /// IGHG1 /// |
| | IGHG3 /// |
| | IGHM /// |
| | IGHV4-31 /// |
| | LOC100290320 |
| | /// |
| | LOC100291190 |
| 211643_x_at | IGK@/// IGKC |
| | /// IGKV3D-15 |
| 211644_x_at | IGK@/// IGKC |
| | /// IGKV3-20 /// |
| | LOC100291682 |
| 211645_x_at | — |
| 211649_x_at | IGH@/// |
| | IGHA1 /// |
| | IGHG1 /// |
| | IGHM |
| 211650_x_at | IGHA1 /// IGHD |
| | /// IGHG1 /// |
| | IGHG3 /// |
| | IGHM /// |
| | IGHV1-69 /// |
| | IGHV3-23 /// |
| | IGHV4-31 /// |
| | LOC100126583 |
| | /// |
| | LOC100290375 |
| 211654_x_at | HLA-DQB1 |
| 211656_x_at | HLA-DQB1 /// |
| | LOC100294318 |
| 211663_x_at | PTGDS |
| 211742_s_at | EVI2B |
| 211748_x_at | PTGDS |
| 211795_s_at | FYB |
| 211796_s_at | TRBC1 |
| 211798_x_at | IGLJ3 |
| 211822_s_at | NLRP1 |
| 211824_x_at | NLRP1 |
| 211868_x_at | IGH@/// |
| | IGHA1 /// |
| | IGHA2 /// IGHD |
| | /// IGHG1 /// |
| | IGHG2 /// |
| | IGHG3 /// |
| | IGHM /// |
| | IGHV4-31 /// |
| | LOC100126583 |
| | /// |
| 213293_s_at | TRIM22 |
| 213309_at | PLCL2 |
| 213415_at | CLIC2 |
| 213416_at | ITGA4 |
| 213475_s_at | ITGAL |
| 213539_at | CD3D |
| 213566_at | RNASE6 |
| 213603_s_at | RAC2 |
| 213618_at | ARAP2 |
| 213620_s_at | ICAM2 |
| 213666_at | |
| 213733_at | MYO1F |
| 213830_at | TRD@ |
| 213888_s_at | TRAF3IP3 |
| 213915_at | NKG7 |
| 213958_at | CD6 |
| 213975_s_at | LYZ |
| 213982_s_at | RABGAP1L |
| 214032_at | ZAP70 |
| 214054_at | DOK2 |
| 214084_x_at | NCF1C |
| 214181_x_at | LST1 |
| 214298_x_at | |
| 214339_s_at | MAP4K1 |
| 214369_s_at | RASGRP2 |
| 214450_at | CTSW |
| 214467_at | GPR65 |
| 214470_at | KLRB1 |
| 214567_s_at | XCL1 /// XCL2 |
| 214574_x_at | LST1 |
| 214582_at | PDE3B |
| 214617_at | PRF1 |
| 214669_x_at | IGKC |
| 214677_x_at | CYAT1 /// |
| | IGLV1-44 |
| 214735_at | IPCEF1 |
| 214768_x_at | — |
| 214777_at | IGKV4-1 |
| 214836_x_at | IGK@/// IGKC |
| 214916_x_at | IGH@/// IGHA1 |
| | /// IGHA2 /// |
| | IGHG1 /// |
| | IGHG3 /// IGHM |
| | /// IGHV3-23 /// |
| | IGHV4-31 /// |
| | LOC100290375 |
| 214973_x_at | IGHD /// |
| | LOC100290059 |
| | /// |
| | LOC100292999 |
| 214995_s_at | APOBEC3F /// |
| | APOBEC3G |
| 215051_x_at | AIF1 |
| 215118_s_at | IGHA1 |
| 215121_x_at | CYAT1 /// |
| | IGLV1-44 |
| 215147_at | — |
| 215176_x_at | IGK@/// IGKC |
| | /// |
| | LOC100291464 |
| 215193_x_at | HLA-DRB1 /// |
| | HLA-DRB3 /// |
| | HLA-DRB4 |
| 215214_at | IGL@ |
| 215346_at | CD40 |
| 215379_x_at | IGLV1-44 |
| 215565_at | LOC100289053 |
| 215633_x_at | LST1 |
| 215806_x_at | TARP /// TRGC2 |
| 215946_x_at | IGLL3 |
| 215949_x_at | IGHM /// |
| | LOC652494 |
| 215967_s_at | LY9 |
| 216033_s_at | FYN |
| 216191_s_at | TRA@/// |
| | TRD@ |
| 216207_x_at | IGKV1D-13 |
| 216250_s_at | LPXN |
| 216365_x_at | IGLV3-19 |
| 216401_x_at | LOC652493 |
| 216412_x_at | LOC100290557 |
| 216430_x_at | IGLV1-44 /// |
| | LOC100290557 |
| 216491_x_at | IGHM |
| 216510_x_at | IGHA1 /// |
| | IGHG1 /// IGHM |
| | /// IGHV3-23 /// |
| | IGHV4-31 /// |
| | LOC100290375 |
| 216542_x_at | IGHA1 /// |
| | IGHG1 /// IGHM |
| | /// |
| | LOC100290293 |
| 216557_x_at | IGHA1 /// IGHD |
| | /// IGHG1 /// |
| | IGHG3 /// IGHM |
| | /// IGHV4-31 /// |
| | LOC100290320 |
| | /// |
| | LOC100291190 |
| 216560_x_at | IGL@ |
| 216576_x_at | IGK@/// IGKC |
| | /// LOC652493 |
| | /// LOC652694 |
| 216829_at | IGK@/// IGKC |
| | /// LOC652493 |
| | /// LOC652694 |
| 216853_x_at | IGLV3-19 |
| 216920_s_at | TARP /// TRGC2 |
| 216984_x_at | IGLV2-23 /// |
| | LOC100293440 |
| 217028_at | CXCR4 |
| 217143_s_at | TRA@/// |
| | TRD@ |
| 217147_s_at | TRAT1 |
| 217148_x_at | LOC100293440 |
| 217157_x_at | IGK@/// IGKC |
| | /// LOC652493 |
| 217179_x_at | — |
| 217227_x_at | IGLV1-44 /// |
| | LOC100290557 |
| 217235_x_at | IGLL5 /// IGLV2- |
| | 23 |
| 217258_x_at | IGLV1-44 /// |
| | LOC100290557 |
| 217281_x_at | IGH@/// IGHA1 |
| | /// IGHA2 /// |
| | IGHG1 /// |
| | IGHG2 /// |
| | IGHG3 /// IGHM |
| | /// IGHV4-31 /// |
| | LOC100126583 |
| | /// |
| | LOC100290036 |
| 217360_x_at | IGHA1 /// |
| | IGHG1 /// |
| | IGHG3 /// IGHM |
| | /// IGHV4-31 /// |
| | LOC652494 |
| 217378_x_at | LOC100130100 |
| | /// |
| | LOC100291464 |
| 217418_x_at | MS4A1 |
| 217436_x_at | HLA-J |
| 217456_x_at | HLA-E |
| 217478_s_at | HLA-DMA |
| 217480_x_at | LOC100287723 |
| | /// LOC642424 |
| | /// LOC642838 |
| 217549_at | — |
| 217933_s_at | LAP3 |
| 218223_s_at | PLEKHO1 |
| 218232_at | C1QA |
| 218322_s_at | ACSL5 |
| 218805_at | GIMAP5 |
| 218870_at | ARHGAP15 |
| 218999_at | TMEM140 |
| 219014_at | PLAC8 |
| 219045_at | RHOF |
| 219159_s_at | SLAMF7 |
| 219183_s_at | CYTH4 |
| 219191_s_at | BIN2 |
| 219243_at | GIMAP4 |
| 219279_at | DOCK10 |
| 219282_s_at | TRPV2 |
| 219385_at | SLAMF8 |
| 219386_s_at | SLAMF8 |
| 219505_at | CECR1 |
| 219528_s_at | BCL11B |
| 219551_at | EAF2 |
| 219574_at | |
| 219667_s_at | BANK1 |
| 219690_at | TMEM149 |
| 219777_at | GIMAP6 |
| 219812_at | PVRIG |
| 220059_at | STAP1 |
| 220068_at | VPREB3 |
| 220132_s_at | CLEC2D |
| 220330_s_at | SAMSN1 |
| 220560_at | C11orf21 |
| 220577_at | GVIN1 |
| 220704_at | IKZF1 |
| 221004_s_at | ITM2C |
| 221059_s_at | COTL1 |
| 221080_s_at | DENND1C |
| 221087_s_at | APOL3 |
| 221286_s_at | MGC29506 |
| 221601_s_at | FAIM3 |
| 221602_s_at | FAIM3 |
| 221658_s_at | IL21R |
| 221875_x_at | HLA-F |
| 221903_s_at | CYLD |
| 221969_at | PAX5 |
| 221978_at | HLA-F |
| 222592_s_at | ACSL5 |
| 222838_at | SLAMF7 |
| 222859_s_at | DAPP1 |
| 222868_s_at | IL18BP |
| 222895_s_at | BCL11B |
| 223082_at | SH3KBP1 |
| 223280_x_at | MS4A6A |
| 223303_at | FERMT3 |
| 223322_at | RASSF5 |
| 223501_at | TNFSF13B |
| 223502_s_at | TNFSF13B |
| 223533_at | LRRC8C |
| 223553_s_at | DOK3 |
| 223562_at | PARVG |
| 223565_at | MGC29506 |
| 223583_at | TNFAIP8L2 |
| 223640_at | HCST |
| 223751_x_at | TLR10 |
| 223980_s_at | SP110 |
| 224342_x_at | LOC96610 |
| 224356_x_at | MS4A6A |
| 224404_s_at | FCRL5 |
| 224406_s_at | FCRL5 |
| 224451_x_at | ARHGAP9 |
| 224583_at | COTL1 |
| 224709_s_at | CDC42SE2 |
| 224833_at | ETS1 |
| 224927_at | KIAA1949 |
| 224964_s_at | GNG2 |
| 225282_at | SMAP2 |
| 225364_at | STK4 |
| 225373_at | C10orf54 |
| 225502_at | DOCK8 |
| 225622_at | PAG1 |
| 225626_at | PAG1 |
| 225646_at | CTSC |
| 225647_s_at | CTSC |
| 225701_at | AKNA |
| 225763_at | RCSD1 |
| 225973_at | TAP2 |
| 226068_at | SYK |
| 226218_at | IL7R |
| 226219_at | ARHGAP30 |
| 226436_at | RASSF4 |
| 226459_at | PIK3AP1 |
| 226474_at | NLRC5 |
| 226525_at | STK17B |
| 226603_at | SAMD9L |
| 226633_at | RAB8B |
| 226641_at | — |
| 226659_at | DEF6 |
| 226711_at | FOXN2 |
| 226818_at | MPEG1 |
| 226841_at | MPEG1 |
| 226875_at | DOCK11 |
| 226878_at | HLA-DOA |
| 226879_at | HVCN1 |
| 226906_s_at | ARHGAP9 |
| 226991_at | NFATC2 |
| 227002_at | FAM78A |
| 227030_at | — |
| 227087_at | INPP4A |
| 227178_at | CELF2 |
| 227189_at | CPNE5 |
| 227265_at | FGL2 |
| 227266_s_at | FYB |
| 227344_at | IKZF1 |
| 227346_at | IKZF1 |
| 227353_at | TMC8 |
| 227354_at | PAG1 |
| 227458_at | CD274 |
| 227552_at | |
| 227606_s_at | STAMBPL1 |
| 227607_at | STAMBPL1 |
| 227609_at | EPSTI1 |
| 227645_at | PIK3R5 |
| 227677_at | JAK3 |
| 227726_at | RNF166 |
| 227749_at | — |
| 227791_at | SLC9A9 |
| 227877_at | C5orf39 |
| 228007_at | C6orf204 |
| 228055_at | NAPSB |
| 228071_at | GIMAP7 |
| 228094_at | AMICA1 |
| 228167_at | KLHL6 |
| 228258_at | TBC1D10C |
| 228372_at | C10orf128 |
| 228410_at | GAB3 |
| 228426_at | CLEC2D |
| 228442_at | NFATC2 |
| 228471_at | ANKRD44 |
| 228532_at | C1orf162 |
| 228592_at | MS4A1 |
| 228599_at | MS4A1 |
| 228641_at | CARD8 |
| 228677_s_at | RASAL3 |
| 228826_at | — |
| 228869_at | SNX20 |
| 228964_at | PRDM1 |
| 229041_s_at | — |
| 229367_s_at | GIMAP6 |
| 229383_at | |
| 229390_at | FAM26F |
| 229391_s_at | FAM26F |
| 229437_at | MIR155HG |
| 229560_at | TLR8 |
| 229597_s_at | WDFY4 |
| 229625_at | GBP5 |
| 229629_at | — |
| 229670_at | — |
| 229686_at | P2RY8 |
| 229723_at | TAGAP |
| 229750_at | POU2F2 |
| 229937_x_at | LILRB1 |
| 230011_at | MEI1 |
| 230036_at | SAMD9L |
| 230110_at | MCOLN2 |
| 230261_at | ST8SIA4 |
| 230383_x_at | — |
| 230391_at | CD84 |
| 230499_at | — |
| 230550_at | MS4A6A |
| 230753_at | PATL2 |
| 230805_at | — |
| 230836_at | ST8SIA4 |
| 230917_at | — |
| 230925_at | APBB1IP |
| 231093_at | FCRL3 |
| 231124_x_at | LY9 |
| 231577_s_at | GBP1 |
| 231647_s_at | FCRL5 |
| 231776_at | EOMES |
| 232024_at | GIMAP2 |
| 232234_at | SLA2 |
| 232375_at | — |
| 232383_at | TFEC |
| 232543_x_at | ARHGAP9 |
| 232583_at | — |
| 232617_at | CTSS |
| 232843_s_at | DOCK8 |
| 233302_at | — |
| 233411_at | — |
| 233500_x_at | CLEC2D |
| 233510_s_at | PARVG |
| 234050_at | TAGAP |
| 234260_at | — |
| 234366_x_at | CYAT1 |
| 234419_x_at | IGH@/// IGHA1 |
| | /// IGHG1 /// |
| | IGHG3 /// IGHM |
| | /// IGHV4-31 /// |
| | LOC100293211 |
| 234764_x_at | IGLV1-44 |
| 234884_x_at | CYAT1 |
| 234987_at | — |
| 235175_at | GBP4 |
| 235229_at | — |
| 235276_at | EPSTI1 |
| 235291_s_at | FLJ32255 |
| 235306_at | GIMAP8 |
| 235372_at | FCRLA |
| 235385_at | |
| 235529_x_at | — |
| 235574_at | GBP4 |
| 235879_at | MBNL1 |
| 235964_x_at | — |
| 236191_at | — |
| 236198_at | — |
| 236280_at | — |
| 236295_s_at | NLRC3 |
| 236341_at | CTLA4 |
| 236539_at | PTPN22 |
| 236782_at | SAMD3 |
| 236921_at | — |
| 237104_at | — |
| 237176_at | — |
| 237625_s_at | — |
| 237753_at | — |
| 238025_at | MLKL |
| 238531_x_at | — |
| 238581_at | GBP5 |
| 238668_at | — |
| 238725_at | IRF1 |
| 239237_at | — |
| 239294_at | — |
| 239409_at | — |
| 239629_at | CFLAR |
| 239979_at | — |
| 240070_at | TIGIT |
| 240154_at | — |
| 240413_at | PYHIN1 |
| 240481_at | — |
| 240665_at | — |
| 240890_at | LOC643733 |
| 241435_at | — |
| 241891_at | — |
| 241917_at | — |
| 242020_s_at | ZBP1 |
| 242268_at | CELF2 |
| 242388_x_at | TAGAP |
| 242521_at | — |
| 242814_at | SERPINB9 |
| 242827_x_at | — |
| 242907_at | — |
| 242943_at | ST8SIA4 |
| 242946_at | — |
| 243006_at | — |
| 243271_at | — |
| AFFX- | STAT1 |
| HUMISGF3A/ | |
| M97935_3_at | |
| AFFX- | STAT1 |
| HUMISGF3A/ | |
| M97935_MA_at | |

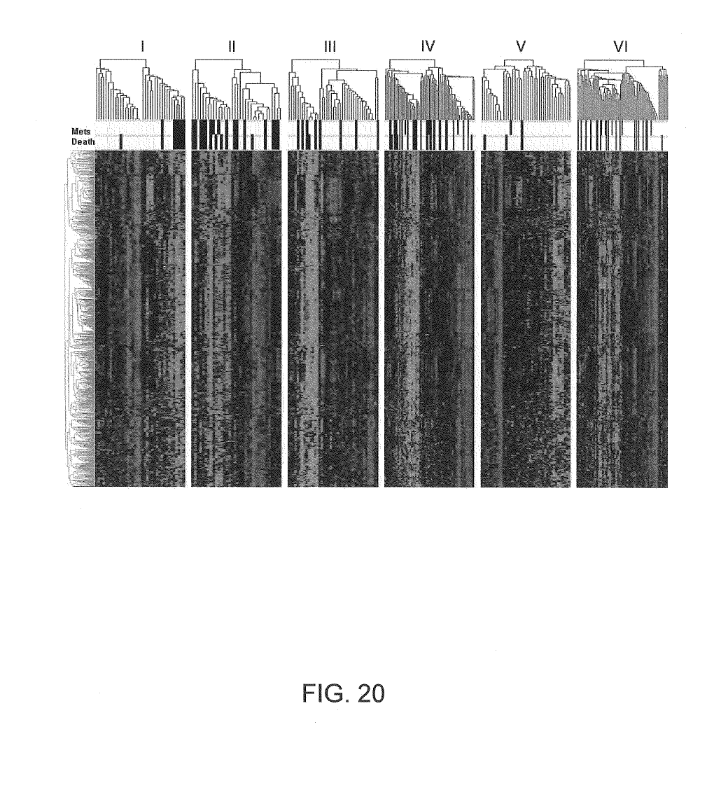

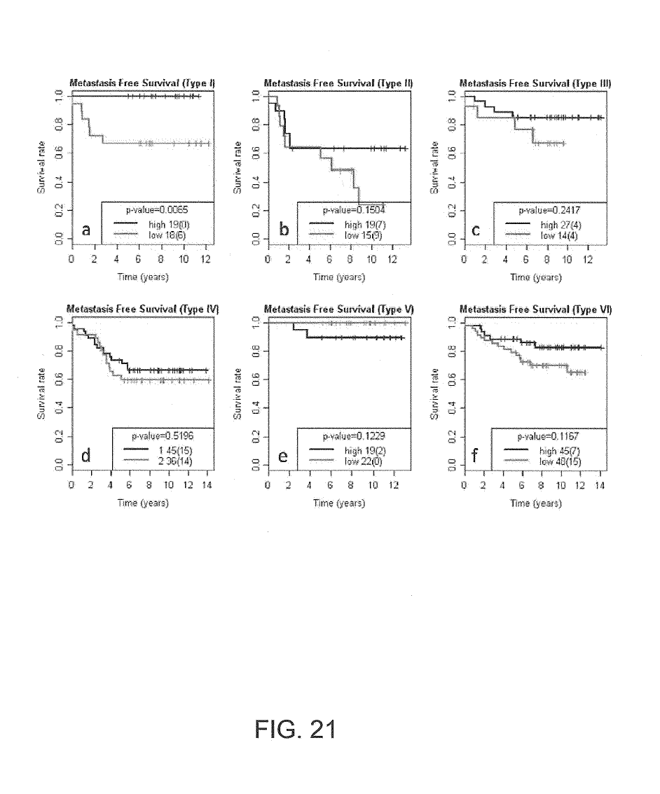

Identification of breast cancer cases of high or low immune responses in each molecular subtypes. To learn how the differential expression of immune response genes is associated with the metastasis-free survival outcome in each molecular subtype of breast cancer. We conducted hierachical clustering analyses using the selected immune response probe-sets on each molecular subtype of our 327 breast cancer cases. The hierachical clustering analyses identified two subgroups with high and low expression of immune response genes in each molecular subtype (FIG. 20). Next, metastasis-free survival was compared between the two subgroups by log-rank test. The results showed that the subgroup with higher expression of the immune response genes had significantly better survival in subtypes I cancer patients (FIG. 21a). A trend of better survival towards those with higher expression of immune response probe-sets was also noted in subtypes II and VI breast cancer (FIGS. 21b and 21e).

To confirm the trends observed for subtypes II and IV, we increased sample numbers by including additional 180 patients recently studied by us to increase sample number, and conducted Cox regression analysis between immune response scores and metastasis-free survival in each molecular subtypes. The results are summarized in Table 23. Our results demonstrated that high immune responders of subtypes I, II and III had significantly better metastasis-free survival with respective p values of 0.0003, 0.0037 and 0.0074 (Table 23 Pooled KFCC results).

TABLE 23
Cox regression results of immune response scores with metastasis-free survival for patients in each different
molecular subtype of breast cancer in our datasets of 327 patients (KFCC 327), 507 patients (KFCC 327 +
180) and 860 patients pooled from five published datasets available from GEO database [TRANSBIG (GSE7390),
MSKCC(GSE2603), Oxford(GSE2990), EMC(GSE2034), and Mainz(GSE11121)] (http://www.ncbi.nlm.nih.gov/geo/).

| | I | | II | | III | | IV | | V | | VI | |
|---|---|---|---|---|---|---|---|---|---|---|---|---|
| | Corre- | | Corre- | | Corre- | | Corre- | | Corre- | | Corre- | |
| | lation co- | | lation co- | | lation co- | | lation co- | | lation co- | | lation co- | |
| Dataset | efficient | p | efficient | p | efficient | p | efficient | p | efficient | p | efficient | p |
| KFCC 327 | −3.6048 | 0.0013 | −0.5796 | 0.0902 | −1.0613 | 0.0372 | −0.4449 | 0.1034 | 0.2309 | 0.8405 | −0.7650 | 0.0966 |
| KFCC 327 + 180 | −1.6233 | 0.0003 | −0.7752 | 0.0037 | −0.9680 | 0.0074 | −0.2439 | 0.2420 | 0.4023 | 0.6579 | −0.1566 | 0.5969 |
| Pooled 5 public | −0.5310 | 0.0110 | −0.6904 | 0.0246 | −0.3671 | 0.2782 | −0.5722 | 0.0008 | 0.4062 | 0.3332 | −0.4065 | 0.2042 |
| datasets | | | | | | | | | | | | |

The number of patients in each molecular subtype for the three datasets is shown in Table 24.

TABLE 24
Number of patients in each molecular subtype for
the Cox-regression study described in Table 23.

| | Molecular Subtype | | | | | |
|---|---|---|---|---|---|---|
| | I | II | III | IV | V | VI |
| KFCC 327 | 37 | 34 | 41 | 81 | 41 | 93 |
| KFCC 327 + 180 | 53 | 56 | 62 | 123 | 55 | 158 |
| Pooled 5 public | 141 | 64 | 59 | 211 | 138 | 247 |
| datasets | | | | | | |

Next, we used a pool of 860 breast cancer samples from five published independent datasets to validate our findings. Again, we conducted Cox regression analysis between the immune response scores and the metastasis survival. The results of this validation study confirmed that the higher score of immune response related genes is associated with better metastasis-free survival for both subtype I and II breast cancer patients (Table 23). The association between higher score of immune response genes and better distant metastasis survival in subtype III and IV was not confirmed between our pooled dataset and the pooled independent datasets (Table 23). Thus, we conclude that the score of immune response related genes is associated with risk of distant metastasis in breast cancer patients of molecular subtype I and II and can be used to consistently predict risk of distant metastasis in these molecular subtypes of breast cancer.

10.3: Conclusion

The results of this supplemental study demonstrate that the expression of immune response genes can be used to identify patients with the increased risk of distant metastasis in molecular subtype I and II breast cancer patients. Such application will provide oncologists invaluable information to customize treatment of breast cancer patients, and underscores the clinical importance of our breast cancer molecular subtyping method.

For instance, molecular subtype I breast cancer is chemosensitive and can be effectively treated with CMF or CAF adjuvant chemotherapy regimen for excellent long-term survival outcome, if their expression scores of immune response related genes are high. In contrast, those patients of molecular subtype I patients with low expression of immune response genes should be treated with more intense chemotherapy regimen or new experimental drugs to improve their survival outcome. Similarly, we can identify high risk patients in molecular subtype II breast cancer patients with over-expression of HER2 to receive Herceptin, tyrosin-kinase receptor inhibitors or other more intense experimental chemotherapy.

The following exemplifications complement that of Examples 1-9.

### Example 11

### Additional Validation and Analysis

11.1: Additional Statistical Analysis

Additional Clustering Analysis for Identification of Breast Cancer Molecular Subtypes:

We applied the method proposed by Smolkin and Ghosh (BMC Bioinformatics 4:36-42, 2003) to assess stability of sample clusters determined at different Pearson correlation values.

The first assessment was performed as following:

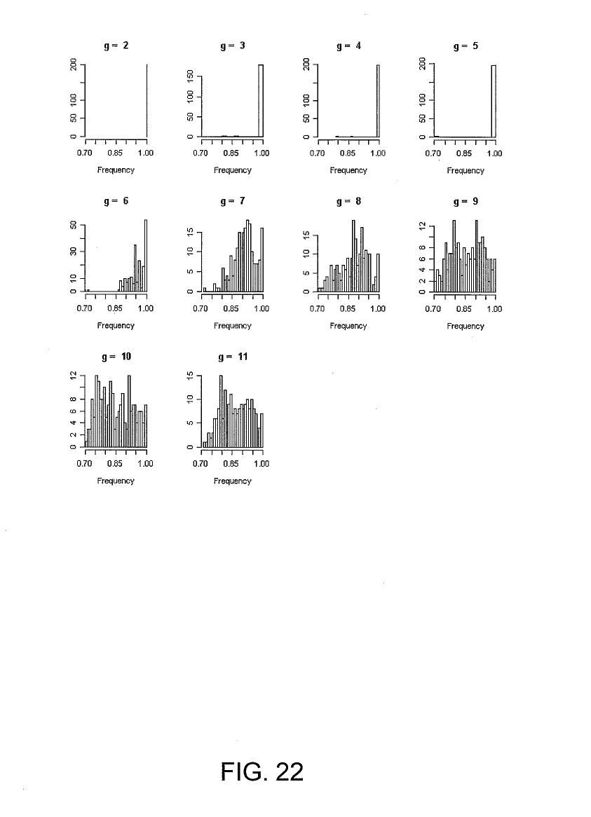

Eighty percent of 327 samples were randomly sampled twice to generate a pair of sub-datasets. The 2000 cluster labels generated for each sample by k-means clustering analyses as described earlier were used to conduct hierachical clustering analysis for each pair of sub-datasets, separately. The samples were clustered into different numbers of groups (e.g. g=2, 3, 4 . . . , 11) according to different Pearson correlation values as described above (see materials and methods of Example 1). The similarity between results of each pair for each number of groups (g=2, 3, 4 . . . , 11) was measured by calculation of Jaccard coefficient (JC). The closer the JC is to 1, the more similar two separate clustering results are. This process was repeated 200 times. The histograms of 200 sets of JCs for each number of groups (g=2 to 11) are shown in FIG. 22.

The second assessment was also conducted to determine average stability of different number of breast cancer groups generated at different height (1-r). For this assessment, a hierarchical clustering analysis was conducted using 2000 k-means cluster labels for each sample to create a full dendrogram of 327 samples. Samples were clustered into different number of groups by cutting the dendrogram at different height levels (1-r).

Next, a hierarchical clustering analysis was conducted using 80% of the 2000 k-means cluster labels which were randomly selected for each sample to create a dendrogram of 327 samples. Samples were clustered into different number of groups at different heights (1-r). This clustering analysis was repeated 200 times. The percentage for cases remain in the same group by the full dendrogram was calculated as a stability measurement of the groups

The average of stability measurements for each cluster (sample group) was taken as the average group stability score reflecting how unlikely the group was due to chance The stability scores of each groups for different number of groups from 4 to 11 are shown in Table 25.

TABLE 25

| | | | | | | | | | | | | Average |
|---|---|---|---|---|---|---|---|---|---|---|---|---|
| k = 8 | Group 1 | Group 2 | Group 3 | Group 4 | Group 5 | Group 6 | Group 7 | Group 8 | Group 9 | Group 10 | Group 11 | Stability |
| 4 Groups | 81 | 134 | 37 | 75 | | | | | | | | |
| Group Stability | 92.5 | 71.5 | 100 | 96.5 | | | | | | | | 90.1 |
| 5 Groups | 81 | 93 | 37 | 75 | 41 | | | | | | | |
| Group Stability | 92.5 | 98.5 | 100 | 96.5 | 72 | | | | | | | 91.9 |
| 6 Groups | 81 | 93 | 37 | 34 | 41 | 41 | | | | | | |
| Group Stability | 92 | 98 | 100 | 100 | 96.5 | 72 | | | | | | 93.1 |
| 7 Groups | 47 | 93 | 37 | 34 | 41 | 34 | 41 | | | | | |
| Group Stability | 75.5 | 64 | 100 | 100 | 65 | 66 | 72 | | | | | 77.5 |
| 8 Groups | 47 | 33 | 37 | 34 | 60 | 41 | 34 | 41 | | | | |
| Group Stability | 58.5 | 100 | 100 | 100 | 98.5 | 96.5 | 100 | 72 | | | | 90.7 |
| 9 Groups | 46 | 33 | 37 | 34 | 60 | 41 | 34 | 41 | 1 | | | |
| Group Stability | 64.5 | 97 | 97 | 97 | 95.5 | 96.5 | 97 | 26 | 45 | | | 79.5 |
| 10 Groups | 46 | 33 | 37 | 34 | 60 | 41 | 34 | 40 | 1 | 1 | | |
| Group Stability | 67.5 | 98 | 98 | 96.5 | 59 | 95.5 | 98 | 98 | 59 | 59 | | 82.9 |
| 11 Groups | 46 | 33 | 37 | 34 | 53 | 41 | 34 | 40 | 7 | 1 | 1 | |
| Group Stability | 59 | 95.5 | 95.5 | 94 | 95.5 | 67 | 95.5 | 95.5 | 86 | 92.5 | 69 | 85.9 |

Based on the results from the method proposed by Smolkin and Ghosh (BMC Bioinformatics 4:36-42, 2003), we chose groups of 6 for our breast cancer molecular subtypes.

11.2 Scoring of Relative Risk for Distant Recurrence Using the OncotypeDX and MammaPrint Predictors.

We applied the predictive models of van't Veer et al. (Nature 2002, 415:530-536) (MammaPrint) and Paik et al. (New Engl J Med 351:2817-2826, 2004) (OncotypeDX) to our dataset and the datasets of EMC and NKI to determine the relative risk for distant recurrence. To calculate the recurrence score of Oncotype DX, the model of Paik et al. involving 16 genes associated with distant recurrence was directly applied all three datasets. Probe-sets of Affymetrix U133A GeneChip and genes of NKI DNA microarray corresponding to the 16 genes were identified and are shown in Table 26:

| OncotypeDX Predictor Genes | | | MammaPrint Predictor Genes | | |
|---|---|---|---|---|---|
| Gene | Affymetrix | | Gene | Affymetrix | |
| Symbol | Probeset ID | NKI ID | Symbol | Probeset ID | NKI ID |
| BAG1 | 202387_at | ID5227 | AKAP2 | 202759_s_at | ID12009 |
| CD68/EIF4A1 | 203507_at | ID22119 | ALDH4 | 211552_s_at | ID6556 |
| BCL2 | 203685_at | ID22945 | AP2B1 | 200612_s_at | ID22282 |
| ESR1 | 205225_at | ID18904 | BBC3 | 211692_s_at | ID12695 |
| PGR | 208305_at | ID630 | CCNE2 | 205034_at | ID8994 |
| SCUBE2 | 219197_s_at | ID10658 | CEGP1 | 219197_s_at | ID10658 |
| GSTM1 | 204550_x_at | ID22320 | CENPA | 204962_s_at | ID1944 |
| GRB7 | 210761_s_at | ID7930 | COL4A2 | 211964_at | ID2146 |
| ERBB2 | 216836_s_at | ID6424 | DC13 | 218447_at | ID3476 |
| CTSL2 | 210074_at | ID22839 | DCK | 203302_at | ID23739 |
| MMP11 | 203878_s_at | ID13284 | DHX58 | 219364_at | ID18440 |
| CCNB1 | 214710_s_at | ID14976 | DIAPH3 | 220997_s_at | ID22739 |
| MKI67 | 212023_s_at | ID1161 | ECT2 | 219787_s_at | ID23213 |
| MYBL2 | 201710_at | ID1354 | ESM1 | 208394_x_at | ID10260 |
| AURKA | 208079_s_at | ID5281 | EXT1 | 201995_at | ID18906 |
| BIRC5 | 202094_at | ID21371 | FGF18 | 211029_x_at | ID7474 |
| | | | FLJ11190 | 219958_at | ID19709 |
| | | | FLT1 | 204406_at | ID22706 |
| | | | GMPS | 214431_at | ID7504 |
| | | | GNAZ | 204993_at | ID22879 |
| | | | GSTM3 | 202554_s_at | ID24348 |
| | | | HEC | 204162_at | ID8746 |
| | | | HSA250839 | 219686_at | ID20335 |
| | | | IGFBP5 | 211959_at | ID22447 |
| | | | IGFBP5 | 211959_at | ID12587 |
| | | | KIAA0175 | 204825_at | ID14112 |
| | | | KIAA1067 | 212248_at | ID16531 |
| | | | L2DTL | 218585_s_at | ID16238 |
| | | | LOC51203 | 218039_at | ID15405 |
| | | | LOC57110 | 219983_at | ID5373 |
| | | | MCM6 | 201930_at | ID13145 |
| | | | MMP9 | 203936_s_at | ID10842 |
| | | | MP1 | 205273_s_at | ID14907 |
| | | | NMU | 206023_at | ID13324 |
| | | | ORC6L | 219105_x_at | ID10243 |
| | | | OXCT | 202780_at | ID21365 |
| | | | PECI | 218025_s_at | ID8797 |
| | | | PECI | 218025_s_at | ID9171 |
| | | | PK428 | 203794_at | ID5308 |
| | | | PRC1 | 218009_s_at | ID8523 |
| | | | RAB6B | 210127_at | ID16966 |
| | | | RFC4 | 204023_at | ID5529 |
| | | | SERF1A | 219982_s_at | ID20881 |
| | | | SLC2A3 | 202499_s_at | ID15609 |
| | | | TGFB3 | 209747_at | ID1846 |
| | | | TSPYL5 | 213122_at | ID10904 |
| | | | UCH37 | 219960_s_at | ID17793 |
| | | | WISP1 | 206796_at | ID7524 |

Probe-set IDs and genes from the OncotypeDX and MammaPrint predictors that were used to score risk of distant recurrence. Sixteen genes in the OncotypeDX predictor can be matched to Affymetrix probe-set IDs and NKI-ID. Forty eight out of seventy MammaPrint predictor genes can be matched to Affymetrix probe-set IDs in the U133A GeneChip and used for the study.

Expression intensities of these 16 genes were fed into the model directly to calculate the recurrence score of each case. For the NKI dataset, quantile-normalized red channel data were used to determine gene expression intensities. To calculate the score correlated with low risk of distant recurrence using the genes of MammaPrint predictor, we identified 48 Affymetrix probe-sets matched to the Mammaprint predictor (Table 26). We then determined the Pearson correlation coefficient of each sample with the average good prognosis profile of the NKI dataset. The average good prognosis profile was established by calculation of the average gene expression intensity of the 44 low-risk cases reported in the study of van't Veer et al. for each gene used in the predictor.

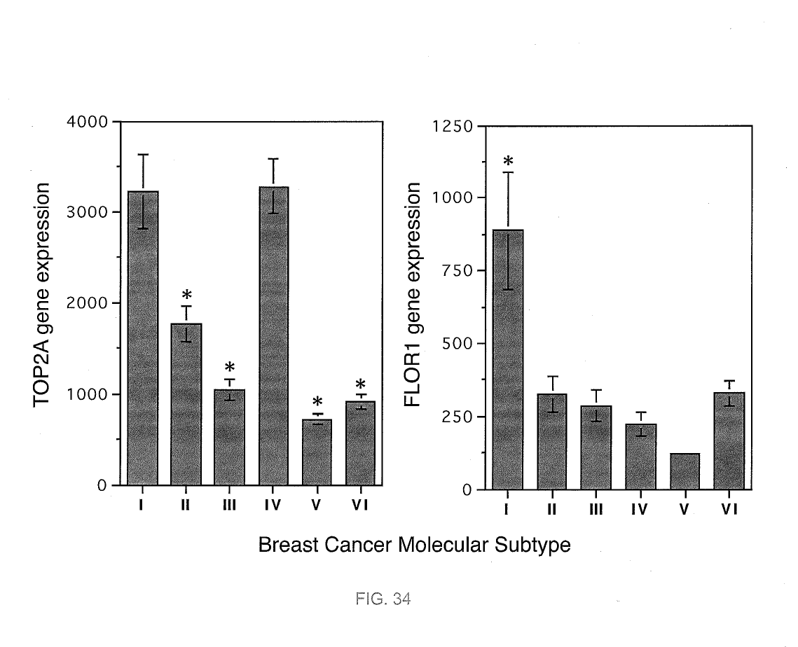

Results are summarized in FIG. 33.

11.3: Statistical Comparison for Concordance of Differential Gene Expression Patterns Between KFSYSCC Dataset and Public Datasets from EMC, Uppsala, and TRANSBIG.

The primary purpose of this study was to determine the concordance of differential gene expression pattern of four signatures associated with cell cycle/proliferation (A), wound response (B), stromal reaction (C), and tumor vascular endothelial normalization (D) among six breast cancer molecular subtypes between our cohort and each of the three published independent cohorts. For each cohort, we used genes in each signature to draw a heat map according to the results of one-way hierachical clustering analysis (FIG. 17). The concordance of the heat map patterns between KFSYSCC cohort and each of Uppsala, EMC, and TRANSBIG cohorts was statistically measured and tested as described below.

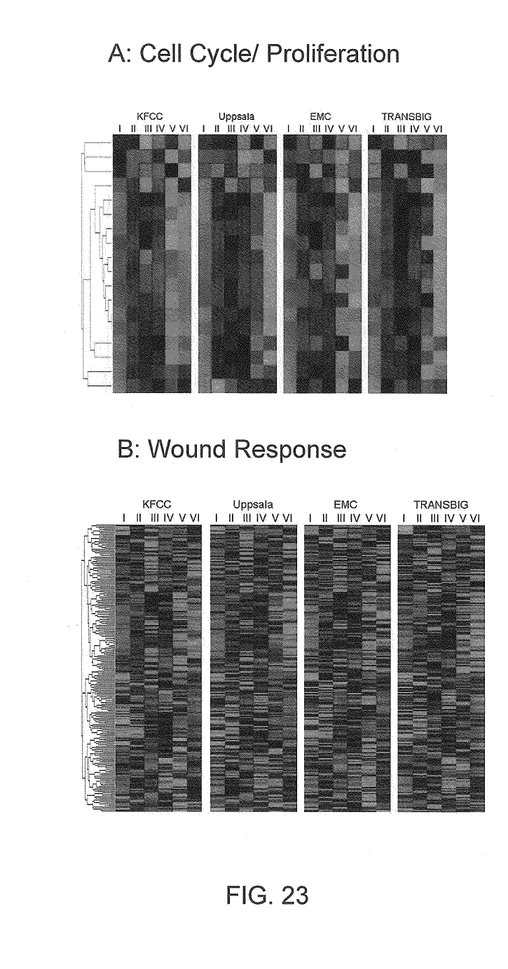

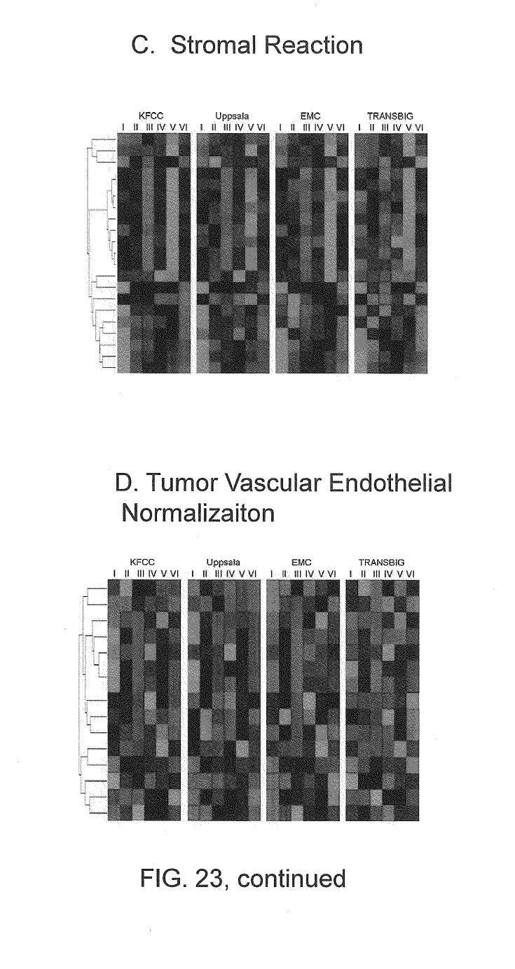

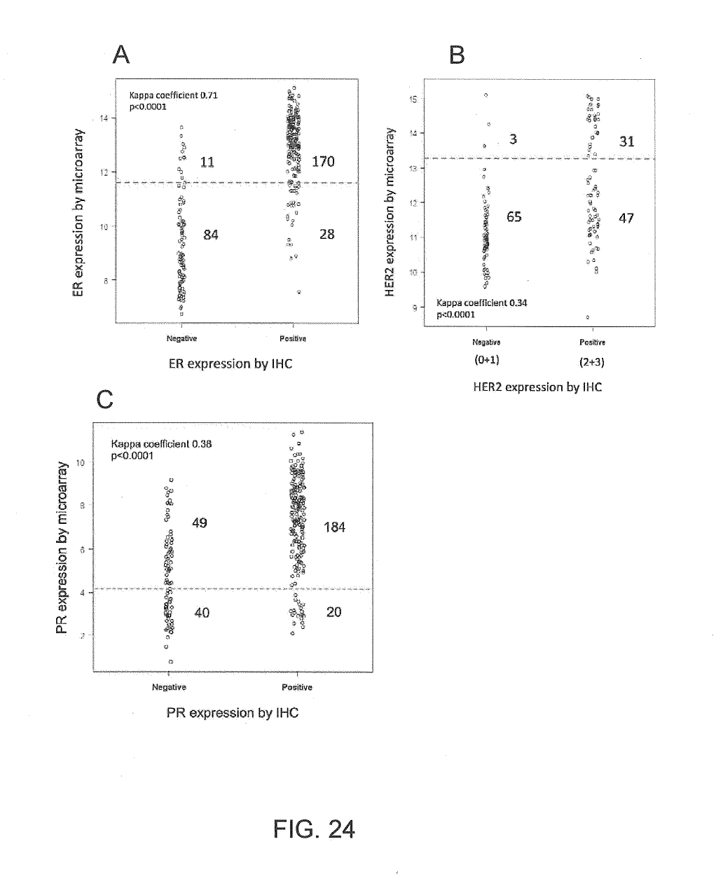

The gene expression data were quantile-normalized. Z score of each gene for each sample was calculated in each cohort. Next, we determined the average of Z scores for each molecular subtype in each cohort. The average Z scores were used to draw a heat map for each signature and cohort. The heat map was drawn according to the dendrogram of genes in each signature as shown in FIG. 17 for each cohort. All heat maps are shown in FIG. 23 A-D.

The concordance of gene expression pattern at the molecular subtype level for each gene signature between 2 cohorts was determined by Pearson correlation. The correlation coefficients are summarized in Table 27.

TABLE 27
Pearson correlation coefficients for each signature between the
KFSYSCC cohort and each of the three cohorts (EMC, Uppsala and
TRANSBIG). P-values for all correlation coefficients are <10⁻⁴.

| Signature | Uppsala | EMC | TRANSBIG |
|---|---|---|---|
| Cell Cycle/Proliferation | 0.92 | 0.94 | 0.87 |
| Wound Response | 0.84 | 0.85 | 0.78 |
| Stromal Reaction | 0.91 | 0.94 | 0.87 |
| Vascular Normalization | 0.86 | 0.86 | 0.83 |

The significance of each correlation coefficient was tested by comparing the correlation coefficient to the empirical null distribution of the correlation coefficients derived from 10,000 permutations of molecular subtypes at sample level.

The heat maps of average Z scores for each gene and molecular subtype are shown in FIG. 23 A-D. FIG. 23 shows that there are similar expression patterns at molecular subtype level among different cohorts. The levels of concordance between KFSYSCC cohort and other cohorts for four different gene signatures were analyzed by Pearson correlation. The results summarized in Table 27 showed high degrees of concordance between our cohort and three other independent cohort. The p values for all coefficients are highly significant (p<10⁻⁴). The results validate the molecular subtypes determined with our classification genes.

### Example 12

### Additional Data

TABLE 28
Statistical comparison of pertinent clinical parameters between subtype
I patients treated with CAF and CMF adjuvant chemotherapy.

| | CAF | | CMF | | Fisher exact |
|---|---|---|---|---|---|
| | n = 10 | | n = 13 | | test p value |
| Age at diagnosis | | | | | 1 |
| <50 yr | 7 | 70.0% | 9 | 69.2% | |
| >=50 yr | 3 | 30.0% | 4 | 30.8% | |
| TNM Path T | | | | | 0.38 |
| 1 | 2 | 20.0% | 6 | 46.2% | |
| 2 | 8 | 80.0% | 7 | 53.8% | |
| TNM Path N | | | | | 0.17 |
| 0 | 5 | 50.0% | 11 | 84.6% | |
| 1 | 5 | 50.0% | 2 | 15.4% | |
| TNM Path M | | | | | |
| 0 | 10 | 100.0% | 13 | 100.0% | |
| Positive Lymph | | | | | 0.17 |
| Nodes | | | | | |
| 0 | 5 | 50.0% | 11 | 84.6% | |
| 1-3 | 5 | 50.0% | 2 | 15.4% | |
| TNM Stage | | | | | 0.09 |
| I | 1 | 10.0% | 6 | 46.2% | |
| II | 9 | 90.0% | 7 | 53.8% | |
| Nuclear Grade | | | | | |
| 1 | 0 | 0.0% | 1 | 7.7% | 0.49 |
| 2 | 1 | 10.0% | 2 | 15.4% | |
| 3 | 9 | 90.0% | 9 | 69.2% | |
| Hormonal Therapy | | | | | 0.62 |
| No | 7 | 70.0% | 11 | 84.6% | |
| Yes | 3 | 30.0% | 2 | 15.4% | |
| Post-op Radiation | | | | | 0.65 |
| No | 6 | 60.0% | 10 | 76.9% | |
| Yes | 4 | 40.0% | 3 | 23.1% | |

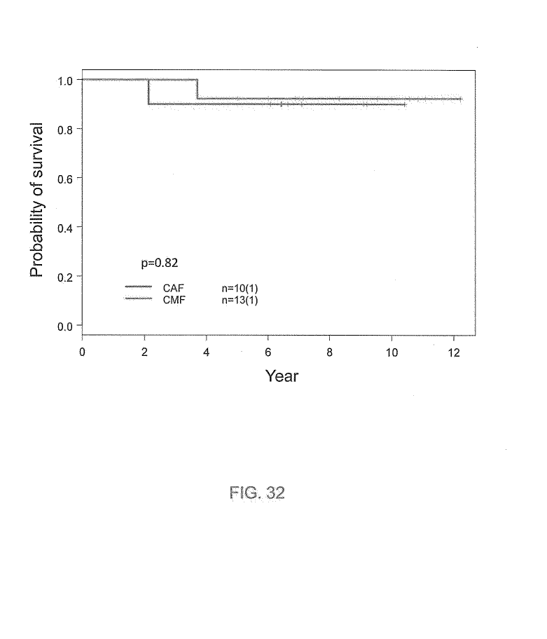

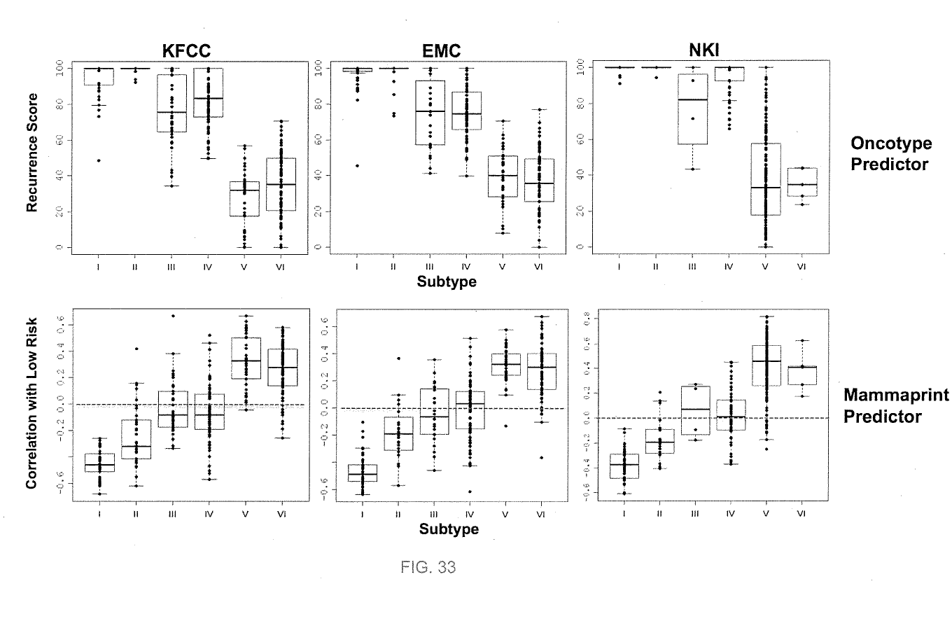

Table 28 is related to FIG. 32.

### REFERENCES

1. Parkin D M, Bray F, Ferlay J, et al. Estimating the world cancer burden: Globalcan 2000. Int J Cancer 94:153-6, 2001.
2. Chlebowski R T, Kuller L H, Prentice R L, et al. Breast cancer after use of estrogen plus progestin in postmenopausal women. New Eng J Med 360:573-587, 2009.
3. Stratton M R and Rahman N. The emerging landscape of breast cancer susceptibility. Nature Genet 40:17-22. 2008.
4. Kurose K, Gilley K, Matsumoto S, Watson P H, Zhou X P, Eng C. Frequent somatic mutations in PTEN and TP53 are mutually exclusive in the stroma of breast carcinomas. Nature Genet. 32:355-7, 2002.
5. Widschwendter M, Jones P A: DNA methylation and breast carcinogenesis. Oncogene. 21:5462-5482, 2002.
6. Albertson, D G, Collins C, McCormick F, and Gray J W. Chromosome aberrations in solid tumors. Nat. Genet. 34, 369-376, 2003.
7. Jones P A. Overview of cancer epigenetics. Semin. Hematol. 42, S3-S8, 2005.
8. Betsill W L, Rosen P P, Lieberman P H, Robbins G F. Intraductal carcinoma: long-term follow-up after treatment by biopsy alone. JAMA. 1978; 239:1863-1867.
9. Dupont W D, Parl F F, Hartmann W H, et al. Breast cancer risk associated with proliferative breast disease and atypical hyperplasia. Cancer 71:1258-1265, 1993.
10. Leonard G D and Swain S M. Ductal carcinoma in situ, complexities and challenges. J Natl Can Inst 96:906-920, 2004.
11. Sanders M E, Schuyler P A, Dupont W D and Page D L. The natural history of low grade ductal carcinoma in situ of the breast in women treated by biopsy only revealed over 30 years of long-term follow-up. Cancer 103:2481-2484, 2005.
12. Allred D C, Wu Y, Mao S, et al. Ductal carcinoma in situ and the emergence of diversity during breast cancer evolution. Clin Cancer Res 14:370-378, 2008.
13. Polyak K. Is breast tumor progression really linear? Clin Cancer Res 14:339-341, 2008.
14. Key T J, Verkasalo P K and Banks E. Epidemiology of breast cancer. Lancet Oncol 2:133-140, 2001.
15. Jensen, E. V., Block, G. E., et al.: Estrogen Receptors and Breast Cancer Response to Adrenalectomy. In: Prediction of Response in Cancer Therapy. Monograph 34. Edited by Hall, T. C. Bethesda, National Cancer Institute, 1971; p. 55.
16. Block G E, Jensen E V and Polley T Z, Jr. The prediction of hormonal dependency of mammary cancer. Ann Surg 182-342-351, 1975.
17. DeSombre E R, Thorpe S M, Rose C, et al. Prognostic usefulness of estrogen receptor immunocytochemical assays for human breast cancer. Cancer Research (suppl.) 46:4256s-4264s, 1986.
18. Slamon D J, Clark G M, Wong S G, et al. Human breast cancer: correlation of relapse and survival with amplification of the HER-2/neu oncogene. Science 135; 277-282, 1982.
19. Ross J S, Fletcher J A, Linette G P, et al. HER-2/neu gene and protein in breast cancer 2003: biomarker and target of therapy. Oncologist 8:307-325, 2003.
20. Paik S, Hazan R, Fisher E R, et al. Pathologic findings from the national surgical adjuvant breast and bowel project: prognostic significance of erbB-2 protein overexpression in primary breast cancer. J Clin Oncol 8:103-112, 1990.
21. Tovey S M, Brown S, Doughty J C, et al. Poor survival outcomes in HER2-positive breast cancer patients with low-grade, node-negative tumours. Br J Cancer 100; 680-683, 2009.
22. Slamon D J, Leyland-Jones B, Shak S, et al. Use of chemotherapy plus a monoclonal antibody against HER2 for metastatic breast cancer that overexpresses HER2. N Eng J Med 344:783-792, 2001.
23. Anderson W F and Matsuno R. Breast cancer heterogeneity. J Natl Cancer Inst 98:948-51, 2006.
24. van't Veer L J, Dai H, van de Vijver M J, et al. Gene expression profiling predicts clinical outcome of breast cancer. Nature 415; 530-536, 2002.
25. Rosenwald A, Wright G, Chan W C, et al. The use of molecular profiling to predict survival after chemotherapy for diffuse large B-cell lymphoma. New Eng J Med 346; 1937-1947, 2002.
26. Beer D G, Kardia S L R, Huang C C, et al. Gene-expression profiles predict survival of patients with lung adenocarcinoma. Nature Med 8:816-824, 2002.
27. Perou C M, Sorlie T, Eisen M B, et al. Molecular portraits of human breast tumours. Nature 406:747-752, 2000.
28. Sørliea T, Perou C M, Tibshirani R, et al. Gene expression patterns of breast carcinomas distinguish tumor subclasses with clinical implications. Proc Natl Acad Sci, USA 98:10869-10874, 2001.
29. Sorlie T, Tibshirani R, Parker J, et al. Repeated observation of breast tumor subtypes in independent gene expression data sets. Proc Natl Acad Sci, USA 100:8418-8423, 2003.
30. Calza S, Hall P, Auer G, et al. Intrinsic molecular signature of breast cancer in a population-based cohort of 412 patients. Breast Cancer Res 8: R34, 2006.
31. Huang E, Cheng S H, Dressman H, et al. Gene expression predictors of breast cancer outcomes. Lancet 361:1590-1596, 2003.
32. Paik S, Shak S, Tang G, et al. A multigene assay to predict recurrence of tamoxifen-treated, node-negative breast cancer. New Eng J Med 351:2817-2826, 2004.
33. Chang H Y, Nuyten D S A, Sneddon J B, et al. Robustness, scalability and integration of a wound-response gene expression signature in predicting breast cancer survival. Proc Natl Acad Sci, USA 102:3738-3734, 2005.
34. Wang Y, Klijn J G M, Zhang Y, et al. Gene-expression profiles to predict distant metastasis of lymph-node-negative primary breast cancer. Lancet 365:671-679, 2005.
35. Ma Y, Qian Y, wei L, et al. population-based molecular prognosis of breast cancer by transcriptional profiling. Clin Cancer Res 13; 2014-2022, 2007.
36. Liu R, Wang X, Chen G Y, et al. The prognostic role of a gene signature from tumorigenic breast-cancer cells. New Eng J Med 356; 217-226, 2007.
37. Naderi A, Teschendorff, Barbosa-morais N L, et al. A gene-expression signature to predict survival in breast cancer across independent data sets. Oncogene 26:1507-1516, 2007.
38. Bogaerts J, Cardoso F, Buyse M, et al. TRANSBIG consortium: clinical application of the 70-gene profile: the MINDACT trial. J Clin Oncol 26:729-735, 2008.
39. North American Breast Cancer Intergroup accessible at web address www.cancer.gov/clinicaltrials/digestpage/Tailorx.
40. Irizarry R A, Hobbs B, Collin F, et al. Exploration, normalization, and summaries of high density oligonucleotide array probe level data. Biostatistics 4:249-264, 2003.
41. Tanaka K, Iwamoto S, Gon G, Nohara T, Iwamoto M, Tanigawa N. Expression of survivin and its relationship to loss of apoptosis in breast carcinomas. Clin Cancer Res. 6:127-34, 2000.
42. Nasu S, Yagihashi A, Izawa A, Saito K, Asanuma K, Nakamura M, Kobayashi D, Okazaki M, Watanabe N. Survivin mRNA expression in patients with breast cancer. Anticancer Res. 22:1839-43, 2002.
43. Brennan D J, Rexhepaj E, O'Brien S L, et al. Altered cytoplasmic-to-nuclear ratio of survivin is a prognostic indicator in breast cancer. Clin Cancer Res. 14:2681-9, 2008.
44. Black D M, Nicolai H, Borrow J, Solomon E. A somatic cell hybrid map of the long arm of human chromosome 17, containing the familial breast cancer locus (BRCA1). Am J Hum Genet. 52:702-10, 1993.
45. Narod S, Lynch H, Conway T, Watson P, Feunteun J, Lenoir G. Increasing incidence of breast cancer in family with BRCA1 mutation. Lancet. 341:1101-2, 1993.
46. Langston A A, Malone K E, Thompson J D, Daling J R, Ostrander E A. BRCA1 mutations in a population-based sample of young women with breast cancer. N Engl J Med. 334:137-42, 1996.
47. Fogel M, Friederichs J, Zeller Y, et al. CD24 is a marker for human breast carcinoma. Cancer Lett. 143:87-94, 1999.
48. Abraham B K, Fritz P, McClellan M, Hauptvogel P, Athelogou M, Brauch H. Prevalence of CD44+/CD24−/low cells in breast cancer can not be associated with clinical outcome but can favor distant metastasis. Clin Cancer Res. 11:1154-9, 2005.
49. Honeth G, Bendahl P O, Ringnér M, et al. The CD44+/CD24− phenotype is enriched in basal-like breast tumors. Breast Cancer Res. 10:R53, 2008.
50. Sheridan C, Kishimoto H, Fuchs R K, et al. CD44+/CD24− breast cancer cells exhibit enhanced invasive properties: an early step necessary for metastasis. Breast Cancer Res. 8:R59, 2006.
51. Poola I, Shokrani B, Bhatnagar R, DeWitty R L, Yue Q, Bonney G. Expression of carcinoembryonic antigen cell adhesion molecule 6 oncoprotein in atypical ductal hyperplastic tissues is associated with the development of invasive breast cancer. Clin Cancer Res 12:4773-83, 2006.
52. Maraqa L, Cummings M, Peter M B, Shaaban A M, Horgan K, Hanby A M, Speirs V. Carcinoembryonic antigen cell adhesion molecule 6 predicts breast cancer recurrence following adjuvant tamoxifen. Clin Cancer Res 14:405-11, 2008.
53. O'Brien S L, Fagan A, Fox E J, et al. CENP-F expression is associated with poor prognosis and chromosomal instability in patients with primary breast cancer. Int J Cancer. 120:1434-43, 2007.
54. Tokés AM, Kulka J, Paku S, et al. Claudin-1, -3 and -4 proteins and mRNA expression in benign and malignant breast lesions: a research study. Breast Cancer Res. 7:R296-305, 2005.
55. Morohashi S, Kusumi T, Sato F Decreased expression of claudin-1 correlates with recurrence status in breast cancer. Int J Mol Med. 20:139-43, 2007.
56. Knoop A S, Bentzen S M, Nielsen M M, Rasmussen B B, Rose C. Value of epidermal growth factor receptor, HER2, p53, and steroid receptors in predicting the efficacy of tamoxifen in high-risk postmenopausal breast cancer patients. J Clin Oncol. 19:3376-84, 2001.
57. Hoadley K A, Weigman V J, Fan C, et al. EGFR associated expression profiles vary with breast tumor subtype. BMC Genomics 31; 8:258, 2007.
58. Asanuma H, Torigoe T, Kamiguchi K, Hirohashi Y, Ohmura T, Hirata K, Sato M, Sato N. Survivin expression is regulated by coexpression of human epidermal growth factor receptor 2 and epidermal growth factor receptor via phosphatidylinositol 3-kinase/AKT signaling pathway in breast cancer cells. Cancer Res 65:11018-25, 2005.
59. Knoop A S, Bentzen S M, Nielsen M M, et al. Value of epidermal growth factor receptor, HER2, p53, and steroid receptors in predicting the efficacy of tamoxifen in high-risk postmenopausal breast cancer patients. J Clin Oncol 19:3376-84, 2001.
60. Eccles S A. The role of c-erbB-2/HER2/neu in breast cancer progression and metastasis. J Mammary Gland Biol Neoplasia. 6:393-406, 2001.
61. Kun Y, How L C, Hoon T P, et al. Classifying the estrogen receptor status of breast cancers by expression profiles reveals a poor prognosis subpopulation exhibiting high expression of the ERBB2 receptor. Human Mol Genetics, 12:3245-3258, 2003.
62. Palmieri D, Bronder J L, Herring J M, et al. Her-2 overexpression increases the metastatic outgrowth of breast cancer cells in the brain. Cancer Res 67:4190-8, 2007.
63. Asanuma H, Torigoe T, Kamiguchi K, Hirohashi Y, Ohmura T, Hirata K, Sato M, Sato N. Survivin expression is regulated by coexpression of human epidermal growth factor receptor 2 and epidermal growth factor receptor via phosphatidylinositol 3-kinase/AKT signaling pathway in breast cancer cells. Cancer Res 65:11018-25, 2005.
64. Thorpe S M, Rose C, Pedersen B V, Rasmussen B B. Estrogen and progesterone receptor profile patterns in primary breast cancer. Breast Cancer Res Treat 3:103-10, 1983.
65. Rebbeck T R, DeMichele A, Tran T V, Panossian S, Bunin G R, Troxel A B, Strom B L. Hormone-dependent effects of FGFR2 and MAP3K1 in breast cancer susceptibility in a population-based sample of post-menopausal African-American and European-American women. Carcinogenesis. 30:269-74, 2009.
66. Easton D F, Pooley K A, Dunning A M, et al. Genome-wide association study identifies novel breast cancer susceptibility loci. Nature. 447:1087-93, 2007.
67. Lacroix M, Leclercq G. About GATA3, HNF3A, and XBP1, three genes co-expressed with the oestrogen receptor-alpha gene (ESR1) in breast cancer. Mol Cell Endocrinol 219:1-7, 2004.
68. Wolf I, Bose S, Williamson E A, et al. FOXA1: Growth inhibitor and a favorable prognostic factor in human breast cancer. Int J Cancer. 120:1013-22, 2007.
69. Badve S, Turbin D, Thorat M A, et al. FOXA1 expression in breast cancer-correlation with luminal subtype A and survival. Clin Cancer Res 13:4415-21, 2007.
70. Yamaguchi N, Ito E, Azuma S, et al. FoxA1 as a lineage-specific oncogene in luminal type breast cancer. Biochem Biophys Res Commun 365:711-7, 2008.
71. Bloushtain-Qimron N, Yao J, Snyder E L, et al. Cell type-specific DNA methylation patterns in the human breast. Proc Natl Acad Sci, USA. 105:14076-81, 2008.
72. L Carrivick, S Rogers, J Clark, et al. Identification of prognostic signatures in breast cancer microarray data using Bayesian techniques. J. R. Soc. Interface 3:367-381, 2006.
73. Accili, D., and Arden, K. C. FoxOs at the crossroads of cellular metabolism, differentiation, and transformation. Cell 117, 421-426, 2004.
74. Greer, E., and Brunet, A. FOXO transcription factors at the interface between longevity and tumor suppression. Oncogene 24, 7410-7425, 2005.
75. Stein D, Wu J, Fuqua S A, Roonprapunt C, et al. The SH2 domain protein GRB-7 is co-amplified, overexpressed and in a tight complex with HER2 in breast cancer. EMBO J 13:1331-40, 1994.
76. Chiappetta G, Botti G, Monaco M et al. HMGA1 Protein Overexpression in Human Breast Carcinomas Correlation with ErbB2 Expression Clinical Cancer Research 10:7637-7644, 2004.
77. Treff N R, Pouchnik D, Dement G A, Britt R L, Reeves R. High-mobility group Ala protein regulates Ras/ERK signaling in MCF-7 human breast cancer cells. Oncogene 23:777-85, 2004.
78. Baldassarre G, Battista S, Belletti B, et al. Negative regulation of BRCA1 gene expression by HMGA1 proteins accounts for the reduced BRCA1 protein levels in sporadic breast carcinoma. Mol Cell Biol 23:2225-38, 2003.
79. Rebbeck T R, DeMichele A, Tran T V, et al. Hormone-dependent effects of FGFR2 and MAP3K1 in breast cancer susceptibility in a population-based sample of post-menopausal African-American and European-American women. Carcinogenesis 30:269-74, 2009.
80. Warmka J K, Mauro L J, Wattenberg E V. Mitogen-activated protein kinase phosphatase-3 is a tumor promoter target in initiated cells that express oncogenic Ras. J Biol Chem 279:33085-92, 2004.
81. Remmele W, Dietz M, Schmidt F, Schicketanz K H. Relation of elastosis to biochemical and immunohistochemical steroid receptor findings, Ki-67 and epidermal growth factor receptor (EGFR) immunostaining in invasive ductal breast cancer. Virchows Arch A Pathol Anat Histopathol 422:319-26, 1993.
82. Silvestrini R. Proliferation markers in breast cancer. Eur J Cancer 29A:1501-2, 1993.
83. Trihia H, Murray S, Price K, Gelber R D, Golouh R, Goldhirsch A, Coates A S, Collins J, Castiglione-Gertsch M, Gusterson B A; International Breast Cancer Study Group. Ki-67 expression in breast carcinoma: its association with grading systems, clinical parameters, and other prognostic factors—a surrogate marker? Cancer 97:1321-31, 2003.
84. de Azambuja E, Cardoso F, de Castro G Jr, Colozza M, Mano M S, Durbecq V, Sotiriou C, Larsimont D, Piccart-Gebhart M J, Paesmans M. Ki-67 as prognostic marker in early breast cancer: a meta-analysis of published studies involving 12,155 patients. Br J Cancer 96:1504-13, 2007.
85. Easton D F, Pooley K A, Dunning A M, et al. Genome-wide association study identifies novel breast cancer susceptibility loci. Nature 447:1087-93, 2007.
86. Thorpe S M, Rose C, Pedersen B V, Rasmussen B B. Estrogen and progesterone receptor profile patterns in primary breast cancer. Breast Cancer Res Treat 3:103-10, 1983.
87. McGuire W L, Horwitz K B. A role for progesterone in breast cancer. Ann N Y Acad Sci 286:90-100, 1977.
88. Shimo A, Nishidate T, Ohta T, et al. Elevated expression of protein regulator of cytokinesis 1, involved in the growth of breast cancer cells. Cancer Sci 98:174-81, 2007.
89. Yun H J, Cho Y H, Moon Y, et al. Transcriptional targeting of gene expression in breast cancer by the promoters of protein regulator of cytokinesis 1 and ribonuclease reductase Exp Mol Med 40:345-53, 2008.
90. Hadad S M, Fleming S, Thompson A M. Targeting AMPK: a new therapeutic opportunity in breast cancer. Crit Rev Oncol Hematol 67:1-7, 2008.
91. Li J, Yen C, Liaw D, Podsypanina K, et al. PTEN, a putative protein tyrosine phosphatase gene mutated in human brain, breast, and prostate cancer. Science 275:1943-7, 1997.
92. Bose S, Wang S I, Terry M B, Hibshoosh H, Parsons R. Allelic loss of chromosome 10q23 is associated with tumor progression in breast carcinomas. Oncogene 17:123-7, 1998.
93. Ghosh A K, Grigorieva I, Steele R, Hoover R G, Ray R B PTEN transcriptionally modulates c-myc gene expression in human breast carcinoma cells and is involved in cell growth regulation. Gene 235:85-91, 1999.
94. Depowski P L, Rosenthal S I, Ross J S. Loss of expression of the PTEN gene protein product is associated with poor outcome in breast cancer. Mod Pathol 14:672-6, 2001.
95. Järvinen TA, Liu E T. opoisomerase IIalpha gene (TOP2A) amplification and deletion in cancer—more common than anticipated. Cytopathology 14:309-13, 2003.
96. Hannemann J, Kristel P, van Tinteren H, et al. Molecular subtypes of breast cancer and amplification of topoisomerase II alpha: predictive role in dose intensive adjuvant chemotherapy. Br J Cancer 95:1334-41, 2006.
97. Depowski P L, Rosenthal S I, Brien T P, Stylos S, Johnson R L, Ross J S. Topoisomerase IIalpha expression in breast cancer: correlation with outcome variables. Mod Pathol 13:542-7, 2000.
98. Woolcott C G, Maskarinec G, Haiman C A, et al. The association between breast cancer susceptibility loci and mammographic density: the Multiethnic Cohort. Breast Cancer Res 11:R10, 2009.
99. Easton D F, Pooley K A, Dunning A M, et al. Genome-wide association study identifies novel breast cancer susceptibility loci. Nature 447:1087-93, 2007.
100. John A. Rice 1997 Mathematical Statistics and Data Analysis 2nd ed., Publisher: Duxbury Advanced, Belmont, Calif.
101. Smolkin M and Ghosh D. Cluster stability scores for microarray data in cancer studies. BMC Bioinformatics 4:36-42, 2003.
102. Calza S, Hall P, Auer G, et al. Intrinsic molecular signature of breast cancer in a population-based cohort of 412 patients. Breast Cancer Research 8:R34, 2006.
103. Black M M and Speer F D. Nuclear structure in cancer tissue. Sug Gynecol Surg 153:483-498, 1957.
104. Kouros-mehr H, Slorach E M, Sternlicht M D and Werb Z. Gata-3 maintains the differentiation of the luminal cell fate in the mammary gland. Cell 127-1041-1055, 2006.
105. Yuan B, Xu Y, Woo J H, et al. Increased expression of mitotic checkpoint genes in breast cancer cells with chromosomal instability. Clin Cancer Res. 12:405-410, 2006.
106. Zhai X, Gao J, Hu Z, et al. Polymorphisms in thymidylate synthase gene and susceptibility to breast cancer in a Chinese population: a case-control analysis. BMC Cancer 6:138-144, 2006.
107. Kittiniyom K, Gorse K M, Dalbegue F, et al. Allelic loss on chromosome band 18p11.3 occurs early and reveals heterogeneity in breast cancer progression. Breast Cancer Res 3:192-198, 2001.
108. Levine R M, Rubalcaba E, Lippman M E and Cowan K H. Effects of Estrogen and Tamoxifen on the Regulation of Dihydrofolate Reductase Gene Expression in a Human Breast Cancer Cell Line. Cancer Research 45:1644-1650, 1985.
109. Ohta T, Fukuda M, Arima K, et al. Breast Cancer. Analysis of Cdc2 and Cyclin D1 Expression in Breast Cancer by Immunoblotting. Breast Cancer 4:17-24, 1997.
110. Bouras T, Lisanti M P, Pestell R G. Caveolin-1 in breast cancer. Cancer Biol Ther. 3:931-41, 2004.
111. Makretsov N A, Hayes M, Carter B A, et al. Stromal CD10 expression in invasive breast carcinoma correlates with poor prognosis, estrogen receptor negativity, and high grade. Mod Pathol. 20:84-9, 2007.
112. Kao K J, Huang T Y, Chen D Y, et al. Identification of common neoplastic signature genes through study of paired hepatocellular carcinoma and adjacent non-tumorous tissue. AACR Meeting Abstracts, April 2008, 4260.
113. Phasing out anthracyclines in breast cancer: Is it time? (http://www.hemonctoday.com/article.aspx?rid=41512) HemOnco Today July, 2009.
114. Tewey K M, Chen G L, Nelson E M, and Liu L F. Intercalativeantitumor drugs interfere with the breakage reunion reaction of mammalian DNA topoisomerase II. J Biol Chem 259:9182-9187, 1984.
115. Pritchard K I, Messersmith H, Elavathil L, et al. HER-2 and topoisomerase II as predictors of response to chemotherapy. J Clin Oncol. 26:736-44, 2008.
116. Wood A J J. Intrinsic and acquired resistance to methotrexate in acute leukemia. New Eng J Med 335:1042-1048, 1996.
117. van de Vijver M J, He Y D, van 't Veer L J, et al. A Gene-Expression Signature as a Predictor of Survival in Breast Cancer. New Engl J Med, 347:1999-2009, 2002.
118. Miller L D, Smeds J, George J, et al. An expression signature for p53 status in human breast cancer predicts mutation status, transcriptional effects, and patient survival. Proc Natl Acad Sci, USA, 102:13550-13555, 2005.
119. Haibe-Kains B, Desmedt C, Piette F, et al. Comparison of prognostic gene expression signatures for breast cancer. BMC Genomics 9:394-402, 2008.
120. Desmedt C, Piette F, Loi S. et al. Strong time dependence of the 76-gene prognostic signature for node-negative breast cancer patients in the TRANSBIG multicenter independent validation series. Clin Cancer Res. 3207-3214, 2007.
121. Rakha E A, Reis-Filho J S, and Ellis I O. Basal-like breast cancer: a critical review. J Clin Oncol 26:2568-2581, 2008.
122. Carey L A, Dees E C, Sawyer L, et al. The Triple Negative Paradox: Primary Tumor Chemosensitivity of Breast Cancer Subtypes. Clin Cancer Res 13:2329-2334, 2007.
123. Diallo-Danebrock R, Ting E, Gluz O, et al. Protein expression profiling in high-risk breast cancer patients treated with high-dose or conventional dosedense chemotherapy. Clin Cancer Res 13:488-497, 2007.
124. Aigner K, Dampier B, Descovich L, et al. The transcription factor ZEB1 (δEF1) promotes tumour cell dedifferentiation by repressing master regulators of epithelial polarity. Oncogene 26:6979-6988, 2007.
125. Dandachi N, Hauser-Kronberger C, More E, et. al. Co-expression of tenascin-C and vimentin in human breast cancer cells indicates phenotypic transdifferentiation during tumour progression: correlation with histopathological parameters, hormone receptors, and oncoproteins. J Pathol 193:181-189, 2001.
126. Foekens J A, Romain S, Look M P, et al. Thymidine kinase and thymidylate synthase in advanced breast cancer: response to tamoxifen and chemotherapy. Cancer Res 61:1421-1425, 2001.
127. Bertino J R and Banerjee D. Is the measurement to determine suitability for treatment with 5-fluoropyridines ready for prime time? Clin Cancer Res 9:1235-1239, 2003.
128. Finak G, Bertos N, pepin F, et al. Stromal gene expression predicts clinical outcome in breast cancer. Nature Med. 14:518-527, 2008.
129. Bautch V. Endothelial cells form a phalanx to block tumor metastasis. Cell 136:810-812, 2009.
130. Mazzone M, Dettori D, de Oliveira R L, et al. Heterozygous deficiency of PHD2 restores tumor oxygenation and inhibits metastasis via endothelial normalization. Cell 136:839-851, 2009.

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2,1-3, 1-4,1-5, 2-3,2-4, 2-5,3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs or accession numbers, including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

While this invention has been particularly shown and described with references to example embodiments thereof, it will be understood by those skilled in the art that various changes in form and details can be made therein without departing from the scope of the invention encompassed by the appended claims.

1. A method of treating a breast cancer in a subject, comprising:
a) determining the molecular subtype of the breast cancer in the subject, wherein the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer; and
b) administering to the subject a therapy that is effective for treating the molecular subtype of the breast cancer determined in step a).

2. The method of claim 1, wherein the molecular subtype of the breast cancer is molecular subtype I and a therapy that includes an adjuvant chemotherapy is administered to the subject.

3. The method of claim 2, wherein the adjuvant chemotherapy comprises administering methotrexate, wherein before determining the molecular subtype of the breast cancer in the subject, the subject was a candidate for receiving an adjuvant chemotherapy comprising anthracycline and after determining the molecular subtype of the breast cancer in the subject, anthracycline is not administered to the subject.

4. (canceled)

5. The method of claim 1, wherein the molecular subtype of the breast cancer is molecular subtype II and a therapy that includes at least one member selected from the group consisting of administration of a HER2/EGFR signaling pathway antagonist, a high intensity chemotherapy and a dose-dense chemotherapy is administered to the subject.

6. The method of claim 5, wherein the therapy comprises administering a HER2/EGFR signaling pathway antagonist.

7. (canceled)

8. The method of claim 1, wherein the breast cancer is a molecular subtype I or a molecular subtype II, and wherein the method further comprises determining an immune response score, wherein adjuvant chemotherapy is administered to a subject with a low immune response score.

9. The method of claim 8, wherein the breast cancer is a molecular subtype I and the therapy comprises adjuvant chemotherapy comprising anthracycline.

10. The method of claim 1, wherein the molecular subtype of the breast cancer is selected from the group consisting of molecular subtype III and molecular subtype VI and a therapy that includes at least one anti-estrogen therapy is administered to the subject.

11. The method of claim 1, wherein the molecular subtype of the breast cancer is molecular subtype IV and a therapy that includes an adjuvant chemotherapy comprising at least one anthracycline is administered to the subject.

12. (canceled)

13. The method of claim 11, wherein before determining the molecular subtype of the breast cancer in the subject the subject is a candidate for adjuvant chemotherapy comprising administering methotrexate and after determining the molecular subtype of the breast cancer in the subject, anthracycline is administered to the subject.

14. The method of claim 11, wherein before determining the molecular subtype of the breast cancer in the subject the subject is a candidate for adjuvant chemotherapy comprising administering a HER2/EGFR signaling pathway antagonist and after determining the molecular subtype of the breast cancer in the subject, a HER2/EGFR signaling pathway antagonist is not administered to the subject.

15. (canceled)

16. (canceled)

17. The method of claim 1, wherein the molecular subtype of the breast cancer is molecular subtype V and a therapy that includes anti-estrogen therapy is administered to the subject.

18. (canceled)

19. The method of claim 17, wherein before determining the molecular subtype of the breast cancer in the subject the subject is a candidate for adjuvant chemotherapy and after determining the molecular subtype of the breast cancer in the subject, the subject is not administered adjuvant chemotherapy.

20. (canceled)

21. (canceled)

22. The method of claim 1, wherein before determining the molecular subtype of the breast cancer in the subject, the subject is a candidate for adjuvant chemotherapy.

23. (canceled)

24. The method of claim 22, wherein an adjuvant chemotherapy is not administered to the subject.

25. A method of identifying a subject with a breast cancer as a candidate for a therapy having efficacy for treating a breast cancer molecular subtype, comprising:
a) determining the molecular subtype of the breast cancer in the subject, wherein the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer; and
b) identifying the subject as a candidate for a therapy that is effective for treating the molecular subtype determined in step a).

26.-30. (canceled)

31. A method of selecting a therapy for a breast cancer in a subject, comprising:
a) determining the molecular subtype of the breast cancer in the subject, wherein the molecular subtype is selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer; and
b) selecting a therapy that is effective for treating the molecular subtype determined in step a).

32.-36. (canceled)

37. A method of classifying a breast cancer, comprising:
a. comparing the gene expression profile of the breast cancer to one or more reference gene expression profiles for a breast cancer molecular subtype selected from the group consisting of a molecular subtype I breast cancer, a molecular subtype II breast cancer, a molecular subtype III breast cancer, a molecular subtype IV breast cancer, a molecular subtype V breast cancer and a molecular subtype VI breast cancer; and
b. classifying the breast cancer as a molecular subtype I breast cancer, a molecular subtype II breast cancer, a molecular subtype III breast cancer, a molecular subtype IV breast cancer, a molecular subtype V breast cancer or a molecular subtype VI breast cancer.

38. The method of claim 37, wherein the gene expression profile is generated from the expression level of at least about 30% of the genes in Table I.

39.-47. (canceled)

48. A method of prognosing a subject suspected of having breast cancer for one or more clinical indicators, comprising the steps of the method of classifying a breast cancer of claim 37, wherein the prognosis is based on the classification step (b) and wherein the one or more clinical indicators are selected from the group consisting of metastasis risk, T stage, TNM stage, metastasis-free survival, and overall survival.

49. The method of claim 48, further comprising determining the immune response score of the subject, wherein a low immune response score indicates reduced metastasis-free survival.
